SEQUENCE LISTING (12 sequences)

SEQ ID NO: 1 - protein, 610 aa, Saccharomyces castellii
MNREKSADLSKTSDAPYNELDAKNLRNFYKVQNACAQLRESIKVIYENGLSSDQLNTMAKHGNDLEKSIANSPAMSVASCLNQVRPTLDIKNIFDHYKFENNSSPVDPYVHYPVCSDQNLENLAFIHRSLPNMNVKLTELQKTVMSNERLEFLGDSWLGALVAYIIYKKYPYANEGALSKMKEAIVNNNNLEKICEKLGFKERLKENIPRSSMKIKDRLTKNYADCVEAYIGALVIDRFSTEFNDVALWLEELSEEHFIELGPMMVKEPLNKNAKGELGAFLQFNNIGAKISYKRLNDKSPFKVEVRLGNNLLGIGDGSNVREAEQRAAMEALAQPKLIQKYSLHDIELERGMIEEADNVPQLLKSAEIPNQTPQSHHSITEEIDEEEGLTFPHSNPFLKGTPPAVLPEIVTNQIPQASADHQEQNVPDTDKIVNDVMERMSKILSVMVSEAVSNALGRPAPKNAPIPIPVTTPVSPLATSPVSVSVPAPAPAVAVTPSVVNNPPQPKMVTQPVSTNLNKTPEVALLNSVYDKEASGRLYALLGKYKLYPEYNTEQLGMTDFYTVCFIKGVGVEIGKGHGRSKKISQHKSAEDALNGKALKEYLRHCNNL

SEQ ID NO: 2 - protein, 558 aa, Kluyveromyces polysporus
MSDQVYVPPTSAPMSNELKVREFYRLHNACVKLKESIKLIYENPLVTDQNVLNLGTAENTIDYTILNTPTLNVAKTLLGNRYSLDLIDLFQSHDFKDSNTDVDMFIKYPVVYDENLENLAFMHKSFPNMNAHLNDAQKTQLSNERLEFLGDSWLGALVSYIVYTRFPSANEGMLSQMKESIVNNNNLFDWSTKLNFTKRLQGNIATPTRVVKDKMSKRYADCVEAYIGALVIDRFGTEFLDIKEWLEELSEKKLAKSSQMVIKEPLNKNAKNELAELLQINKLGHKLHYRKLTEMPPFRVEVKIGDILLDEAEGNSIREAEHRAAMKVLENDELLRKYSVYDLEDGNSKKYSIEETDLKHDIESSGISNENFLQNEGNGTENIESKFKQLNISNDSNKDDLVNNITNSVFSKLQDVVSKIVIDAISESIPNNPHKIEQSRNGKVGTQTNTSATTSDVLSVQTSIPKSESTSADKAPITCDPNAKQALYTLLSRSQVFPDYEMVQHSMDNFTANCIIKGSGIFLASGRGRSKKIAQQNAASAALRSPILKSILEEQNNF

SEQ ID NO: 3 - protein, 611 aa, Candida albicans
MSSFSQKNLAAQNIAETANKGFMSYINNWTSTSQQEDNKVISSNLDDPLSVSDIDSDTSMSSNNSSKNHHSSKRSSPAPVPSTVTKKTKITDKYLKELSAYHEPVFDKPPPTTIGILDMNQLEHSTKTMQKCVSSILEKAPNCNQLQYLIESDEIDNGIRLELQDNPLIDTASKLKSLHEIGKLPILDQIINDEVKISDDDLKKLKKIPAKSNSVKVNSPRDVVPEIASTGVHHDMPPLPPINDPKLFERVFIHKSTVNNKQYLEANHLLHSHNERLEFYGDSILNNLATLIIFKEFPDSTEGDLSKIRSKLVSNKTLIKIAFQYGFDKKLRSRISDDVLKTGDKKIFADVFEAYIGALAMERGFDLEDVKEWLAKVYAPLIHELKIEYIEEPIDREAKSKLYGLIGQDDLHPVYNTLQMGDGIHNKFIVQCTINGEELGRGTANNLKEAGLRAAMAGLNNNEVLKKYFVARQQIERPIKLQRLERENAKKREVAERLEQERNNLPSTPSSPIRTSMFPISVNENEPLDIDAKNKLYGILGRKIGDKPAYLTSQINGNLFKVVLCIRGITICETTDPSKKKAMARAAQTLLENESAMREICKDFRTKDSPE

SEQ ID NO: 4 - protein, 1299 aa, Saccharomyces castellii
MSSNSEENSQVPPLDATAAATKPKKAKKPKVKKPKDSAEASSSPAAEGTAEAKPKKAKKSKTKKSKESAEVSPAPADETTSAGVDAKPKKAKKSKVKKPKDSTESSPAPSNEPPAAEVAAEDAKSKKVKKPKAKKPKESTESSPAPGQEAAATEGATEDKPKKVKKSKAKKAKESVESSPAATESVSEKTAKKSKKPKAKKSTSPETTEEITEESTESKEKKTKTKKPKEKKSSPSTATSTAASKPVTSIAGVTIPGKTFDLTDVPPSKPAPKMVPEAYKLQTRVDYGTKGTKVDVLTNHILLSVGDDVPQDERASQLDPWWKSAFVYTYNITFAVPQSNSPRKGPAPALSKPKKYELVESLFHEDETLFKYKDRISFNGEDTLYSHVPLEEFTLFDGCWDVSNKQKKRRQEVVGLNSRAKEINDLAAQVTLKFADKVPLGDIYKATTSKDPEEQENKMANADKVALLSLMGVKFLNTKEQIFQLNGNKFFIFNEHAIATPFQIGGFLMHGFTVSLRYAYGSVLLNTVNVCLPFVKWTKYLPGDAKFKENEKTQYSLLDWIIECMHQASAQRGQKLRGPPSAKDINFFIDKNRDIKDLLKGLKCYRPYINYSVNPDGTPKPPKKMQAKGIVGFVRETPDSMKFRTLPSNMEKNGVPKPGEKEIMVTTTAYFAKKYDIKLKYPDVKMVSLGGSNVVPAECLTIVPGQKLKGLVYDEKAVIDFTALRPSEKFRAITNLALPAIKRALSTEEENAKAPHDSGYTFMKVPSRVIDAPVVQFKNTTVTYVDKPFGTKNGKNNHEETKGNWNLKDHKFITVPKEPMHLRAIFINDSDKSPPVSVMDELKASLSKFAEDVADVGVNFDVSMAPILINNFNAPIKKVTGGFGGRGGRGGRGGRGGRGGRGGRGGFGGGRGETTYELTPGEEKLRHLLANVPEKTYVLFVLGRGDDSAIYNRLKYLADLTYGVINNCVIWNKFRKCSTQYNVNVVMKMNLKLEGANHSLCAEDINLLKDEKSGLPFMILGADVTHYPEKDQNSISALVGSFDDKFAQFPGSYMLQSGPGEEIIAGIGNMVLQRLKLYQKHNNGKLPPKILFYRDGVSESQFSQIVQIEVKGLKQALKKFGSELNKGVNYNPSVTTICVVKRNQIRFMPLEQNAINEKGEVAAVQSFENVMPGTVVDRGITSSAHFDFFLQSQQPLKGTGVPCHYWCIYDENQFNSDYLQQVTHALCYLFGRSSTSIKVASPVYYADLLCERGAAFFKANFELAQYEFSKERKNRDDVIPTGKLLQPVHKNVTDIMYYI

SEQ ID NO: 5 - protein, 1251 aa, Kluyveromyces polysporus
MATLKPDTQIIAGNAAETEKPIVKKSSSSKPDAGDGEVESKPKKDKKKSKKENSDGKDETSTAKKTKKSKNTKKSKDKTESSSTEKLDETSTSESPSEESSEKPKKSKKSKKKSSEGNENVEANSENVKEKPEKKKKSKKSSKESTPETESENVETKSEKKKKSKKPKKSSKESTPEPNSSTSTEQSSEAKSQSFGFKYSDKVFDLTEKTVDQPKEDTHAIYKVENRHDYGTKGTKVDILTNHILLAVGNDVPTEKIDKELVPKLDGWWKTAFIFTYHIDFKPQQKGPPRRGKPVPPQELSKPKKYELIEALLDEDEILYKYRDRIAFNGEDTIYSHVPLEEFTLFDGCWEVSNKQKKKVVPGMGAPSNKASLQKKIDPELEEMVSQITLKFSGKVGLKDIYNDTTTQDTEVQESRMSAIDKTCLLSLLGAKFMSTDDLIFQVQGNKFFIFNNFAKAIPFQIGGYLLQGFTVSLTHVYGGVALNTVSVPAPFIKHTKYLPGDPRFKNNEKEQFTLMDWIIECYHQSKAIRDIRYNPKTAPPPSVKDLNYFVEKNTDISALLKGLKVYRPYINYSINKDGTPKPPRKRSSKGIVGFTRESAVSMRFNVLESSLKKNSAPKPNEKPININTIDYFKRKYDITLKYPDMKLVNLGGKNDVVPPECLTIVPGQKLKGQIFDTKTYIDFSAIRPTEKFDLISRLSMPAIKRGLTDSEKEESSAPHNSAYQFMRVPSRILDAPVVQFKESTFEYKDKSYGTKHEESKGNWNMKGHQFISTPAKQVNLRAIFINNANTAPPASMESELDISMDKFASDVKQLGVDFNVSGKPILINQFGPPIKKFQGGGRGGRGGRGSRGGRGGRGAPSGPPTFETSPGEISLLNLLENIPSNTYILYVLRRGNDSAVYDRLKYITDLKFGALNSCVVWDNFKKNSIQYNSNVVMKMNLKLLGSNHSLSIENNKLLIDKESNLPILVLGSDVTHYPEKDQNSIASLVGSYDDKFTQFPGDYMLQDGPGEEIITNVGSLMLNRLKIYQKHNNGKLPTKIMYFRDGVSVDQFSQVVKIEVKSIKESVRKFGPQLNGGNKYDPPVTCIATVKRNQVRFIPIQENAKNEKGEEVAVQSMGNVMPGTVVDRGITSVAHFDFFIQSHQALKGTGVPCHYWCLYDENQSTSDYLQEICNNLCYIFGRSTTSVKVPAPVYYADLLCTRATCFFKAGFELNMAQAPKEKGSKDQPTVSKNVLLPQVNDNIKSVMYYI

SEQ ID NO: 6 - protein, 917 aa, Candida albicans
MSDLVKFSTPTKINDKALVAIDTYKKTFQKQIKNEQNGILLRPSFGTEGVPTIVGVNYFKYNVNGLKLYSYRVDLLEDPNVKTRLSIKTAVEKYLMELEPFKSKKAIIYYRDHNHLYSRMPLPIEDVVIYPLELGGDPKREKKIVELKIQFIKQLNFSDLLNYTQLKNYTPDFLETVEYTNALVAVMGSQVMKNTNVVGLGPNKFFLMDGATKTTDLDKGLYVAMGTFASVRCSFDDVRINLNPTPAIFYKSSKPNGQPMNVLDLIQEFLKIRNVPTERDIRRAQSFIKGCKIYRTYLNRKTTKPILGFDYSNNSETLKFKDANDKLVNVKQYFQERWNIRLKHPTLPLIKIGPEAFLPMKLGIIAPHQQYKGDFADPAKIIKITATRPNQKAELISKTNRNLFQKQVDFGTIESQFTVVPARVLNAPTIEYANNQMVTYRPAPFNGRTEKQKGNWNLERYQFVDGAKLTKPMGKPFTFGVVILKDEFVAKRIGDLQRAVPTFLTELGRLGIKIGKNFKKYSIDLNHASVQSQTGLESNIISIAKKAKTEDQCNFLLFILPRQDTPLYSAIKRACDLKVGILNSCSILNTFTKKRRGTENFDAMTYAQMAMKINIKLGGSNHKLSKKDSQGLFDKNNVPIFILGADVTHPTGEINSESVSIASIVGSEDGIFNKFPGSVRIQTGGQEVIADVKSMVLERLENFHKKIGKLPSKVLFYRDGVSEGQYTTILKEELTKIKAAFNEYGKLKNIPKYSPTITFMIVVKRHHTRFIPIHDNADDPKTKKQIAVTSNENVIAGTTVDREITSPAYFDFYVQSQQSLQGTGIPAHYYVLHDENNYTSDTIQKITYDLCHTFSRATKSVKVVPAAYYADLLCTRGRDYIYGFAKDPNLKGSPIERARTKFGENVNPSIKNTMFYI

SEQ ID NO: 7 - DNA, 1833 bp, Saccharomyces castellii
atgaatagagaaaaaagcgccgatctaagtaaaacatcagatgctccatacaatgaactcgatgccaaaaatttaagaaatttctataaggttcaaaatgcatgtgcgcaattgagggaatccataaaagtaatttacgaaaacgggttatcatccgatcaattgaatactatggcgaaacatgggaacgacctagaaaaatctattgcaaacagtcctgctatgtccgtagcaagttgcttgaaccaagttagacccaccctagacatcaagaatatctttgatcattataaatttgaaaataattcatcaccagttgatccttatgtccattatcccgtatgttctgatcaaaatttggaaaatttggctttcattcatagatctttgccgaatatgaatgtcaaattgactgaattgcaaaagaccgtaatgagtaatgaacgtttggaatttttaggtgatagttggcttggcgccttagtggcgtatattatttataagaaatatccttatgctaatgaaggtgccttatcaaagatgaaggaagctatcgttaacaacaacaatttggaaaagatctgtgaaaaacttggctttaaagaaagattaaaggaaaatattccacgttcttcaatgaaaatcaaagatagattaaccaagaattacgcagactgtgtggaagcctacattggcgcattggttattgatagattttcaacagaatttaatgatgttgctctttggcttgaagaactatctgaggaacatttcattgaactaggacctatgatggtcaaagagcctttaaacaaaaatgcaaagggtgaactaggtgcgttcttacaatttaataatattggagccaaaatatcatataaaagattgaatgataaatctccgtttaaggtagaagtaagattggggaataatttgttaggtattggagatggttcaaatgtgagagaagctgaacagagagccgctatggaagcacttgctcaaccgaaattgattcaaaaatattctctacatgatattgaactagaaagaggcatgattgaagaagcagataatgttccccaattactcaaaagtgcggaaattcccaatcaaacaccacaaagtcatcattctatcacagaagaaattgatgaagaagaaggtcttacattcccccattcgaatccatttttgaaaggtacaccaccagcagttttacctgaaattgtaactaatcaaattccacaggcttctgcggatcatcaagaacaaaatgtgcctgacactgataaaattgtgaatgatgttatggaacgtatgagtaagatactgtcggtgatggtttctgaggctgtttcaaatgcacttggtagaccagctccaaaaaatgcacctatccctatcccagtaacaaccccagtctcaccgctagcaacatccccagtctcagtctcagtcccagcgccagctccagccgtagctgtcaccccatcagttgtgaataatccaccacaacccaaaatggtcacacaaccggtaagcacaaacctaaataaaacgcctgaagttgcacttttaaattctgtgtatgataaagaagcctcagggagactttatgcattgttaggtaaatataaattgtatccagaatataataccgagcaactaggaatgacagatttttatacggtatgctttataaagggtgttggtgtagaaattggtaaaggccatggtagaagtaaaaagatatcccaacataaatctgcagaagatgctttgaatggaaaagcactgaaggaatacttgaggcattgcaacaatctgtga

SEQ ID NO: 8 - DNA, 1677 bp, Kluyveromyces polysporus
atgtctgatcaagtttatgttccgccaacatcagcaccaatgagcaacgagttaaaagttagagaattttatagactccacaatgcttgtgtgaaattgaaagagtctattaaattgatttatgaaaacccattggtaactgaccaaaatgttttgaatttgggaactgcagaaaatactattgattatacaattttaaacacacctacactaaatgttgctaaaactttactgggcaatagatattcattagatctaatagatctttttcaatctcatgatttcaaagattcaaacactgatgtggatatgtttattaaatatcctgtggtttatgatgaaaatttggaaaatttagcatttatgcataaaagttttccaaatatgaatgcacatctaaatgatgctcaaaagactcaattatctaatgaaaggttagaatttttgggtgatagttggctaggtgctttagtttcatatattgtatacaccagatttccttctgcaaatgaaggtatgctttctcaaatgaaagaatcaatagtaaataataataatctatttgattggagtacaaaattaaacttcaccaagagattacaaggtaatatcgctacacctactagagtggtcaaagataaaatgtctaaacgttatgctgattgtgttgaagcctatattggtgcattggttattgatagatttggcactgagtttttggatatcaaggaatggttagaagaactatcagaaaaaaaattggccaaatcgagccaaatggtgataaaggaaccattaaataaaaatgctaaaaatgaattagccgaattactgcaaattaacaaattaggacataaacttcattacagaaagcttacagaaatgcctccattcagagttgaagttaaaataggtgacattctgttagacgaagctgaaggtaacagtataagggaagcagaacacagagctgcgatgaaggtattggaaaatgatgaattattgaggaaatattcggtttatgatttagaggatggaaactcgaagaaatattctattgaagagacggatcttaaacatgatattgaatcctctggaatatctaatgaaaattttttgcaaaatgagggaaatggaactgaaaatatagaatcaaaatttaaacaattgaatatatcaaatgatagcaacaaagatgatttagttaataatattacaaattctgtttttagtaaattgcaggatgtcgtaagtaaaattgtcattgacgctatttccgaatcaatcccaaataatccccacaaaatagaacaatctagaaatggtaaagtaggaacacaaacaaatactagtgctacaacttcagatgtcctttcagttcaaacgtctattcctaaaagcgagtcaacttcggctgataaagctcctatcacatgtgatcccaatgccaaacaggcattgtataccctgttatctagatcacaagtgttcccagactatgaaatggttcaacattccatggataatttcaccgctaactgtatcattaaaggcagcggtatatttttagcgtccggaagaggtcgcagtaagaagattgctcagcaaaatgcagcatcagcggcacttcggagcccaatcttaaaatctattttggaagaacaaaacaatttttaa

SEQ ID NO: 9 - DNA, 1836 bp, Candida albicans
atgtcatcgttttctcaaaaaaatctagcagcacaaaatatagcagaaactgccaacaaagggtttatgagttacatcaacaactggacatctaccagtcaacaagaagataataaagtaatttcatcaaatcttgacgatcctttaagtgtatcagatatagattccgacaccagtatgctgagtaataacagttcgaaaaaccatcactcatcgaaaagatcgagtcccgctccagtaccttctactgttacaaagaagacaaagatcaccgataaatatttgaaagaacttctggcatatcatgaaccggtgtttgataagccgcctccaacaacaataggaatcttagatatgaatcagttagagcattctactaaaaccatgcaaaaatgcgtgtccagtatacttgaaaaggcacctaattgcaaccaattacaatatctcattgaatcagacgagatagataatggtattagacttgaattacaggacaatccattgatagacacggcatcaaaattgaaatcattacatgaaattgggaagttaccaattttggaccaaatcatcaatgatgaagtcaaaataagtgacgatgatttgaaaaaattgaaaaaaatacctgctaaatctaattcagtcaaagtaaattcacctcgtgatgttgtaccagaaattgcatctacaggggttcaccatgatatgccaccgttaccaccaataaatgaccctaaattgtttgaacgggtctttattcataaatccacagtcaacaacaagcaatatcttgaagctaaccatttattacatagtcataatgaaagattggaattctacggtgactctattttaaataatttagcgactcttataattttcaaagagtttcctgattctactgaaggtgacttatcgaaaattagatcaaaattagttagcaacaaaacattaattaaaattgcctttcaatatggatttgacaaaaagttacgttccagaataagtgatgatgtattgaaaactggtgataaaaaaatatttgctgatgtatttgaagcatatatcggagcattagcaatggagaggggtttcgatttggaagatgtcaaagaatggttagcaaaagtgtatgcaccattgattcatgagttgaaaattgaatacattgaggagccaattgatagagaagccaaatccaaattatatggacttattggccaagatgatttacatccagtatataatactcttcagatgggtgatggtattcataataaatttatagttcaatgcaccatcaatggagaagagttgggtcgtggtacagcaaacaatttgaaagaggcagggttaagagctgccatggcaggattaaataataatgaagttttgaaaaaatactttgtggcacgccaacaaattgaaagaccaattaaattacaacgactcgaaagagaaaatgccaaaaagagggaggtcgcagaaagattagaacaagaaaggaataaccttccttctacaccgctgtcgccaattagaacctcaatgtttcctatttcagttaatgaaaatgaaccattggacattgatgcgaaaaacaaattatatggtatccttggtagaaaaattggtgacaaaccagcttaccttacatctcagataaatggtaatttgttcaaagttgtgttatgtattagaggcattacgatctgtgaaacaactgacccatcgaaaaagaaagccatggccagagcagctcaaacattgttagaaaatgaaagtgcaatgagagaaatctgtaaagatttccgaactaaggactcaccagaataa

SEQ ID NO: 10 - DNA, 3900 bp, Saccharomyces castellii
atgtcatccaattcggaggagaacagtcaagttcctccccttgatgccaccgctgctgcaactaaaccaaaaaaggccaagaaaccaaaggttaagaagccaaaggattccgctgaggcatcttcatcaccagctgctgagggcactgctgaagccaagccaaaaaaggctaagaaatcaaagactaagaagtcaaaggagtctgccgaggtatcgccagctccagctgatgaaactacttctgctggtgtagatgctaaacctaagaaggctaaaaagtcaaaggtcaagaagccaaaggattctactgaatcatctcctgctccaagtaatgaacctccagccgccgaagttgccgccgaagatgcaaagtctaaaaaggttaagaaaccaaaggccaagaaaccaaaggaatctactgaatcatctcctgctccaggtcaggaagccgctgctactgaaggtgccacagaagataaacctaagaaggttaagaaatcaaaggctaagaaggcaaaggaatctgttgaatcatctcctgccgccactgaatctgtctctgagaagactgcaaagaaatctaagaagccaaaggctaagaaatctacttctcctgaaacaactgaagaaataactgaagaatctaccgaatcaaaggaaaagaagacaaagacaaagaaaccaaaggaaaagaagtcatctccatctactgccacttcgactgctgcttcaaaaccagttacttccattgcgggagtcaccattccaggcaagacttttgatttgacagatgttcctccatctaaacctgctccaaagatggtcccggaagcctataaattacaaactagagtggattacggtactaagggtaccaaagtggacgtcttgactaatcatatactactatctgttggtgatgatgtccctcaagatgaaagagcctctcaattggatccatggtggaaatctgcatttgtatatacctataacattacttttgccgttccacagagtaattcaccacgtaaaggtccagcgcctgctttatccaaaccaaagaaatacgaattggtagaatctttgtttcacgaggatgaaaccttgttcaaatataaagatcgtatctccttcaatggtgaagatactctatactctcatgtcccattggaggaattcactttatttgatggttgttgggatgtcagtaacaagcaaaagaaaagaagacaagaagttgttggtctcaacagtagagccaaggaaattaatgacttggctgctcaagttactttaaaatttgctgataaagtcccattgggtgatatttataaggctaccacttcaaaggatccagaagaacaagaaaataagatggctaacgctgataaggttgctttattgtctttgatgggtgtcaaattcttaaacacaaaggaacaaatcttccaacttaacggtaataaattctttatttttaatgaacacgcaattgctactccattccaaattggtgggttcttaatgcatgggttcacagtttccctacgatatgcttacgggtccgttttgttaaatactgttaacgtctgcctcccatttgttaagtggactaaatatttaccaggtgatgctaaattcaaggaaaatgaaaagactcaatacagtttgttagattggattattgaatgtatgcaccaggccagtgctcaaagaggtcaaaagctaagaggccctccatctgccaaggatatcaattttttcattgacaagaatagagacattaaagatctattgaagggtttgaaatgttacagaccatacattaattattctgttaacccagatggaactccaaaaccaccaaaaaagatgcaagctaagggtatcgttggtttcgtaagagagactccagattctatgaaattcagaactttaccaagtaacatggaaaagaatggtgtaccaaaaccaggtgaaaaggaaatcatggtcacaacaaccgcctactttgccaagaagtatgacatcaaattgaaatacccagatgttaaaatggtgagtctaggtggttcgaatgttgtcccagctgaatgtttaaccattgttccaggtcagaaattaaagggtttggtttatgacgaaaaagccgttattgatttcactgctttaagaccaagtgaaaaattcagagccatcaccaacttggcattacctgccattaagagagctttatcaactgaagaagaaaatgctaaggctccacatgattctggttacactttcatgaaggtcccatctcgtgtcattgacgctcctgtggttcaattcaagaacactacagtcacatatgtggataaaccatttggtactaagaatggtaagaataatcatgaagaaactaagggtaattggaatttaaaggatcataaattcattactgttccaaaggaaccaatgcacttgagagctatctttattaatgattctgataagtctccaccagtttctgtcatggatgaattgaaggcctctttgagcaaattcgctgaagatgtcgctgatgtcggtgttaactttgacgtatccatggccccaattttaattaacaattttaatgccccaataaagaaggttactggtggctttggtggtagaggtggacgtggtggtagaggcggccgtggtggccgtggtggtagaggaggtcgtggtggtttcggcggtggccgtggtgaaactacttatgaattaactccaggtgaagaaaaactacgtcacttgttagccaacgtcccagaaaagacttatgtcttattcgtcttgggtcgtggggatgactctgctatttacaacagattgaaatatttagctgatttgacgtacggtgtcattaacaattgtgttatttggaacaagttcagaaagtgttccactcaatacaatgttaatgtggtaatgaagatgaacttgaagttagaaggtgctaaccactccttatgtgcagaagatataaacttactgaaggatgaaaaatcaggattaccattcatgattttaggtgctgatgttacacattatccagaaaaggatcagaattctatctctgcattggtgggttcttttgatgacaaatttgctcaattcccaggttcatatatgttacaaagtggtccaggtgaagaaataattgctggtatcggtaacatggttctacagagattgaaactgtatcaaaagcacaacaatggcaaactacctccaaagattctattctacagagatggtgtttctgaatcccaattctcacaaattgttcaaattgaagttaagggtttgaaacaagcattgaagaaattcggtagtgaattaaacaagggtgtcaactataacccatctgtcaccacaatttgtgttgtcaagagaaatcaaattagatttatgccacttgagcaaaatgctataaatgaaaagggtgaagttgctgccgttcaatcattcgagaacgttatgccaggtactgttgttgatcgtggtatcacctctagtgctcacttcgatttcttcttgcaatctcaacaaccattgaagggtactggtgttccatgtcattactggtgtatctacgacgaaaatcaattcaattctgattacttgcaacaagttactcacgctttgtgttacttattcggtagatcgagtacaagtattaaggtcgcatctcctgtttattacgctgatttattgtgtgaacgtggtgctgcattcttcaaggctaactttgagcttgctcaatacgaattttctaaggagaggaagaacagagatgacgttataccgaccggtaaattactacaacctgttcataagaatgtcactgacatcatgtactacatatga

SEQ ID NO: 11 - DNA, 3756 bp, Kluyveromyces polysporus
atggctactttaaagcctgatacccaaataattgctggaaatgcagctgagaccgaaaaacctattgtgaagaaatcaagttcttcaaaacctgatgctggagatggtgaagttgaaagtaaaccaaagaaggacaagaagaaatccaaaaaggaaaattccgatggaaaggatgaaaccagtactgctaaaaagacaaaaaaatctaaaaacacaaaaaaatccaaggataaaacagaatcttcttcaactgagaaattagatgagacatccacaagtgaaagtccatctgaagaaagttcagaaaaacctaaaaaatccaaaaaatccaaaaagaaatcatctgaaggtaatgaaaatgtagaagcaaattctgaaaatgttaaagaaaaacctgaaaagaagaagaaatcaaagaaatcttcaaaagaatctactcctgaaacagagtctgaaaatgtagaaacaaaatctgaaaagaagaagaaatcaaagaaaccaaagaaatcttcaaaagaatctacccctgaaccaaattcttcaacatctacagaacaatcttctgaagctaaatctcaatctttcggtttcaaatattctgataaagtgtttgacttaaccgagaaaactgtagatcaacctaaggaagatactcatgcaatctataaagttgaaaatagacatgattatggtactaaaggtactaaagttgacattttgacaaaccatattttgcttgcggtaggtaacgatgttcctactgaaaaaatcgacaaagaattggttcctaaattagatggttggtggaaaactgccttcatctttacttatcatatcgatttcaaaccccaacaaaaaggccctccacgtagaggcaaaccagttccaccacaggaattatcaaagccaaagaaatatgaattaattgaagctttactggatgaagatgaaatcttgtacaagtatagggatcgtatcgctttcaatggtgaagatactatttattctcatgtgccactagaagaattcactttgtttgatggttgttgggaagtcagtaacaagcaaaagaagaaggttgttccaggtatgggtgctccatccaacaaagcttctctacaaaaaaaaatagatccagaattagaagaaatggtttctcaaatcactttgaaattcagtggtaaagtaggcttgaaagatatttataacgacaccactactcaagacaccgaagtacaggaaagtagaatgtctgcaatcgataagacatgtttgctatctttattaggtgctaagttcatgagtacagatgatttgattttccaagttcaaggtaacaagttctttattttcaataattttgctaaagctatcccattccaaatcggtggttatttgttgcagggttttactgtttcattaactcatgtttatggtggtgtcgctttaaacactgtcagtgttcctgctccatttattaaacataccaagtacttgccaggtgatccaagatttaaaaacaatgaaaaggaacaatttacattaatggactggatcattgaatgttaccaccaatctaaggctataagagatatcagatataatccaaaaacagctcctccaccatcagttaaagatttgaactattttgtcgaaaagaacactgatatttcagctttgttaaagggtttaaaggtttacagaccgtacatcaattatagtatcaataaagatggtaccccaaaaccaccaagaaagagatcttctaagggtattgttggatttacccgtgaatctgcggtatccatgaggtttaatgttcttgaaagtagtttgaagaagaacagtgctccaaaacctaatgaaaaacctatcaatatcaatactattgattatttcaagaggaaatatgacattactttgaaatatcctgatatgaagttagtaaacttgggcggtaaaaatgatgtcgttcctcctgaatgtttgactattgtgccgggtcaaaaattgaagggtcaaatttttgatacaaaaacttatatcgacttcagtgcaattagaccaactgaaaagtttgatttaatctccaggttatctatgccagctataaaaagagggttaactgattctgaaaaggaagaatcatcagctcctcacaatagtgcttatcaatttatgagagtaccatctcgtattctagatgccccagttgttcagttcaaagaatctacctttgaatataaggacaagagttatggaactaagcatgaagaatctaaaggtaactggaacatgaaaggtcaccaatttatttccactccagccaaacaggtcaacttaagagcaatatttattaataatgctaacacagctccaccagcatctatggaaagtgaacttgacatctctatggataaattcgcatctgatgttaaacaattaggtgtggacttcaacgtatcaggtaaaccaattctaattaatcaatttggtcccccaattaagaaattccaaggtggtggccgtggtggccgtggtggtcgtggtagccgtggtggccgtggtggccgtggtgctccatctggtcctccaactttcgaaacctctccaggtgagatatctttgttaaacttattagaaaatattccaagcaatacctatattttgtatgtattgcgccgtggtaacgattctgctgtttatgatagattgaaatatatcactgatttgaaatttggtgcattgaattcctgtgttgtttgggacaacttcaaaaagaattctattcaatataattccaatgttgttatgaagatgaacttgaagttattaggtagtaaccactctctatctattgaaaacaacaaactattaattgataaggaatctaacttgccaatattagtgttgggttctgatgtgacacattatcctgaaaaggatcaaaactctattgcctcgttagtaggttcatacgatgacaaatttacccaattccctggtgattacatgcttcaagatggtccaggtgaagaaataattactaatgtcggttcattaatgttgaacagattaaagatatatcaaaaacataataatggtaaattaccaacgaagatcatgtacttcagagatggtgtttcagttgaccaattctctcaagttgttaagattgaagttaagtctattaaggaatcagtccgtaaatttggtcctcaattaaatggtggtaacaaatacgatccaccagttacatgtattgccactgtcaaaagaaatcaagtcagatttattcctatccaagaaaatgcgaagaatgaaaagggtgaagaagttgctgttcaatccatgggtaatgttatgccaggtactgttgtagaccgtggtatcacatctgtggcacactttgatttctttatccaatctcatcaagctttgaagggtactggtgtcccatgccactattggtgtctatatgacgaaaaccaatctacttctgactacttacaagaaatatgtaacaacttatgttacattttcggtagatctactaccagtgtaaaagtcccagccccagtatattatgccgatttgttgtgtacccgtgctacatgctttttcaaggcaggttttgaacttaatatggctcaagcaccaaaggagaagggttctaaggatcaacctactgtttccaagaatgttctattaccacaagttaacgataacattaaatccgtaatgtattacatttga

SEQ ID NO: 12 - DNA, 2754 bp, Candida albicans
atgagtgatttggttaaattttcaacaccaaccaaaatcaatgataaagctttagtagctattgatacttacaaaaagacttttcaaaaacaaataaagaatgaacaaaatggaattttgttgagaccatcttttggtactgaaggtgtccccactattgtaggagtcaattatttcaaatataatgttaatggattgaaattatactcctatcgtgtagatttattggaagatcctaatgttaaaaccagattatcaattaaaactgctgttgaaaaatatttaatggaattggaaccatttaaaagtaaaaaagccattatttattatcgtgatcataatcatctttattcaagaatgccccttccaattgaagatgtggttatttatccattggaacttggtggtgatccaaaaagagaaaagaaaattgttgaattgaaaattcaatttattaaacaattgaatttttctgatttactcaattatactcaattgaaaaattatactcccgattttttggaaaccgttgaatataccaatgcgttggttgcggttatgggatcccaagttatgaaaaatactaatgttgttggtcttggtcccaataaatttttcttgatggatggagccaccaagactactgatttagataaaggattatatgttgctatggggacttttgcatcggttagatgttcatttgatgatgttcgaatcaatttgaatccaaccccagcgattttttataaatcttcaaaaccaaatggacaaccaatgaatgttttggatttaattcaagaatttttgaaaattagaaatgtccctacggaaagagatattagaagagctcaatcatttattaaaggttgtaaaatttatcgtacttatcttaatcgtaaaactactaaaccaattcttggatttgattatagtaacaattccgaaacattaaaatttaaagatgctaatgataaattggttaatgttaaacaatatttccaagaaagatggaatattagattgaaacatcctactttaccattaattaaaattggtcctgaagcatttttaccaatgaaattgggtattattgctcctcatcaacaatataagggggattttgctgatccagcgaaaatcattaaaattactgctactagacctaatcaaaaggctgaattaattagtaaaaccaatagaaatttatttcaaaaacaagttgattttggtaccattgaatctcaattcaccgttgttccagcaagagttttaaatgctccaacaattgaatatgctaataatcaaatggttacttatagaccagcaccttttaatggtagaaccgaaaaacaaaaggggaattggaatcttgaaagatatcaatttgtcgatggagcaaaactcactaaaccaatggggaaacctttcacatttggtgttgttattttaaaagatgaatttgttgctaaacgtattggtgatttacaaagagctgtcccaacttttttaaccgaacttggtcgattagggattaaaattgggaaaaattttaaaaagtattctattgatttaaatcatgctagtgttcaatctcaaaccggtttagaatctaatataatttctattgccaaaaaagccaaaactgaagatcaatgtaatttcttgttgtttattttacctagacaagatactccattatattcagctattaaaagagcttgtgatcttaaagttgggattcttaatagttgttccattttgaatacttttactaagaagagacgtggaacagaaaattttgatgctatgacatatgcacaaatggccatgaaaattaatattaaattgggtggatctaatcataaattatcgaaaaaagatagtcaaggattatttgataaaaataatgtccccattttcattcttggtgctgatgttactcatccaacgggagaaattaattctgaatcagtttctattgcttctattgttggtagtgaagatggaattttcaataaattccctggatcagttcgtattcaaactggtggacaagaagttattgctgatgttaaaagtatggttttggaacgattagaaaatttccataaaaaaattgggaaattacctagtaaagtattattttatcgtgatggagtctctgaaggacaatataccaccatattaaaagaagaattaacaaaaattaaagccgcttttaatgaatatggtaaattgaaaaacatccctaaatattctccaaccattacatttatgattgttgttaaaagacatcatacaagatttattcctattcatgataatgccgatgatcccaaaaccaaaaaacaaatagctgtcaccagtaatgaaaatgtcattgctggtacaaccgttgatcgagaaatcacttctccagcatattttgatttttacgttcaatctcaacaatcattacaaggtactggaataccagctcattattatgttttacatgatgaaaataattatacttcagatactatacaaaaaatcacttatgatttatgtcatacttttagtagagctactaaatcagtcaaagttgtccccgcagcttattatgccgatttattatgtactagaggtagagattatatttatggttttgctaaagatccaaatttaaaaggatcacctattgaaagagcaagaactaaatttggtgaaaatgttaatccaagtattaaaaacaccatgttttatatttaa

(19) United States
(12) Patent Application Publication
(10) Pub. No.: US 2012/0309073 A1
(43) Pub. Date: Dec. 6, 2012

(54) RNAI IN BUDDING YEAST

(75) Inventors: David P. Bartel, Brookline, MA (US); Ines A. Drinnenberg, Cambridge, MA (US); David E. Weinberg, Cambridge, MA (US); Kathleen T. Xie, North Billerica, MA (US); Kenneth H. Wolfe, Dublin (IE); Gerald Fink, Cambridge, MA (US)

(73) Assignees: Whitehead Institute for Biomedical Research, Cambridge, MA (US); of Queen Elizabeth Near Dublin, Dublin (IE); The Provost, Fellows and Scholars of the College of the Holy and Undivided Trinity

(21) Appl. No.: 13/394,834

(22) PCT Filed: Sep. 10, 2010
(86) PCT No.: PCT/US10/02469

§ 371 Date: Aug. 7, 2012

Related U.S. Application Data
(60) Provisional application No. 61/241,356, filed on Sep. 10, 2009.
(60) Provisional application No. 61/347,034, filed on May 21, 2010.

(51) Int. Cl. C12N 1/19; C12N 9/22; C12N 15/52

(52) U.S. Cl. 435/199; 435/254.2; 536/23.2

(57) ABSTRACT

The invention provides budding yeast that have a functional RNAi pathway. The invention provides RNAi pathway polypeptides derived from budding yeast that have an endogenous RNAi pathway. In some embodiments the invention provides functional budding yeast Dicer polypeptides and variants thereof. In some embodiments the invention provides functional budding yeast Argonaute polypeptides and variants thereof. Also provided are isolated nucleic acids encoding the polypeptides of the invention, vectors comprising such nucleic acids, and methods of making the polypeptides and nucleic acids. The invention further provides genetically engineered cells that comprise a functional RNAi pathway polypeptide derived from budding yeast. In some embodiments such cells lack a functional endogenous RNAi pathway and are genetically engineered to have a functional RNAi pathway by introducing nucleic acid(s) encoding one or more functional RNAi pathway polypeptides derived from budding yeast. The invention provides methods of using RNAi in budding yeast and/or in cells of other types, wherein the cells have been genetically engineered to express one or more RNAi pathway polypeptides of the invention. Also provided are methods of producing siRNA, either in vitro or in vivo, using a Dicer polypeptide derived from budding yeast.

### GOVERNMENT FUNDING

The present invention was supported at least in part by NIH grants GM040266, GM0305010, and GM067031. The government has certain rights in the invention.

### BACKGROUND OF THE INVENTION

Since its discovery more than a decade ago, RNA interference (RNAi) has found use in applications ranging from functional genomics to development of therapeutic agents. Proteins that function in RNAi have been identified and characterized from a number of different eukaryotic species.

### SUMMARY OF THE INVENTION

The present invention relates in some aspects to the discovery of the RNAi pathway in budding yeast. The invention further relates to budding yeast RNAi pathway polypeptides Dicer and Argonaute. In one aspect, the invention provides a yeast cell that comprises a nucleic acid segment that encodes a non-endogenous RNAi pathway polypeptide that is functional in the yeast cell. In some embodiments, the yeast cell lacks an endogenous RNAi pathway. In some embodiments, the nucleic acid segment is operably linked to an expression control element capable of directing transcription in the yeast cell. In some embodiments, the expression control element comprises an inducible promoter. In some embodiments, the nucleic acid segment is a DNA segment that is integrated into the genome of the yeast cell. In some embodiments, the nucleic acid segment is present in an episome, which, in some embodiments, is a plasmid. In some embodiments, the yeast cell lacks an endogenous functional counterpart of the non-endogenous RNAi pathway polypeptide. In some embodiments, the yeast cell has a functional RNAi pathway when the non-endogenous RNAi pathway polypeptide is expressed. In some embodiments, the yeast cell is a budding yeast cell. In some embodiments, the yeast cell is a member of the subphylum Saccharomycotina. In some embodiments, the yeast cell is a member of the genus Saccharomyces. In some embodiments, the yeast cell is a Saccharomyces cerevisiae cell. In some embodiments, the yeast cell is a member of an industrially important yeast strain. In some embodiments, the yeast cell is a member of a pathogenic yeast species. In some embodiments, the non-endogenous RNAi pathway protein is derived from a budding yeast species that has a functional RNAi pathway. In some embodiments, the budding yeast species that has a functional RNAi pathway is a member of the subphylum Saccharomycotina, e.g., Saccharomyces castellii. In some embodiments, the budding yeast species that has a functional RNAi pathway is Kluveromyces polysporus. In some embodiments, the non-endogenous RNAi pathway protein is a Dicer polypeptide. In some embodiments, the non-endogenous RNAi pathway protein is an Argonaute polypeptide. In some embodiments, the yeast cell comprises (i) a first nucleic acid segment that encodes a non-endogenous Dicer polypeptide, wherein the first nucleic acid segment is operably linked to an expression control element capable of directing transcription in the yeast cell; and (ii) a second nucleic acid segment that encodes a functional non-endogenous Argonaute polypeptide, wherein the second nucleic acid segment operably linked to an expression control element capable of directing transcription in the yeast cell. In some embodiments, at least one of the expression control elements comprises an inducible promoter. In some embodiments, the yeast cell comprises a non-endogenous nucleic acid segment that can be transcribed to yield dsRNA that has sequence correspondence to mRNA of a gene. In some embodiments, the non-endogenous nucleic acid segment that is flanked by expression control elements in opposite orientation, so that convergent transcription occurs to yield RNAs that hybridize to form dsRNA that has sequence correspondence to mRNA of a gene. In some embodiments, transcription of the non-endogenous nucleic acid segment yields a transcript that comprises a sense portion and an antisense portion, wherein the antisense portion has complementarity to the mRNA, and wherein the sense portion and the antisense portion hybridize to form a dsRNA comprising a hairpin. In some embodiments, the non-endogenous nucleic acid segment is operably linked to an expression control element capable of directing transcription in the yeast cell. In some embodiments, the expression control element comprises an inducible promoter In some embodiments, the nucleic acid segment that can be transcribed to yield dsRNA is integrated into the genome of the yeast cell. In some embodiments, the nucleic acid segment that can be transcribed to yield dsRNA is present in an episome. In some embodiments, the gene is an endogenous gene of the yeast. In some embodiments, the gene is a non-endogenous gene. In some embodiments, the gene is a gene whose silencing results in improved ability of the yeast cell to produce a product of interest.

The invention further provides a library comprising a multiplicity of yeast cells as described in any of the above embodiments, wherein the library comprises cells in which mRNAs of at least 10 different genes are targeted for silencing by RNAi, wherein each of said genes is targeted in a different cell or population of cells. The invention further provides a library comprising a multiplicity of yeast cells as described in any of the above embodiments, wherein the library comprises cells in which mRNAs of at least 10% of the genes of the yeast are targeted for silencing by RNAi, wherein each of said genes is targeted in a different cell or population of cells. In some embodiments of the inventive libraries, the yeast cells are budding yeast cells.

The invention provides a budding yeast cell that lacks an endogenous RNAi pathway, wherein the budding yeast cell is genetically engineered so that it has a functional RNAi pathway. In some embodiments, the budding yeast cell lacks a functional endogenous Dicer polypeptide and is genetically engineered to contain a nucleic acid that encodes a functional Dicer polypeptide. In some embodiments, the budding yeast cell has a functional endogenous Dicer polypeptide but lacks a functional endogenous Argonaute polypeptide and is genetically engineered to contain a nucleic acid that encodes a functional Argonaute polypeptide. In some embodiments, the budding yeast cell lacks a functional endogenous Dicer polypeptide and lacks a functional endogenous Argonaute polypeptide, wherein the yeast cell is genetically engineered to contain a nucleic acid that encodes a functional Dicer polypeptide and a nucleic acid that encodes a functional Argonaute polypeptide.

The invention provides a budding yeast cell that has a functional RNAi pathway, wherein the budding yeast cell is genetically engineered to contain a nucleic acid segment that can be transcribed to yield a dsRNA that has sequence correspondence to mRNA of a gene. In some embodiments, the gene is an endogenous gene. In some embodiments, the gene is a non-endogenous gene. In some embodiments, the gene is an essential gene. In some embodiments, the nucleic acid segment is operably linked to an inducible promoter. In some embodiments, the budding yeast cell lacks a functional endogenous RNAi pathway. In some embodiments, the budding yeast cell is a member of the subphylum Saccharomycotina. In some embodiments, the budding yeast cell is a member of the genus Saccharomyces. In some embodiments, the budding yeast cell is an S. cerevesiae cell. In some embodiments, the yeast cell is a member of the genus Kluveromyces. In some embodiments, the yeast cell is a Kluveromyces polysporus cell. In some embodiments, the budding yeast cell is a member of the genus Pichia. In some embodiments, the budding yeast cell is a Pichia pastoris cell. In some embodiments, the budding yeast cell is a member of the genus Candida. In some embodiments, the budding yeast cell is a Candida albicans cell.

The invention further provides kits comprising a yeast cell of the invention, e.g., a yeast cell as described in any of the above embodiments (which may be further described elsewhere herein), wherein the kit optionally further comprises at least one of the following: (i) instructions for silencing a gene in the yeast cell using RNAi; (ii) a nucleic acid construct for use in engineering the yeast cell to express a dsRNA corresponding to a gene of interest; and (iii) a nucleic acid construct for use in engineering the yeast cell to express a control dsRNA.

In another aspect, the invention provides a method of producing a yeast cell that has a functional RNAi pathway, the method comprising: (a) providing a yeast cell that lacks a functional endogenous RNAi pathway; and (b) introducing into the yeast cell a nucleic acid that encodes a non-endogenous RNAi pathway polypeptide functional in the yeast cell, wherein the nucleic acid is operably linked to an expression control element capable of directing transcription in the yeast cell. In some embodiments, the non-endogenous RNAi pathway polypeptide is a functional Dicer polypeptide. In some embodiments, the non-endogenous RNAi pathway polypeptide is a functional Argonaute polypeptide. In some embodiments, the method comprises introducing into the yeast cell (i) a first nucleic acid segment that encodes a functional Dicer polypeptide, wherein the first nucleic acid segment is operably linked to an expression control element capable of directing transcription in the yeast cell; and (ii) a second nucleic acid segment that encodes a functional Argonaute polypeptide, wherein the second nucleic acid segment is operably linked to an expression control element capable of directing transcription in the yeast cell. In some embodiments, the yeast cell is a budding yeast cell. In some embodiments, the yeast cell is an S. cerevesiae cell. In some embodiments, the non-endogenous RNAi pathway polypeptide is derived from a budding yeast cell that has a functional endogenous RNAi pathway. In some embodiments, the expression control element comprises an inducible promoter.

In another aspect, the invention provides a method of silencing a gene in a budding yeast cell comprising: (a) providing a budding yeast cell that has a functional RNAi pathway; and (b) delivering siRNA to the budding yeast cell, thereby resulting in silencing of the gene. In some embodiments, the budding yeast cell lacks a functional endogenous RNAi pathway and is genetically engineered to have a functional RNAi pathway. In some embodiments, the budding yeast cell is genetically engineered to express a non-endogenous RNAi pathway polypeptide. In some embodiments, the budding yeast cell comprises a nucleic acid that can be transcribed to yield a dsRNA that has sequence correspondence to mRNA of the gene; and step (b) comprises maintaining the cell under conditions in which the dsRNA is expressed and is cleaved to siRNA, thereby resulting in silencing of the gene, e.g., targeting the mRNA of the gene for degradation. In some embodiments, the nucleic acid that can be transcribed to yield a dsRNA is non-endogenous. In some embodiments, the nucleic acid that can be transcribed to yield a dsRNA is operably linked to an inducible promoter. In some embodiments, the dsRNA is cleaved by a Dicer protein to yield siRNA that target the mRNA of the gene for silencing. In some embodiments, the budding yeast cell is a member of the genus Saccharomyces. In some embodiments, the budding yeast cell is an S. cerevesiaie cell. In some embodiments, the yeast cell is a member of the genus Kluveromyces. In some embodiments, the yeast cell is a Kluveromyces polysporus cell. In some embodiments, the budding yeast cell is a member of the genus Pichia. In some embodiments, the budding yeast cell is a Pichia pastoris cell. In some embodiments, the budding yeast cell is a member of a pathogenic yeast species. In some embodiments, the budding yeast cell has multiple copies of the gene. In some embodiments, the budding yeast cell has one or more parologs of the gene in its genome. In some embodiments, the gene is an endogenous gene. In some embodiments, the gene is an essential gene.

In another aspect, the invention provides a method of examining the function of a gene in a budding yeast cell comprising: (a) providing a budding yeast cell that has a functional RNAi pathway; and (b) delivering siRNA to the budding yeast cell, wherein the siRNA is targeted to a gene, thereby resulting in silencing of the gene. In some embodiments, the yeast cell comprises a nucleic acid that can be transcribed to yield a dsRNA that has sequence correspondence to mRNA of a gene; and wherein step (b) comprises maintaining the budding yeast cell under conditions in which the dsRNA is produced and cleaved to siRNA that results in silencing of the gene, thereby producing a budding yeast cell in which the gene is silenced. In some embodiments, the method further comprises (c) observing the phenotype of the budding yeast cell produced in (b), thereby providing information about the function of the gene. In some embodiments, the method comprises introducing into the budding yeast cell a nucleic acid construct that comprises a nucleic acid that can be transcribed to yield dsRNA that has sequence correspondence to mRNA of the gene. In some embodiments, the budding yeast cell is a member of the genus Saccharomyces. In some embodiments, the budding yeast cell is an S. cerevesiaie cell. In some embodiments, the budding yeast cell is a member of the genus Kluveromyces. In some embodiments, the budding yeast cell is a Kluveromyces polysporus cell. In some embodiments, the budding yeast cell is a member of the genus Pichia. In some embodiments, the budding yeast cell is a Pichia pastoris cell.

In another aspect, the invention provides a method of identifying a budding yeast cell with an altered phenotype relative to a control, the method comprising: (a) providing a budding yeast cell that has a functional RNAi pathway, (b) delivering siRNA to the budding yeast cell, thereby resulting in silencing of the gene; (c) comparing the phenotype of the budding yeast cell with the phenotype of an appropriate control; and (d) identifying the budding yeast cell as having an altered phenotype relative to a control if the phenotype of the budding yeast cell differs from that of the control. In some embodiments, the yeast cell comprises a nucleic acid that can be transcribed to yield a dsRNA that has sequence correspondence to mRNA of a gene; and wherein step (b) comprises maintaining the budding yeast cell under conditions in which the dsRNA is produced and cleaved to yield siRNA targeted to the gene, thereby producing a budding yeast cell in which the gene is silenced. In some embodiments, the phenotype is ability to produce a product of interest. In some embodiments, the product of interest comprises a biofuel. In some embodiments, the method further comprises isolating a yeast cell in which the gene is mutated.

In another aspect, the invention provides a method of identifying a gene that affects a phenotype of a budding yeast cell comprising: (a) providing a budding yeast cell that has a functional RNAi pathway; (b) delivering siRNA to the budding yeast cell, thereby resulting in silencing of a gene; (c) comparing the phenotype of the budding yeast cell with the phenotype of an appropriate control; and (d) identifying the gene as a gene that affects the phenotype if the budding yeast cell differs from the control with respect to the phenotype. In some embodiments, the budding yeast cell comprises a nucleic acid that encodes dsRNA that has sequence correspondence to mRNA of a gene of the yeast cell, and step (b) comprises maintaining the budding yeast cell under conditions in which the dsRNA is expressed and cleaved to yield siRNA targeted to the gene.

In another aspect, the invention provides a method of identifying a budding yeast cell with an altered phenotype relative to a control, the method comprising: (a) providing a budding yeast cell that has a functional RNAi pathway, (b) delivering siRNA to the budding yeast cell, thereby resulting in silencing of a gene; (c) comparing the phenotype of the budding yeast cell with the phenotype of an appropriate control; and (d) identifying the budding yeast cell as having an altered phenotype relative to a control if the phenotype of the budding yeast cell differs from that of the control. In some embodiments, the phenotype is ability to produce a product of interest. In some embodiments, the product of interest comprises a biofuel. In some embodiments, the method further comprises isolating a yeast cell in which the gene is mutated. In some embodiments, the method further comprises identifying a mammalian homolog of the gene.

In another aspect, the invention provides a method of producing a product of interest comprising: (a) providing a budding yeast cell that has a functional RNAi pathway; (b) delivering siRNA to the budding yeast cell, thereby resulting in silencing of a gene; and (c) maintaining the yeast cell under conditions suitable for production of the product by the yeast cell. In some embodiments, the budding yeast cell expresses a dsRNA that has sequence correspondence to mRNA of a gene whose inhibition improves production of the product; and wherein step (b) comprises maintaining the cell under conditions in which the dsRNA is expressed and cleaved to siRNA that is targeted to the gene, e.g., that targets mRNA of the gene for degradation. In some embodiments, the budding yeast cell is a member of the genus Saccharomyces. In some embodiments, the budding yeast cell is an S. cerevesiaie cell. In some embodiments, the budding yeast cell is a member of the genus Pichia. In some embodiments, the budding yeast cell is a Pichia pastoris cell. In some embodiments, the method further comprises isolating the product. In some embodiments, the product comprises a biofuel.

In another aspect, the invention provides a method of producing siRNA comprising: (a) providing cells that express a functional budding yeast Dicer polypeptide, wherein the cells express a dsRNA at least 50 nucleotides long; and (b) maintaining the cells under conditions in which the dsRNA is cleaved to form siRNA; and (c) isolating siRNA formed in step (b). In some embodiments, the cells are budding yeast cells. In some embodiments, the cells are bacterial cells. In some embodiments, the dsRNA corresponds to a mammalian gene. In some embodiments the method further comprises isolating siRNA from the composition.

In another aspect, the invention provides a composition comprising: (a) an extract derived from cells that express a functional budding yeast Dicer polypeptide; and (b) a dsRNA comprising a portion at least 40 base pairs long; provided that, if the cells are budding yeast cells that express an endogenous Dicer polypeptide, then the dsRNA is not endogenous to said budding yeast cells. In some embodiments, the extract is derived from bacterial cells. In some embodiments, the extract is derived from budding yeast cells that lack a functional endogenous RNAi pathway and are genetically engineered to express a functional budding yeast Dicer polypeptide. In some embodiments, the dsRNA corresponds to a mammalian gene.

In another aspect, the invention provides a method of producing siRNA comprising: (a) providing the afore-mentioned composition; and (b) maintaining the composition of (a) under conditions under which the dsRNA is processed to siRNA. In some embodiments, the method further comprises isolating siRNA from the composition of (b).

In another aspect, the invention provides a method of silencing a gene in a cell comprising contacting the cell with siRNA produced according to any of the methods of producing siRNA described above (which may be further described elsewhere herein).

In another aspect, the invention provides an isolated nucleic acid comprising a polynucleotide that has a sequence at least 80% identical to the sequence of a naturally occurring polynucleotide that encodes an RNase III domain of functional budding yeast Dicer polypeptide. In some embodiments, the polynucleotide has a sequence identical to the sequence of a naturally occurring polynucleotide that encodes an RNase III domain of a functional budding yeast Dicer polypeptide. In some embodiments, the sequence further comprises a sequence at least 80% identical to a dsRNA binding domain of a functional budding yeast Dicer polypeptide. In some embodiments, the polynucleotide has a sequence at least 80% identical to the sequence of a naturally occurring polynucleotide that encodes functional budding yeast Dicer polypeptide. In some embodiments, the polynucleotide is operably linked to an expression control element that is not operably linked to the polynucleotide in nature. In some embodiments, the polynucleotide is operably linked to an expression control element capable of directing transcription in S. cerevesiae. In some embodiments, the polynucleotide is operably linked to an expression control element capable of directing transcription in a bacterial cell. In some embodiments, the isolated nucleic acid comprises a portion that encodes a selectable marker. In another aspect, the invention provides a cell containing any one or more of the isolated nucleic acids set forth above. In some embodiments, the cell is a bacterial cell. In some embodiments, the cell is a yeast cell. In some embodiments, the cell is a budding yeast cell. In some embodiments, the cell is a budding yeast cell that is a member of the genus Saccharomyces. In some embodiments, the cell is an S. cerevesiae cell. In some embodiments, the cell is a budding yeast cell that is a member of the genus Pichia. In some embodiments, the cell is a Pichia pastoris cell. In some embodiments, the cell is a bacterial cell.

In another aspect, the invention provides an isolated polypeptide comprising a polypeptide that has a sequence at least 80% identical to the sequence of an RNase III domain of a functional budding yeast Dicer polypeptide. In some embodiments, the isolated polypeptide comprises a polypeptide that has a sequence at least 90% identical to the sequence of an RNase III domain of a functional budding yeast Dicer polypeptide. In some embodiments, the isolated polypeptide comprises a polypeptide that has a sequence at least 80% identical to the sequence of an RNase III domain of S. castellii Dicer polypeptide. In some embodiments, the isolated polypeptide comprises a polypeptide that has a sequence at least 80% identical to the sequence of an RNase III domain of K. polysporus Dicer polypeptide. In some embodiments, the isolated polypeptide further comprises a dsRNA binding domain. In some embodiments, the sequence of the dsRNA binding domain is at least 80% identical to the sequence of a dsRNA binding domain of a functional budding yeast Dicer polypeptide. In some embodiments, the isolated polypeptide comprises a polypeptide that has a sequence at least 80% identical to the sequence of a functional budding yeast Dicer polypeptide. In some embodiments, the isolated polypeptide comprises a polypeptide that has a sequence at least 80% identical to the sequence of S. castellii Dicer polypeptide. In some embodiments, the isolated polypeptide comprises a polypeptide that has a sequence at least 80% identical to the sequence of K. polysporus Dicer polypeptide. In some embodiments, the polypeptide comprises a tag.

In another aspect the invention provides an isolated nucleic acid comprising a polynucleotide that encodes any of the isolated polypeptides set forth above (which may be further described elsewhere herein). In some embodiments, the sequence of the polynucleotide is comprises a sequence that in nature encodes an RNase III domain of a functional budding yeast Dicer polypeptide. In some embodiments, the sequence of the polynucleotide is comprises a sequence that in nature encodes a functional budding yeast Dicer polypeptide. In some embodiments, the sequence of the polynucleotide is codon optimized for expression in a bacterial cell. In some embodiments, the polynucleotide is operably linked to an expression control element. In some embodiments, the isolated nucleic acid comprises a portion that encodes a selectable marker. In some embodiments, the isolated nucleic acid further comprises a polynucleotide that encodes a polypeptide at least 80% identical to an Argonaute polypeptide, wherein said Argonaute polypeptide is optionally a budding yeast Argonaute polypeptide. In some embodiments, the polynucleotide that encodes a polypeptide at least 80% identical to an Argonaute polypeptide is operably linked to an expression control element. In some embodiments, the isolated nucleic acid further comprises a polynucleotide that can be transcribed to yield a dsRNA comprising a portion at least 20 base pairs long that has sequence correspondence to mRNA of the gene.

In another aspect, the invention provides a method of producing an isolated polypeptide of the invention comprising (i) providing a cell that comprises a polynucleotide that encodes the polypeptide, wherein the polynucleotide is operably linked to an expression control element capable of directing transcription in the cell; (ii) maintaining the cell under conditions in which the polypeptide is expressed; and (iii) isolating the polypeptide from the cell.

In another aspect, the invention provides a composition comprising any of the isolated polypeptides set forth above; and (ii) a dsRNA comprising a portion at least 20 base pairs long, e.g., at least 40, 50, 100, 200, 300, 400, 500, 1000 by long. In some embodiments, the isolated polypeptide comprises a polypeptide that has a sequence at least 80% identical to the sequence of a functional budding yeast Dicer polypeptide. In some embodiments, the composition comprises a polypeptide that has a sequence at least 80% identical to the sequence of an RNase III domain of a functional budding yeast Dicer polypeptide. In some embodiments, the composition comprises a polypeptide that has a sequence at least 80% identical to the sequence of an RNase III domain of S. castellii Dicer polypeptide. In some embodiments, the composition comprises a polypeptide that has a sequence at least 80% identical to the sequence of an RNase III domain of K. polysporus Dicer polypeptide. In some embodiments, the composition comprises a polypeptide that has a sequence at least 80% identical to the sequence of a functional budding yeast Dicer polypeptide. In some embodiments, the isolated polypeptide comprises a polypeptide that has a sequence identical to the sequence of S. castellii Dicer polypeptide. In some embodiments the isolated polypeptide comprises a polypeptide that has a sequence identical to the sequence of K. polysporus Dicer polypeptide. In some embodiments, the dsRNA corresponds to a mammalian gene. The invention further provides a method of producing siRNA comprising maintaining the composition under conditions in which the dsRNA is cleaved to siRNA. The method may further comprise isolating siRNA from the composition.

In another aspect, the invention provides a method of silencing a gene in a cell comprising contacting the cell with siRNA produced according to any of the methods for producing siRNA described above (which may be further described elsewhere herein).

In another aspect, the invention provides an isolated nucleic acid comprising a polynucleotide that encodes a polypeptide that has a sequence at least 80% identical to the sequence of a functional budding yeast Argonaute polypeptide, wherein the polynucleotide is operably linked to an expression control element capable of directing transcription in a cell that lacks a functional endogenous Argonaute polypeptide. In some embodiments, the expression control element is capable of directing transcription in a budding yeast cell that lacks a functional Argonaute polypeptide. In some embodiments, the polypeptide has a sequence at least 90% identical to the sequence of a functional budding yeast Argonaute polypeptide. In some embodiments, the polypeptide has a sequence identical to the sequence of a functional budding yeast Argonaute polypeptide. In some embodiments, the isolated nucleic acid comprises a portion that encodes a selectable marker.

In another aspect, the invention provides methods of identifying a budding yeast cell that comprises a functional Dicer polypeptide (e.g., that contains a gene encoding a functional Dicer polypeptide). In some embodiments, the method comprises characterizing short RNAs isolated from a budding yeast cell to determine whether such short RNAs comprise short RNAs having features of siRNA, wherein the presence of short RNAs having features of siRNAs implies that the budding yeast cell comprises a functional Dicer polypeptide.

In another aspect, the invention provides a method of producing a budding yeast cell that has reduced transposition, the method comprising: (i) providing a budding yeast cell that lacks a functional RNAi pathway and exhibits transposition; and (ii) genetically engineering the budding yeast to have a functional RNAi pathway, thereby resulting in a budding yeast cell that exhibits reduced transposition. In some embodiments, the method further comprises monitoring transposition in the genetically engineered budding yeast cell. In some embodiments, the method further comprises using the engineered budding yeast cell to produce a product of interest.

In another aspect, the invention provides a vector comprising any one or more of the isolated nucleic acids set forth above.

In another aspect, the invention provides a cell comprising any one or more of the isolated nucleic acids set forth above. The cell can be a eukaryotic cell or a prokaryotic cell. The cell can be a fungal cell (e.g., a yeast cell, e.g., a budding yeast cell), a bacterial cell, an insect cell, a mammalian or avian cell. Compositions, e.g., cultures, comprising a cell of the invention are provided.

In another aspect, the invention provides an antibody that binds to any of the isolated polypeptides described above.

In another aspect, the invention provides a kit comprising any one or more of the isolated nucleic acids, isolated polypeptides, vectors, antibodies or cells, set forth above and/or elsewhere herein. Optionally the kit comprises instructions for use and one or more reagents for use in performing a method of the invention.

The practice of the present invention will typically employ, unless otherwise indicated, conventional techniques of molecular biology, cell culture, recombinant nucleic acid (e.g., DNA) technology, immunology, nucleic acid and polypeptide detection, manipulation, and quantification that are within the skill of the art. Non-limiting descriptions of certain of these techniques are found in the following publications: Xiao, W. (ed.) Yeast protocols (Methods in molecular biology) (Clifton, N.J.); v. 313. Totowa, N.J.: Humana Press, Clifton, N.J., 2006; Guthrie, C, and Fink, G. (eds.) Guide to Yeast Genetics and Molecular Cell Biology, Part B, Volume 350 (Methods in Enzymology), Academic Press, 2002; Amberg, D., et al., (eds.) Methods in Yeast Genetics: A Cold Spring Harbor Laboratory Course Manual, Cold Spring Harbor Laboratory Press, Cold Spring Harbor, 2005; Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988. All patents, patent applications, and other publications and references mentioned herein are incorporated by reference in their entirety. Standard art-accepted meanings of terms are used herein unless indicated otherwise. Standard abbreviations for various terms are used herein.

### BRIEF DESCRIPTION OF THE DRAWINGS

FIG. 1. Endogenous siRNAs in some budding-yeast species. (A) Cladogram of selected fungal species. Shown are Basidiomycota (blue), Zygomycota (grey) and Ascomycota, which are subdivided into the Saccharomycotina (budding yeasts, orange), the Pezizomycotina (yellow) and the Taphrinomycotina (green). The topology is according to (36, 37). The presence of canonical RNAi genes is indicated (+), according to (6, 7) and references therein. All genomes contain a RNT1 ortholog, and several others contain a second RNaseIII domain-containing gene (*), which has Dicer activity in S. castellii and presumably other species. Pseudogenes are indicated (ψ). S. bayanus, which had a Dicer but not an Argonaute gene, appeared to lack siRNAs (fig. S1). (B) Length distribution of genome-matching sequencing reads representing small RNAs with the indicated 5′ nucleotide. Reads matching rRNA and tRNA are excluded. (C) Classification of loci to which 21-23-nt RNAs map, based on genome annotation, considering those that map to clusters in a pattern suggestive of siRNAs separately from those that do not. (D) A palindromic region generating siRNAs in S. castellii. 5′ termini of 22-23-mers were mapped to the genome and the counts (normalized to the number of genomic matches) are plotted for the plus and minus genomic strands. The top graph considers all reads; the bottom considers those matching the genome at only one locus. The predicted structure of the (+)-strand transcript is represented as a mountain plot (38). (E) Distribution of the genomic intervals separating the 5′ termini of sequenced 23-mers from S. castellii. Plotted is the frequency of each interval, when considering all pairs of reads less than 100 nt apart (excluding reads matching rRNA and tRNA).

FIG. 2. The Dicer of budding yeast. (A) In vitro processing of radiolabeled dsRNA or single-stranded RNA (ssRNA) in extracts from the indicated budding-yeast species. Products were resolved on a denaturing gel, with the migration of markers indicated on the left. The fraction of product normalized to that observed with dsRNA is indicated below as a percentage. (B) RNA blot probing for an endogenous siRNA (sc1056) in the indicated deletion and rescue strains. The blot was reprobed for U6 small nuclear RNA, and the siRNA percent signal normalized to that of U6 is indicated below. (C) Domain architectures of representative Dicer proteins and the two S. castellii proteins containing an RNaseIII domain. (D) Maximum-likelihood tree reconstruction based on amino acid alignment of RNaseIII domains from representative Dicer proteins and Rnt1 homologues. Orange shading highlights budding-yeast Dicer candidates indicated by asterisks in FIG. 1A. Budding-yeast species encoding Argonaute are in listed in red. Bootstrap values higher than 50% are shown. (E) In vitro dicing in extracts from recombinant S. castellii (S. cas), S. cerevisiae (S. cer), or E. coli strains with the indicated deletions and additions, analyzed as in (A).

FIG. 3. The impact of RNAi on the S. castellii transcriptome. (A) Strand-specific mRNA-Seq analysis of annotated ORF transcripts in wild-type (WT) and RNAi-mutant strains. Plotted is the log₂ ratio of transcript abundance in Δago1 versus wild-type (x-axis) and Δdcr1 versus wild-type (y-axis). Colors indicate the density (reads/kb) of antisense small (22-23-nt) RNAs that co-purified with Ago1. A Ty ORF fragment (annotated as Scas_{—}712.50) embedded within a palindromic siRNA-producing locus is indicated (square). Annotated Y′ element ORFs were replaced by one consensus Y′ ORF (triangle, fig S5). Because the mRNA-Seq protocol included poly(A) selection, which would retain the 3′ but not 5′ fragments of cleaved mRNAs, full-length transcript abundance was calculated using tags mapping to the 5′ half of each ORF. Similar trends were observed when transcript abundance was calculated using tags across the entire ORF (fig. S4). (B) Analysis of the S. castellii Y′ element. The number of siRNA 5′ ends (small RNAs) and the number of mRNA tags from wild-type and mutant strains (mRNA-Seq) mapping to the consensus Y′ element is plotted for each position. (C) mRNA-Seq analysis of inferred siRNA-generating transcripts. The plot is as in (A), using the same colors to indicate siRNA-read density and shapes to indicate transcripts mapping to Y′ elements (triangle), palindromes (square), and others (diamonds). (D) A pair of convergent transcripts that generate siRNAs in the region of overlap. Plots are as in (B). (E) Systematic analysis of gene-pair organization and overlap in S. castellii. The inner ring shows the relative orientation of neighboring annotated ORFs. The middle ring shows the fraction of transcript pairs with overlapping 3′ ends (convergent), overlapping 5′ ends (divergent), or continuous transcription in between (tandem). The outer ring shows the fraction of convergent transcript pairs generating siRNAs in the overlapping region.

FIG. 4. Engineering RNAi in S. castellii and reconstituting it in S. cerevisiae. (A) Schematic for silencing of a GFP reporter. The strong silencing construct included inverted repeats of a gfp fragment and was designed to produce a hairpin transcript. The weak silencing construct contained one copy of the fragment, which is transcribed convergently to produce dsRNA. The hairpin and duplex dsRNA are processed into siRNAs targeting a functional GFP (green box). Galactose can induce both constructs. (B) RNA blot probing for siRNAs antisense to GFP, using total RNA from the indicated S. castellii strains with integrated empty vector (Ø) or silencing construct (strong or weak), either induced with galactose (+) or uninduced (−). (C) FACS histograms showing GFP fluorescence in the indicated S. castellii strains expressing the indicated silencing constructs. (D) RNA blot probing for siRNAs antisense to GFP in S. cerevisiae strains expressing either no S. castellii genes (WT) or the indicated integrated S. castellii genes, and either the strong (St), the weak (Wk), or no (Ø) silencing construct. (E) FACS histograms showing GFP fluorescence in the indicated S. cerevisiae strains expressing the indicated silencing constructs. All strains were induced; silencing from uninduced constructs was similar for the strong construct and undetectable for the weak construct (fig. S9). (F) RNA blot probing for GFP mRNA in the indicated S. cerevisiae strains expressing the indicated silencing constructs. As a loading control, the blot was reprobed for PYK1 mRNA. (G) Silencing an endogenous gene. S. cerevisiae strains containing non-functional and functional URA3 genes (ura3 and URA3, respectively) and expressing the indicated S. castellii genes and silencing constructs (labeled as in E) were tested for Ura3 expression by growth of 1:10 serial dilutions on plates lacking uracil (SC-Ura) and on plates containing 5-FOA, to which cells producing Ura3 are sensitive.

FIG. 5. Silencing of Ty1 retrotransposons by RNAi in S. cerevisiae. (A) Ty1his3AI transposition assay. Galactose-induced S. cerevisiae strains expressing the indicated S. castellii genes were tested for transposition by growth on plates lacking histidine (SC-His). When the his3AI-marked Ty1 element retrotransposes, a functional HIS3 is produced, and cells can grow on media lacking histidine. Growth on non-selective media (SC-Ura) is also shown. (B) mRNA-Seq analysis of an S. cerevisiae Ty1 element. Tags mapping to YDRWTy1-5 are shown, with tags contributing to the counts along their entire length. At the left, an antisense transcript (blue) initiates from within the element and extends beyond it; at the right, overlapping convergent transcripts initiate from promoters downstream of some Ty1 integrants and terminate within the element. (C) Ty1 Gag protein levels, as measured by immunoblotting with Ty1-VLP antiserum (39). S. cerevisiae strains expressing the indicated S. castellii genes were grown under standard conditions (30° C.) or transposition-inducing conditions (20° C.). The precursor (p49) and mature Gag (p45) are indicated. The blot was reprobed for actin. (D) Ty1 mRNA levels, as measured by RNA blotting. Strains are as in (C). Ethidium bromide-stained rRNA is shown.

FIG. 6. Sequences of selected budding yeast Dicer polypeptides and Argonaute polypeptides and nucleic acids that encode them. Number in parentheses and italics are coordinates of RNaseIII domain obtained by running NCBI Conserved Domain Search.

FIG. 7. Strategy for using budding yeast Dicer to generate siRNA from dsRNA in vitro.

FIG. 8. Denaturing gel showing siRNA produced by cleavage of dsRNA in vitro using purified K. polysporus Dicer fragment. A variety of different Dicer:dsRNA ratios and reaction times were tested.

FIG. 9. Native polyacrylamide gel showing scaled-up production of siRNA by cleavage of dsRNA in vitro using purified K. polysporus Dicer fragment or E. coli RNase III.

FIG. 10. Potent and specific knockdown of Renilla luciferase gene in mammalian cells using siRNA pool generated by cleavage of dsRNA in vitro using purified K. polysporus Dicer fragment. Error bars indicate the maximum and minimum ratios of a set of three wells.

FIG. S1. Analysis of small RNA library from S. bayanus MCYC 623. Length distribution of genome-matching reads (as percent of reads that do not match tRNA or rRNA) representing small RNAs with the indicated 5′ nucleotide (nt). Reads matching tRNAs and rRNAs were excluded.

Fig. S2. Analysis of small-RNA libraries from RNAi-mutant strains. (A) Length distributions of genome-matching reads (as percent of reads that do not match tRNA or rRNA) representing small RNAs with the indicated 5′ nucleotide (nt). Reads matching tRNAs and rRNAs were excluded. (B) Classification of 21-23-nt reads based on genome annotations and alignments.

Fig. S3. Sequencing of Ago1-associated small RNAs. (A) Length distribution of genome-matching sequencing reads representing small RNAs with the indicated 5′ nucleotide. Reads matching rRNA and tRNA are excluded. (B) Enrichment analysis of 22-23-nt reads based on genome annotation and alignments of their mapped loci. Italicized numbers above bars represent fold-enrichment calculated as (% of total reads in IP)/(% of total reads in Input). (C) Classification of 22-23-nt reads based on genome annotation and alignments of their mapped loci, considering those that map to clusters in a pattern suggestive of siRNAs separately from those that do not. Gray shading indicates the fraction of small RNAs considered to be siRNAs.

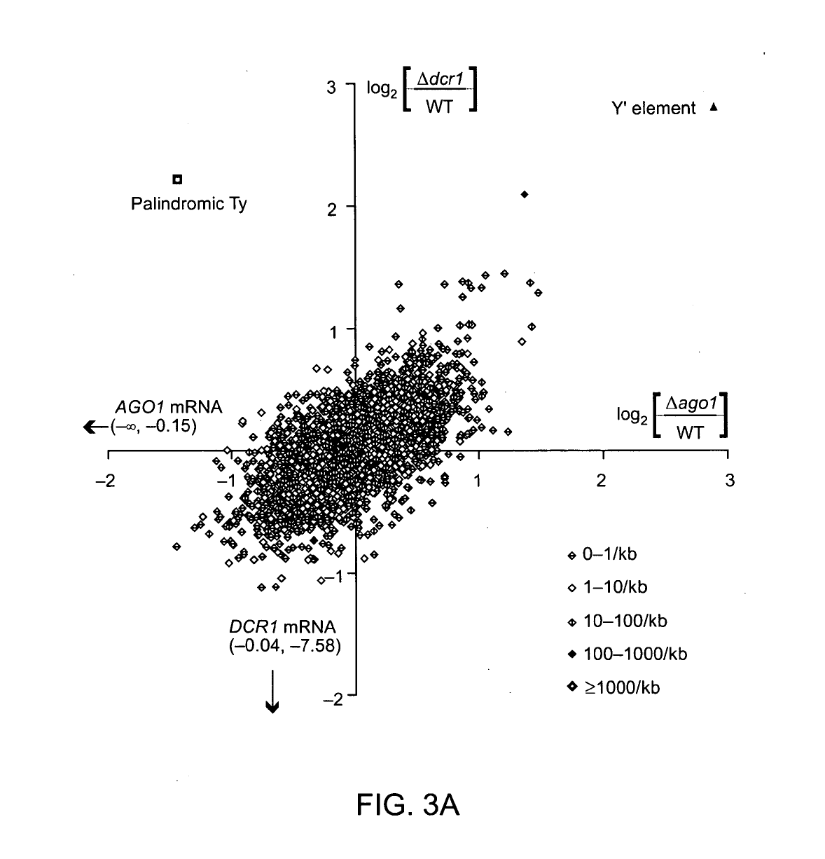

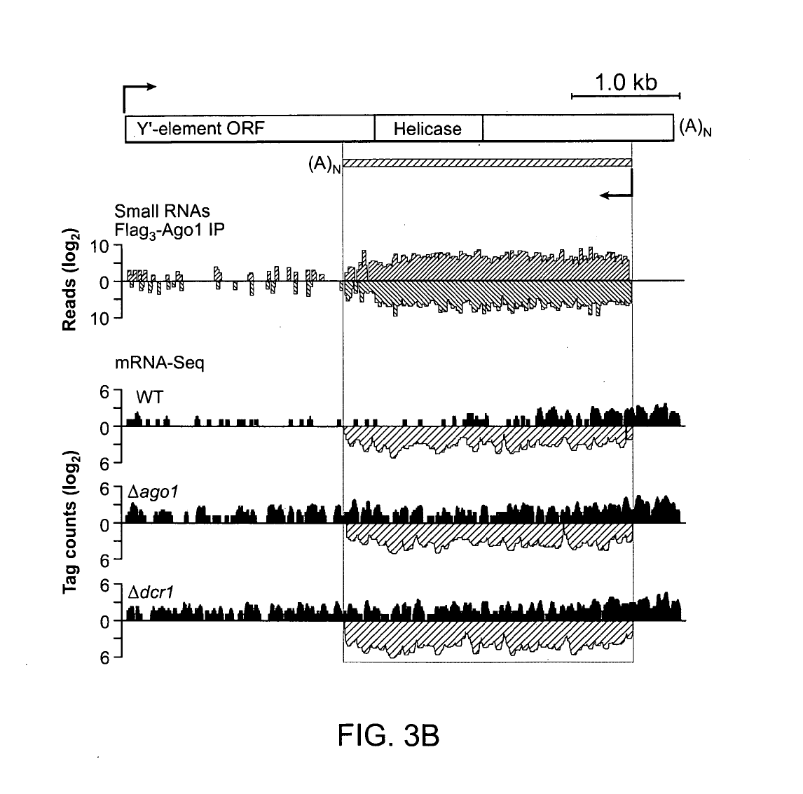

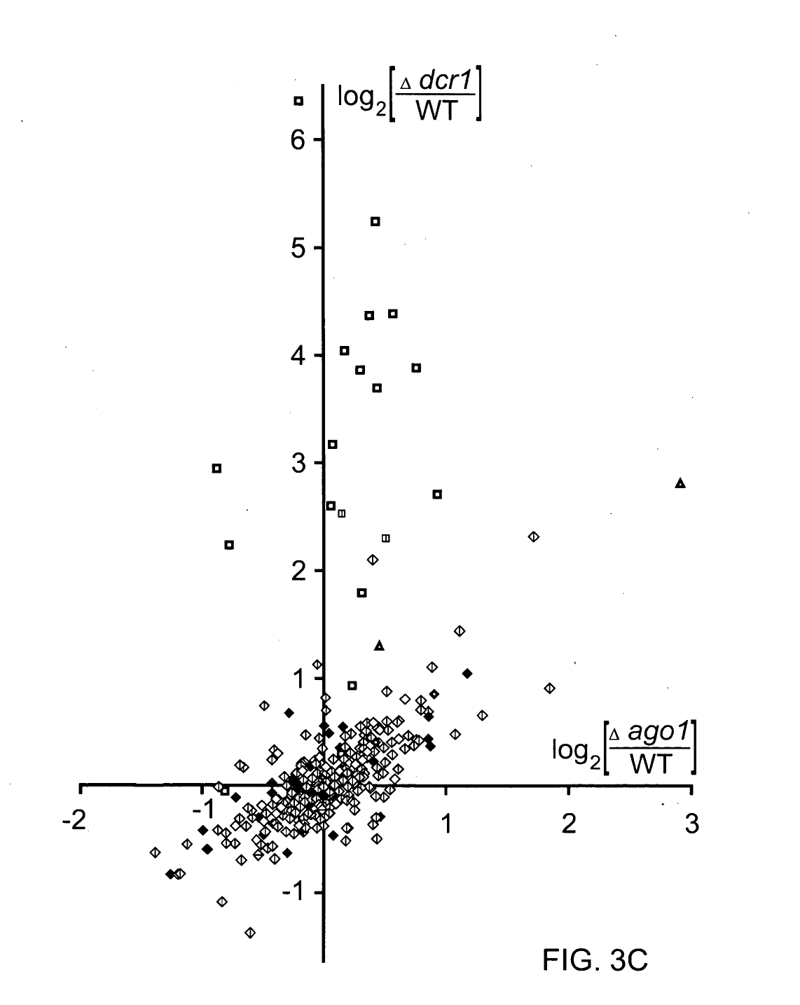

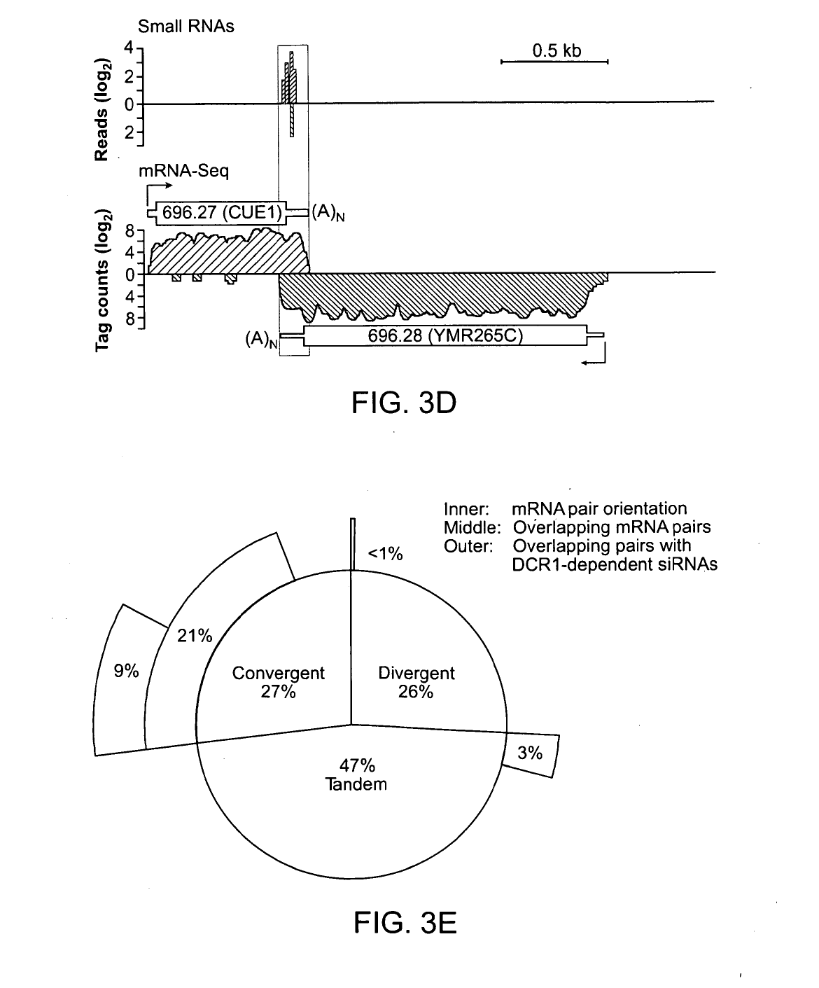

Fig. S4. mRNA-Seq analysis of wild-type and RNAi-mutant strains. (A) Correlation in transcript abundance between biological replicates. The number of tags mapping to the 5′ half of each annotated ORF was used to estimate the abundance of full-length transcripts. Expression level was calculated as tags per kilobase of coding exon. (B) Correlation in transcript abundance between wild-type and RNAi-mutant strains. Plots are as in (A). AGO1 mRNA had 96.77 tags/kb and 0 tags/kb in WT and Δago1 strains, respectively. (C) Plot is as in FIG. 3A, except that transcript abundance was calculated using tags across the entire ORF. (D) Plot is as in FIG. 3C, except that transcript abundance was calculated using tags across the entire transcript.

Fig. S5. Assembly of a S. castellii Y′-element consensus sequence. Y′-element fragments were assembled into a single consensus sequence as described in Materials and Methods. Vertical bars represent single-nucleotide polymorphisms with respect to the majority sequence, many of which fell at the ends of contigs and are presumed to include sequencing errors.

Fig. S6. Impact of siRNAs on ORF-containing transcripts. (A) Statistical analysis of the impact of small RNAs (sRNAs) mapping antisense to annotated ORFs. ORFs were sorted descending by antisense sRNAs per kb and the significance of transcript down regulation for the ORFs with greater numbers of small RNAs was calculated for the full range of cutoff values. A one-sided KS test was used to compare the distribution of Δago1/WT (blue) or Δdcr1/WT (green) transcript ratios for ORFs above and below each cutoff. Plotted are the resulting P-values as a function of the cutoff (expressed as the fraction of all antisense-sRNA-containing ORFs included above the cutoff). The red line indicates the P=0.05 significance cutoff. (B) Statistical analysis of the impact of sRNAs generated by overlapping convergent gene pairs. ORFs were sorted descending by overlapping-transcript-derived antisense sRNAs/kb and analyzed as in (A).

Fig. S7. Gene-pair organization and overlap in S. castellii. (A) Distribution of gene-pair inter-transcript distances. Gene pairs were binned by the distance between 3′-ends (convergent), 5′-ends (divergent), or 3′-end of the upstream gene and 5′-end of the downstream gene (tandem). Plotted is the fraction of gene pairs of a given orientation category that fall within each bin. † For overlapping tandem gene pairs, transcript ends for both genes represent the 5′ and 3′ ends of the contiguous signal observed by mRNA-Seq. Therefore, tandem gene pairs are depicted as overlapping across their length. (B) Correlation between transcript abundance and small RNA density for annotated ORFs. ORFs were binned according to inferred duplex abundance (estimated as the abundance of the limiting strand; top) or total transcript abundance (sum of sense and antisense tags; bottom). Plotted is the fraction of ORFs within a given bin that have at least as many uniquely matching small RNA reads (on either strand) as the x-axis value. As expected if siRNAs in coding sequences derived from dsRNA precursors formed by sense-antisense transcript pairs, the abundance of ORF siRNAs correlated with the abundance of the inferred duplex. Filtered data excludes all convergent overlapping gene pairs that give rise to small RNAs in the overlap region.

Fig. S8. mRNA-Seq analysis of the S. cerevisiae Y′ elements. (A) Transcripts mapping to chromosome XVI subtelomeres. mRNA-Seq tags were mapped to the reference genome. Tags mapping to the subtelomeric regions of chromosome XVI are shown, with tags contributing to the counts along their entire length. Positions of the vertical axes correspond to the ends of the chromosome. Y′-L and Y′-S represent the inferred genes corresponding to the long and short isoforms of S. cerevisiae Y′ elements, respectively. In S. cerevisiae, the telomeres are transcriptionally silenced by Sir2-dependent heterochromatin but still give rise to low levels of cryptic transcripts that are rapidly degraded by the TRAMP and exosome complexes (29). The previously characterized S. cerevisiae cryptic telomeric transcripts are ˜6.5 kb in length, and begin near chromosome ends and run antisense through the entire Y′-element ORF. The antisense reads we detected across S. cerevisiae subtelomeric regions may represent these previously identified cryptic transcripts. (B) Transcripts mapping to chromosome XII subtelomeres. Plots are as in (A).

Fig. S9. Reconstituting RNAi in S. cerevisiae. (A) Northern blot for siRNAs antisense to GFP in a S. cerevisiae strain expressing S. castellii AGO1, DCR1, and either no silencing construct (Ø), an integrated strong silencing construct (St), or an integrated weak silencing construct (Wk). Cells were induced in SC media with galactose and raffinose or uninduced in SC media with glucose. (B) FACS histograms of GFP fluorescence in S. cerevisiae expressing S. castellii AGO1 and DCR1 and the indicated silencing constructs. The same cultures were used here for sorting as for RNA collection in (A). In principle the siRNAs and silencing observed under uninduced conditions could be due to leaky expression from the GAL1 promoter, but these effects are probably attributable to constitutive antisense transcription from a downstream promoter.

Fig. S10. Analysis of GFP mRNA in reconstituted RNAi in S. cerevisiae. Aliquots from RT-PCR reactions were removed after increasing numbers of PCR cycles (GFP: 28, 32, 36; ACT1: 24, 28, 32) and visualized by ethidium bromide staining.

Fig. S11. Plasmid instability in RNAi mutants. (A) Number of colonies obtained upon transformation of each strain with the plasmid indicated, sum of three independent transformations (table S6). The CEN plasmid was pRS316; 2μ was a 2-micron origin plasmid; 2μ Ago1 and 2μ Dcr1 were 2-micron plasmids expressing Ago1 or Dcr1, respectively, under the S. cerevisiae GAL1 promoter. (B) Southern blot for abundance of the indicated plasmid in each of the indicated strains. Plasmids (CEN, 2μ) were detected with a probe against the ampicillin-resistance gene; loading controls (thin panels) were probed for a genomic locus. DNA was isolated from cells grown in SC-ura (selective) or YPD (non-selective). (C) Southern blot probed as in (B) monitoring rescue of plasmid maintenance phenotype using DNA isolated from cells grown in YPD (uninduced) or YP-galactose (induced). Δago1 and Δdcr1 mutants yielded fewer colonies upon plasmid transformation than did wild-type S. castellii (fig. S11A, top two rows). This effect was observed for CEN plasmids (which contained an S. cerevisiae centromere sequence and an S. cerevisiae chromosomal origin of replication) as well as 2-micron plasmids (which contained the origin of the S. cerevisiae endogenous 2-micron circle but no centromere sequence). To distinguish whether this effect reflected a defect in plasmid transformation (plasmid entering the cell) or plasmid maintenance (propagation of the plasmid after entering the cell), we attempted to rescue the defect by transforming wild-type, Δago1, and Δdcr1 strains with plasmids expressing either Δgo1 or Dcr1 from an inducible promoter. If the mutant strains were defective in transformation, then these Ago1 and Dcr1 expression plasmids would not enter the cell and thus could not rescue the mutant phenotype. Alternatively, if the mutant strains were defective in plasmid maintenance, then these plasmids would enter the cell, and expression of plasmid-borne Ago1 or Dcr1 in the cognate mutant could rescue maintenance of the expression plasmid itself. When the Δago1 mutant was transformed with the Ago1-expression plasmid and the cells were plated on inducing media, wild-type numbers of colonies were obtained. The same was observed for the Δdcr1 mutant transformed with the Dcr1-expression plasmid. This rescue was not observed with the non-cognate plasmids or when expression was not induced (fig. S11A), thereby demonstrating the specificity of the rescue. These results show that RNAi is required for maintenance of S. cerevisiae plasmids in S. castellii. We then used Southern blots to monitor plasmid levels. For the CEN plasmid, Δago1 and Δdcr1 mutants carried, on average, fewer plasmids per cell relative to wild-type cells, even when grown in selective media (fig. S11B, top). For the 2-micron plasmid, Δago1 and Δdcr1 mutants maintained the plasmid at wild-type abundance in selective media, although growth was considerably slower. When allowed to lose plasmid by growth in rich, non-selective media, the mutants lost more plasmid than the wild-type cells did (fig. S11B, bottom). Consistent with the rescue observed when counting colonies (fig. S11A), expressing the relevant protein from the plasmid being monitored rescued the plasmid-maintenance phenotype (fig. S11C). Partial rescue was observed without induction due to leaky expression, but full rescue required induction.

Fig. S12. Approximate copy numbers of retroelements in budding yeast species. Copy numbers were estimated by TBLASTN searches using the Gag-Pol polyprotein as a search query. Intact genes and pseudogenes were counted, but not solo LTRs. S. castellii and K. polysporus have many more Ty3/gypsy elements (18 and 24 elements, respectively) than those budding yeast species that have lost the RNAi pathway (0-3 elements). Most notably, a subfamily of gypsy elements more similar to C. albicans Tca3 (30) than to S. cerevisiae Ty3 is found exclusively in species that have retained the RNAi pathway: S. castellii and K. polysporus, as well as several Candida species. The two gypsy subfamilies have been proposed to have different mechanisms for priming minus-strand RNA synthesis (30). As in C. albicans, many of the members of the gypsy families in S. castellii and K. polysporus appear to be structurally rearranged. It is possible that selection has favored the retention of these structures as templates for defensive siRNA production.

### DETAILED DESCRIPTION OF CERTAIN EMBODIMENTS OF THE INVENTION

The following non-limiting definitions are provided here for convenience. Art-accepted abbreviations are used herein.

“About” in reference to a numerical value generally refers to a range of values that fall within ±10%, in some embodiments ±5%, in some embodiments ±1%, in some embodiments ±0.5% of the value unless otherwise stated or otherwise evident from the context.

“Antibody” as used herein refers to immunoglobulin molecules or portions thereof capable of specifically binding to an antigen. An antibody can be polyclonal or monoclonal. Antibodies or purified fragments having an antigen binding region, e.g., fragments such as Fv, Fab′, F(ab′)2, Fab fragments, single chain antibodies (which typically include the variable regions of the heavy and light chains of an immunoglobulin, linked together with a short (usually serine, glycine) linker, chimeric or humanized antibodies, and complementarily determining regions (CDR) may be identified and prepared by conventional procedures. An antibody could be of mammalian, e.g., rodent (e.g., murine), or avian (e.g., chicken) origin and could be of any of the various immunoglobulin classes or subclasses (e.g., IgG, IgM).

An “expression control element” as used herein can be any regulatory nucleotide sequence, such as a promoter sequence or promoter-enhancer combination, that facilitates the expression of a nucleic acid. The expression control element may, for example, be a yeast, bacterial, mammalian or viral (e.g., phage) promoter. An expression control element, e.g., promoter, can be constitutive or conditional, e.g., regulatable (e.g., inducible or repressible). Inducible promoters direct expression in the presence of an inducing agent (e.g., an appropriate small molecule) or inducing condition (e.g., increased temperature), while in the absence of such agent or condition expression is usually much lower or undetectable above background. In some embodiments the promoter is titratable, e.g., the level of expression can be regulated by varying the concentration of an inducing or repressing agent. For example, a higher concentration of inducing agent typically results in higher expression level. It will be understood that induction in some instances may be achieved by relieving repression. Tetracycline controlled transcriptional activation is a method of inducible expression where transcription is reversibly turned on or off in the presence of the antibiotic tetracycline or a derivative (e.g., doxycycline). Two “Tet” systems (Tet-off and Tet-on) are widely used. Expression control elements capable of directing transcription in cells are known in the art. Exemplary expression control elements are mentioned herein. In some embodiments of the invention, transcription of a sequence of interest can be irreversibly turned on or off using the Cre/Lox or Flp/FRT recombinase system. For example, a nucleic acid “stuffer sequence” can be positioned between sites for a recombinase. Delivering the recombinase to a cell (e.g., by expressing it therein or by introducing it from outside the cell), results in excision of the stuffer sequence. Such excision can bring an expression control element, e.g., a promoter, into operable association with a nucleic acid segment of interest, resulting in its transcription.

“Identity” refers to the extent to which the sequence of two or more nucleic acids or polypeptides is the same. The percent identity between a sequence of interest A and a second sequence B may be computed by aligning the sequences, allowing the introduction of gaps to maximize identity, determining the number of residues (nucleotides or amino acids) that are opposite an identical residue, dividing by the minimum of TG_{A} and TG_{B} (here TGA and TGB are the sum of the number of residues and internal gap positions in sequences A and B in the alignment), and multiplying by 100. When computing the number of identical residues needed to achieve a particular percent identity, fractions are to be rounded to the nearest whole number. Sequences can be aligned with the use of a variety of computer programs known in the art. For example, computer programs such as BLAST2, BLASTN, BLASTP, Gapped BLAST, etc., generate alignments. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and)(BLAST programs of Altschul et al. (Altschul, et al., J. MoI. Biol. 215:403-410, 1990). In some embodiments, to obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. See the Web site having URL www.ncbi.nlm.nih.gov. Other suitable programs include CLUSTALW (Thompson J D, Higgins D G, Gibson T J, Nuc Ac Res, 22:4673-4680, 1994) and GAP (GCG Version 9.1; which implements the Needleman & Wunsch, 1970 algorithm (Needleman S B, Wunsch C D, J Mol Biol, 48:443-453, 1970.)

As used herein, “non-endogenous” refers to genes, molecules, pathways, processes, that are not naturally found in a particular context, e.g., in or associated with a cell or organism. For example, a “non-endogenous” nucleic acid could be derived at least in part from a different organism or could be at least in part invented by man and not found in nature. “Non-endogenous” can include modifying an endogenous molecule. For example, homologous recombination could be used to modify an endogenous gene (e.g., alter its sequence), with resulting gene being considered “non-endogenous”. “Non-endogenous” also encompasses introducing a nucleic acid that has the same sequence as an endogenous nucleic acid into a cell, wherein said introduction genetically modifies the recipient cell. For example, the introduced nucleic acid may be joined to a nucleic acid to which it is not joined in nature, e.g., an expression control element, or integrated into the genome in a position in which it is not found in nature.

As used herein, the term “nucleic acid” is used to mean one or more nucleotides, i.e. a molecule comprising a sugar (e.g., ribose or deoxyribose) linked to a phosphate group and organic base, which may be a substituted pyrimidine (e.g. cytosine (C), thymidine (T) or uracil (U)) or a substituted purine (e.g. adenine (A) or guanine (G)). The term “nucleic acid” is used interchangeably with “polynucleotide” or “oligonucleotide” as those terms are ordinarily used in the art, i.e., polymers of nucleotides, where oligonucleotides are generally shorter in length than polynucleotides (e.g., 60 nucleotides or less). A series of nucleotides bonded together, i.e., within a polynucleotide or an oligonucleotide can be referred to as a “nucleic acid sequence” or “nucleotide sequence”, and the nucleotide subunits are typically indicated using the abbreviation of the base, e.g., A, G, C, T, U. Where the present invention provides a nucleotide sequence, it is understood that the complementary sequence is also provided, and both single- and double-stranded forms are provided. Purines and pyrimidines include, but are not limited to, natural nucleosides (for example, adenosine, thymidine, guanosine, cytidine, uridine, deoxyadenosine, deoxythymidine, deoxyguanosine and deoxycytidine), nucleoside analogs, chemically or biologically modified bases (for example, methylated bases), modified sugars (2′-fluororibose, arabinose, or hexose), modified phosphate groups (for example, phosphorothioates or 5′-N-phosphoramidite linkages), and other naturally and non-naturally occurring nucleobases, including substituted and unsubstituted aromatic moieties. Other modifications are well-known to those of skill in the art. In some embodiments a nucleic acid comprises non-nucleotide material, such as at the end(s) or internally (at one or more nucleotides). A nucleic acid can be single-stranded, double-stranded, or partially double-stranded. In some embodiments a nucleic acid is composed of RNA. In some embodiments a nucleic acid is composed of DNA. In various embodiments a double-stranded nucleic acid may have one or more overhangs (5′ and/or 3′ overhangs). In some embodiments a nucleic acid comprises standard nucleotides (A, G, C, T, U). In other embodiments a nucleic acid comprises one or more non-standard nucleotides. In some embodiments, one or more nucleotides are non-naturally occurring. A nucleic acid may comprise a detectable label, e.g., a fluorescent dye.

A “polypeptide” refers to a polymer of amino acids. A protein is a molecule comprising one or more polypeptides. A peptide is a relatively short polypeptide, typically between about 2 and 60 amino acids in length. The terms “protein”, “polypeptide”, and “peptide” may be used interchangeably. Polypeptides of interest herein typically contain standard amino acids (the 20 L-amino acids that are most commonly found in nature in proteins). However, other amino acids and/or amino acid analogs known in the art can be used in certain embodiments of the invention. One or more of the amino acids in a polypeptide may be modified, for example, by addition, e.g., covalent linkage, of a non-peptide moiety, such as a carbohydrate group, a phosphate group, a linker for conjugation, etc. A polypeptide sequence presented herein is presented in an N-terminal to C-terminal direction unless otherwise indicated. “Polypeptide domain” refers to a segment of amino acids within a longer polypeptide. A polypeptide domain may exhibit one or more discrete binding or functional properties, e.g., a catalytic activity. Often a domain is recognizable by its conservation among polypeptides found in multiple different species.

“Purified” or “substantially purified” may be used herein to refer to an isolated nucleic acid or polypeptide that is present in the substantial absence of other biological macromolecules, e.g., other nucleic acids and/or polypeptides. In some embodiments a purified nucleic acid (or nucleic acids) is substantially separated from cellular polypeptides. In some embodiments, the ratio of nucleic acid to polypeptide is at least 5:1 or at least 10:1 by dry weight. In some embodiments a purified polypeptide is separated from cellular nucleic acids. In some embodiments, the ratio of nucleic acid to polypeptide is at least 5:1 or at least 10:1 by dry weight. In some embodiments, a nucleic acid or polypeptide is purified such that it constitutes at least 75%, 80%, 85%, or 90% by weight, e.g., at least 95% by weight, e.g., at least 99% by weight, or more, of the total nucleic acid or polypeptide material present. In some embodiments, water, buffers, ions, and/or small molecules (e.g., precursors such as nucleotides or amino acids), can optionally be present in a purified preparation. A purified molecule may be prepared by separating it from other substances (e.g., other cellular materials), or by producing it in such a manner to achieve purity. In some embodiments, a purified molecule or composition refers to a molecule or composition comprising one or more molecules, that is prepared using any art-accepted method of purification. In some embodiments “partially purified” means that a molecule produced by a cell is no longer present within the cell, e.g., the cell has been lysed and, optionally, at least some of the cellular material (e.g., cell wall, cell membrane(s), cell organelle(s)) has been removed.

A “variant” of a particular polypeptide or polynucleotide has one or more alterations (e.g., amino acid or nucleotide additions, substitutions, and/or deletions, which may be referred to collectively as “mutations”) with respect to the polypeptide or polynucleotide, which may be referred to as the “original polypeptide or polynucleotide”. Thus a variant can be shorter or longer than the polypeptide or polynucleotide of which it is a variant. In some embodiments a “variant” comprises a “fragment”. The term “fragment” refers to a portion of a polynucleotide or polypeptide that is shorter than the original polynucleotide or polypeptide. In certain embodiments of the invention a variant comprises a portion that has at least 50%, 60%, 70%, 75%, 80%, 85%, 90%, 95%, or more sequence identity to the original polypeptide or polynucleotide over a portion of the original polypeptide or polynucleotide having a length at least 20%, 30%, 40%, 50%, 60%, 70%, 80%, 90%, 95%, or more, of the length of the original polypeptide or polynucleotide. In a non-limiting embodiment a variant polypeptide has at least 80%, 90%, 95%, 96%, 97%, 98%, or 99% identity to the original polypeptide over a portion of the original polypeptide having a length at least 100 amino acids. In a non-limiting embodiment a variant polypeptide has at least 80%, 90%, 95%, 96%, 97%, 98%, or 99% identity to the original polypeptide over a functional domain of the original polypeptide. In some embodiments, a variant polynucleotide or polypeptide is generated using recombinant DNA techniques. In some embodiments amino acid “substitutions” replace one amino acid with another amino acid having similar structural and/or chemical properties, e.g., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Insertions or deletions may range in size from about 1 to 20 amino acids, e.g., 1 to 10 amino acids. In some instances larger domains may be removed without substantially affecting function. In certain embodiments, the sequence of a variant can be obtained by making no more than a total of 5, 10, 15, or 20 amino acid additions, deletions, or substitutions to the sequence of a naturally occurring enzyme. In some embodiments, not more than 1%, 5%, 10%, or 20% of the amino acids in a polypeptide are insertions, deletions, or substitutions relative to the original polypeptide. Guidance in determining which amino acid residues may be replaced, added, or deleted without eliminating or substantially reducing an activity of interest, may be obtained, e.g., by aligning and comparing the sequence of the particular polypeptide with that of homologous functional polypeptides (e.g., orthologs from other organisms). One of skill in the art will be aware that amino acid residues that are conserved among various species are, in general, more likely to be important for activity than amino acids that are not conserved.

“Isolated” as used herein refers to a molecule, e.g., a nucleic acid or polypeptide, separated from at least some other components (e.g., nucleic acid or polypeptide) that are present with the nucleic acid or polypeptide as found in its natural source (or a molecule produced from such an isolated molecule) and/or a molecule prepared at least in part by the hand of man. In some embodiments an isolated nucleic acid or polypeptide is at least in part synthesized using recombinant DNA technology, e.g., using in vitro transcription or translation, respectively, or an isolated nucleic acid sequence is synthesized using amplification (e.g., PCR). In some embodiments an isolated nucleic acid or polypeptide is chemically synthesized. In some embodiments, an isolated nucleic acid is removed from its genomic context. In some embodiments, an isolated nucleic acid is joined to a nucleic acid to which it is not joined in nature. For example, an isolated nucleic acid may be joined to a sequence comprising an expression control element to which the nucleic acid is not operably linked in nature. In some embodiments, an isolated nucleic acid is present in a vector which, in some embodiments, is not a sequencing vector. “Isolated” can also refer to a cell that is removed from its natural habitat, e.g., a cell maintained in a laboratory, e.g., in culture, or a descendant of the cell.

As used herein, the term “selectable marker” (sometimes termed “marker” herein) typically refers to a gene that encodes an enzymatic or other activity that confers on a cell the ability to grow in medium lacking what would otherwise be an essential nutrient or confers resistance to an antibiotic or drug upon the cell in which the selectable marker is expressed or otherwise renders a cell specifically detectable or selectable. The term “selectable marker” can also refer to the gene product itself. In some embodiments expression of a selectable marker by a cell confers a significant growth or survival advantage on the cell (relative to cells not expressing the marker) under certain defined culture conditions (selective conditions) such that maintaining the cell under such conditions allows the identification (and optionally the isolation) or elimination of cells that express the marker. Antibiotic resistance markers include genes encoding enzymes that provide resistance to neomycin, zeocin, hygromycin, kanamycin, puromycin, chloramphenicol, etc. A second non-limiting class of selectable markers is nutritional markers. Such markers are generally enzymes that function in a biosynthetic pathway to produce a compound that is needed for cell growth or survival. Examples in yeast include enzymes that participate in biosynthetic pathways for synthesis of amino acids such as uracil, leucine, histidine, tryptophan, etc. It will be appreciated that selectable markers encompass those in which negative selection is employed. Optically detectable molecules, e.g., fluorescent or luminescent proteins, are another class of marker, sometimes termed “detectable marker”. Enzymes with a readily assayed activity such as alkaline phosphatase (AP), beta galactosidase (LacZ), beta glucoronidase (GUS), chloramphenicol acetyltransferase (CAT), horseradish peroxidase (HRP), luciferase (Luc) can also be used. Such genes can also be used as reporters or controls, e.g., to assess the efficiency of RNAi-mediated silencing.

As used herein, a first sequence is “substantially complementary” to a second sequence if at least 75% of the nucleotides in the two sequences are capable of forming hydrogen bonded base pairs (bp) with oppositely located nucleotides (i.e., a nucleotide is capable of base pairing with a nucleotide located at the opposite position in the other strand) when the sequences are aligned in opposite orientation. In some embodiments, the two sequences are at least 80%, 85%, 90%, 95%, 96%, 97%, 98%, 99%, or 100% complementary. As known in the art, in the canonical Watson-Crick base pairing, adenine (A) forms a base pair with thymine (T), as does guanine (G) with cytosine (C) in DNA. In RNA, thymine is replaced by uracil (U). Non-Watson-Crick base pairing with alternate hydrogen bonding patterns also occur, especially in RNA; common among such patterns are Hoogsteen base pairs and wobble base pairs. In some embodiments of the invention, a dsRNA or siRNA comprises only Watson-Crick base pairs, while in other embodiments at least some of the base pairs are non-Watson-Crick base pairs.

A “small interfering RNA” or “siRNA” as used herein, refers in some embodiments to an RNA molecule derived from the successive cleavage of longer double-stranded RNA (dsRNA), e.g., within a cell by an enzyme comprising an RNase III domain, to produce an RNA molecule composed of two at least substantially complementary strands generally having a length of between 15 and 30 nucleotides, and more often between 20 and 25 nucleotides, e.g., 20, 21, 22, 23, 24, or 25 nucleotides, wherein each strand typically comprises a 5′ phosphate group and a 3′ hydroxyl (—OH) group. Naturally occurring siRNAs typically comprise a duplex structure between about 18 and 23 base pairs (bp) long, e.g., 18, 19, 20, 21, 22, 23 by long. Often the portions of the strands that form the duplex are perfectly (100% complementary), but in some embodiments the strands of the duplex are, e.g., at least 80%, 90%, or 95% complementary, e.g., the duplex comprises between 1-5 mismatches, e.g., 1, 2, 3, 4, 5 mismatches (referring to a pair of nucleotides located opposite one another that do not form a base pair) or bulges, which mismatches or bulges may be located, e.g., near one or both ends of the duplex. The term “siRNA” also encompasses molecules of similar structure that are generated extracellularly, e.g., in a cell extract, in a composition comprising an isolated Dicer polypeptide, or using chemical synthesis. Such siRNAs, e.g., those generated using chemical synthesis, can comprise a variety of different nucleotides and internucleoside linkages, as known in the art. siRNAs can be blunt-ended or have overhangs, e.g., 3′ overhangs. In some embodiments an overhang is from 1-10 nucleotides in length, e.g., 1, 2, 3, 4, or 5 nucleotides long, e.g., 2 nucleotides long. One of skill in the art will be aware of various approaches to generating synthetic siRNAs that have, for example, increased resistance to degradation. In some embodiments, one or more nucleotides at the 3′ end of an siRNA, e.g., 2 nucleotides, is/are deoxyribonucleotide(s), e.g., dT.

“Transfection” refers to the introduction of a nucleic acid into a cell. The term is intended to encompass nucleic acid transfer into prokaryotic (e.g., bacterial), fungal, and plant cells (sometimes termed “transformation”). Cells may be transiently or stably transfected. Stable cell lines can be generated using standard selection methods. A cell has been “stably transfected” with a nucleic acid construct when the nucleic acid construct is capable of being inherited by daughter cells over many generations, e.g., is integrated into the genome of the cell. “Transient transfection” refers to cases where exogenous nucleic acid does not integrate into the genome of a transfected cell and is progressively lost as cells divide.

A “vector” as used herein, refers to a nucleic acid or a virus or portion thereof (e.g., a viral capsid) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication) in a cell and/or may include sequences sufficient to allow integration of part or all of the nucleic acid into host cell DNA. Useful nucleic acid vectors include, for example, DNA or RNA plasmids, cosmids, artificial chromosomes, and naturally occurring or modified viral genomes or portions thereof or nucleic acids (DNA or RNA) that can be packaged into viral capsids. Vectors often include one or more selectable markers. “Expression vectors” typically include regulatory sequence(s), e.g., expression control sequences such as a promoter, sufficient to direct transcription of an operably linked nucleic acid. An expression vector often comprises sufficient cis-acting elements for expression; other elements for expression can be supplied by the host cell or in vitro expression system. Vectors often include one or more appropriately positioned sites for restriction enzymes, e.g., to facilitate introduction of the nucleic acid to be transported or expressed into the vector.

RNAi in Budding Yeast

RNA interference (RNAi) and related RNA-silencing pathways contribute to transposon silencing, viral defense, DNA elimination, heterochromatin formation, and posttranscriptional repression of cellular genes (1, 2). These pathways produce short (21-30-nt) guide RNAs that are loaded into a protein of the Argonaute/Piwi family, where they pair with target transcripts to direct silencing of specific mRNAs or genomic regions (3). Forms of RNA silencing are found in plants, animals, fungi, and protists, suggesting origins in an early eukaryotic ancestor. In RNAi, the RNaseIII endonuclease Dicer successively cleaves double-stranded RNA (dsRNA) into siRNAs, which are loaded into the effector protein Argonaute to guide the silencing of target transcripts. Silencing (also termed “inhibition”) is sequence-specific in that genes corresponding in sequence to the duplex (base-paired) region of the RNA (dsRNA or siRNA) are targeted for inhibition. As well known in the art, 100% sequence identity between a siRNA or dsRNA and the target gene is not required for silencing, provided that the correspondence is sufficient to enable the siRNA (or siRNAs derived by cleavage of the dsRNA) to direct silencing of the mRNA, e.g., to direct RNAi cleavage of the target mRNA by the RNAi machinery. See, e.g., U.S. Pat. No. 6,506,559 and U.S. Ser. No. 09/821,832. As used herein, a gene or mRNA whose expression is silenced by RNAi is said to be “targeted” and may be referred to as a “target gene” or “target mRNA”, and the siRNA that mediates such silencing is said to “target” the gene or mRNA.

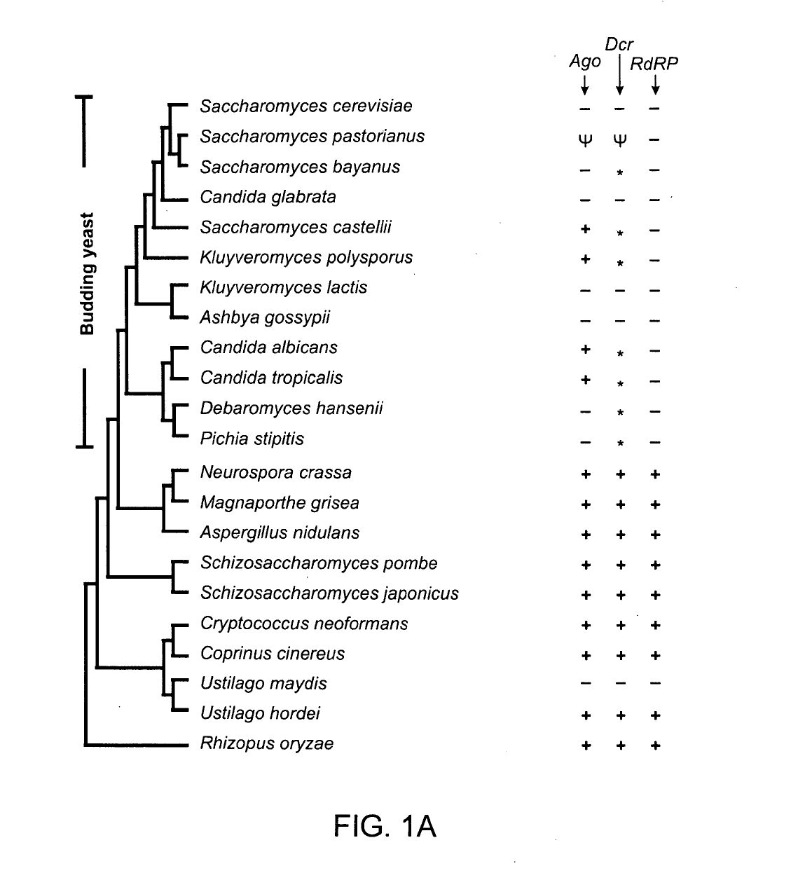

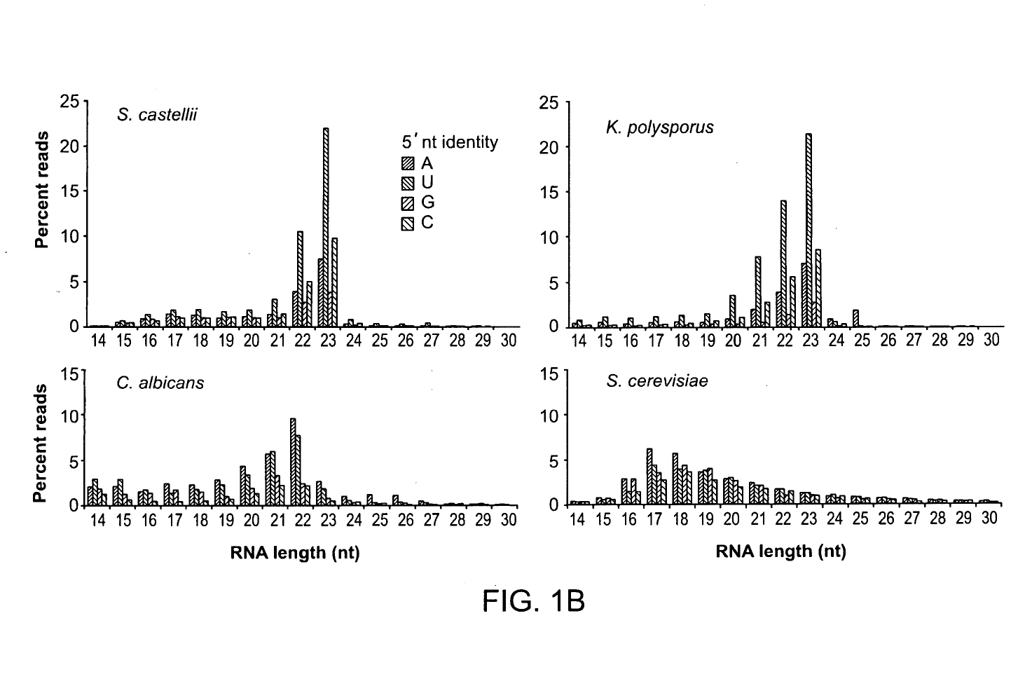

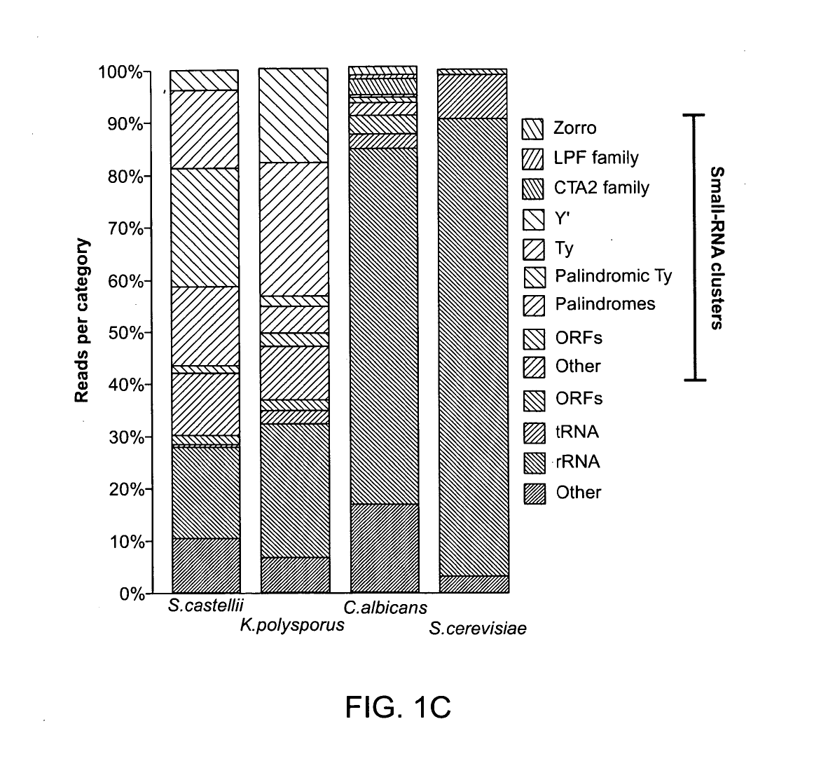

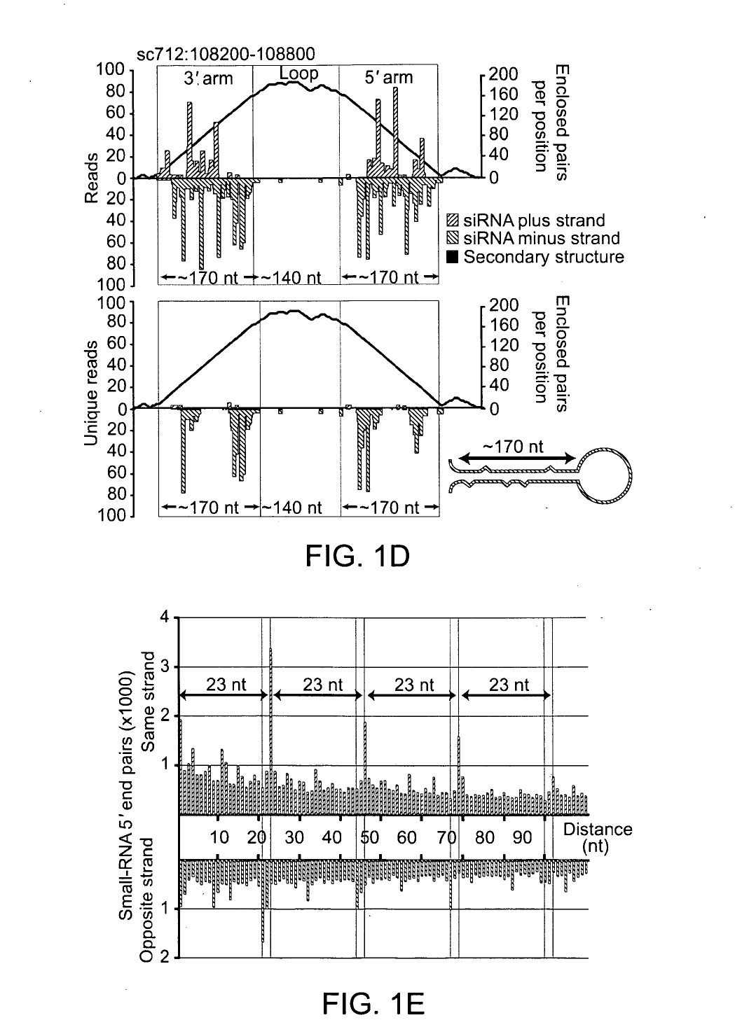

Some of the earliest reports of RNA silencing were in fungi. In Neurospora crassa, posttranscriptional gene silencing known as quelling involves the classic endogenous RNAi pathway (4). In the fission yeast Schizosaccharomyces pombe, the pathway helps establish and maintain heterochromatin (5). RNA silencing appears to be conserved throughout most of the fungal kingdom (6, 7) as indicated by the presence of genes for Argonaute, Dicer, and an RNA-dependent RNA polymerase (RdRP), which in some RNAi pathways, including those of N. crassa and S. pombe, produces dsRNA (FIG. 1A). A prominent exception is the budding yeast Saccharomyces cerevisiae, which lacks recognizable homologs of Argonaute, Dicer, and RdRP. Indeed, prior to the present invention, RNAi had been presumed lost in all budding yeasts.

The present invention encompasses the recognition that a functional RNAi pathway exists in certain budding yeast. The invention also encompasses the recognition that a functional RNAi pathway can be reconstituted using genetic engineering in budding yeast that lack an endogenous functional RNAi pathway. As described in Examples 1 and 2, it was discovered that short RNAs with lengths and chemical features consistent with Dicer products exist in a variety of budding yeast species, and that cells extracts derived from these species contained an activity that produced 22-23-nt RNAs from dsRNAs added to such extracts (see Examples 1 and 2). Although these yeast lack genes with the domain architecture of known Dicer proteins, a gene encoding a candidate Dicer polypeptide containing an RNase III domain was identified in S. castellii. siRNA failed to accumulate in a strain in which this gene was deleted, while accumulation was restored by ectopic expression, thereby confirming the protein as being a functional Dicer. Furthermore, orthologs of S. castellii Dicer were identified in each of the other Argonaute-containing budding yeasts analyzed. These species use noncanonical Dicer proteins to generate small interfering RNAs (siRNAs), most of which correspond to transposable elements and Y′ subtelomeric repeats. In S. castellii, RNAi mutants are viable but have excess Y′ mRNA levels. In S. cerevisiae, introducing Dicer and Argonaute of S. castellii reconstitutes RNAi, and the reconstituted pathway silences endogenous retrotransposons. Among other things, these results expand the definition of Dicer, bring the tool of RNAi to the study of budding yeasts, and bring the tools of budding yeast to the study of RNAi.

Accordingly, the invention relates to the discovery of a functional RNAi pathway in budding yeast. Existence of an endogenous “functional RNAi pathway” in a budding yeast can be evidenced, in some embodiments of the invention, by one or more, e.g., all, of the following: (i) presence in the budding yeast of short RNAs having a characteristic structure indicative of cleavage of a dsRNA by an RNase III enzyme, such RNAs being distinct from other cellular short RNA species in their abundance and/or structural features; (ii) appearance of non-endogenous short RNA having the structure of siRNAs when a yeast cell is genetically engineered to express a dsRNA (such siRNAs resulting from cleavage of the dsRNA); (iii) a change in steady state level of an mRNA and/or its encoded protein in the yeast cell when the cell is genetically engineered to express a dsRNA that corresponds to a gene that is transcribed to yield mRNA in the cell.

The invention also relates to functional budding yeast Dicer genes and polypeptides. A “functional” Dicer polypeptide is capable of cleaving a dsRNA to yield siRNAs under appropriate conditions, e.g., within a cell in which it is naturally found and optionally, in at least some embodiments, in a cell in which its expression is achieved by genetic engineering and/or, in at least some embodiments, in vitro. Interestingly, in budding yeasts, Dicer has two double-stranded RNA binding domains (dsRBDs) but only a single RNaseIII domain and no helicase or PAZ domains, whereas in other fungi, known Dicer genes resemble those in plants and animals, encoding proteins with tandem RNaseIII domains, 2-3 dsRBDs, a PAZ domain, and an N-terminal helicase domain. Furthermore, budding yeast Dicer appears not to require cofactors containing one or more dsRBDs for activity. The invention thus provides functional Dicer polypeptides that contain only a single RNase III domain and/or that lack a PAZ domain or an N-terminal helicase domain and/or that function without requiring additional dsRBD-containing co-factors. The invention also relates to the functional budding yeast Argonaute genes and polypeptides. A “functional” Argonaute polypeptide is capable of binding at least the guide strand of an siRNA (also termed the “antisense strand”) and has endonuclease activity directed against mRNA strands that are complementary to a the guide strand of a bound siRNA under appropriate conditions, e.g., within a cell in which it is naturally found and optionally, in at least some embodiments, in a cell in which its expression is achieved by genetic engineering. Dicer as used herein includes non-endogenous Dicer and endogenous Dicer.

Certain methods of the invention comprise delivering an siRNA to a cell of interest, e.g., a budding yeast cell. “Delivering” as used herein in reference to an siRNA, encompasses making an siRNA available within a cell using any suitable method. In some embodiments, “delivery” refers to introducing a nucleic acid that can be transcribed to yield an siRNA precursor, e.g., a dsRNA, into a cell, and maintaining the cell under conditions in which the siRNA precursor is expressed and cleaved to yield siRNA. If the nucleic acid is under control of an inducible expression control element, such maintaining could comprise maintaining the cell under inducing conditions. It will be appreciated that “delivery” to a cell of interest encompasses introducing a nucleic acid into an ancestor of the cell of interest in a manner such that the nucleic acid is inherited by daughter cells. In some embodiments, “delivery” refers to contacting a cell with an siRNA. In some embodiments, “delivery” refers to introducing an siRNA precursor, e.g., a dsRNA, into a cell, and maintaining the cell under conditions in which the siRNA precursor is cleaved to yield siRNA.

In some embodiments of the invention, RNAi in budding yeast involves intracellular cleavage of an siRNA precursor, e.g., a dsRNA, by a functional budding yeast Dicer, to yield siRNA. The siRNA precursor, e.g., dsRNA, can be endogenous to (i.e., “native to”) the yeast cell or can be a non-endogenous dsRNA whose expression in the cell is achieved by genetic engineering of the cell or an ancestor of the cell. Thus siRNA can be delivered to a budding yeast cell (or other cell) by engineering the cell to express an siRNA precursor, e.g., a dsRNA. Any siRNA precursor, e.g., any dsRNA can be used, provided that it has sufficient homology to the targeted gene such that the resulting siRNAs direct silencing by RNAi, e.g., to direct degradation of an mRNA of the gene to which it corresponds. In many embodiments, the sequence of the siRNA precursor, e.g., dsRNA, is selected to correspond to a known sequence, such as a portion of an mRNA of a gene, or the entire mRNA of a gene whose silencing is desired. For example, the dsRNA can comprise a double-stranded portion at least 15 by long that corresponds to mRNA of the gene. In some embodiments, a dsRNA comprises a double-stranded portion at least 20, 25, 40, 50, 100, 200, 300, 400, 500 by long. In some embodiments the dsRNA comprises a longer duplex region, e.g., up to 1, 2, or 3 kbp long, or more. In some embodiments a dsRNA comprises a duplex portion that corresponds to at least 25%, 50%, 75%, 90%, up to 100% of the length of a targeted mRNA. As mentioned above, not all of the nucleotides in the double-stranded portion need to participate in base pairs. The strands of the double-stranded portion can be substantially complementary. For example, there can be about 75%, 80%, 85%, 90%, 95%, or 100% complementarity. Thus “base pairs” as used in reference to a duplex refers to the number of nucleotides between the first and last base pairs of a duplex, and does not imply that all nucleotides are paired. In many embodiments, the lengths of the two sequences that form the duplex portion are the same or about the same. In embodiments in which the two strand portions that form a duplex have different numbers of nucleotides (e.g., resulting in a bulge) an average of the lengths of the two portions can be used. It will be understood that the dsRNA in some embodiments may comprise multiple duplex portions of any of the afore-mentioned lengths, which portions may be separated, e.g., by regions with few or no base pairs, or wherein a strand contains a large unpaired “bulge” separating two portions of the strand that are base paired with the opposite strand. In some embodiments, a dsRNA contains duplex portions having correspondence to 2 or more different genes, e.g., 3, 4, or 5 genes so that the dsRNA is cleaved to form siRNAs that target multiple different genes for silencing. In some embodiments, a dsRNA comprises multimers of a particular sequence that corresponds to a portion of a gene or mRNA. For example, such multimers may be between 20 and 200 nucleotides long. In some embodiments, the sequence of the dsRNA is selected so that only one or a few (e.g., 2, 3, or 4) different siRNA species are produced. In some embodiments, a yeast cell is engineered to express multiple dsRNAs, so that multiple genes can be silenced by siRNAs derived by cleavage of the dsRNA. As noted, above, there need not be 100% correspondence between the dsRNA duplex and the targeted gene or mRNA. For example, such correspondence could be at least 70%, 75%, 80%, 85%, 90%, 95%, or more, up to 100%. It will be understood that the duplex could also comprise nucleic acids that do not correspond to a gene whose silencing is desired.

In some embodiments, the dsRNA is a single RNA strand that comprises two portions that are complementary to each other (i.e., the strand is self-complementary) and hybridize to form a double-stranded structure referred to in the art as a “hairpin”. The end of the hairpin may be blunt or may have an overhang that extends beyond the double-stranded portion. The hairpin may comprise a single-stranded “loop” comprising at least 1 unpaired nucleotide up to, e.g., about 5, 10, 20, 50, 70, 100, 150, 200, or more nucleotides. In some embodiments the loop comprises an intron, which may be spliced out when the dsRNA is expressed in a cell. The intron need not originate from the same organism in which the dsRNA is to be expressed. In some embodiments, the dsRNA is in the form of two separate, complementary strands. The strands can have the same length or different lengths. Each of the ends of the dsRNA may be blunt or either or both ends may have an overhang.

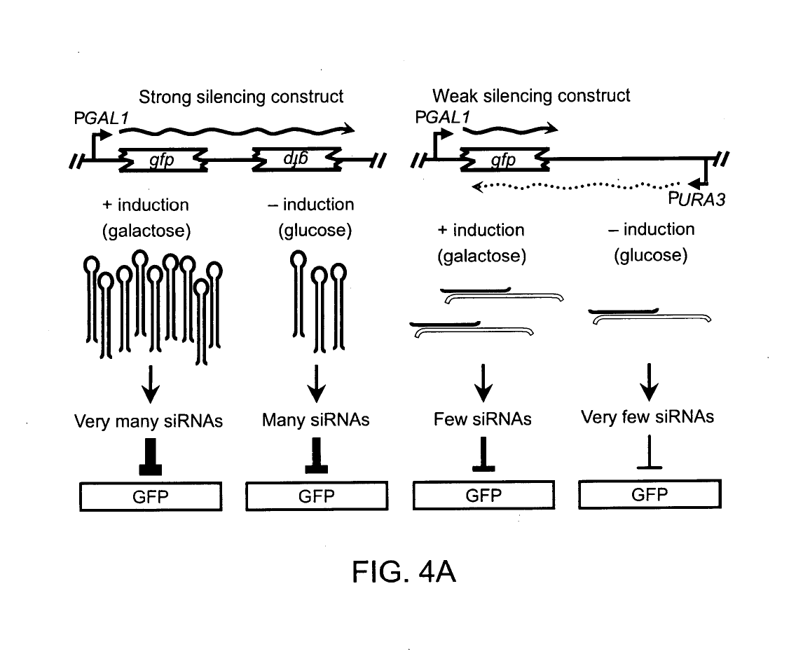

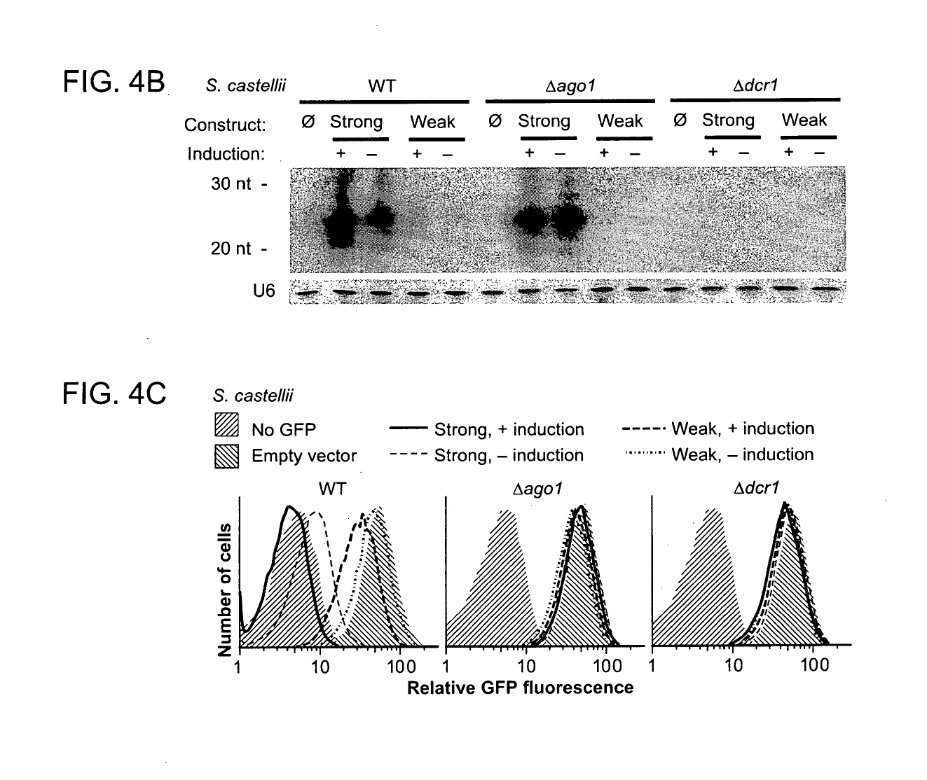

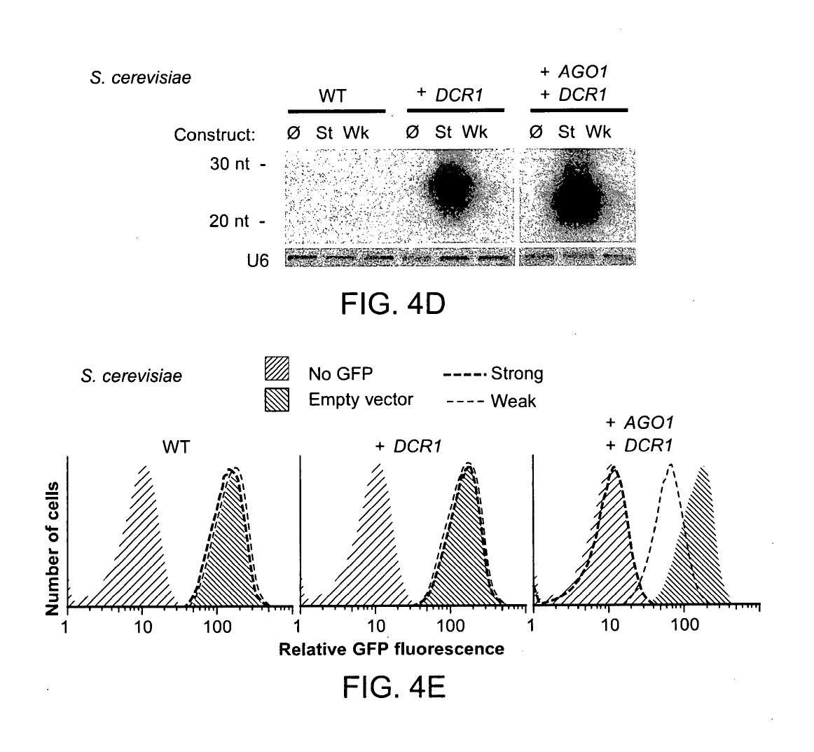

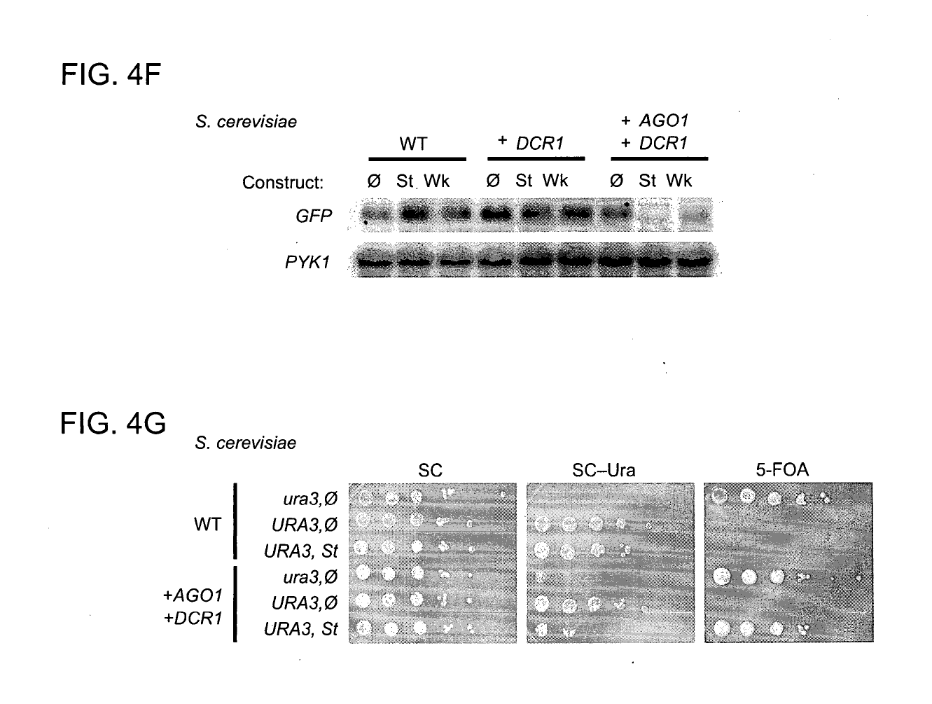

A dsRNA formed by self-hybridization can be transcribed from a single expression control element, e.g., promoter. For example, a DNA sequence may be cloned in the sense orientation and in the antisense orientation downstream of, and operably linked to, an expression control element (see, e.g., FIG. 4A showing inverted repeat of GFP sequence operably linked to a GAL1 promoter). In other embodiments, a dsRNA formed from two separate strands is produced by convergent transcription directed by expression control elements flanking a DNA sequence to be transcribed to RNA, wherein the promoters are oriented in opposite orientation so that both strands of the intervening sequence are used as templates. In another embodiment, two separate templates in opposite orientation, each operably linked to an expression control element, are provided, so that transcription yields complementary sense and antisense transcripts. The templates may, but need not be, introduced into the cell using a single nucleic acid, e.g., vector. In some embodiments, a nucleic acid that provides a template for transcription of a dsRNA is integrated into the genome of a cell. In some embodiments, a nucleic acid that provides a template for transcription of a dsRNA is provided on an episome that is maintained extrachromosomally. The invention thus provides methods that comprise generating a budding yeast cell capable of expressing an siRNA precursor, e.g., dsRNA. Also provided are nucleic acids comprising a template for transcription of a dsRNA is operably linked to an expression control element capable of directing transcription in a yeast cell. In some embodiments the dsRNA corresponds to an endogenous gene of a budding yeast. In other embodiments the dsRNA corresponds to a heterologous gene, which may be a gene that a yeast is genetically engineered to express. The invention further provides collections of nucleic acids that comprise templates for transcription of a multiplicity of dsRNA, the dsRNAs corresponding to at least 10 genes of a budding yeast, e.g., S. cerevesiae. In some embodiments the collection comprises nucleic acids that comprise templates for transcription of dsRNAs corresponding to at least 20, 50, 100, 500, 1000, 2000, 3000, 4000, 5000, 6000, or more genes. In some embodiments, each template is provided as part of a separate nucleic acid, e.g., a vector. In some embodiments two or more templates are provided as part of a single nucleic acid. In some embodiments the collection comprises dsRNAs corresponding to at least 10%, 20%, 50%, 75%, 90%, 95%, 98%, 99%, or 100% of the genes of a budding yeast, e.g., S. cerevesiae.

In other embodiments, siRNA is delivered to a cell, e.g., a budding yeast cell, by contacting the cell with siRNA or longer dsRNA externally (e.g., in a liquid medium), wherein the RNA is taken up by the cell. Any method known in the art for causing nucleic acid uptake by a yeast cell may be used. In some embodiments, a yeast cell may have a cell wall that has increased permeability relative to a normal yeast cell, or the cell wall may be removed (e.g., by spheroplasting). For example, the yeast cell may have a mutation that renders it defective in cell wall synthesis or may be treated with an agent that weakens the cell wall, creates holes in it, or inhibits synthesis of a cell wall component. In some embodiments electroporation is used to deliver siRNA to a cell.

In some embodiments, the extent of inhibition of a gene by RNAi in accordance with the invention, e.g., in a budding yeast cell that has an endogenous functional RNAi pathway or a cell that is genetically engineered to have a functional RNAi pathway using functional budding yeast RNAi pathway gene(s), is at least 10%, 25%, 33%, 50%, 60%, 75%, 80%, 85%, 90%, 95% or 99% as compared to a control cell (e.g., a comparable cell in which the endogenous RNAi pathway is non-functional, has been disabled by mutating an RNAi pathway gene such as Dicer, or in which a dsRNA targeted to the gene is not expressed). In some embodiments expression is reduced to background levels and/or is undetectable. The extent of inhibition can be controlled in a variety of ways. For example, the strength of the expression control element, e.g., promoter, directing expression of a dsRNA, and its position relative to the start site, can be varied. In embodiments in which a regulatable promoter is used, the concentration of inducing agent (or extent to which the inducing condition is present), can be varied to control the amount of dsRNA produced. This allows the extent of silencing to be modulated while cells are being cultured. Also, in general, embodiments in which a dsRNA hairpin is formed by hybridization of self-complementary portions of a single RNA may provide stronger silencing than embodiments in which two separate strands are transcribed. Varying the length of the dsRNA and/or its degree of correspondence to the target mRNA can result in different degrees of silencing, e.g., the mRNA can be degraded to different extents. In some embodiments, a “weak” silencing construct is generated in which a low level of transcription of one strand occurs due to read-through from a promoter operably linked to a different gene. The invention provides sets of isogenic budding yeast strains in which a gene of interest is silenced to varying extents. For example, a set may comprise two or more strains, wherein the extent of silencing differs by a factor of 2-fold, 5-fold, 10-fold, or more, among different members of the set. Thus strains having a gradient of silencing levels can be provided.

The level of gene expression and its inhibition may be quantified at the level of accumulation of target mRNA or translated protein. For example, the extent of inhibition may be determined by assessing the amount of gene product in the cell. Standard methods for such quantation can be used, e.g., hybridization or amplification-based methods can be used for RNA, e.g., RNA solution hybridization, nuclease protection, Northern blots, reverse transcription, microarrays, or PCR. For proteins, antibody or other affinity-based methods can be used, e.g., Western blots, enzyme linked immunosorbent assay (ELISA), Western blotting. For proteins that are readily detectable, e.g., fluoroscent or having an enzymatic activity, appropriate methods such as fluorescence activated cell sorting (FACS) or enzymatic detection may be used. In some embodiments, an alteration in gene expression results in a change in morphology (e.g., cell shape) or cell properties that may be detected using visual observation (e.g., using a microscope).

The invention further relates to cells that are genetically engineered to express one or more functional budding yeast RNAi pathway polypeptides, e.g., functional budding yeast Dicer and/or Argonaute polypeptides. In some embodiments, the cells are genetically engineered budding yeast cells, wherein the cells lack a functional endogenous RNAi pathway, and wherein expression of the one or more functional budding yeast RNAi pathway polypeptide, e.g., a Dicer polypeptide and an Argonaute polypeptide, reconstitutes the RNAi pathway in the cells. For example, the cells may be budding yeast cells that lack an endogenous RNAi pathway, e.g., S. cerevesiae cells.

A variety of different yeast are of use in the invention, e.g., budding yeast that have an endogenous RNAi pathway (which can serve as sources of functional RNAi pathway genes and proteins, e.g., Dicer and/or Argonaute), and budding yeast that are genetically engineered to have a functional RNAi pathway or to express a non-endogenous RNAi pathway polypeptide (and, in some embodiments, lack a functional endogenous RNAi pathway). Exemplary budding yeast are discussed herein. It will be understood that embodiments of the invention encompass other budding yeast as well. In some embodiments of interest, the budding yeast is a member of the subphylum Saccharomycotina. In some embodiments, the budding yeast is a member of the genus Saccharomyces, e.g., S. castelli, the genus Kluveromyces, e.g., Kluveromyces polysporus, the genus Candida, e.g., Candida albicans, or the genus Pichia, e.g., Pichia pastoris. In some embodiments, a yeast of interest is dimorphic. Such yeast exhibits budding under some environmental conditions. For example, Arxula adeninivorans (Blastobotrys adeninivorans) is a dimorphic yeast of interest in various biotechnological applications.

In some embodiments, the yeast is a laboratory strain. Exemplary laboratory strains of S. cerevesiae include strains S288c, W303, and derivatives thereof. See, e.g., Sherman, F., Getting started with yeast, Methods Enzymol. 350, 3-41 (2002); Mortimer and Johnston, Genetics 113:35-43 (1986); van Dijken et al., Enzyme Microb Technol 26:706-714 (2000); Winzeler et al., Genetics 163:79-89 (2003). In some embodiments the yeast is a strain that is present in the American Type Culture Collection (ATCC) yeast collection, e.g., a strain listed in the Yeast Genetics Stock Center catalog, 10^{th} ed. (1999). In some embodiments the yeast is a member of a species or strain whose genome has at least in part been sequenced. See, e.g., http://www.ncbi.nlm.nih.gov/sites/entrez under “Genome Project”. See also, Yeast Gene Order Browser, available at http://wolfe.gen.tcd.ie/ygob/ (e.g., Version 3.0). See Byrne K P and Wolfe K H, The Yeast Gene Order Browser: combining curated homology and syntenic context reveals gene fate in polyploid species, Genome Research, 15(10):1456-61, 2005. The Candida Genome Browser (http://www.candidagenome.org/) is also of use. Availability of a partial or complete genome sequence facilitates construction of libraries of nucleic acids comprising templates from which dsRNA corresponding to all or a substantial fraction of the genes can be transcribed, so that such genes can be targeted for silencing by RNAi. In some embodiments the yeast is a wild strain. In some embodiments the yeast is a strain derived by crossing a laboratory strain and a wild strain. In some embodiments the yeast is of an industrially important species or strain. In some embodiments the yeast is polyploid. In some embodiments the yeast is aneuploid. In some embodiments the yeast is diploid.

In some embodiments, the cells in which a functional RNAi pathway is engineered using budding yeast Dicer and/or Argonaute are genetically engineered prokaryotic cells, e.g., bacterial cells. Bacterial cells of interest can be gram positive, gram negative, or acid-fast and can have various morphologies, e.g., spherical (cocci) or rod-shaped. They can be laboratory strains or isolated from nature. In some embodiments, the bacteria colonize an animal or plant host. The bacteria can be pathogenic or non-pathogenic. See, e.g., Madigan, M., et al., Brock Biology of Microorganisms (12th Edition), Benjamin Cummings, 2008, for discussion of various prokaryotes (as well as eukaryotic microorganisms).

A cell of the invention, e.g., a cell genetically engineered to comprise a nucleic acid encoding an RNAi pathway polypeptide and/or genetically engineered to comprise a nucleic acid comprising a template for transcription of a dsRNA that corresponds to a gene, or a population of cells descended from such a cell, may be referred to as a “strain”. The invention provides cells that are derived from any of the inventive cells, e.g., progeny derived therefrom, cells and strains obtained by crossing a cell of one strain with a cell of another strain, cells and strains obtained by crossing a cell of an inventive strain with a strain of interest, etc.

Isolated Nucleic Acids, Polypeptides, Primers, Probes, and Antibodies

The present invention provides isolated Dicer polypeptides derived from budding yeast and variants and fragments thereof. Also provided are polynucleotides encoding the polypeptides, variants, and fragments. The present invention also provides isolated Argonaute polypeptides derived from budding yeast and polynucleotides encoding them. In some embodiments, the sequence of a polynucleotide of the invention comprises a sequence found naturally in a budding yeast, while in other embodiments the invention provides a polynucleotide that, due to the degeneracy of the genetic code, encodes the same polypeptide as a polynucleotide endogenous to (“native to”) a budding yeast.

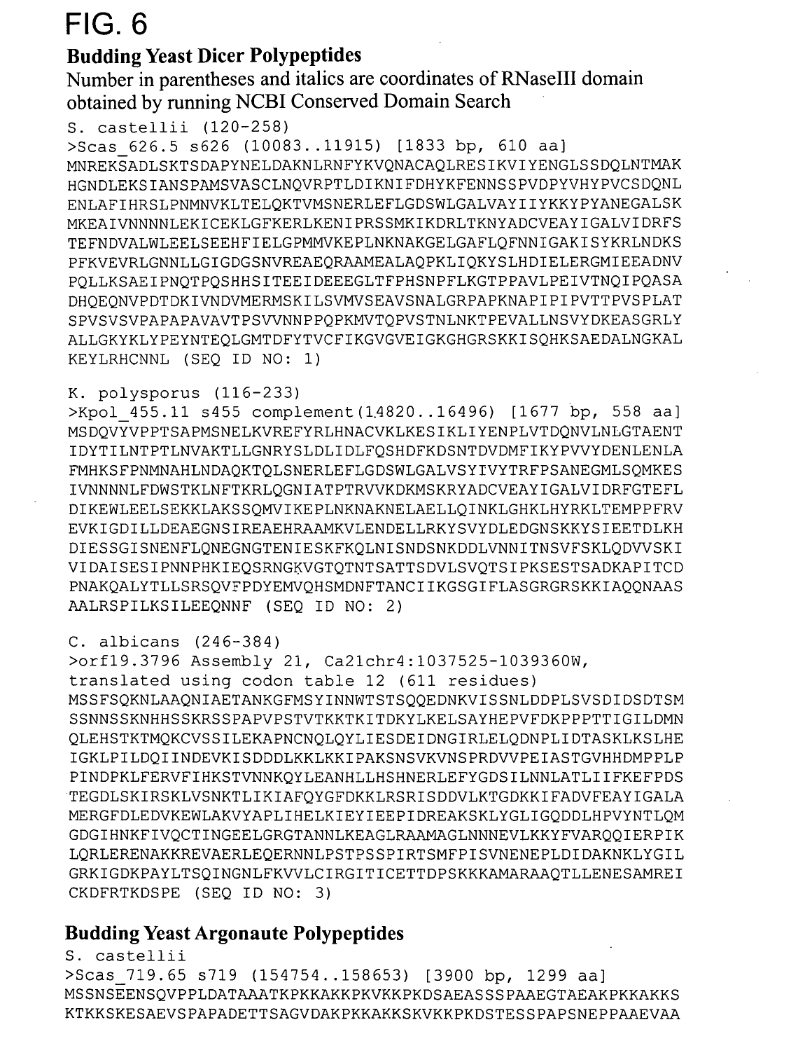

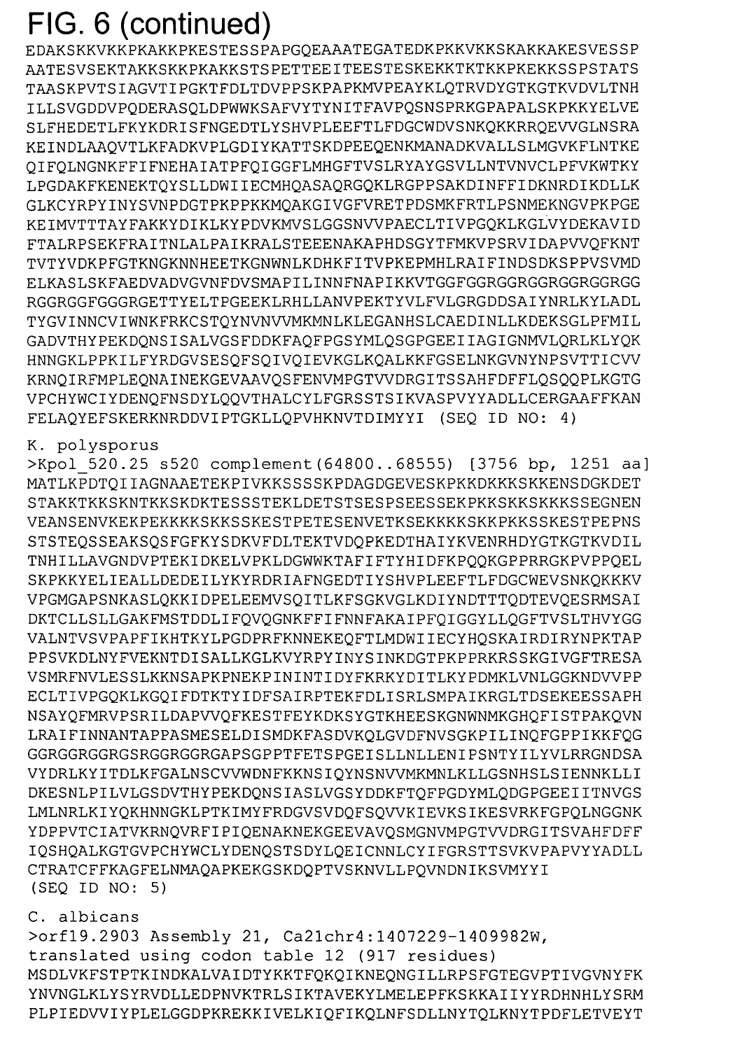

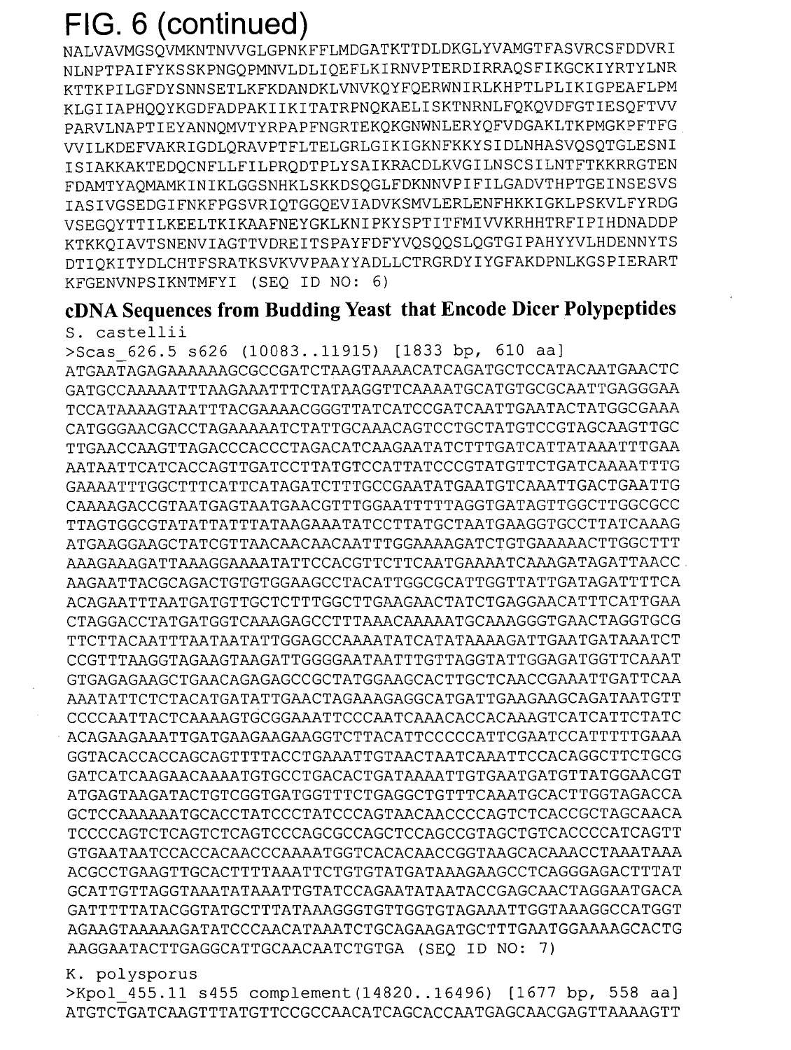

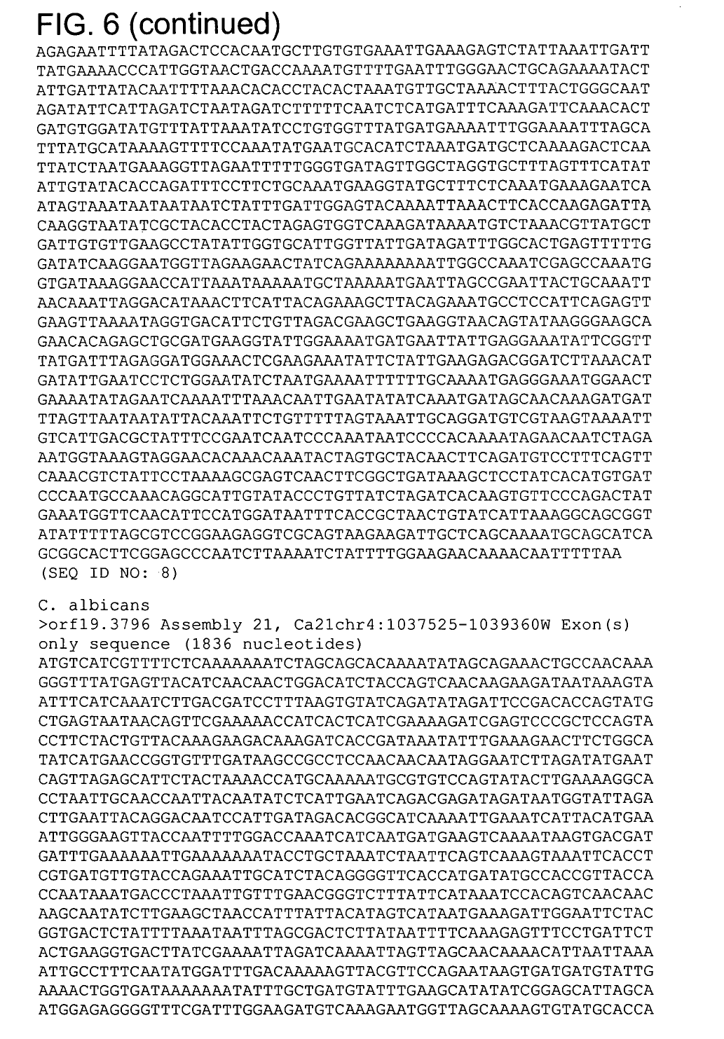

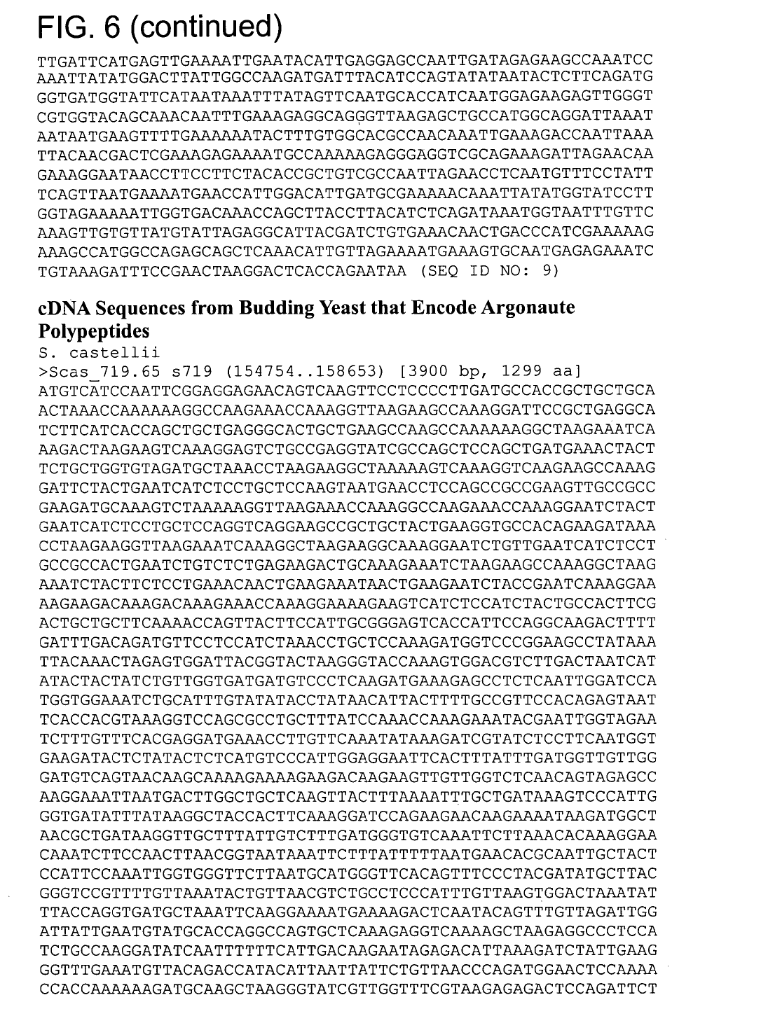

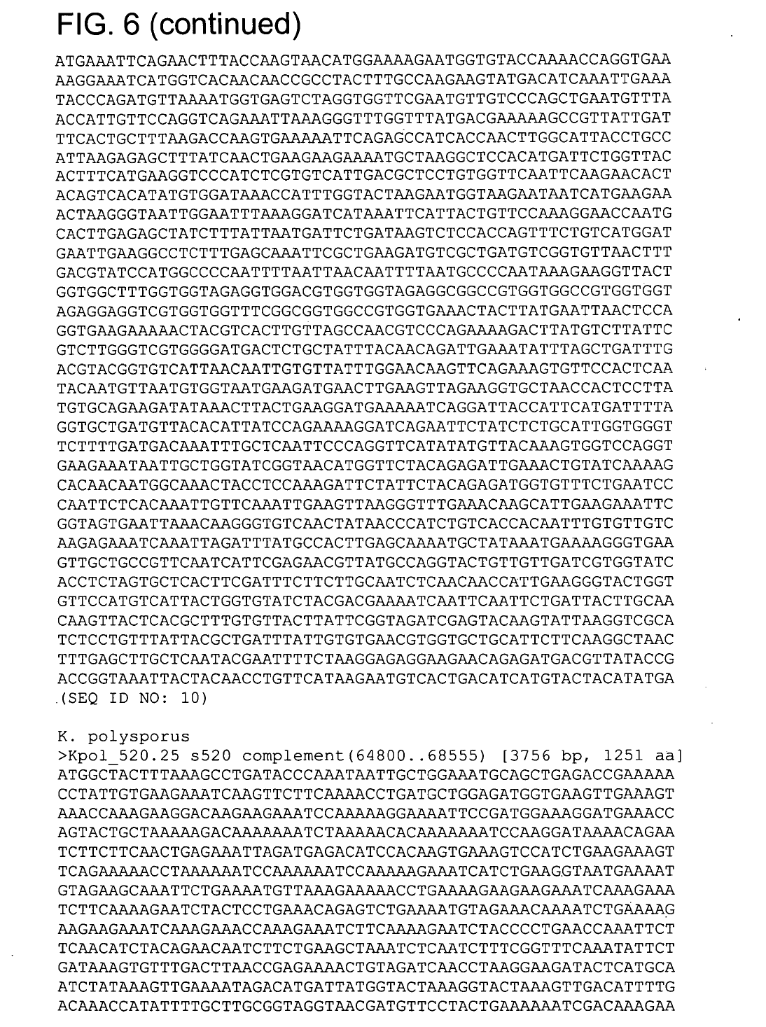

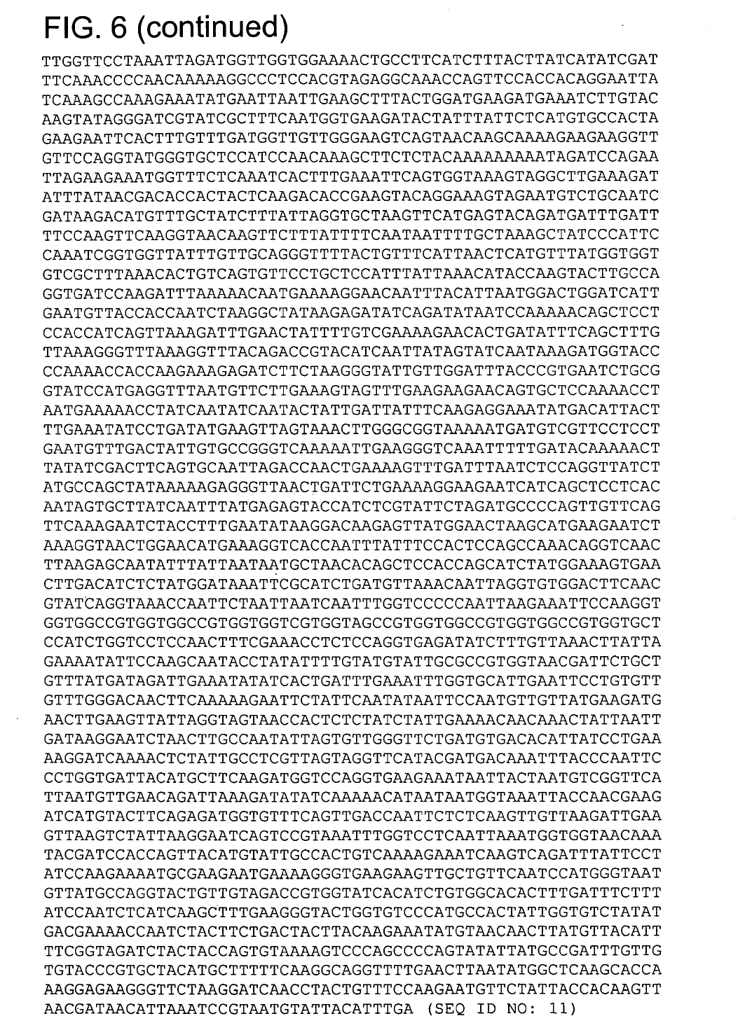

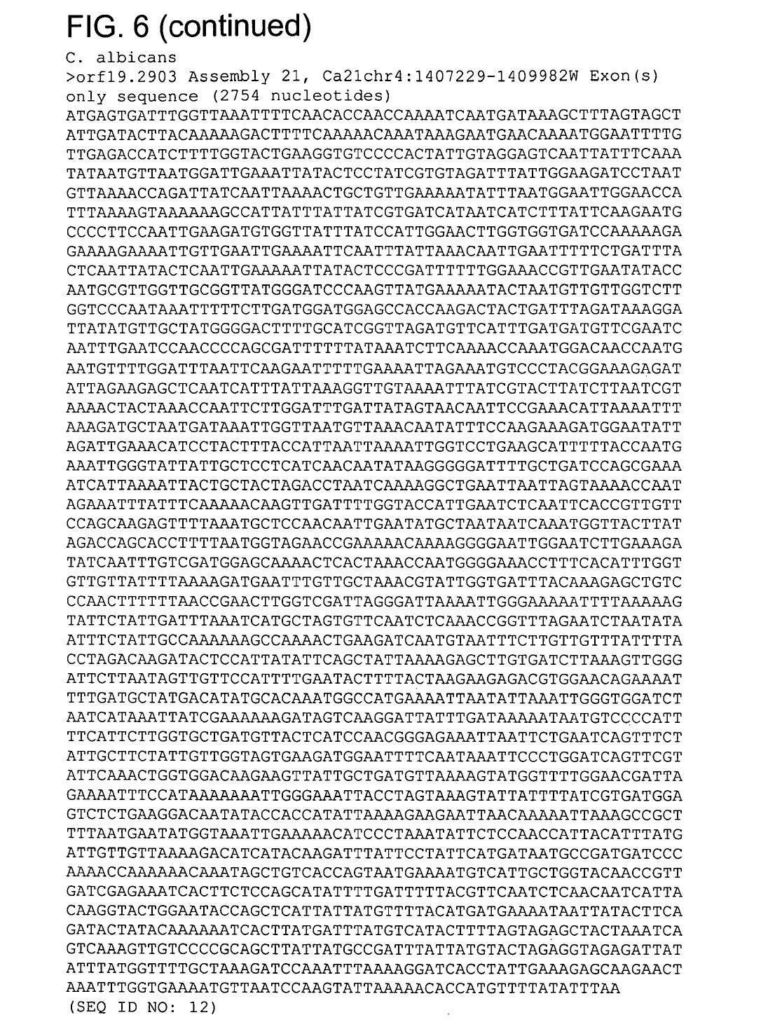

In some aspects, the invention provides an isolated nucleic acid comprising a polynucleotide that has a sequence at least 70% identical to the sequence of a naturally occurring polynucleotide that encodes a functional budding yeast Dicer polypeptide or that encodes a fragment thereof, e.g., an RNase III domain or a dsRNA binding domain. The invention further provides an isolated nucleic acid comprising a polynucleotide that has a sequence at least 70% identical to the sequence of a naturally occurring polynucleotide that encodes a functional budding yeast Argonaute polypeptide or that encodes a fragment thereof, e.g., a Piwi domain or a PAZ domain. A polynucleotide that encodes a functional budding yeast Dicer polypeptide can be derived from a budding yeast whose genome contains a gene that encodes a functional Dicer polypeptide (see, e.g., FIG. 1A). For example, the polynucleotide may be at least 70% identical to an open reading frame (ORF) derived from S. castellii, K. polysporus, or C. albicans, wherein said ORF encodes a Dicer polypeptide. See, e.g., SEQ ID NOs: 7, 8, 9 (FIG. 6) for exemplary sequences. In some embodiments, the polynucleotide is at least 75%, 80%, 85%, 90%, 91%, 92%, 93%, 94%, 95%, 96%, 97%, 98%, 99%, or 100% identical to an open reading frame (ORF) derived from S. castellii, K. polysporus, or C. albicans, wherein said ORF encodes a Dicer polypeptide. A polynucleotide that encodes a functional budding yeast Argonaute polypeptide can be derived from a budding yeast that comprises a gene that encodes a functional Argonaute polypeptide. For example, the polynucleotide may be at least 70% identical to an open reading frame (ORF) derived from S. castellii, K. polysporus, or C. albicans, wherein said ORF encodes an Argonaute polypeptide. See, e.g., SEQ ID NOs: 10, 11, 12 (FIG. 6) for exemplary sequences. In some embodiments, the polynucleotide is at least 75%, 80%, 85%, 90%, 91%, 92%, 93%, 94%, 95%, 96%, 97%, 98%, 99%, or 100% identical to an open reading frame (ORF) derived from S. castellii, or K. polysporus, or C. albicans, wherein said ORF encodes an Argonaute polypeptide. The invention further provides polynucleotides that are complementary to any of the afore-mentioned polynucleotides. Isolated nucleic acids that differ from SEQ ID NOs. 7, 8, 9, 10, 11, or 12 due to degeneracy in the genetic code are within the scope of the invention. For example, a number of amino acids are designated by more than one triplet. Codons that specify the same amino acid may result in “silent” mutations that do not affect the amino acid sequence of the polypeptide. However, it is to be expected that DNA sequence polymorphisms that lead to changes in the amino acid sequences of the subject polypeptides may exist among cells of a given yeast species, e.g., between different isolates or strains. Variations in one or more nucleotides of the nucleic acids encoding a particular polypeptide may exist among individuals of a given strain (e.g., a diploid cell can have alleles that differ in sequence).

It will also be understood that certain yeast use alternate version of the genetic code. For example, C. albicans (and various other Candida species, e.g., C. cylindracea, C. melibiosica, C. parapsilosis, and C. rugosa) as well as certain other species, e.g., certain Pichia species such as Pichia farinose, utilize an alternate genetic code that differs from the standard code in that CUG in the alternate code codes for serine while in the standard code CUG codes for leucine. [However, C. azyma, C. diversa, C. magnoliae, and C. rugopelliculosa use the standard code.] See, e.g., Ohama, T, et al. Nucleic Acids Res., 21(17):4039-45, 1993; Sugita, et al., J Gen Appl Microbiol. 45(4):193-197, 1999; Miranda, I., et al, Yeast, 23(3):203-13, 2006. One of skill in the art will take this feature into consideration in the context of the invention e.g., when using such species as a source of a nucleic acid encoding a functional Dicer or Argonaute or when genetically engineering such yeast to express a Dicer or Argonaute polypeptide or other polypeptide of interest and will make appropriate modifications. For example, one would recognize that a CUG codon in a nucleic acid encoding a C. albicans polypeptide should be translated as serine and that, if such nucleic acid is to be used to express that polypeptide in S. cerevesiae, the CUG should be changed to a codon that encodes serine in S. cerevesiae.

In some aspects of the invention the nucleic acids, or fragments thereof, may be used to screen genomic or cDNA libraries to identify nucleic acids encoding Dicer or Argonaute polypeptides in additional budding yeasts. The invention thus provides a method of identifying a budding yeast Dicer or Argonaute gene, and the Dicer or Argonaute polypeptides encoded thereby. In other embodiments, a nucleotide sequence of a budding yeast that encodes a Dicer or Argonaute polypeptide is used to search databases to identify homologous nucleic acids encoding Dicer or Argonaute polypeptides in additional species.

In some embodiments, a polynucleotide that encodes a functional budding yeast Dicer polypeptide is derived by modifying a budding yeast ORF that encodes a non-functional Dicer polypeptide. In some embodiments, the budding yeast ORF that encodes a non-functional Dicer polypeptide contains a stop codon. For example, S. pastorianus comprises a DCR1 pseudogene comprising an ORF that is intact except for a single internal stop codon. Modifying a nucleotide of the stop codon results in an ORF that encodes a functional Dicer polypeptide. The stop codon may be modified so that it encodes an amino acid found at a corresponding position in a functional budding yeast Dicer polypeptide (e.g., from S. castellii) or to any codon that encodes an amino acid consistent with allowing the resulting Dicer polypeptide to function. A similar approach may also be used to generate polynucleotides that encode functional Argonaute polypeptides from a polynucleotide that encodes a non-functional budding yeast Argonaute polypeptide. Any codon that encodes an amino acid that differs from the amino acid located at a corresponding position in a functional budding yeast Dicer or Argonaute polypeptide can be modified so that it encodes the amino acid present at the corresponding position in a functional polypeptide.

The invention further provides primers (e.g., primer pairs) useful to amplify or sequence a nucleic acid encoding a functional budding yeast Dicer polypeptide. The invention further provides primers (e.g., primer pairs) useful to amplify or sequence a nucleic acid encoding a functional budding yeast Argonaute polypeptide. The invention further provides probes (e.g., oligonucleotide probes) useful to detect a nucleic acid encoding a functional budding yeast Dicer polypeptide. The invention further provides probes useful to detect a nucleic acid encoding a functional budding yeast Argonaute polypeptide. Probes of the invention may be used for a variety of purposes. For example, they may be used to determine whether a cell expresses a Dicer or Argonaute mRNA and/or to quantify such expression. In some embodiments a primer or probe is perfectly complementary to at least about 12, at least about 15, at least about 25, or at least about 40 consecutive nucleotides of a Dicer or Argonaute polynucleotide found in a budding yeast, e.g., perfectly complementary to any of SEQ ID NOs: 7-12 (or a complement thereof), or perfectly complementary to an allele of any of SEQ ID NOs: 7-12 (or a complement thereof), wherein the allele, in some embodiments, encodes the same polypeptide as the polypeptide encoded by any of SEQ ID NOs: 7-12. In some embodiments, a primer or probe is labeled (e.g., with a fluorescent moiety, enzyme, radioisotope). In some embodiments, a primer or probe is attached to a solid support, e.g., a microparticle (“bead”), resin, or support having a substantially planar surface such as a slide, chip, etc. The invention also provides a microarray comprising any of the inventive probes, e.g., a microarray useful for measuring mRNA expression.

The invention provides a method for detecting the presence of a nucleic acid whose sequence comprises all or part of a budding yeast Dicer polynucleotide sequence in a sample. The method comprises: (a) contacting the sample with a probe or primer that binds to a budding yeast Dicer polynucleotide; and b) determining whether the probe or primer binds to the budding yeast Dicer polynucleotide in the sample. In certain embodiments, the invention provides a method for detecting the presence of a nucleic acid whose sequence comprises all or part of a budding yeast Argonaute polynucleotide sequence in a sample. The method comprises: (a) contacting the sample with a probe or primer that binds to a budding yeast Argonaute polynucleotide; and b) determining whether the probe or primer binds to the budding yeast Argonaute polynucleotide in the sample.

The invention further provides isolated Dicer polypeptides of budding yeast and variants and fragments thereof. In some embodiments, the invention provides an isolated polypeptide comprising a polypeptide that has a sequence at least 70%, 80%, 90%, 95%, or 99% identical to the sequence of a functional Dicer polypeptide found in budding yeast. FIG. 6 presents the sequences of budding yeast Dicer polypeptides present in S. castellii (SEQ ID NO: 1), K. polysporus (SEQ ID NO: 2), and C. albicans (SEQ ID NO: 3). Thus in some embodiments the isolated polypeptide comprises a sequence at least 70%, 75%, 80%, 85%, 90%, 91%, 92%, 93%, 94%, 95%, 96%, 97%, 98%, 99%, or 100% identical to SEQ ID NO: 1. In some embodiments the isolated polypeptide comprises a sequence at least 70%, 75%, 80%, 85%, 90%, 91%, 92%, 93%, 94%, 95%, 96%, 97%, 98%, 99%, or 100% identical to SEQ ID NO: 2. In some embodiments the isolated polypeptide comprises a sequence at least 70%, 75%, 80%, 85%, 90%, 91%, 92%, 93%, 94%, 95%, 96%, 97%, 98%, 99%, or 100% identical to SEQ ID NO: 3. In some embodiments, the isolated polypeptide comprises a sequence at least 70%, 75%, 80%, 85%, 90%, 91%, 92%, 93%, 94%, 95%, 96%, 97%, 98%, 99%, or 100% identical to Dicer polypeptide found in S. bayanus. In some embodiments the isolated polypeptide comprises a sequence at least 70%, 75%, 80%, 85%, 90%, 91%, 92%, 93%, 94%, 95%, 96%, 97%, 98%, 99%, or 100% identical to Dicer polypeptide found in S. bayanus, C. tropicalis, Pichia stipitis, or Debaromyces hansenii (see Examples for accession numbers).

In one embodiment, the invention provides a polypeptide comprising an N-terminal fragment of a Dicer polypeptide found in budding yeast. The fragment may comprise, e.g., an RNase III domain and the adjacent dsRNA binding domain. In some embodiments a Dicer polypeptide lacks a C-terminal region of a Dicer polypeptide found in a budding yeast.

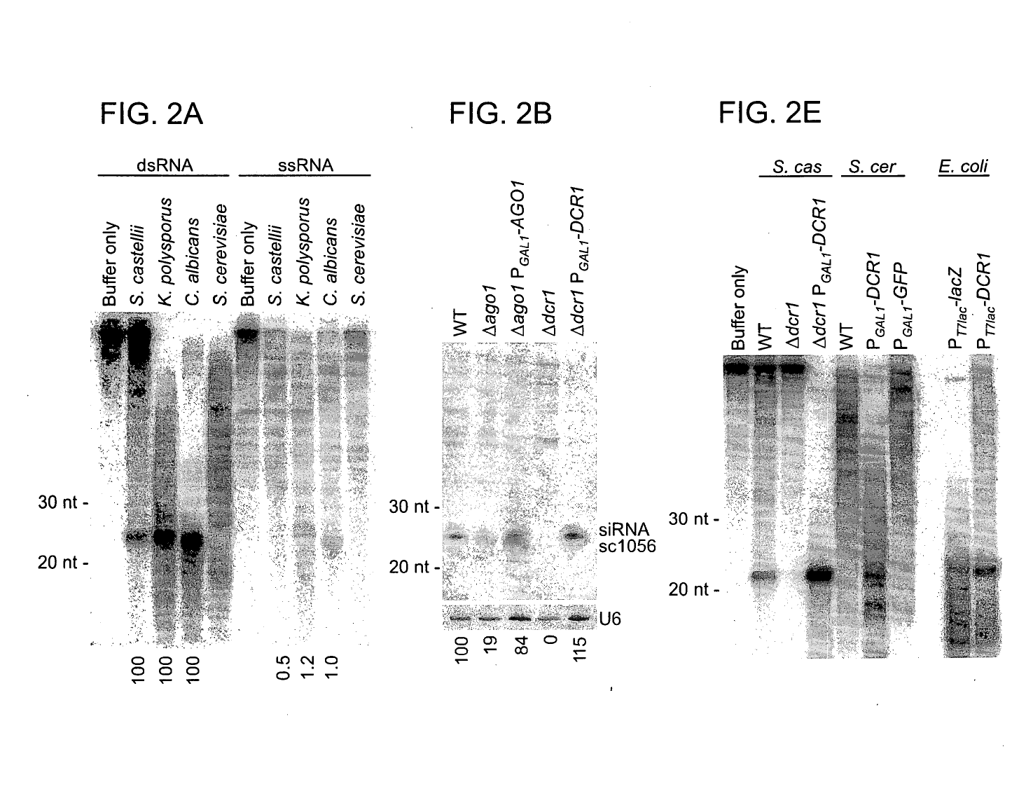

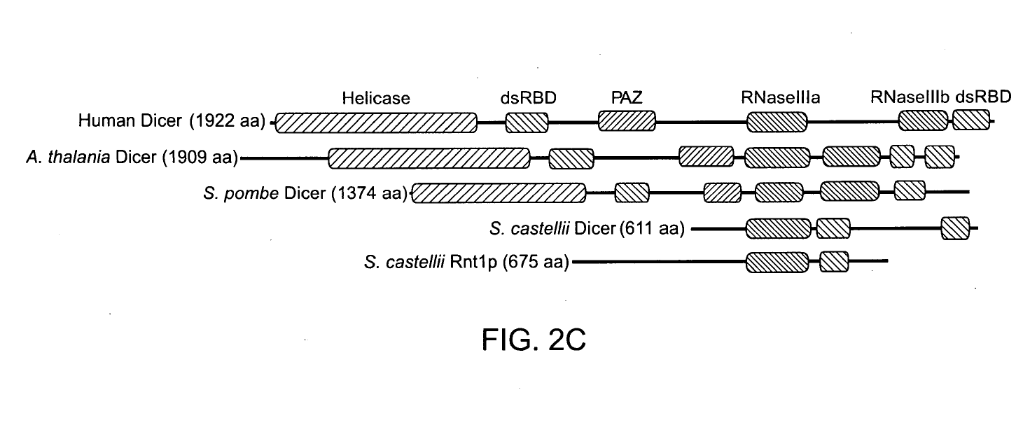

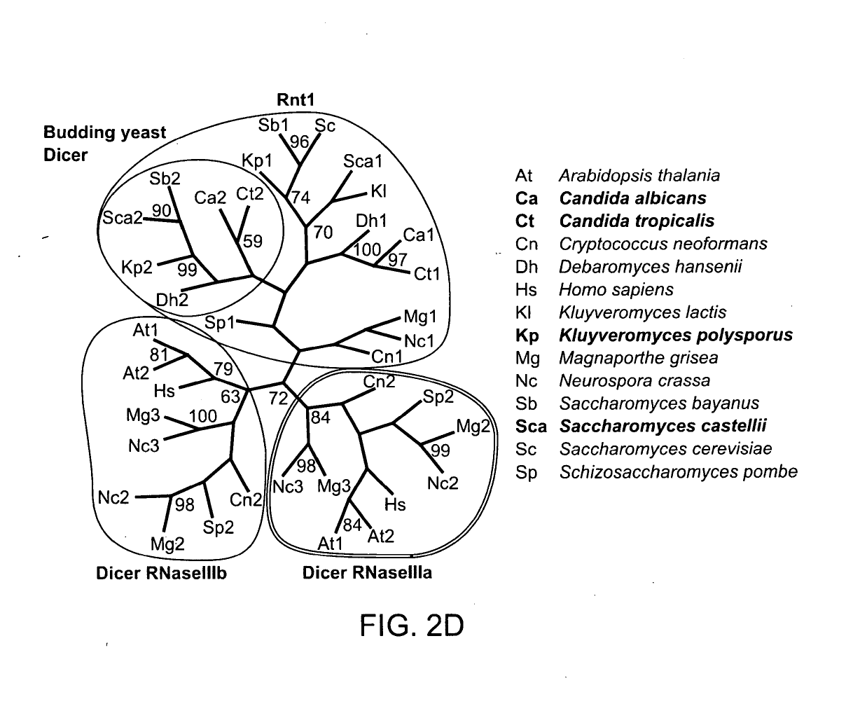

In some embodiments, the invention provides an isolated polypeptide that comprises an RNase III domain of a Dicer polypeptide of a budding yeast or that comprises a variants or fragment thereof. In some aspects, the invention provides an isolated polypeptide comprising a polypeptide that has a sequence at least 70%, 80%, 90%, 95%, 99%, or 100% identical to the sequence of an RNase III domain of a functional budding yeast Dicer polypeptide, e.g., an RNase III domain of a Dicer polypeptide of S. castellii, K. polysporus, C. albicans, C. tropicalis, P. stipitis, or Debaromyces hansenii. An RNase III domain of a functional budding yeast Dicer polypeptide can be identified based on, e.g., homology to known RNase III domains, e.g., as described in the Examples. In some embodiments, the isolated polypeptide comprises an RNase domain that is between about 110 and about 200 amino acids long, e.g., between about 110 and about 150 amino acids long. In some embodiments, the RNase III domain consists of about amino acids 120-258 of SEQ ID NO: 1. In some embodiments, the RNase III domain consists of about amino acids 116-233 of SEQ ID NO: 2. In some embodiments, the RNase III domain consists of about amino acids 246-384 of SEQ ID NO: 3. The afore-mentioned positions are coordinates of RNaseIII domains obtained by running NCBI Conserved Domain Search using SEQ ID NOs: 1, 2, and 3. It will be appreciated that slightly different coordinates may be used, e.g., as identified using different domain search programs. For example, RNaseIII domains may be predicted using SMART (22, 23). In some embodiments the borders of the RNase III domain are shifted, e.g., by up to about 5 or about 10 amino acids in either direction. The invention further provides alignments of RNase III domains of Dicer polypeptides isolated from budding yeast. In some embodiments, amino acid sequences of the RNaseIII domains are used to compute a multiple sequence alignment, e.g., using TCOFFEE (24). Such alignment identifies amino acid residues that are identical or similar (e.g., conservative substitutions) among multiple RNase III domains. A variety of alignment programs are known in the art and may be employed. In some embodiments, the polypeptide that has a sequence at least 70% identical to the sequence of an RNase III domain of a functional budding yeast Dicer polypeptide clusters within the budding yeast Dicer RNase III domain cladogram of FIG. 2D. In some embodiments, the invention provides an isolated polypeptide comprising a minimal Dicer polypeptide comprising an RNase III domain, e.g., a polypeptide that is at least 80% identical to an RNase III domain found in a functional budding yeast Dicer polypeptide, optionally further comprising a dsRNA binding domain. In some embodiments, a “minimal Dicer polypeptide” represents the minimal amount of sequence needed to retain dsRNA cleaving ability.

The isolated polypeptide may further comprise one or more additional domains, e.g., a dsRNA binding domain. The isolated polypeptide may further comprise additional sequence identical or homologous to SEQ ID NO: 1, 2, 3 (or other Dicer polypeptides of budding yeast). In some embodiments the polypeptide further comprises a domain at least 70%, 80%, 90%, 95%, or 99% identical to a dsRNA binding domain of the Dicer polypeptide of S. castellii, K. polysporus, C. albicans, C. tropicalis, P. stipitis, or Debaromyces hansenii. In some embodiments the dsRNA binding domain is derived from an organism other than a budding yeast, e.g., a fission yeast or other fungus, insect, animal (e.g., mammal) or plant, e.g., is at least 70% 80%, 90%, 95%, 99%, or 100% identical to such a dsRNA binding domain.

In some embodiments, the isolated polypeptide comprises or consists of amino acids 15-355 of K. polysporus Dicer or corresponding amino acids of Dicer from a different budding yeast, such as S. castellii, C. albicans, C. tropicalis, P. stipitis, or Debaromyces hansenii.

In some embodiments, the invention provides an isolated polypeptide that comprises a Piwi or PAZ domain of an Argonaute polypeptide of a budding yeast or that comprises a variant or fragment thereof. In some aspects, the invention provides an isolated polypeptide comprising a polypeptide that has a sequence at least 70%, 80%, 90%, 95%, 99%, or 100% identical to the sequence of a Piwi or PAZ domain of a functional budding yeast Argonaute polypeptide, e.g., a Piwi or PAZ domain of an Argonaute polypeptide of S. castellii, K. polysporus, C. albicans, or C. tropicalis. In some embodiments, the isolated polypeptide comprises a sequence at least 70%, 80%, 90%, 95%, 99%, or 100% identical to the sequence of a Piwi domain and a sequence at least 70%, 80%, 90%, 95%, 99%, or 100% identical to the sequence of a PAZ domain of an Argonaute polypeptide found in a budding yeast, e.g., S. castellii, K. polysporus, C. albicans, or C. tropicalis. In some embodiments, the invention provides an isolated polypeptide that comprises a polypeptide that has a sequence at least 70%, 80%, 90%, 95%, 99%, or 100% identical to the sequence of an Argonaute polypeptide found in a budding yeast. In some embodiments, the invention provides an isolated polypeptide comprising a polypeptide that has a sequence at least 70%, 75%, 80%, 85%, 90%, 91%, 92%, 93%, 94%, 95%, 96%, 97%, 98%, 99%, or 100% identical to SEQ ID NO: 4. In some embodiments, the invention provides an isolated polypeptide comprising a polypeptide that has a sequence at least 70%, 75%, 80%, 85%, 90%, 91%, 92%, 93%, 94%, 95%, 96%, 97%, 98%, 99%, or 100% identical to SEQ ID NO: 5. In some embodiments, the invention provides an isolated polypeptide comprising a polypeptide that has a sequence at least 70%, 75%, 80%, 85%, 90%, 91%, 92%, 93%, 94%, 95%, 96%, 97%, 98%, 99%, or 100% identical to SEQ ID NO: 6.

In some embodiments, the sequence of an isolated polypeptide or nucleic acid of the invention (e.g., a full length Dicer or Argonaute polypeptide, variant, or fragment) differs from that of a Dicer or Argonaute polypeptide or nucleic acid encoding such polypeptide, that is found in a eukaryote other than a budding yeast and is known in the art as of the filing date hereof For example, the sequence may differ from that of a Dicer or Argonaute polypeptide or nucleic acid encoding such polypeptide found in, e.g., human, mouse, D. melanogaster, C. elegans, A. thaliana, N. crassa, A. nidulans, S. pombe, etc. In some embodiments, the sequence of an isolated polypeptide or nucleic acid of the invention differs from that of an RNase III polypeptide (or nucleic acid encoding it) found in a prokaryote, e.g., a bacteria. In some embodiments, the sequence of an isolated polypeptide or nucleic acid of the invention differs from that of a RNT1 polypeptide (or nucleic acid encoding it) of a budding yeast. Any sequence can, if desired, be explicitly excluded from any aspect or embodiment of the invention.

In some embodiments, a Dicer or Argonaute polypeptide is a functional variant or fragment. One of skill in the art will appreciate that functional variants may be readily obtained based on the sequences of the identified Dicer and Argonaute polypeptides found in budding yeast. In some embodiments, a variant comprises one or more conservative substitutions. It will be appreciated that regions that are poorly conserved and/or absent in at least some functional polypeptides found in budding yeast may be more tolerant of alterations (e.g., substitutions) than regions that are more highly conserved. It will also be appreciated that regions outside the recognized functional domains (e.g., RNase III, dsRNA binding, Piwi, PAZ) are candidates for making modifications consistent with preserving function. Further, one could use structural information to select regions for modification. One could, for example, utilize structural and functional information available regarding Dicer and Argonaute polypeptides from other eukaryotes, if desired.

It will be appreciated that a functional variant or fragment can have a different level of functional activity as the original polypeptide. In some embodiments, at least one function of a variant or fragment is substantially similar in activity to that of the corresponding function of the original molecule. For example, a variant or fragment may be considered to have a functional activity substantially similar to that of the original molecule if the activity of the variant or fragment is at least 20%, 50%, 60%, 70%, 80%, 90%, 95% of the activity of the original molecule, up to approximately 100%, approximately 125%, or approximately 150% of the activity of the original molecule (on a molar basis). In other nonlimiting embodiments an activity of a variant or fragment is considered substantially similar to the activity of the original molecule if the amount or concentration of the variant needed to produce a particular effect is within 0.5 to 5-fold of the amount or concentration of the original molecule needed to produce that effect. In some embodiments, a fragment or variant may have a higher activity than an original polypeptide. In some embodiments, a fragment has a higher activity than an original polypeptide on a per weight basis. In some embodiments, the activity is, e.g., up to 2-, 5-, or 10-fold higher. In some embodiments, a function is assessed in vitro (e.g., ability of a Dicer polypeptide to cleave a dsRNA in vitro under suitable conditions). In some embodiments a function is assessed in vivo. For example, the ability of a variant or fragment to restore siRNA producing ability, silencing activity, and/or target mRNA degrading ability to an S. castellii or C. albicans DCR1 or AGO deletion mutant can be assessed. In another embodiment, the ability of a variant or fragment Dicer or Argonaute to confer siRNA producing ability, silencing activity, and/or target mRNA degrading ability, on cell, e.g., an S. cerevesiae cell, that lacks functional Dicer or Argonaute, respectively, can be assessed.

The invention further provides isolated nucleic acids that encode any of the polypeptides of the invention, and cells containing them (e.g., integrated into the genome or on an episome). In some embodiments, the isolated nucleic acid is codon optimized for expression in a budding yeast that lacks an endogenous functional Dicer polypeptide. In some embodiments, the isolated nucleic acid is codon optimized for expression in an organism other than a budding yeast, e.g., a bacterium.

The invention provides vectors that comprise any of the isolated nucleic acids of the invention. In some embodiments, the isolated nucleic acid is in a vector used in the art in genetic engineering of a budding yeast. In some embodiments the vector is a plasmid. Other vectors include artificial chromosomes and linear nucleic acid molecules that are distinct from linearized plasmids. In some embodiments the vector is an integrating vector. In some embodiments the vector comprises an expression control element operably linked to a nucleic acid to be transcribed (e.g., a nucleic acid that encodes a polypeptide of the invention or that provides a template for transcription of a dsRNA). Three well known plasmid systems used for recombinant expression and replication in yeast cells include integrative plasmids, low-copy-number ARS-CEN plasmids, and high-copy-number 2μ plasmids. See, e.g., Christianson T W, et al., “Multifunctional yeast high-copy-number shuttle vectors”. Gene. 110:119-22 (1992); Sikorski, “Extrachromosomal cloning vectors of Saccharomyces cerevisiae”, in Plasmid, A Practical Approach, Ed. K. G. Hardy, IRL Press, 1993; Parent, S. A., and Bostian, K. A., Recombinant DNA technology: yeast vectors, p. 121-178. In Wheals, A. E., et al. (eds.) The yeasts, vol. 6. Yeast genetics. Academic Press, Longon, UK (1995). An example of integrating plasmids of use in budding yeast are YIp plasmids, which are maintained at one copy per haploid genome and inherited in Mendelian fashion. Such a plasmid, containing a nucleic acid of interest, a bacterial origin of replication and a selectable gene (typically an antibiotic-resistance marker), is typically produced in bacteria. The purified vector may be linearized and used to transform competent yeast cells. YCp plasmids, which contain the autonomous replicating sequence (ARS1) and a centromeric sequence (CEN4), are examples of low-copy-number ARS-CEN plasmids. These plasmids are usually present at 1-2 copies per cell. An example of the high-copy-number 2μ plasmids are YEp plasmids, which contain a sequence approximately 1 kb in length (named the 2μ sequence). The 2μ sequence acts as a yeast replicon giving rise to higher plasmid copy number. These plasmids may require selection for maintenance.

In some embodiments, an integrating plasmid is a pRS plasmid (e.g., pRS303, pRS304, pRS305 or pRS306 or other integrative plasmids). In some embodiments, the plasmid is an extrachromosomal plasmid (e.g., pRS313, pRS314, pRS315, pRS316, pRS413, pRS414, pRS415, pRS416, pRS423, pRS424, pRS425, pRS426). In some embodiments the plasmid is a member of the YES™ Vector Collection, e.g., pYES (Invitrogen, Carlsbad, Calif.). In some embodiments, the plasmid is a Gateway plasmid. See, e.g., Geiser J R. Recombinational cloning vectors for regulated expression in Saccharomyces cerevisiae. Biotechniques, 38:378-382 (2005); Van Mullem V, et al., Construction of a set of Saccharomyces cerevisiae vectors designed for recombinational cloning. Yeast. 20:739-46 (2003); Alberti, S., et al., A suite of Gateway cloning vectors for high-throughput genetic analysis in Saccharomyces cerevisiae. Yeast, 24(10):913-9 (2007).

A nucleic acid encoding a functional RNAi pathway polypeptide or providing a template for transcription of a dsRNA may be introduced into a cell, e.g., a yeast cell, using any suitable method. Yeast cells are often transformed by chemical methods (e.g., as described by Rose et al., 1990, Methods in Yeast Genetics, Cold Spring Harbor Laboratory Press, Cold Spring Harbor, N.Y.). The cells are typically treated with lithium acetate to achieve transformation efficiencies of approximately 10⁴ colony-forming units (transformed cells)/μg of DNA. In some embodiments, yeast perform homologous recombination such that the cut, selectable marker recombines with the mutated (usually a point mutation or a small deletion) host gene to restore function. Transformed cells are then isolated on selective media. Of course, any suitable means of introducing nucleic acids into yeast cells can be used, such as electroporation. See, e.g., Gietz, R. D. and Woods, R. A., Genetic transformation of yeast. BioTechniques, 30:816-820; 822-826,828 (2001). Many yeast vectors (e.g., plasmids) typically contain a yeast origin of replication, an antibiotic resistance gene, a bacterial origin of replication (for propagation in bacterial cells), multiple cloning sites, and a yeast nutritional marker gene to promote maintenance and/or genomic integration in yeast cells. The yeast nutritional gene (or “auxotrophic marker”) is often one of the following: 1) TRP1 (Phosphoribosylanthranilate isomerase); 2) URA3 (Orotidine-5′-phosphate decarboxylase); 3) LEU2 (3-Isopropylmalate dehydrogenase); 4) HIS3 (Imidazoleglycerolphosphate dehydratase or IGP dehydratase); or 5) LYS2 (α-aminoadipate-semialdehyde dehydrogenase). An antibiotic resistance gene can facilitate maintenance and propagation of the plasmid in bacteria and/or to identify yeast transformants and/or promote maintenance of the plasmid in yeast. Exemplary antibiotic resistance markers include the kanamycin (G418) resistance gene, chloramphenicol resistance gene, and hygromycin resistance gene. See, e.g., U.S. Pat. No. 6,214,577. A number of other selectable markers of use in yeast are known. See, e.g., U.S. Pat. No. 4,626,505. The ARO4-OFP and FZF1-4 genes, which confer p-fluoro-DL-phenylalanine resistance and sulfite resistance, respectively, may also be used as dominant selectable markers, e.g., in laboratory and wine yeast S. cerevisiae strains (Cebollero, E. and Gonzalez, R. Applied and Environmental Microbiology, 70 (12): 7018-7023, 2004). One of skill in the art can select an appropriate marker based on considerations such as whether the yeast is auxotrophic or prototrophic, convenience, and the particular application.

Yeast vectors (e.g., plasmids) described herein may also contain expression control sequences, e.g., promoter sequences. A “promoter” is a control sequence that is a region of a nucleic acid sequence at which initiation and rate of transcription are controlled. It may contain genetic elements at which regulatory proteins and molecules may bind, such as RNA polymerase and transcription factors, to initiate the transcription of a nucleic acid sequence. The phrase “operably linked” indicates that an expression control element, e.g., a promoter, is in an appropriate location and/or orientation in relation to a nucleic acid to control transcriptional initiation and/or expression of the nucleic acid. A promoter may be one that is naturally associated with a nucleic acid sequence, as may be obtained by isolating the 5′ non-coding sequences located upstream of the coding segment. Alternatively, a promoter may be a recombinant or heterologous promoter, which refers to a promoter that is not normally associated with a nucleic acid segment in its natural environment. Such promoters may include promoters of other genes and promoters that are not naturally occurring. An expression control element may be derived from a yeast of the species or strain in which RNAi is to be used or in which the RNAi pathway is to be engineered. For example, if RNAi is to be used in C. albicans, it may be desirable to use a C. albicans promoter to direct expression of a dsRNA. However, any expression control element capable of directing transcription in the cell of interest may be used.

The promoters employed may be either constitutive or inducible. For example, various yeast-specific promoters may be employed to regulate the expression in yeast cells. Examples of inducible yeast promoters include GALL-10, GAL1, GALL, GALS, TET, CUP1, VP16 and VP16-ER. Examples of repressible yeast promoters include Met25. Examples of constitutive yeast promoters include glyceraldehyde 3-phosphate dehydrogenase promoter (GPD), phosphoglycerate kinase (PGK), alcohol dehydrogenase promoter (ADH), translation-elongation factor-1-alpha promoter (TEF), cytochrome c-oxidase promoter (CYC1), and MRP7. Promoters containing steroid response elements (e.g., glucocorticoid response element) inducible by glucocorticoid or other steroid hormones can also direct expression in yeast. Yet other yeast constitutive or inducible promoters such as those of the genes for alpha factor, phosphate pathway genes (e.g., PHO5), or alcohol oxidase may be used. In some embodiments a vector of the invention comprises an expression control element known as an upstream activating sequence (UAS). Such elements, which are considered functional equivalents of metazoan enhancers, can activate gene transcription from remote positions, e.g., up to about 1,000-1,200 by from the promoter. See, e.g., Petrascheck, M., et al., Nucleic Acids Res., 33(12): 3743-3750, 2005, for discussion. The level of expression achieved using an inducible promoter can be regulated, e.g., by controlling the amount of inducing agent or the length of exposure. Further, mutant promoters that result in lower expression levels than a wild type promoter can be used. In some embodiments, an expression control element originates from a species in which the expression control element is to be used to direct expression while in other embodiments the expression control element originates from a different species. In some embodiments, expression control elements that are in nature operably linked to genes encoding functional budding yeast Dicer polypeptides are used. For example, an S. castellii DCR1 . . . promoter may be used to direct expression of S. castellii Dicer in, e.g., a budding yeast that lacks an endogenous functional Dicer.

In other aspects, the invention provides vectors suitable for mutating, e.g., at least in part deleting an endogenous Dicer or Argonaute polypeptide of a budding yeast, e.g., a budding yeast that has a functional RNAi pathway. Optionally, such mutation renders the gene or encoded polypeptide non-functional. Exemplary vectors for generating deletion strains in S. castelli are described in the Examples.

The invention further provides vectors suitable for expressing a budding yeast RNAi pathway polypeptide, e.g., Dicer, in a variety of different cells that are not budding yeast cells, e.g., bacterial cells, insect cells, mammalian cells, or fungal cells other than budding yeast cells. In some embodiments a vector contains an origin of replication that supports replication in bacterial cells such as a ColE1 origin. Any of a number of other origins of replication, such as those present on various different plasmids, can be used (see, e.g., del Solar, G., et al. Microbiol. and Mol Biol Rev, 62(2): 434-464, 1998). The origin of replication can be a high copy number origin (such as that found within pUC-based plasmids) or a medium or low copy number origin (such as that found in plasmids based on pBR322). In some embodiments a vector also contains a bacterial promoter (i.e., a promoter effective to express a protein of interest, e.g., a budding yeast Dicer polypeptide, in bacteria). The promoter can be constitutive or regulatable, e.g., inducible. An exemplary promoter for expression in bacteria is a T7 promoter, which is inducible upon the addition of IPTG to culture medium, but any of a number of other promoters such as other phage promoters (e.g., pL, etc.) could be used. Other suitable bacterial promoters include Lac, Trp, Tac, and pBAD. It will be appreciated that where a phage promoter such as the T7 promoter is used, the appropriate RNA polymerase (e.g., T7 RNA polymerase) should be expressed within the host cell. A sequence encoding the polymerase operably linked to a promoter can be provided by the host cell genome (many such bacterial hosts are known in the art) but can alternatively be included on an inventive vector, or provided by a different vector present in the host cell. An operator sequence, e.g., the lac operator, can be included, to allow repression of the bacterial promoter. For example, the plasmid may comprise T7 promoter and a downstream Lac operator (i.e., a site for binding of the lac repressor), forming a unit found in many standard prokaryotic expression vectors and commonly referred to as the T7lac promoter. The vector can comprise a ribosome binding site (RBS) downstream of the bacterial promoter or promoter/operator portion. A consensus sequence for an effective ribosome binding site is AGGAGG, but many variants, including both shorter and longer sequences, support efficient translation. Typically these sequences are AG rich.

In some embodiments the invention provides vectors suitable for expressing a polypeptide of the invention in animal cells, e.g., mammalian or insect cells, or plant cells. Expression control sequences useful for directing expression in mammalian cells include the early and late promoters of SV40, adenovirus or cytomegalovirus immediate early promoter, or viral promoter/enhancer sequences as well as promoters or promoter/enhancers from mammalian genes, e.g., actin, EF-1 alpha, metallothionein. Certain mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. The polyhedrin promoter of the baculovirus system is of use to express proteins in insect cells. Examples of baculovirus expression systems include pVL-derived vectors (such as pVL1392, pVL1393 and pVL941), pAcUW-derived vectors (such as pAcUW1), and pBlueBac-derived vectors (such as pBlueBac III). Other sequences known to control the expression of genes of prokaryotic or eukaryotic cells or their viruses, and various combinations thereof, may be used, and may be selected based on the particular host cell of interest. For example, many vectors for expressing polypeptides in plants are available, e.g., those based on plant viruses such as cauliflower mosaic virus, or on bacteria such as Agrobacteria. One of skill in the art will be aware of methods for transforming bacteria or plant cells, transfecting animal cells, and deriving stable cell lines or transgenic animals or plants, if desired.

Certain vectors of the invention include a cloning site for insertion of a nucleic acid of interest (e.g., a nucleic acid that encodes a Dicer polypeptide, or a nucleic acid that can be transcribed to yield a dsRNA). In general, any restriction enzyme site may serve this purpose. Certain embodiments include a multiple cloning site, or polylinker. In some embodiments, the cloning site is positioned so that an inserted nucleic acid is operably linked to expression control element(s), e.g., a promoter, already present in the vector. In other embodiments, a nucleic acid cassette comprising one or more expression control elements and a nucleic acid to be transcribed is inserted into a vector. The vector or nucleic acid cassette may further comprise a transcriptional terminator (e.g., the yeast CYC1 terminator).

In some embodiments, the invention provides a nucleic acid, e.g., a vector, comprising (i) a first polynucleotide that encodes a Dicer polypeptide found in a budding yeast or a variant or fragment thereof; (ii) a second polynucleotide that encodes an Argonaute polypeptide found in a budding yeast or a variant or fragment thereof; (iii) and, optionally, a third polynucleotide that comprises a template for transcription of a dsRNA. In some embodiments, the polynucleotide of (i) is at least 80% identical to a Dicer polypeptide found in a budding yeast. In some embodiments, the polynucleotide of (i) is at least 80% identical to an Argonaute polypeptide found in a budding yeast. In some embodiments, the polynucleotide of (i) encodes a polypeptide at least 80% identical to an RNase III domain of a Dicer polypeptide found in a budding yeast; and/or the polynucleotide of (ii) encodes a polypeptide that comprises a first portion at least 80% identical to a Piwi domain of an Argonaute polypeptide found in a budding yeast and a second domain at least 80% identical to a PAZ domain of an Argonaute polypeptide found in a budding yeast. In some embodiments, the first polynucleotide further comprises a portion that encodes a dsRNA binding domain. In some embodiments, polynucleotides of (i), (ii), and/or (iii) are each operably linked to at least one expression control element, e.g., a promoter, so that they are transcribed when the nucleic acid is introduced into a cell. In some embodiments the promoter for the dsRNA is inducible. The invention further provides libraries of such vectors, wherein the vectors differ in that they comprise dsRNAs that correspond to different genes, e.g., at least 10 different genes or at least 10% of the genes of a genome. In some embodiments the dsRNAs correspond to genes of a budding yeast.

In some embodiments, the nucleic acid, nucleic acid cassette, or vector comprises a portion that encodes a tag. The tag may be useful for, e.g., enhancing expression, detection, and/or purification of a polypeptide. For example, the tag can be an affinity tag (e.g., HA, TAP, Myc, H is, Flag, GST), fluorescent or luminescent protein (e.g., EGFP, ECFP, EYFP, Cerulean, DsRed, mCherry), solubility-enhancing and/or expression-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006). A tag is often relatively small, e.g., ranging from a few amino acids up to about 100 amino acids long. In some embodiments a tag is more than 100 amino acids long, e.g., up to about 500 amino acids long. In some embodiments, an RNAi pathway polypeptide has an N- or C-terminal fusion to the tag. The polypeptide could comprise multiple tags. For example, a polypeptide could comprise an affinity tag and a solubility-enhancing or expression-enhancing tag. In some embodiments, a tag is cleavable, so that it can be removed from the polypeptide, e.g., by a protease. In some embodiments, this is achieved by including a sequence encoding a protease cleavage site between the sequence encoding the RNAi pathway polypeptide and the tag. In some embodiments, a “self-cleaving” tag is used. See, e.g., PCT/US05/05763. Sequences encoding a tag can be located 5′ or 3′ with respect to a polynucleotide encoding the polypeptide (or both). In some embodiments, a protease cleavage site comprises an amino acid sequence that is not present within the polypeptide. In some embodiments, the polypeptide has the formula [affinity tag]−[solubility or expression-enhancing tag]−[protease cleavage site]−Dicer polypeptide. For example, the polypeptide can have the following formula: [His-tag]-[SUMO-tag]-[Upl1 protease cleavage site]-Dicer polypeptide, where “Dicer polypeptide” represents, e.g., a full length Dicer polypeptide of budding yeast or a variant or fragment thereof.

The invention provides methods of producing a budding yeast Dicer or Argonaute polypeptide. The invention further provides polypeptides produced using such methods and compositions comprising them. In some embodiments of the invention, a polypeptide of the invention, e.g., a budding yeast Dicer or Argonaute polypeptide, is expressed in cells and isolated from the cells or isolated from the medium in which the cells are cultured. Any suitable host cell can be used to express the polypeptides of the invention. In some embodiments, bacterial cells are used. In some embodiments, gram negative bacterial cells (e.g., Escherichia species, e.g., E. coli) are used, while in other embodiments gram positive cells (e.g., Bacillus species, e.g., B. subitilis) are used. In some embodiments, a protease deficient host cell is used. See, e.g., US Pat. Pub. Nos. 20020142388; 20090075332. In some embodiments, the polypeptide is expressed using a coding sequence that has been codon optimized for expression in the host cell. In some embodiments, a polypeptide of the invention is expressed in fungal, insect, plant, or mammalian cells. Standard methods of cell culture, protein expression, and purification may be used. The cell may stably or transiently express the protein. See, e.g., Doyle, S. (ed.) High Throughput Protein Expression and Purification: Methods and Protocols (Methods in Molecular Biology) Humana Press, 2008; Higgins, S J and Hames, B D., Protein Expression: A Practical Approach (Practical Approach Series) Oxford University Press, 1999. Exemplary procedures for expressing and purifying budding yeast Dicer polypeptide are provided in the Examples. Suitable methods include, e.g., Ni-affinity, ion-exchange, hydrophobic-interaction, Heparin-affinity and/or gel-filtration columns. For example, application of these purification steps as described in Example 8 resulted in protein that was extremely pure, e.g., of a suitable quality for crystallography. For standard dicing reactions, sufficient purity can be achieved using far fewer steps. In some embodiments, a polypeptide of the invention may be produced using chemical means such as conventional solid phase peptide synthesis, in vitro translation, and/or using methods involving chemical ligation of synthesized peptides (see, e.g., Kent, S., J Pept ScL, 9(9):574-93, 2003 and U.S. Pub. No. 20040115774), or a combination of these. The invention provides extracts from cells that express a budding yeast Dicer polypeptide of the invention and methods of use thereof, e.g., to produce siRNA. Thus the invention further relates to an in vivo or in vitro system for producing siRNA.

The invention further provides an antibody that binds to a Dicer polypeptide of the invention, e.g., an antibody that binds to a Dicer polypeptide of a budding yeast that has a functional RNAi pathway. The invention further provides an antibody that binds to an Argonaute polypeptide of a budding yeast that has a functional RNAi pathway. An antibody of the invention may be monoclonal or polyclonal. Antibodies of the invention can be used for a variety of purposes. For example, they may be used to determine whether a cell expresses a Dicer or Argonaute polypeptide, to quantify the polypeptide, and/or to isolate the polypeptide. An antibody may be a labeled, e.g., with a detectable moiety, may be attached to a solid support, and/or may be provided as part of a protein array. It will be appreciated that the binding need not be completely specific. In some embodiments the antibody allows one to distinguish between a budding yeast Dicer polypeptide and a Dicer polypeptide from another eukaryote. In some embodiments the antibody allows one to distinguish between budding yeast Dicer polypeptides from different budding yeast.

The invention provides a method for detecting the presence of a polypeptide of the invention. In some embodiments method comprises: (a) contacting the sample with an antibody that binds to the polypeptide; and (b) determining whether the antibody binds to a polypeptide in the sample. In some embodiments the method comprises: (a) contacting the sample with an antibody that selectively binds to a budding yeast Dicer polypeptide; and (b) determining whether the antibody binds to a budding yeast Dicer polypeptide in the sample. In some embodiments the method comprises: (a) contacting the sample with an antibody that binds to adding yeast Argonaute polypeptide; and (b) determining whether the antibody binds to an Argonaute polypeptide in the sample. The sample may comprise, e.g., a cell, population of cells, cell extract, partially purified preparation of the polypeptide, etc. Standard antibody-based methods of detection can be used, e.g., Western blots, immunoprecipitation followed by Western blot, etc.

The invention provides budding yeast cells that lack at least one functional endogenous RNAi pathway polypeptide and are genetically engineered to have a functional version of such RNAi pathway polypeptide. In some aspects the invention provides budding yeast cells that lack a functional endogenous RNAi pathway and are genetically engineered to have a functional RNAi pathway. In some embodiments, the budding yeast lacks an endogenous gene encoding a functional Dicer polypeptide and is engineered to contain a nucleic acid encoding a functional Dicer polypeptide of the invention. The budding yeast may have an endogenous gene that encodes a functional Argonaute polypeptide, so that the resulting genetically engineered yeast has a functional RNAi pathway. In some embodiments, the budding yeast lacks an endogenous gene encoding a functional Argonaute polypeptide and is engineered to contain a nucleic acid encoding a functional Argonaute polypeptide of the invention. The budding yeast may have an endogenous gene that encodes a functional Dicer polypeptide, so that the resulting genetically engineered yeast has a functional RNAi pathway. In some embodiments, the budding yeast lacks an endogenous gene encoding a functional Dicer polypeptide and lacks an endogenous gene encoding a functional Argonaute polypeptide and is engineered to contain a nucleic acid encoding a functional Dicer polypeptide of the invention and to contain a nucleic acid that encodes a functional Argonaute polypeptide of the invention. The genetically engineered yeast may further comprise a nucleic acid that comprises a template for transcription of a dsRNA that is cleaved by Dicer to yield siRNA that silence a gene of interest, e.g., that direct cleavage of the mRNA of the gene.

The invention provides libraries (collections) of budding yeast strains, in which one or more genes are targeted for silencing by RNAi. Such strains could be of a species that has an endogenous RNAi pathway or of a species that is engineered to have a functional RNAi pathway, e.g., S. cerevesaie. In some embodiments, the strains are “bar-coded’. As known in the art, a DNA barcode is a short DNA sequence that uniquely identifies a certain linked feature such as a gene or a mutation (see, e.g., Xu, Q, et al., Proc Natl Acad Sci USA, 106(7):2289-94, 2009, and references therein). In the libraries of the invention, a bar code can identify a gene (or a group of genes) that is silenced by RNAi. DNA barcodes built into the yeast deletion collection have facilitated identification of genes whose mutants are depleted or enriched under various growth conditions or drug treatments. The invention encompasses use of the collection of RNAi strains for similar purposes, among others, in a variety of different yeast species and strains. In some embodiments, the library comprises strains in which at least 10 different genes are targeted, i.e., a different gene is targeted in each of the strains. In other embodiments, the library comprises at least 20, 50, 100, 500, 1000, 2000, 3000, 4000, 5000, 6000 or more strains in each of which a different gene is targeted. In some embodiments, the library comprises strains in which at least 10%, 20%, 30%, 50%, 75%, 80%, 85%, 90%, or more of the genes of the library species are targeted. In some embodiments, the strains of the library are isogenic except with respect to the dsRNA that targets a gene for silencing. In some embodiments, the constructs that provide the template for dsRNA are integrated into the same locus of the genome in different strains. In one embodiment, the level of silencing in the strains under certain conditions may be above a predetermined level, e.g., above 50%. The invention encompass such libraries in any species (yeast or non-yeast) that is genetically engineered to have a functional budding yeast RNAi pathway.

In some embodiments, a library comprises strains such that a group of yeast genes of interest are silenced in different strains of the library. The group may comprise or consist of at least some genes that encode products that function in a major biological process, fall into a functional category of interest, or relate to a cellular component of interest (e.g., a subcellular structure, location, or macromolecular complexes), e.g., as defined by the Gene Ontology (GO) project (http://www.geneontology.org/index.shtml). See, e.g., Christie, K R, et al., “Functional annotations for the Saccharomyces cerevisiae genome: the knowns and the known unknowns”, Trends in Microbiology, 17(7): 286-294, 2009, for a review. For example, the group may comprise genes encoding enzymes of a particular biosynthetic pathway, proteins having a particular biochemical activity, etc.

In some embodiments, a library of yeast strains may be generated using a library of nucleic acids, e.g., vectors, each of which comprises a template for transcription of a dsRNA that corresponds to a different gene, wherein the template is operably linked to an expression control element. Optionally, such nucleic acids, e.g., vectors, also comprise polynucleotides that encode an RNAi pathway polypeptide, e.g., a Dicer or Argonaute polypeptide. Such libraries of nucleic acids and vectors are aspects of the invention. Members of a library (e.g., a library of nucleic acids, vectors, yeast strains) may, e.g., be contained in individual receptacles (e.g., tubes, wells of a microtiter plate, culture vessels, etc.), which individual receptacles may be labeled for identification. The invention further provides genetically engineered budding yeast in which a gene that encodes a functional RNAi pathway polypeptide, e.g., Dicer or Argonaute, is at least in part mutated, e.g., at least in part deleted and, optionally, rendered non-functional.

Kits

The invention provides a kit comprising any one or more of the genetically engineered yeast that have a functional RNAi pathway, isolated nucleic acids, vectors, polypeptides, antibodies, siRNA pools, or libraries described herein. In one embodiment, the kit comprises genetically engineered S. cereveisiae cells that have a functional RNAi pathway. In some embodiments, a kit comprises (i) instructions for silencing a gene in the yeast cell using RNAi; (ii) a nucleic acid construct for use in engineering the yeast cell to express an siRNA precursor, e.g., a dsRNA, corresponding to a gene of interest; and/or (iii) a nucleic acid construct for use in engineering the yeast cell to express a control dsRNA. In some embodiments, a kit comprises: (i) a nucleic acid that encodes a functional budding yeast Dicer polypeptide; (ii) a nucleic acid that encodes a functional budding yeast Argonaute polypeptide; and/or (iii) instructions for producing a budding yeast cell that has a functional RNAi pathway. In some embodiments, nucleic acids (i) and (ii) are provided as part of a single nucleic acid, e.g., in a vector, and are each optionally operably linked to an expression control element, e.g., a promoter. In some embodiments the kit further comprises (iv) a nucleic acid construct for use in engineering the yeast cell to express a control dsRNA, which may be used to verify that RNAi is functioning in the cell. In some embodiments, a kit comprises: (i) a budding yeast Dicer polypeptide; (ii) a nucleic acid encoding a budding yeast Dicer polypeptide; (iii) a cell (e.g., a yeast or bacterial cell) that expresses a budding yeast Dicer polypeptide; (iv) reagent(s) for purifying a budding yeast Dicer polypeptide; and/or (v) instructions for purifying a budding yeast Dicer polypeptide and/or instructions for producing siRNA by cleaving dsRNA using a budding yeast Dicer protein in vivo and/or in vitro. In some embodiments, the functional Dicer polypeptide has the sequence of a naturally occurring full length budding yeast Dicer polypeptide. In some embodiments, the functional Dicer polypeptide has the sequence of a variant or fragment of a naturally occurring full length budding yeast Dicer polypeptide. In some embodiments, the nucleic acid, e.g., vector, that encodes budding yeast Dicer polypeptide further encodes a tag, so that the encoded polypeptide comprises a Dicer polypeptide with an N- or C-terminal tag. Optionally, a cleavage site for a protease is positioned between the tag and the functional Dicer polypeptide, so that the tag can be removed. In some embodiments, the tag is removed after purification of the polypeptide. In some embodiments, cleavage of the tag is used as a purification step. A kit may comprise an inducer, a restriction enzyme, a ligation mix, a protease, an affinity matrix, a culture medium, an antibody, a buffer, and/or a control vector.

In some embodiments, a kit comprises (i) a budding yeast Dicer polypeptide; and at least one of the following items: (ii) reagent(s) for in vitro transcription to produce RNA that can hybridize to produce dsRNA; (iii) one or more substances useful for hybridization (“annealing”) of complementary RNA to produce dsRNA; (iv) one or more substances useful for an in vitro reaction in which the budding yeast Dicer polypeptide cleaves dsRNA; (v) reagent(s) for isolating siRNA produced by cleaving dsRNA in vitro using the budding yeast Dicer polypeptide; (vi) reagent(s) useful for detecting siRNA; (vi) one or more substances useful for storing a polypeptide, dsRNA, or siRNA.

Reagents for in vitro transcription could comprise, for example, (a) one or more RNA polymerases (e.g., a phage RNA polymerase such as T3, T7, or SP6 polymerase), (b) a vector (e.g., a plasmid) useful for synthesizing dsRNA by in vitro transcription, (c) primers that hybridize to the plasmid, wherein the primers can be used to amplify a DNA fragment inserted into the plasmid to produce a DNA template for transcription by the RNA polymerase, (d) a reagent useful to isolate or immobilize a DNA fragment, (e) one or more substances useful for an in vitro transcription reaction using the RNA polymerase, (f) ribonucleotide triphosphates, (g) a reagent useful to purify dsRNA; and/or (h) a reagent useful to quantify dsRNA.

A typical vector useful for synthesizing dsRNA by in vitro transcription contains a promoter for an RNA polymerase and a site for inserting a DNA fragment of interest. The DNA fragment of interest contains a portion that corresponds in sequence to at least a portion of an mRNA to be silenced by RNAi. In some embodiments, the vector contains two oppositely directed promoters for the RNA polymerase, wherein the promoters flank a site for inserting a DNA fragment of interest. The two promoters could correspond to the same RNA polymerase or to different polymerases. The site for inserting a DNA fragment typically comprises a restriction site, e.g., a multiple cloning site. Dual, oppositely directed promoters allow transcription of both strands of the insert, and the resulting transcripts can then be annealed to form dsRNA. Suitable vectors are known in the art. In some embodiments, the vector may be used directly as a template for in vitro transcription of both strands. The vector may be linearized by restriction enzyme digestion prior to use as a template and/or may contain appropriately positioned transcription terminators to ensure production of RNA transcripts of a defined length and sequence. In some embodiments, the vector does not necessarily contain dual opposing promoters. For example, two plasmids, with the insert cloned in opposite orientation, could be used to synthesize two separate strands. Alternately, the insert could comprise first and second portions that form an inverted repeat (with the two portions of the inverted repeat optionally being separated by a spacer portion), so that the resulting transcript contains two complementary portions capable of annealing to each other to form a stem-loop structure.

In some embodiments, the vector is used as a template for amplification, e.g., by PCR, to generate a DNA fragment that serves as a template for transcription. In such embodiments, primers hybridize to sequences flanking at least a portion the insert, typically sequences outside the insert. For example, if the vector contains dual oppositely directed RNA polymerase promoters, the primers can hybridize to these promoters so that each strand of the amplified DNA fragment contains a promoter for the polymerase. In other embodiments, the primers can contain the RNA polymerase promoter sequence, e.g., at the 5′ end so that the resulting product contains promoters at each end. In some embodiments the primers contain an affinity tag such as biotin to allow convenient isolation and/or immobilization of an amplified DNA fragment containing the primer by contacting the DNA fragment with a binding partner for the affinity tag. For example, amplified DNA generated using a biotinylated primer can be readily isolated by binding to streptavidin. Optionally, the binding partner for the affinity tag is attached to a support such as a bead, e.g., a magnetic bead, allowing immobilization of the DNA template so that transcribed RNA can be readily separated from the DNA template after in vitro transcription. The kit can include one or more substance(s) useful for an in vitro transcription reaction using the RNA polymerase. Following reverse transcription, the transcribed RNA can be isolated, e.g., by separating it from salts, unincorporated nucleotides, the DNA template, and/or the RNA polymerase. The kit can contain substances useful for hybridizing (“annealing”) complementary RNA (either two individual RNA molecules or two complementary portions of a single molecule) to produce dsRNA. Typical components include, e.g., a salt such as NaCl and a buffer such as Tris-HCl, as known in the art. Substances useful for hybridization of complementary RNA may be provided together in a composition (which may be referred to as an “annealing buffer”).

The kit may contain reagent(s) useful to isolate RNA. In some embodiments, a reagent useful to isolate RNA, e.g., ssRNA or dsRNA comprises a precipitating reagent or a spin column. In some embodiments, the kit comprises a spin column that allows separation of the RNA from salts, proteins, and/or unincorporated ribonucleotides. In some embodiments, a spin column allows separation of RNA of the desired size from larger or smaller nucleic acids, e.g., larger or smaller RNAs. Optionally, the dsRNA is quantified. In some embodiments, a reagent useful to quantify dsRNA binds to RNA and comprises a detectable label such as a fluorescent moiety. For example, a fluorescent dye such as RiboGreen® can be used (Invitrogen, Carlsbad, Calif.).

In some embodiments, a kit contains one or more substances useful for an in vitro reaction in which a budding yeast Dicer polypeptide cleaves dsRNA. Such substances could include, e.g., a divalent cation such as Mg⁺⁺ (e.g., as MgCl₂), an energy source such as ATP, a salt such as NaCl, a buffer such as Tris-HCl, a reducing agent such as DTT, and/or a calcium chelating agent such as EDTA. In some embodiments, at least some of the substances are provided together in a composition, which may be referred to as a “reaction buffer”. An exemplary 5× reaction buffer for an in vitro cleavage reaction contains 150 mM Tris-HCl pH 7.5, 150 mM NaCl, 25 mM MgCl₂, 5 mM DTT, and 0.5 mM EDTA.

The kit can contain one or more substances useful for storing a polypeptide, dsRNA, or siRNA. Such substances could include, e.g., a buffer such as Tris-HCl, a salt such as NaCl, a reducing agent such as dithiothreitol (DTT), a stabilizing agent such as glycerol, a carrier protein such as albumin and may be provided together in a composition (which may be referred to as a “storage buffer”). One of skill in the art will be aware of suitable compositions for storing RNA or polypeptides. An exemplary composition for storing a Dicer polypeptide contains 10 mM Tris-HCl pH 7.5, 200 mM NaCl, and 5 mM DTT. Another exemplary composition for storing a Dicer polypeptide contains 5 mM Tris-HCl pH 7.5, 100 mM NaCl, 2.5 mM DTT, 50% glycerol, and 1 mg/ml Ultrapure bovine serum albumin (Ambion).

In some embodiments, a kit contains reagent(s) for isolating siRNA. Such reagent(s) could comprise, for example, (a) one or more spin columns or adsorbents suitable for isolating dsRNA of about 23 nucleotides in length, (b) a solution for eluting dsRNA from a gel (which may be referred to as an “elution buffer”). In some embodiments, the kit comprises a spin column that allows removal of salts and/or unincorporated ribonucleotides and/or a spin column that allows removal of uncleaved dsRNA, e.g., dsRNA longer than about 100, 200, or 300 nt in length. A spin column could comprise a resin or adsorbent suitable for separating moieties based, e.g., on size or affinity. In some embodiments, a kit comprises one or more markers or standards (e.g., a marker suitable for detecting siRNA of about 23 nucleotides in length).

In some embodiments, a kit contains one or more reagent(s) useful for detecting siRNA. Such reagent(s) could comprise, e.g., a dye that binds to RNA such as SYBR® Gold.

A kit could comprise one or more items useful for control purposes, e.g., a control plasmid, control primer(s), control siRNA. The control plasmid could contain an insert such as a coding sequence for GFP. The control plasmid and/or control primers could be used to confirm that amplification, transcription, and/or cleavage by Dicer are occurring appropriately. A control plasmid could be transfected into cells together with siRNA generated in a control reaction to confirm that silencing is occurring appropriately and/or to quantitate the relative degree of silencing. In some embodiments, a kit contains a transfection reagent, e.g., a reagent of use to transfect animal cells, e.g., insect cells, avian cells, mammalian cells, etc. with siRNA and, optionally, plasmid(s). Transfection reagents suitable for transfecting mammalian cells are known in the art. For example, a variety of chemical agents such cationic and/or neutral lipids, liposomes, cationic polymers such as DEAE-dextran or polyethylenimine, and cationic peptides are of use. Electroporation with a suitable electroporation buffer can also be used. Transfection reagents suitable for transfecting siRNA into animal cells are commercially available. Examples of chemical transfection reagents include FuGene HD (Roche Applied Biosciences), DharmaFECT transfection reagent (Thermo Fisher), Lipofectamine 2000 (Invitrogen), HiPerFect Transfection Reagent and RNAiFect Transfection Reagent (both from Qiagen), among others. siPORTT™ siRNA electroporation buffer (Ambion) is useful for electroporating siRNA into cells. In some embodiments, a transfection reagent has been optimized for transfecting siRNA, e.g., into mammalian cells.

Instructions for performing and/or troubleshooting (i) an in vitro transcription reaction, (ii) a Dicer polypeptide-mediated cleavage reaction, (iii) isolation of DNA, dsRNA, or siRNA, and/or (iv) a transfection can be included.

In another embodiment, the invention provides a kit for detecting a budding yeast Dicer polypeptide. The kit comprises an antibody of the invention that selectively binds to a budding yeast Dicer polypeptide and, optionally, a detection reagent or secondary antibody for detecting the antibody, a sample of Dicer polypeptide for use as a control, and instructions for use. In another embodiment, the invention provides a kit for detecting a budding yeast Argonaute polypeptide. The kit comprises an antibody of the invention that selectively binds to a budding yeast Argonaute polypeptide and, optionally, a detection reagent or secondary antibody for detecting the antibody, a sample of Argonaute polypeptide for use as a control, and instructions for use.

Components of a kit can be packaged together in a single container or may be provided in multiple containers. A composition for annealing, reaction, storage, elution, etc., may be provided in concentrated form (e.g., as a 5×, 10×, 50× concentrate), which can be diluted to lx to provide a suitable concentration for the intended use. In some embodiments, two or more individual kits (which may be packaged together in a single larger container) are provided. For example, an in vitro transcription kit and a kit for cleaving dsRNA using a functional budding yeast Dicer polypeptide can be provided.

It will be understood that the invention encompasses the use of reagents and methods described in this section, or similar reagents and methods, in various other aspects of the invention.

Selected Applications

This section describes certain applications of interest, e.g., relating to budding yeast RNAi pathway genes and polypeptides, cells that express them, and/or uses of RNAi and/or siRNA in budding yeast or other organisms. Other applications of interest are described above and/or in the Examples, and it will be understood that the invention may be used for other purposes as well.

With the discovery and characterization of the budding-yeast pathway, RNAi can be used, e.g., in budding yeast for a variety of different purposes. For example, RNAi may be used as a tool in the study of gene function in budding yeast such as S. castellii or Kluyveromyces polysporus that have an endogenous functional RNAi pathway or in budding yeast such as S. cerevisiae that lack a functional endogenous RNAi pathway and are genetically engineered to have a functional RNAi pathway as described herein. Without wishing to be bound by theory, the use of RNAi in budding yeast could provide easier, more flexible, and finer control for gene silencing relative to the existing genetic technologies for reducing gene expression in S. cerevisiae. RNAi might provide a particularly convenient approach in species that are obligate diploids, such as C. albicans (8), polyploid strains, and/or strains or species that have multiple copies of a gene whose silencing is desired. RNAi also provides a convenient way to silence multiple genes in a particular cell. In some embodiments, RNAi is used to silence members of repetitive gene families in budding yeast. In some embodiments RNAi is used to silence a gene positioned at a recombination-resistant location. In some embodiments, RNAi is used to silence a gene in a yeast species in which homologous recombination techniques are not available or are less reliable than in S. cerevisiae.

RNAi enables a constitutive or inducible knock-down system that provides an alternative to existing technologies for generating yeast with reduced expression, such as technologies that involve either non-physiological expression of the gene of interest (e.g. the GAL/GLU repression system), generation of temperature-sensitive mutations, transcriptional shutoff, or conditional protein destabilization. See, e.g., Pan, X., et al. Mol Cell, 16(3):487-96 (2004); Kanemaki, K., et al., Nature, 423: 720-724 (2003). RNAi may also be used together with such technologies. Thus in some embodiments, the RNAi system is used together with methods available in the art for generating budding yeast with reduced expression.

The invention encompasses use of budding yeast RNAi pathway polypeptides in any cell of interest. Without wishing to be bound by theory, the relatively small size of budding yeast Dicer and/or its apparent ability to function without co-factor proteins found in RNAi pathways described in other organisms may facilitate the genetic engineering of RNAi pathway in a variety of different species. As described in the Examples, it was shown that budding yeast Dicer can be produced in bacterial cells and retain its dicing activity. This discovery opens the way to constituting a Dicer-based RNAi pathway in prokaryotes, e.g., bacteria or Archaea, by engineering such cells to be capable of expressing a budding yeast Dicer and, optionally, Argonaute polypeptide. The invention thus provides methods of silencing a gene in prokaryotes, e.g., bacteria, using RNAi. In some embodiments, budding yeast Dicer and/or Argonaute polypeptides can be introduced into fungi that apparently lack a functional RNAi pathway (e.g., Ustilago maydis). The invention thus provides methods of silencing a gene using RNAi in fungi that lack an endogenous functional RNAi pathway.

Any gene of interest can be targeted for silencing in various embodiments of the invention. The target gene can be an endogenous gene or a non-endogenous gene. The target gene can encode a protein that has at least one known function or a protein whose function(s) are unknown. In some embodiments the protein is an enzyme. The enzyme can be of any of the following classes (according to the International Union of Biochemistry and Molecular Biology nomenclature for enzymes, the EC numbers in which each enzyme is described by a sequence of four numbers preceded by “EC” and the first number broadly classifies the enzyme based on its mechanism: EC 1 Oxidoreductases: catalyze oxidation/reduction reactions; EC 2 Transferases: transfer a functional group (e.g. a methyl or phosphate group); EC 3 Hydrolases: catalyze the hydrolysis of various bonds; EC 4 Lyases: cleave various bonds by means other than hydrolysis and oxidation; EC 5 Isomerases: catalyze isomerization changes within a single molecule; EC 6 Ligases: join two molecules with covalent bonds. In some embodiments the enzyme is a kinase or phosphatase. In some embodiments the enzyme is a protease.

In some embodiments the target gene encodes a transcription factor. In some embodiments the target gene encodes a structural protein. In some embodiments the target gene encodes a protein that localizes to the cell wall. In some embodiments the target gene encodes a protein that localizes to an organelle. In some embodiments the gene encodes a protein involved in a biological pathway or process of interest. In some embodiments the target gene encodes a protein involved in the secretory pathway, the cell cycle, protein degradation, chromatin remodeling, transcription, splicing, aging, mRNA transport, or mRNA translation. In some embodiments the target gene encodes a protein that metabolizes a product of interest.

In some embodiments the target gene encodes an endogenous yeast protein that has a human homolog. In some embodiments, the human homolog is associated with a disease. In some embodiments, a protein “associated with a disease” is a protein whose mutation, over-expression, or under-expression contributes at least in part to development or progression of the disease and/or increased susceptibility to the disease. In some embodiments the target gene is a non-endogenous human protein. In some embodiments said non-endogenous human protein is associated with disease. In some embodiments the disease is a neurodegenerative disease. In some embodiments the disease is a cancer. In some embodiments the disease is a metabolic disease, e.g., diabetes. In some embodiments the disease is an infectious disease.

In some embodiments of the invention the budding yeast target gene is an essential gene. More than 1,000 essential genes have been identified in S. cerevesiae, of which about 40% have counterparts in human (Mnaiamneh, S., et al., Cell, 118: 31-44, 2004). Such genes are of considerable interest and potential medical relevance but can be challenging to study using existing techniques since, e.g., haploid deletions strains cannot be constructed. In an effort to address these limitations, libraries of genetic hypomorphs (yeast containing hypomorphic alleles) have been generated using an approach termed DAmP in which a gene's 3′ untranslated region is disrupted with an antibiotic resistance cassette, thus destabilizing the corresponding transcript and reducing mRNA amount usually but about 2- to 10-fold. See, e.g., Breslow, D. K., et al., Nat. Methods, 5(8): 711-718 (2008), and references therein. Another approach, which may be combined with DAmP, involves use of a C-terminal degradation tag that targets the protein for proteasomal degradation. RNAi provides an alternative approach to use of genetic hypomorphs or may be used to complement the use of genetic hypomorphs. For example, RNAi could be used to further reduce gene expression in hypomorphs, optionally in an inducible manner. Without wishing to be bound by theory, RNAi may allow production of a library of strains that have less variability in the degree to which mRNA level is reduced. RNAi also affords a way to study essential genes for which it has not been possible to isolate genetic hypomorphs, which include a number of essential genes. A recent report indicated that only 739 haploid strains out of the 1033 essential genes could be tested using the DAmP collection, as the DAmP methodology evidently resulted in lethality when applied to the other almost 300 essential genes (Ungar, L., et al., Nucleic Acids Res., 37(12): 3840-3849). RNAi may be used to generate strains in which the mRNA of these genes is reduced but is sufficient for viability.

In some embodiments, RNAi is used to study gene interactions. RNAi can be used to identify a gene (or group of genes) that shows “synthetic lethality” with a gene of interest. In some embodiments, expression of a gene of interest is partly or completely silenced by RNAi, and mutants (either generated using RNAi or using conventional techniques) that are unable to grow are identified. In some embodiments, a conventionally generated mutant is used, and RNAi is used to identify a gene whose partial or complete silencing results in lethality. In some embodiments, RNAi is used in epistasis analysis. In some embodiments RNAi is used to identify genes that have additive or synergistic effects on a phenotype or process of interest. In some embodiments RNAi is used to identify genes that have opposing effects. In some embodiments RNAi is used to identify a first gene whose silencing alleviates the effect of silencing or mutating a second gene.

RNAi in budding yeast can be used for drug discovery and for identification of drug targets. In some embodiments, RNAi is used in identifying an anti-fungal agent. Fungal infections are significant causes of disease in animals (e.g., humans) and plants. Fungal contamination of organic materials, e.g., foods, paper products, bedding, etc., and of buildings, is also of considerable concern. Fungal infections can be a particular problem in individuals who are immunocompromised (e.g., as a result of administration of immunosuppressive drugs, genetic immunodeficiencies, HIV infection, etc.), or who have implantable devices such as catheters. A variety of anti-fungal agents are in use for therapeutic purposes. However, such agents are not always effective and can have severe side effects. Resistance to anti-fungal agents is an increasing problem. In some embodiments, RNAi is used to identify a gene, gene product, or biological pathway or process that is a target for discovery of an anti-fungal agent. In some methods, RNAi is used to silence expression of a gene in a budding yeast, which may be a pathogenic (e.g., an opportunistic pathogen) or non-pathogenic yeast. The effect of such silencing on viability, pathogenesis, or a phenotype that correlates with pathgenesis is assessed. If silencing the gene reduces viability or reduces pathogenesis or a phenotype that correlates with pathogenesis, the gene or a gene product encoded by the gene is a target for discovery of an anti-fungal agent. In some embodiments, RNAi is used to identify a gene that contributes to drug resistance of a drug resistant budding yeast species or strain. Yeast cells are contacted with an anti-fungal agent (e.g., amphotericin, an echinocandin (e.g., micafungin, caspofungin, anidulafungin), an azole (e.g., fluconazole, itraconazole, ketoconazole, voriconazole) or another anti-fungal agent. RNAi is used to silence a gene, and the effect of such silencing on susceptibility or resistance to the anti-fungal agent is assessed. If silencing the gene reduces resistance or enhances susceptibility to the anti-fungal agent, the gene or a gene product encoded by the gene is a target for discovery of an agent that reduces resistance or enhances susceptibility to an anti-fungal agent. The identified agent can be used in the treatment of individuals, e.g., humans, suffering from an infection with a pathogenic yeast, e.g., a pathogenic budding yeast such as certain Candida species (C. albicans, C. glabrata, C. krusei) or to reduce fungal contamination of surfaces, foods, etc. Since many fungal genes and proteins are well conserved and play analogous roles in different fungal species, it is reasonable to expect such agents to be effective against other fungi that are not budding yeast, e.g., other yeast species, or fungi such as Aspergillus species (e.g., Aspergillus fumigatus).

In some embodiments, RNAi is used to identify a target for development of a drug to treat a disease other than a fungal infection. In some embodiments, RNAi is used to identify a target of a drug whose mechanism of action and/or cellular target is unknown. In some embodiments the disease is cancer. In some embodiments the disease is a metabolic disease such as diabetes. In some embodiments the disease is a neurodegenerative disease, e.g., Alzheimer's disease or Parkinson's disease. In some embodiments, RNAi is used to identify a gene that is affected by a drug and results in an undesired “off-target effect” or “side effect”. In some embodiments, RNAi is used to silence a gene, and the effect of such silencing on drug sensitivity is assessed. In some embodiments, if silencing the gene eliminates drug sensitivity, it can be inferred that the drug acts on the gene or on a product encoded by the gene (e.g., converting it into a toxic agent) or that the gene or gene product is required for activity of the drug. In some embodiments, if partially (but not completely) silencing the gene increases drug sensitivity, it can be inferred that the drug acts on the gene or on a product encoded by the gene, or on a gene or gene product that functions in the same biological pathway as the gene. In some embodiments, if partially or completely silencing the gene increases drug sensitivity, it can be inferred that the drug acts on the gene or on a product encoded by the gene or on a gene or gene product that functions in the same biological pathway as the gene.

Fitness analysis of yeast strains with heterozygous deletions of drug target genes can be used to monitor compound activities in vivo. For example, reducing the gene copy number of drug targets in a diploid cell can result in sensitization to the drug of interest, e.g., diminishing the ability of the cell to reproduce. The haploinsufficient phenotype thereby identifies the gene product of the heterozygous locus as the likely drug target (Giaever, G., et al. Genomic profiling of drug sensitivities via induced haploinsufficiency, Nat. Genet. 21: 278-283, 1999; Lum, P Y, et al. Cell. 116 (1): 121-137, 2004). The present invention provides methods of using RNAi to perform fitness analysis of strains in which gene silencing is achieved using RNAi. In some embodiments, RNAi is used to perform fitness analysis in haploid cells and/or in strains in which essential genes are partially silenced. In some embodiments, RNAi is used to perform fitness analysis in diploid or polyploid strains. In some embodiments, fitness analysis is performed using strains that have weak silencing, strong silencing, or using strains with a range of different silencing levels. In some embodiments, isogenic yeast strains having a functional RNAi pathway are used, each strain being engineered to express a dsRNA corresponding to a gene and a strain-specific molecular barcode tag. A mixture or “pool” of these strains is produced. Competitive growth of the pool is carried out (e.g., for about 15-25 generations) in media containing selected compounds of interest. Strain abundance is measured before and after outgrowth, e.g., by hybridization of differentially labeled (Cy3/Cy5) barcode tags to DNA microarrays. Strains that are sensitive to a given compound are outcompeted by unaffected strains in the pool. The gene that is silenced in such strains is a candidate target of the drug. The number of false positives can be reduced by confirming the fitness results with individual strains in which the gene is inhibited by RNAi or is mutated or deleted and testing whether overexpression of the candidate gene confers resistance to the drug of interest. Fitness analysis can also be used in a similar manner to identify genes whose silencing improves resistance to a harmful environmental condition or stress. In some embodiments, the invention envisions performing flow cytometry based growth competition assays using RNAi strains, e.g., as described for DAmP strains in Breslow, supra.

Any drug or other compound of interest, or combination thereof, can be evaluated. The drug can be one that has been approved by a government regulatory agency such as the US Food and Drug Administration or a compound that is in preclinical or clinical development, or under consideration for development, as a therapeutic agent. The drug may be, e.g., an antineoplastic, antibacterial, antiviral, antifungal, antiprotozoal, antiparasitic, antidepressant, antipsychotic, anesthetic, antianginal, antihypertensive, antiarrhythmic, antiinflammatory, analgesic, antithrombotic, antiemetic, immunomodulator, antidiabetic, lipid- or cholesterol-lowering (e.g., statin), anticonvulsant, anticoagulant, antianxiety, hypnotic (sleep-inducing), hormonal, or anti-hormonal drug, etc. The compound may be a known or suspected toxin, mutagen, carcinogen, environmental pollutant. Various types of candidate drugs may be screened, identified, or evaluated using the methods described herein, such as small organic molecules, inorganic molecules, nucleic acids, polypeptides, and peptidomimetics (e.g., peptoids). In some embodiments, nucleic acids and polypeptides are screened by contacting the yeast cell with a nucleic acid construct, e.g., a vector, designed such that the yeast cell contacted with the vector expresses the nucleic acid or polypeptide. For example, cDNA libraries encoding a variety of proteins (which may be of yeast or non-yeast origin and may be naturally occurring or artificial) may be screened or evaluated. Small organic molecules typically have a molecular weight in the range of 50 to 2,500 daltons. These compounds often contain multiple carbon-carbon bonds and can comprise functional groups important for structural interaction with proteins (e.g., hydrogen bonding), and typically include at least an amine, carbonyl, hydroxyl, or carboxyl group, and in some embodiments at least two of the functional chemical groups. These compounds often comprise one or more cyclic carbon or heterocyclic structures and/or aromatic or polyaromatic structures, optionally substituted with one or more of the above functional groups. Compounds may comprise nucleotides, amino acids, sugars, fatty acids, and derivatives or structural analogs thereof. Nucleotides and amino acids may be standard or non-standard. If non-standard, they may be naturally occurring or non-naturally occurring (i.e., not found in nature). Similarly, nucleic acids and polypeptides may comprise standard or non-standard nucleotides and amino acids, respectively, and may have non-standard inter-subunit linkages.

Compounds can be members of, e.g., chemical libraries, natural product libraries, combinatorial libraries, etc. Chemical libraries can comprise diverse chemical structures, some of which may be known compounds, analogs of known compounds, or analogs or compounds that have been identified as “hits” or “leads” in other drug discovery screens, while others are derived from natural products, and still others arise from non-directed synthetic organic chemistry. Compounds from chemical libraries are often arrayed in multi-well plates (e.g., 96- or 384-well plates). Natural product libraries can be prepared from collections of microorganisms, animals, plants, or marine organisms which are used to create mixtures for screening by, e.g.,: (1) fermentation and extraction of broths from soil, plant or marine microorganisms, or (2) extraction of plants or marine organisms. Compound libraries are commercially available from a number of companies. In addition, various government and non-profit research institution have compound libraries that are available to the scientific community. For example, the Molecular Libraries Small Molecule Repository (MLSMR), a component of the National Institutes of Health (NIH) Molecular Libraries Program is designed to identify, acquire, maintain, and distribute a collection of >300,000 chemically diverse compounds with known and unknown biological activities for use, e.g., in high-throughput screening (HTS) assays (see https://mli.nih.gov/mli/). The NIH Clinical Collection (NCC) is a plated array of approximately 450 small molecules that have a history of use in human clinical trials. These compounds are highly drug-like with known safety profiles. The NCC collection is arrayed in six 96-well plates. 50 μl of each compound is supplied, as an approximately 10 mM solution in 100% DMSO.

In some embodiments, methods that involve contacting a yeast cell with a drug are optionally carried out in yeast strains bearing mutations in or deletions of the ERG6 gene, the PDR1 gene, the PDR3 gene, the PDR5 gene, the SNQ2 gene, and/or any other gene which affects membrane efflux pumps and/or increases permeability for drugs, so as to reduce efflux and/or increase permeability. In some embodiments, RNAi is used to inhibit expression of a gene encoding an efflux pump.

Budding yeast are used to produce a wide variety of compounds of interest. For example, various strains of S. cerevesiae or strains whose genome is at least in part derived from S. cerevesiae are used extensively in fermentative production processes. In addition to S. cerevesiae, industrially important yeast include S. pastorianus, and Kluyveromyces lactis. See, e.g., Satyanarayana, T. and Kunze, G. (eds.) Yeast biotechnology: diversity and applications; Springer, 2009, and references therein. In some embodiments of the invention, RNAi is used in metabolic engineering of yeast, e.g., budding yeast, e.g., industrially important budding yeast, to improve cellular activities by manipulating, e.g., enzymatic, transport, and/or regulatory functions with the use of recombinant nucleic acid (e.g., recombinant DNA) technology. Metabolic engineering can result in a product with improved quality, or result in time and/or cost savings, etc. See, e.g., Nevoigt, E., Microbiology and Molecular Biology Reviews, 72(3): 379-412 (2008) and references therein, all of which are incorporated herein by reference.) “Cellular activities” can comprise product formation or cell properties such as stress tolerance (e.g., tolerance to extremes of temperature (e.g., heat stress), osmotic stress, oxidative stress, pH, intracellular or extracellular accumulation of a product), or ability to utilize particular nutrients or substrates.

The invention encompasses the use of RNAi in cells, e.g., yeast, e.g., budding yeast, for purposes of metabolic engineering and/or for identifying genes of use in metabolic engineering. The invention also encompasses the use of RNAi in bacterial cells that express budding yeast RNAi pathway genes (e.g., Dicer) for such purposes. RNAi can be used to reduce expression of a gene, wherein inhibition of the gene improves a cellular activity and/or to identify genes whose inhibition improves a cellular activity. In some embodiments, inducible RNAi is used to silence a gene whose deletion causes a growth defect under some conditions, while being advantageous in other conditions. In some embodiments, RNAi is used during only a portion of a production process. For example, expression of a dsRNA or of an RNAi pathway polypeptide (e.g., Dicer) may be induced at a certain stage of a production process. RNAi provides a means of conveniently evaluating the effect of reducing the expression of one or more genes, optionally in a variety of strain backgrounds. In some embodiments, 2 or more genes, e.g., 3, 4, or 5 genes, are silenced. In some embodiments, RNAi is used together with existing techniques used for metabolic engineering, such as global transcription machinery engineering (see, e.g., PCT/US2006/037597, published as WO/2007/038564).

In some embodiments, RNAi is used in production of a product of interest or to metabolize (e.g., break down, degrade) a product of interest. In some embodiments of the invention, RNAi is used in an industrially important yeast, e.g., a yeast species or strain that is used to produce a product of interest sold or traded in interstate commerce in the U.S. or internationally. In some embodiments, RNAi is used in a yeast species or strain that has been given GRAS (generally recognized as safe) status by the FDA. In some embodiments RNAi is used in a yeast that has been genetically engineered to improve one or more cellular activities by deleting, mutating, or expressing (e.g., overexpressing) a gene. The yeast may express one or more heterologous gene(s) from a different yeast or other fungus, from bacteria, or from a non-fungal eukaryote. For example, Saccharomyces yeasts have been genetically engineered to ferment xylose, one of the major fermentable sugars present in cellulosic biomasses, so that ethanol can be efficiently produced from using less expensive feedstocks.

S. cerevesiae and other budding yeasts are used extensively in the baking, wine, and brewing industries, in the production of products of interest such as biofuels (e.g., ethanol), fine and bulk chemicals such as glycerol, propanediol, organic acids, sugar alcohols, L-G3P, ergosterol and other steroids, and isoprenoids, to name a few. In some embodiments, RNAi is used to improve the production of a food, nutritional supplement, beverage, or component thereof. In some embodiments, RNAi is used in a baker's, wine, brewer's, sake, or distiller's yeast, e.g., S. cerevesiae or S. pastorianus. In some embodiments, RNAi is used in a yeast species or strain that has been given GRAS (generally recognized as safe) status by the FDA. In some embodiments RNAi is used in a yeast that has been genetically engineered to improve one or more cellular activities by deleting, mutating, or expressing (e.g., overexpressing) a gene. For example, the yeast may express one or more heterologous gene(s) from a different yeast or other fungus, from bacteria, or from a non-fungal eukaryote. For example, Saccharomyces yeasts have been genetically engineered to ferment pentose(s), e.g., xylose, one of the major fermentable sugars present in cellulosic biomasses, so that ethanol can be efficiently produced from using less expensive feedstocks.

In some embodiments, the yeast is of the genus Kluveromyces. For example, Kluveromyces lactis and Kluyveromyces marxianus are of use in a variety of biotechnological processes. In some embodiments, the yeast has increased tolerance to an environmental condition, e.g., heat, cold, osmolarity (e.g., salt concentration) relative to S. cerevesiae. In some embodiments, the yeast is of the genus Debaryomyces, e.g., Debaryomyces hansenii, which is a cryotolerant, marine yeast that can tolerate salinity levels up to 24%. Cryo- and osmotolerance account for its important role in several agro-food processes. D. hansenii is common in cheeses (wherein it provides proteolytic and lipolytic activities during cheese ripening) and is also found in dairies and in brine because it is able to grow in the presence of salt at low temperature and to metabolize lactic and citric acids.

In some embodiments the budding yeast is a methylotrophic yeast (yeasts that can grow on methanol). Pichia pastoris is widely used for production of heterologous proteins (see, e.g., Macauley-Patrick S, et al., Yeast. 22(4):249-70 (2005). As a methylotroph, it can grow with the simple alcohol methanol as its only source of energy. Its genome has been sequenced (De Schutter K., et al. Nature Biotechnology 27: 561-566 (2009). Other methylotrophic yeasts includes Candida boidinii, Pichia methanolica, and Hansenula polymorpha (Pichia angusta). See, e.g., Gellissen G et al. New yeast expression platforms based on methylotrophic Hansenula polymorpha and Pichia pastoris and dimorphic Arxula adeninivorans and Yarrowia lipolytica—a comparison. FEMS Yeast Res. 5, 1079-1096 (2005) and Gellissen G (ed) (2005) Production of recombinant proteins—novel microbial and eukaryotic expression systems. Wiley-VCH, Weinheim, 2005, for additional information regarding these yeast and methods of use thereof.

In some embodiments RNAi is used to identify a gene involved in production of a product of interest or that affects production of a product of interest. In some embodiments, the product of interest is a recombinant protein. Exemplary proteins that can be produced in yeast are antibodies, vaccine components, interferons, and insulin. In some embodiments, the product of interest is a pharmaceutical agent, which may be a recombinant protein or a non-protein biomolecule. In some embodiments the product of interest is a small organic molecule. In some embodiments the product of interest is a precursor that may be subsequently used in a process that may, but need not, involve yeast.

In some embodiments, the product of interest is a biofuel. Biofuel is defined as solid, liquid or gaseous fuel obtained from relatively recently lifeless or living biological material and is different from fossil fuels, which are derived from long dead biological material. In some embodiments the biofuel is an alcohol. In some embodiments, the biofuel is a bio-oil. Ethanol is an exemplary biofuel. S. cerevesiae has traditionally been used for ethanol production (Nevoit, supra). Approaches for improving yeast bioethanol production can include, e.g., (i) efforts to improve processes that use starch or sugar as a starting material; (ii) efforts to improve processes that use lignocellulosic biomass substrate, and/or (iii) efforts to improve sugar-to-ethanol conversion efficiency and/or yeast ethanol tolerance. In some embodiments, RNAi is used in yeast to silence genes whose silencing improves ethanol tolerance, increases ethanol yield, and/or allows the use of a broader range of substrates for ethanol production. For example, deregulating glucose repression of galactose utilization can improve galactose utilization in the production of ethanol. Simultaneous deletion of GAL6, GAL80, and MIG1 was shown to result in an increase in specific galactose uptake rate (Ostergaard, S., et al., Nat. Biotechnol., 18:1283-1286, 2000). The present invention envisions silencing these genes by RNAi. In some embodiments, RNAi is used to improve ethanol production in a yeast that naturally utilizes pentoses, e.g., xylose, such as P. stipitis.

In some embodiments, a product of interest is a lipid. In some embodiments the yeast is an oleaginous yeast. In some embodiments the yeast is a Yarrowia. Yarrowia lipolytica is an exemplary yeast that has developed efficient mechanisms for breaking down and using hydrophobic substrates. It has an ability to accumulate large amounts of lipids and has a variety of biotechnological applications.

In some embodiments, a yeast is used to remediate waste or in environmental cleanup. For example a yeast may be used to degrade oil after an oil spill or otherwise decontaminate areas that have accumulation of undesired substances, e.g., pollutants, that can be metabolized by the yeast.

In some embodiments, RNAi is used in production or metabolism (e.g., degradation) of a product of interest by a bacteria that has been engineered to have a functional RNAi pathway using budding yeast Dicer and Argonaute polypeptides of the invention. The uses of bacteria in industrial processes and environmental remediation are legion, ranging from production of chemicals and substances of numerous classes (e.g., pharmaceuticals, biofuels, foods, intermediates of use in other synthetic processes), environmental remediation, etc. Bacteria can be used to produceRNAi may be used to improve such processes and/or cellular properties, e.g., in a generally similar manner as described for yeast.

In some embodiments, RNAi is used to identify a gene whose silencing improves a cellular activity. For example, in some embodiments, RNAi is used to investigate genetic control of variation in ethanol tolerance in natural populations of yeast Saccharomyces cerevisiae. Identification of genes that affect ethanol tolerance, e.g., genes whose expression can be modulated to increase ethanol tolerance, would be of great value for the brewing and biofuel industries. In some embodiments, libraries of yeast strains in which one or more genes are silenced by RNAi are screened to identify those with reduced or increased ethanol tolerance. For example, strains that exhibit growth deficiencies in the presence of ethanol can be identified. The silenced genes are candidates for being involved in ethanol tolerance. Overexpression of such genes can result in increased ethanol tolerance. Similar strategies can be employed to identify genes involved in tolerance to other inhibitory substances or toxins, e.g., in the context of producing any product of interest. In some embodiments, the cellular activity is RNAi itself.

Once a gene whose silencing improves a cellular activity is identified, it can be mutated or deleted using standard genetic engineering approaches (in a strain for which such approaches are available), or a screen can be performed to identify a strain having a mutant allele of the gene. The resulting mutant can be used, e.g., to produce a product of interest, without the use of RNAi. This approach may be of use in situations where it is desired to utilize a non-genetically engineered yeast. In some embodiments, RNAi is used in yeast to identify genes whose silencing improves ethanol tolerance, increases ethanol yield, and/or enables the use of a broader range of substrates for ethanol production.

In some embodiments, RNAi is used to silence a target gene that encodes a selectable marker, e.g., a nutritional marker such as URA3, or an antibiotic resistance marker, or a detectable marker such as GFP. In some embodiments, such silencing can be used as a control, e.g., to verify that the RNAi pathway is functional. In some embodiments, such silencing is used in methods relating to the study of RNAi, e.g., in the identification or characterization of genes that modulate RNAi (see discussion below).

In some embodiments, the RNAi pathway is engineered in a budding yeast that lacks a functional endogenous RNAi pathway, wherein the yeast exhibits transposition (e.g., the yeast genome comprises transposable elements, e.g., DNA transposons, retrotransposons, wherein the elements or copies thereof move from place to place within the genome). Transposition can generate mutations and can alter yeast phenotype in a manner that can be unpredictable and undesirable. Such alteration may, for example, affect a cellular property, e.g., ability of the yeast to produce a product of interest. In accordance with the invention, engineering a budding yeast to have a functional RNAi pathway reduces transposition. Thus the invention provides a method of reducing transposition in a budding yeast that lacks an endogenous gene encoding a functional RNAi pathway polypeptide, the method comprising engineering the yeast to express a functional RNAi pathway polypeptide, e.g., a Dicer polypeptide of the invention. The invention provides a method of reducing transposition in a budding yeast comprising engineering the yeast to have a functional RNAi pathway. The method may be used, e.g., to stabilize the yeast genome. The resulting yeast, and the method, may be used in any context in which a yeast that exhibits transposition is used to produce a product of interest, e.g., to stabilize the yeast genome. Thus the invention provides a budding yeast strain that is genetically engineered to have a functional RNAi pathway exhibits and has reduced transposition relative to a comparable yeast strain that has not been so engineered, e.g., an otherwise isogenic yeast strain. In some embodiments, such strains exhibit less variability over time, e.g., they may have improved maintenance of their ability to produce a product of interest over time, than would otherwise be the case. In some embodiments, this aspect of the invention allows the use of certain species or strains in industrial processes for which use they would otherwise be unsuitable as a result of transposition. The invention encompasses use of RNAi in any manner to stabilize a yeast strain or yeast culture, e.g., to inhibit the strain or culture from changing one or more properties of interest over time.

In some embodiments, the existing tools of budding yeast (e.g., ability to perform forward genetic screens, e.g., loss-of-function screens) are applied to the study of RNAi. For example, screens can be performed to identify mutants with defects in the RNAi pathway and, optionally, the mutated gene or suppressor(s) of the mutant phenotype are identified, thus identifying genes that play a role in the RNAi pathway or modulate RNAi. In some embodiments of the invention the endogenous RNAi pathway is investigated in budding yeast such as S. castellii. In some embodiments, existing tools are used to examine the reconstituted RNAi pathway in S. cerevisiae. Such examination could include, e.g., screens to identify endogenous genes whose mutation or over-expression modulates (e.g., enhances, inhibits, or otherwise alters) RNAi, screens to identify heterologous genes whose expression in budding yeast modulates RNAi, and/or testing different dsRNAs, e.g., to identify those that are cleaved to siRNAs that silence a target gene with high efficiency. Once having identified a gene of interest in budding yeast, homologs can be identified in other eukaryotes (e.g., in other fungi, in plants, or in mammals, e.g., mice or humans). For example, publicly available databases can be searched using the yeast sequence and homologous sequences identified. Manipulating such genes or their encoded gene products can be used to enhance the efficacy of RNAi, e.g., for research or therapeutic purposes.

In certain embodiments, the invention provides a method of screening for compounds that modulate RNAi. Certain of the methods comprise: (a) contacting a budding yeast that has a functional RNAi pathway with a compound; and (b) assessing activity of the RNAi pathway in the budding yeast, wherein if the activity of the RNAi pathway differs from that of a control, the compound modulates the RNAi pathway. Compounds that modulate the RNAi pathway may be used to modulate, e.g., enhance, RNAi in yeast or in other organisms.

The invention further provides methods of reducing viability or proliferation of a budding yeast cell that has a functional RNAi pathway using RNAi. Certain of the methods comprise delivering an siRNA to a budding yeast cell that has a functional RNAi pathway, wherein the siRNA targets a gene for silencing, e.g., by targeting mRNA of the gene for degradation, wherein the gene or a product of the gene is important for cell viability. In some embodiments, the budding yeast is a member of a pathogenic strain or species, e.g., a human, animal, or plant pathogen. In some embodiments, the targeted gene is an essential gene. In some embodiments, the targeted gene encodes a protein that is a target of an existing antifungal agent. For example, the gene may contribute to plasma membrane or cell wall synthesis or maintenance, to cell division, or to synthesis of an essential biomolecule. In some embodiments the gene does not have a homolog in mammals or plants. In some embodiments, the target gene encodes the enzyme 14α-demethylase which is involved in synthesis of ergosterol. In some embodiments the target gene encodes a protein that contributes to synthesis or deposition of glucan in the cell wall. For example, the target gene may encode the enzyme 1,3-β glucan synthase. The siRNA could be delivered by contacting the cell exogenously with siRNA (e.g., in vitro, e.g., by adding the siRNA to a medium in which the cell is maintained, e.g., culture medium) or by expressing an siRNA precursor (dsRNA) in the cell. In some embodiments the siRNA is delivered by administering it to an animal (e.g., human) or plant host that is colonized by the yeast cell. In some embodiments, an siRNA is administered in combination with a conventional antifungal agent. “In combination” (also referred to as “co-administration”) can refer to administration within the same composition or separately, provided that the agents are present simultaneously at detectable levels within the organism to which they are administered. In some embodiments, “co-administration” refers to administration of two agents one or more times within a period of 48 hours. In some embodiments, the conventional anti-fungal agent weakens the yeast cell wall and/or generates pores or channels therein. In some embodiments, co-administration allows for use of a lower dose of the conventional anti-fungal agent and/or allows for use of a lower dose of the siRNA.

In some embodiments, the invention provides pharmaceutical compositions comprising an antifungal siRNA, or siRNA precursor, and methods of treating a fungal infection using RNAi. Certain methods comprise delivering an siRNA to a budding yeast that has a functional RNAi pathway, e.g., as described above, to reduce viability or proliferation of the cell, wherein said delivery comprises administering siRNA to an individual colonized by, or at risk of being colonized by, a budding yeast cell, e.g., a pathogenic budding yeast cell. In some embodiments, the siRNA is co-administered with a conventional anti-fungal agent (e.g., an azole, echinocandin, or amphotericin, e.g., amphotericin B), wherein in some embodiments co-administration allows for use of a lower dose of the conventional anti-fungal agent or siRNA. In some embodiments, the siRNA is administered to treat a systemic infection. In some embodiments, the siRNA is administered to treat a local infection, e.g., a skin infection. In some embodiments, the subject has an indwelling device, e.g., catheter. In some embodiments, an antifungal siRNA of the invention is delivered together with an siRNA targeted to a viral gene. Such co-administration may reduce the risk of fungal superinfection of a host that has a viral infection. For example, an antifungal siRNA could be delivered together with an siRNA targeted to a gene of a respiratory virus, e.g., influenza A or B virus, respiratory syncytial virus, parainfluenza virus, adenovirus, coronavirus, rhinovirus, or human metapneumovirus. See, e.g., .DeVincenzo J, et al., Antiviral Res. 77(3):225-31, 2008; Bank S., Methods Mol. Biol., 487:331-41, 2009. In some embodiments, an antifungal siRNA is administered in combination with a second agent useful to treat a co-existing disorder, e.g., a disorder that increases susceptibility to a fungal infection. The second agent could be, e.g., an antibacterial agent, an antiviral agent, an antiparasitic agent, an anticancer agent, etc.

One of skill in the art will appreciate that siRNA or other agents (e.g., drugs identified according to the invention) could be administered to a mammalian subject using any suitable approach known in the art. A variety of pharmaceutically acceptable carriers and formulations may be used. The agent may be delivered in an effective amount, by which is meant an amount sufficient to achieve a biological response of interest, e.g., reducing gene expression by a certain amount, reducing one or more symptoms or manifestations of a disease or condition. In certain embodiments the agent is administered to a mammalian subject suffering from or at increased risk of a condition mentioned herein in a therapeutically effective amount, e.g., an amount sufficient to ameliorate at least one symptom of the disease to a clinically meaningful extent. An agent can be administered for therapeutic purposes after onset of symptoms or prophylactically, e.g., to an individual at risk. An individual may be at risk if he or she falls into an art-recognized risk category, has been exposed to an infectious agent, is immunocompromised, etc. Pharmaceutically acceptable carriers are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption or uptake of the active agent. The physiologically acceptable compounds include, for example, carbohydrates, such as glucose, sucrose or dextrans, antioxidants, such as ascorbic acid or glutathione, chelating agents, buffers, low molecular weight proteins or other stabilizers or excipients. One skilled in the art would know that the choice of a pharmaceutically acceptable carrier, including a physiologically acceptable compound, depends, for example, on the route of administration of the composition. The pharmaceutical composition could be in the form of a liquid, gel, lotion, tablet, capsule, ointment, transdermal patch, etc. A pharmaceutical composition can be administered to a subject by various routes including, for example, parenteral administration. Examples include intravenous administration; respiratory administration (e.g., by inhalation), nasal administration, intraperitoneal administration, oral administration, subcutaneous administration and topical administration. One skilled in the art would select an effective dose and administration regimen taking into consideration factors such as the patient's weight and general health, the particular condition being treated, etc. Exemplary doses may be selected using in vitro studies, tested in animal models, and/or in human clinical trials as standard in the art.

In some embodiments, the pharmaceutical composition is delivered by means of a microparticle or nanoparticle or a liposome or other delivery vehicle or matrix. A number of biocompatible polymeric materials are known in the art to be of use for drug delivery purposes. Examples include polylactide-co-glycolide, polycaprolactone, polyanhydride, and copolymers or blends thereof. Liposomes, for example, which consist of phospholipids or other lipids, are nontoxic, physiologically acceptable and metabolizable carriers that are relatively simple to make and administer.

Antifungal siRNA could also be used to decontaminate objects, e.g., surfaces, that may be subject to fungal contact and/or colonization. They may be used as components of fungicides intended for use in agriculture.

It will be appreciated that a single siRNA or a combination of multiple siRNAs may be used in the above methods. For example, a composition comprising a multiplicity of siRNAs derived by Dicer-mediated cleavage of a dsRNA may be used.

The invention provides methods of producing siRNA. In accordance with the invention, siRNA may be produced by expressing a dsRNA in a budding yeast that comprises a Dicer polypeptide functional in the cell (either endogenous or engineered to express the polypeptide), whereby the dsRNA is cleaved to siRNA. In some embodiments the cell has a functional RNAi pathway. The siRNA may be isolated from the cell. In some embodiments, siRNA isolation from a cell comprises at least partial removal of the cell wall and/or particulate or membranous material and/or cell organelles, e.g., by centrifugation. In other embodiments, a cell extract is prepared from budding yeast cells that comprise a Dicer polypeptide functional in the cell, and dsRNA is added to the extract. The extract may be a soluble extract (e.g., at least some or the cell wall, membranous, and/or particulate or cell organelle material is removed).

In some embodiments, at least some proteinaceous material is removed during isolation of the siRNA, e.g., using phenol extraction and precipitation or other methods known to those in the art. In some embodiments, large nucleic acids are removed. For example, genomic DNA may be removed. In some embodiments the siRNA may be at least partially purified, e.g., using methods based on size, affinity, charge, or any property of interest. In some embodiments the siRNA is isolated using a gel. In some embodiments the siRNA is isolated using a column. In some embodiments, the methods comprise providing a budding yeast cell that comprises a Dicer polypeptide functional in the cell (either endogenous or engineered) and comprises a template for transcription of a dsRNA; (b) maintaining the cell under conditions in which the dsRNA is expressed and is cleaved to siRNA; and (c) isolating siRNA from the cell. In some embodiments, the methods comprise providing an extract from a budding yeast cell that comprises a Dicer polypeptide functional in the cell (either endogenous or engineered) and comprises a template for transcription of a dsRNA; (b) maintaining the cell under conditions in which the dsRNA is expressed and is cleaved to siRNA; and (c) isolating siRNA from the cell extract. In some embodiments, the Dicer polypeptide comprises or consists of a minimal Dicer polypeptide comprising an RNase III domain, e.g., a polypeptide is at least 80% identical to an RNase III domain found in a functional budding yeast Dicer polypeptide.

The methods of producing siRNA are not limited to budding yeast. In some embodiments, such methods are employed using bacteria that have been engineered to comprise a Dicer polypeptide in the cells in accordance with the present invention, or with extracts derived from such bacteria. In some embodiments, the Dicer polypeptide comprises a minimal Dicer polypeptide comprising an RNase III domain, e.g., a polypeptide that is at least 80% identical to an RNase III domain found in a functional budding yeast Dicer polypeptide, optionally further comprising a dsRNA binding domain.

The invention further provides methods of producing siRNA using at least partially purified Dicer polypeptides of the invention. Certain methods comprise (i) providing a composition comprising an at least partially purified Dicer polypeptide of the invention and a dsRNA; (ii) maintaining the composition under conditions in which the dsRNA is cleaved to siRNA; and (iii) optionally, isolating the siRNA from the composition of (ii). In some embodiments, the isolation step (iii) comprises at least partially removing the Dicer polypeptide. For example, standard methods of removing proteins, such as phenol-chloroform extraction, can be used. siRNA can be isolated, e.g., based on size or affinity. In some embodiments, gel electrophoresis is used, followed by elution of the siRNA from the gel. The Dicer polypeptide may be any functional Dicer polypeptide of the invention and may be produced using any suitable host cell, e.g., bacteria. In one embodiment, the Dicer polypeptide comprises a polypeptide at least 80% identical to the Dicer polypeptide found in S. castelli. In one embodiment, the Dicer polypeptide comprises a polypeptide at least 80% identical to the Dicer polypeptide found in K. polysporus. In some embodiments, the functional Dicer polypeptide comprises at least an RNAse III domain and, optionally, at least one dsRNA-binding domain, of a naturally occurring budding yeast Dicer polypeptide. In some embodiments, the polypeptide comprises or consists of amino acids 15-355, 11-355, 1-376, 1-384, or 1-398 of K. polysporus Dicer or corresponding amino acids of Dicer from a different budding yeast, such as S. castellii, C. albicans, C. tropicalis, P. stipitis, or Debaromyces hansenii. Optionally, the polypeptide can comprise one or more tags. Optionally, one or more tags are removed prior to using the Dicer polypeptide to cleave dsRNA. The composition may further comprise, e.g., a buffer, a salt (e.g., NaCl, KCl), an ion (e.g., a divalent cation, e.g., Mg, which may be provided as MgCl₂), an energy source (e.g., ATP), protease inhibitor, stabilizing agent, etc. In some embodiments, any of the afore-mentioned components may be added to a cell extract used to produce siRNA. In some embodiments, the composition consists of defined components, e.g., is free of unknown or unidentified substances that may be present in cell extracts. The Dicer polypeptide may be purified, e.g., as described herein. In some embodiments, the composition is free or substantially free of non-Dicer proteins that may be present in a cell from which Dicer polypeptide is purified.

In some embodiments, an in vitro cleavage reaction is carried out in a composition (e.g., an aqueous solution) having a pH between about 6.0 and about 8.5, e.g., between about 7.0 and 8.0, e.g., about 7.5. The composition will typically contain one or more buffers, e.g., Tris-HCl, HEPES, etc., to regulate the pH. In some embodiments, a cleavage reaction is carried out in a composition having a monovalent cation concentration between about 10 mM and 200 mM, e.g., about 20-50 mM, e.g., about 30 mM. The monovalent cation could be, e.g., Na+, which can be provided as a salt, e.g., as a chloride or acetate salt. In some embodiments, a cleavage reaction is carried out in a composition having an Mg++ concentration between about 2 mM and 20 mM Mg++, e.g., between about 3 mM and 10 mM Mg++, e.g., about 5 mM Mg++, which can provided as a salt, e.g., as a chloride or acetate salt. The composition could contain other components such as a reducing agent (e.g., DTT) and/or a chelating agent (e.g., EDTA). The composition may be prepared, e.g., by adding the Dicer polypeptide and dsRNA to RNase free water containing the other components. In some embodiments, a cleavage reaction is carried out at about room temperature, e.g., between 22-24 degrees C. In other embodiments, a higher or lower temperature is used, e.g., between about 1° C. and about 40° C., e.g., between about 10° C. and about 37° C. The cleavage reaction is allowed to continue for a suitable period of time. For example, in some embodiments, the composition is maintained for between 1 minute and 24 hours, e.g., between 5 minutes and 4 hours, e.g., for about 30-60 minutes. The ratio of Dicer polypeptide to potential cleavage sites in the dsRNA can vary. For example, the ratio can range between about 1:10 and about 10:1 in exemplary embodiments. In some embodiments, the ratio is about 1:1. The reaction time to achieve a given extent of cleavage may be increased if smaller amounts of enzyme relative to substrate are used. It will be understood that the afore-mentioned conditions are exemplary and non-limiting. Conditions can be selected or optimized for a budding yeast Dicer of interest.

In some embodiments, at least some siRNA produced using the methods comprises strands that are 22 nucleotides in length. In some embodiments, at least some siRNA produced using the methods comprises strands that are 23 nucleotides in length. In some embodiments, the invention provides a mixture of siRNAs (an “siRNA pool”) wherein the siRNAs are generated by cleavage of a dsRNA in vitro by a functional Dicer polypeptide. The pool contains a mixture of siRNA of different sequences, corresponding to portions of the dsRNA. In some embodiments, the pool contains siRNAs of at least 10 different sequences corresponding to a gene of interest. In some embodiments, at least 50%, 60%, 70%, 75%, 80%, 90%, 95% or more of the siRNAs in the pool comprise strands that are 23 nucleotides long. In some embodiments, at least 50%, 60%, 70%, 75%, 80%, 90%, 95% or more of the RNA strands between 18 and 30 nucleotides long are 23 nucleotides long. In an exemplary embodiment, between about 70% and about 80% of the RNA strands between 18 and 30 nucleotides long are 23 nucleotides long. The invention further provides a composition comprising an siRNA pool generated using a functional budding yeast Dicer polypeptide. The composition could comprise a suitable carrier such as water, an alcohol (e.g., ethanol), a buffer such as Tris-HCl, a salt, etc. In some embodiments, the carrier is a physiologically acceptable carrier. In some embodiments the composition is RNase free. In some embodiments, the composition comprises a delivery vehicle, carrier or matrix that enhances delivery of siRNA to cells in whole organisms and/or increases stability of siRNA in whole organisms (e.g., in serum or other biological fluids).

The dsRNA used in the inventive methods of producing siRNA could correspond to any gene of interest. The dsRNA can be produced or obtained using any suitable method. In some embodiments, dsRNA is produced using in vitro transcription. In some embodiments, dsRNA is isolated from cells. In some embodiments, the dsRNA is at least 50, 100, 200, 300, 400, or 500 by in length, and can be up to about 1, 2, 3, 4, or 5 kbp in length. For example, the dsRNA can be between 300 by and 2,000 by long, e.g., between 400 and 1,000 by long. The gene could be endogenous to an organism of any species. In some embodiments, the organism is eukaryotic. In some embodiments, the organism is an animal. In some embodiments the animal is a vertebrate, while in other embodiments the animal is an invertebrate. In some embodiments, the gene is an insect gene, e.g., a Drosophila gene. In some embodiments, the gene is a mammalian gene, e.g., a human gene or a rodent (e.g., mouse or rat) gene. A variety of exemplary, non-limiting types of target genes are discussed herein. In some embodiments, the gene of interest encodes a polypeptide that functions in a biological process or pathway of interest or a polypeptide that is a component of a cell organelle or structure of interest. For example, the polypeptide could function in apoptosis, regulation of the cell cycle, development, a metabolic process, regulation of gene expression, response to stimulus, sensory perception, signaling, or transport. A gene of interest could encode, e.g., a polypeptide that has an activity of interest, e.g., binding activity, catalytic activity, receptor activity, etc. In some embodiments, a polypeptide is, or is as a component of, an enzyme, an oncoprotein, a tumor suppressor protein, a transcription factor, a structural protein, a receptor (e.g., a G protein coupled receptor, receptor tyrosine kinase, hormone receptor, nuclear receptor, cytokine receptor), a channel, a chaperone, a heat shock protein, a hormone, a growth factor, a chemokine, a cytokine, etc. An enzyme could be, e.g., a protease, kinase (e.g., serine, threonine, tyrosine kinase), phosphatase, deubiquitinating enzyme, lipase, deacetylase, acetylase, methyltransferase. An enzyme could act on a substrate of interest, e.g., DNA, RNA, histones, etc. In some embodiments, a gene of interest encodes a polypeptide known to be a drug target. In some embodiments, a gene of interest encodes a drug target. In some embodiments, a drug target is involved in a particular metabolic or signaling pathway that contributes to and/or is specific to a disease condition or pathology, or to the infectivity or survival of a microbial pathogen. In some embodiments, the gene of interest encodes a transmembrane protein. In some embodiments, the gene of interest encodes a secreted protein. In some embodiments, the gene of interest encodes a cytoplasmic protein.

The invention provides collections (libraries) of siRNA pools generated using a functional budding yeast Dicer polypeptide, wherein the siRNA pools correspond to different genes of interest. For example, a library could contain an siRNA pool corresponding to each of at least 10, 50, 100, 500, 1000; 5,000; 10,000; 20,000 or more genes. In some embodiments, the genes are native to an organism type of interest. For example, the genes could be animal genes, e.g., mammalian, avian, or insect genes. In exemplary embodiments the genes are human genes or rodent genes (e.g., mouse genes). In some embodiments, a library contains siRNA pools corresponding to all or substantially all (e.g., at least 95%, 98%, 99%, or more) of known or annotated genes of an organism of interest. In some embodiments, a “known gene” is a gene that is identified in GenBank as containing a coding sequence. In some embodiments, a library contains genes corresponding to a category of interest. For example the genes could encode proteins that participate in a biological process of interest or have a molecular function of interest or are present in a cell organelle or structure of interest. Exemplary categories include, e.g., cell cycle regulators, enzymes (e.g., of any of the types mentioned above), oncoproteins, tumor suppressor proteins, transcription factors, structural proteins, receptors (e.g., G protein coupled receptors, receptor tyrosine kinases, hormone receptors, nuclear receptors, cytokine receptors), channels, chaperones, heat shock proteins, hormones, growth factors, chemokines, cytokines, etc. An enzyme could be, e.g., a protease, kinase (e.g., serine, threonine, tyrosine kinase), phosphatase, deubiquitinating enzyme, lipase, deacetylase, acetylase, methyltransferase. In some embodiments, the library genes encode known or potential drug targets. The siRNA pools could be provided in individual vessels, e.g., microfuge tubes, wells of a multiwell plate (e.g., a 96 or 384 well plate). In some embodiments, siRNA pools are attached to a substrate such as a glass slide, e.g., in array format.

The siRNA, e.g., siRNA pools, may be used in any method of interest. In some embodiments, an siRNA pool is used to silence an endogenous gene in eukaryotic cells, e.g., animal cells, e.g., vertebrate cells or invertebrate cells. In some embodiments, the cells are mammalian cells, e.g., rodent cells or human cells. In some embodiments, the cells are insect cells, e.g., Drosophila cells. In some embodiments, an siRNA pool is used to silence a non-endogenous gene in eukaryotic cells. For example, the non-endogenous gene could be a reporter gene that has been introduced into the cell (or an ancestor of the cell) by the hand of man. The non-endogenous gene could be a viral gene. The virus could be, e.g., any virus capable of infecting a cell of interest, e.g., in a species of interest such as a mammal. In some embodiments, the virus is a pathogenic virus, e.g., a virus that is pathogenic to one or more mammalian species, e.g., humans. See, e.g., Knipe, D M and Howley, P M (eds.) Fields Virology, 5^{th} ed. Lippincott Williams & Wilkins, 2007. In some embodiments, the gene encodes a viral protein that is essential for the viral life cycle. In some embodiments, the siRNA silences a viral RNA that is essential for the viral life cycle.

In some embodiments the cells are cultured cells. The siRNA pool can be contacted with the cells under conditions suitable for uptake of the siRNA. Suitable methods are known in the art. For example, transfection mediated by chemical agents, electroporation, magnet assisted transfection, or microinjection could be used. In some embodiments, siRNA are introduced into cells in order to examine the function of a gene, to identify or characterize a drug target, to determine whether a gene is a suitable target for drug discovery, or for any other purpose for which gene knockdown is of use. The cells could be of a cell type of interest or have a property of interest. In some embodiments, cells are primary cells. In some embodiments, cells are of an immortalized cell line. In some embodiments, cells are adherent cells. In some embodiments, cells are cancer cells. In some embodiments, cells contain a mutation associated with a disease of interest. In some embodiments, cells are pluripotent or multipotent. In some embodiments, cells are of a mature, differentiated cell type of interest.

In some embodiments, the cells are in an animal, e.g., a mammal. In some embodiments, the siRNA pool is administered for therapeutic purposes. For example, an siRNA pool could be used to treat a viral disease, a cancer, an autoimmune disorder, etc. In some embodiments, the siRNA pool is administered in order to examine the function of a gene, to identify a drug target and/or to determine whether a gene is a suitable target for drug discovery, etc.

In some embodiments, one or more reagents or methods described in the section entitled “Kits” are used in the inventive methods of producing siRNA in vitro using purified Dicer polypeptide.

Without wishing to be bound by any theory, (i) RNAi using an siRNA pool may be associated with reduced “off-target” effects for a given degree of silencing as compared with use of a single siRNA (or use of 2-4 distinct siRNAs in combination) since each siRNA in an siRNA pool may be provided at a lower concentration than required when only a single siRNA (or small number such as 2-4 siRNA) are used; and/or (ii) RNAi using an siRNA pool may allow a greater degree of silencing than achievable using a single siRNA (or even as compared with that achievable using 2-3 distinct siRNAs in combination).

### EXAMPLES

### Example 1

### Identification and Isolation of siRNAs in Budding Yeasts

Despite the perceived loss of RNAi in budding yeast, Argonaute genes are present in some budding yeasts, including Saccharomyces castellii and Kluyveromyces polysporus (both close relatives of S. cerevisiae) and Candida albicans (the most common yeast pathogen of humans (8)) (FIG. 1A). These genes contain the defining PAZ and Piwi domains and, as in other fungi, represent the Argonaute Glade of the Argonaute/Piwi family. Noted previously (9, 10), these genes of budding yeast have been enigmatic because other RNA-silencing genes, especially Dicer, have not been found in these species. A similar conundrum appears in prokaryotes, in which certain bacteria have Argonaute homologs yet lack the other genes associated with RNA silencing (11).

To investigate the possibility of RNA silencing in budding yeast, we searched for short guide RNAs, isolating 18-30-nt RNAs from S. castellii, K. polysporus, and C. albicans and preparing sequencing libraries representing the subset of small RNAs with 5′-monophosphates and 3′-hydroxyls (12), which are the chemical features of Dicer products. As a control, we also sequenced RNAs from S. cerevisiae. In contrast to S. cerevisiae, the three yeast species with Argonaute each contained a set of small RNAs with striking length and 5′-nucleotide biases. The RNAs of S. castellii and K. polysporus were most enriched in 23-mers beginning with U, and those of C. albicans were most enriched in 22-mers beginning with A or U (FIG. 1B). These biases were reminiscent of those observed for Argonaute-bound guide RNAs of animals, plants, and other fungi (13-15).

Although some reads from the Argonaute-containing yeasts mapped to ribosomal RNA (rRNA) and tRNA and presumably represented degradation intermediates of abundant RNAs, many reads clustered at other types of genomic loci (FIG. 1C, table S1). The loci generating the most reads had sequence homology to repetitive elements, including LTR retrotransposons (Ty elements), LINE-like retrotransposons (Zorro elements), and subtelomeric repeats (Y′ elements) (FIG. 1C, table S1). Loci of S. castellii were also particularly enriched in long inverted repeats; these palindromic loci generated most of the reads with homology to Ty elements (FIGS. 1C and D). As expected in a species lacking RNAi, essentially all the reads in S. cerevisiae appeared to represent degradation fragments of rRNA, tRNA, and mRNA.

The reads matching inverted repeats fell in patterns suggesting origins from paired regions of transcripts that folded back on themselves to form hairpins (FIG. 1D). These inferred hairpins had 100-400-bp stems with loops ranging from 19 to >1600 nt. In regions of imperfect duplex, where reads could be mapped unambiguously, the small RNAs tended to match only one genomic strand, further supporting the idea that they originated from hairpin transcripts (FIG. 1D, bottom). Other reads did not map to inverted repeats and instead mapped uniquely to both genomic strands in a pattern suggesting origins from long bimolecular duplexes involving transcripts from both strands.

Most siRNAs of the fission yeast S. pombe correspond to the outer repeats of the centromeres and direct heterochromatin formation and maintenance (5). We therefore examined whether any of our sequenced small RNAs matched centromeres. Of the three Argonaute-containing species from which we sequenced (FIG. 1B), only C. albicans had annotated centromeres, and almost none (<0.001%) of our C. albicans reads matched these genomic loci. Moreover, budding yeasts lack recognizable orthologs of the H3K9 methyltransferase Clr4 and recognizable homologs of RdRP, Tas3, and Chp1, and the HP1-like chromodomain protein Swi6—proteins all necessary for RNAi-dependent heterochromatin in S. pombe (5), arguing against a function analogous to that in S. pombe.

Zooming in on the precise genomic positions of 23-mer termini, the 3′-terminus of one 23-mer was often adjacent to the 5′-terminus of the next 23-mer, suggesting that endonuclease cleavage simultaneously generated the 3′-terminus of one small RNA and the 5′-terminus of the next. Consistent with this hypothesis, systematic analysis of the intervals spanning the mapped ends of all 23-mer pairs revealed a clear phasing interval of 23 nt (FIG. 1E). Such phasing implied successive cleavage beginning at preferred starting points. Moreover, pairs from opposite strands had the same phasing interval but in a register 2 nt offset from that of the same-strand pairs. Together, the phasing and offset implied successive cleavage of dsRNA with a 2-nt 3′ overhang—the classic biogenesis of endogenous siRNAs by Dicer (3). Therefore, the small RNAs that appeared to derive from regions of dsRNA, i.e., those mapping in clusters to the arms of predicted hairpins and those mapping in clusters to both genomic strands, were classified as siRNAs.

### Example 2

### Identification of Dicer Gene of Budding Yeast

The presence of siRNAs in Argonaute-containing budding yeasts implied that each of these species also had a Dicer-like activity. To assay for this activity, we monitored processing of a long dsRNA added to whole-cell extracts (16). Consistent with our interpretations of the sequencing data, extracts from S. castellii, K. polysporus, and C. albicans—but not from S. cerevisiae—contained an activity that produced 22-23-nt RNAs, each preferentially from dsRNA rather than from single-stranded RNA (FIG. 2A). Moreover, for each extract the small-RNA length matched that of the most abundant length observed in vivo (FIGS. 1B and 2A).

With both molecular and biochemical evidence of Dicer-like activity, we searched for the Dicer gene. As noted previously (10), a gene with the domain architecture of known Dicers was not found in any budding-yeast genome. Because we had evidence for cleavage of dsRNA with 2-nt 3′ overhangs, a hallmark of RNaseIII activity, we relaxed the search criteria to consider any gene with an RNaseIII domain. S. cerevisiae had only one gene, RNT1, with a recognizable RNaseIII domain. RNT1 helps process rRNA and other noncoding RNAs (17), and presumed orthologs were found throughout the fungal kingdom. S. castellii also had a second RNaseIII-domain-containing gene, and a potential ortholog of this gene was found in each of the other Argonaute-containing budding yeasts (FIG. 1A). Anticipating that this second gene encoded the Dicer of budding yeasts, we named it DCR1.

To test whether the Dicer candidate is required for siRNA accumulation, we generated a knockout in S. castellii, the closest relative to S. cerevisiae among the sequenced Argonaute-containing species. After establishing strains and protocols to supplement those already being used for reverse-genetic analyses in this species with scant experimental history (18), we deleted DCR1 in S. castellii by homologous recombination (16). siRNAs failed to accumulate in the deletion mutant, as demonstrated by RNA blots (FIG. 2B) and high-throughput sequencing (fig. S2 and table S1). Deletion of the Argonaute homolog, which we named AGO1, also reduced siRNA accumulation, as expected if loading into Argonaute protected siRNAs from degradation (FIG. 2B, fig. S1, and table S1). For both mutants, ectopically expressing the deleted gene rescued siRNA accumulation (FIG. 2B). These results show that we had identified the Dicer of budding yeast and indicate that the core components of endogenous RNAi pathways—Dicer, Argonaute, and siRNAs—are present in some budding-yeast species.

In other fungi, known Dicer genes resemble those in plants and animals, complete with tandem RNaseIII domains, 2-3 dsRBDs, a PAZ domain, and an N-terminal helicase domain (16, 19, 20) (FIG. 2C). In budding yeasts, DCR1 has two dsRBDs but only a single RNaseIII domain and no helicase or PAZ domains. Because RNaseIII domains work in pairs to nick both strands of an RNA duplex (20, 21), we suspect that S. castellii Dcr1 acts as a homodimer. Dicers of insects, plants, and mammals, which already have two RNaseIII domains, do not homodimerize but do form heterodimeric complexes with cofactors that provide additional dsRBDs (22-24). A homodimeric S. castellii Dcr1 complex would already possess four dsRBDs, which might obviate the need for such a cofactor.

Except for its second dsRBD, the domain architecture of the budding-yeast Dicer resembled that of RNT1 rather than that of canonical Dicer genes (FIG. 2C). Furthermore, the amino acid sequence of its RNaseIII domain was more similar to that of the RNT1 RNaseIII domain than to that of any previously identified Dicer RNaseIII domain (FIG. 2D). These observations suggest that budding-yeast Dicer might not have descended directly from a canonical Dicer gene but instead emerged from a duplication of RNT1 early in the budding-yeast lineage, perhaps coincident with the loss of canonical Dicer. The unusual ancestry and domain structure of DCR1 might explain why its activity, and thus RNAi more generally, went undetected for so long in budding yeast.

### Example 3

### Biochemical Analyses Dcr1 and Ago1 of Budding Yeast

Dicing activity of S. castellii extracts was lost in the Δdcr1 mutant and restored by Dcr1 overexpression, observations that validated the utility of the in vitro assay for monitoring Dcr1 activity (FIG. 2E). To determine if Dcr1 is active in the absence of S. castellii cofactors, we expressed the protein in S. cerevisiae and E. coli (FIG. 2E). Expression in E. coli conferred robust activity, indicating that S. castellii Dcr1 is sufficient to dice dsRNA at precise intervals. In other Dicers, the PAZ domain is an essential component of a molecular ruler that imparts cleavage precision (20). The budding-yeast Dcr1, which lacks this domain, must achieve this measuring function differently.

To establish a biochemical link between AGO1 and the siRNAs of S. castellii (FIG. 2B), we sequenced the small RNAs that co-purified with tagged Ago1 expressed from its native promoter. Compared to the input RNA, the population of Ago1-associated RNAs was even more enriched for 22-23-nt RNAs and was depleted in matches to both rRNA and tRNA, with concomitant enrichment for matches to palindromes, Ty elements, and elements (fig. S3 and table S2). These biochemical results supported the genetic link between AGO1 and the siRNAs (FIG. 2B) and provided a set of RNAs useful for more precisely annotating the siRNA-producing loci of S. castellii (table S3).

### Example 4

### The S. castellii Transcriptome and its Modulation by RNAi

To determine the impact of RNAi on the transcriptome, we performed high-throughput sequencing of polyadenylated RNA (mRNA-Seq (25)) from wild-type, Δago1, and Δdcr1 strains (table S4). Among annotated open reading frames (ORFs), the two that changed most in RNAi deletion strains were also the two with the highest density of antisense siRNA reads (FIG. 3A, red points). One was the consensus Y′ ORF (fig. S5), which increased >7 fold in both deletion mutants. The other was an ORF within a palindromic Ty fragment, which increased >4 fold in the Δdcr1 mutant but was affected less in the Δago1 mutant. For other ORFs, transcript-abundance changes were modest and not correlated with siRNA density (fig. S6), although changes in Δago1 and Δdcr1 mutants did correlate with each other (R²=0.39, FIG. 3A). This correlation might reflect a general response to the loss of RNAi (although we cannot exclude contributions of a common response to the hygromycin- and kanamycin-resistance genes used to delete AGO1 and DCR1, respectively).

Our mRNA-Seq data revealing the S. castellii polyadenylated transcriptome enabled our analyses to extend beyond the sense strand of annotated ORFs. The broadened scope was important for identifying siRNA precursor transcripts because many siRNAs mapped antisense to or outside of ORFs. We focused on three types of siRNA precursors: sense-antisense transcript pairs at ORF loci, partially overlapping mRNAs, and transcripts producing the most siRNA-like reads, regardless of annotation.

Regarding siRNAs arising from sense-antisense ORF transcripts, we observed widespread low-level antisense transcription of ORFs, with antisense mRNA-Seq tags mapping to over half of all annotated ORFs. Moreover, small RNAs mapped antisense to nearly one-third of ORFs (FIG. 3A) and as a class were reduced in RNAi mutants and enriched by Ago1immunoprecipitation (fig. S3 and table S2). Supporting a precursor-product relationship, the abundance of the sense-antisense duplexes inferred from the mRNA-Seq data correlated with that of small RNAs deriving from these loci (fig. S7). The most striking example of siRNAs arising from sense-antisense transcript pairs was within the Y′ ORF, which was most affected by the loss of the RNAi machinery (FIG. 3A). In S. cerevisiae, Y′ elements are subtelomeric repeats located near both ends of most chromosomes (26) (fig. S7). They encode a large protein of unknown function that contains a DEXDc-family helicase domain conserved in most sequenced fungi (26, 27). The S. castellii repeats had a robustly expressed antisense transcript with many siRNAs mapping to the region of sense-antisense overlap (FIG. 3B).

We considered partially overlapping mRNAs as another potential source of siRNA-generating dsRNA, after using the mRNA-Seq data to extend the 5′ and 3′ boundaries of 5297 S. castellii protein-coding transcripts. Strikingly, 78% of convergent transcript pairs overlapped at their 3′ ends (median overlap of 92 nt, fig. S7), whereas only 1% of divergent transcript pairs overlapped at their 5′ ends, and 7% of tandem transcript pairs overlapped (FIG. 3E). As illustrated for one pair (FIG. 3D), at least 43% of the convergent overlapping pairs (comprising 9% of all gene pairs) generated DCR1-dependent siRNAs in the region of overlap (FIG. 3E and fig. S3). A recent study reported pervasive overlapping transcripts in S. cerevisiae (28). Our results revealing analogous overlap in S. castellii show that, in contrast to previous speculation, this phenomenon is not restricted to RNAi-deficient organisms and moreover is an ancestral feature of these Saccharomyces species (16).

We next inferred precursor transcripts without considering whether or not they overlapped ORFs (table S5). A hidden Markov model analyzing the Δgo1-associated small RNAs identified the genomic loci producing abundant siRNAs, and analysis of the mRNA-Seq data from Δdcr1 strains revealed the corresponding transcripts. In addition to recovering the more prolific ORF-overlapping siRNA precursors, this analysis identified the transcript illustrated in FIG. 1D and transcripts of 84 other non-protein-coding siRNA-generating genes of S. castellii (annotated as NCS1-NCS85, tables S3 and S5). Transcripts producing fewer siRNAs in RNAi-competent cells changed modestly but similarly in both deletion mutants (FIG. 3C), as observed when analyzing only ORF transcripts (FIG. 3A). Transcripts producing the most siRNAs—which were predominantly from palindromic loci—increased dramatically in the Δdcr1 mutant but were relatively unchanged (<2 fold) in the Δago1 mutant (FIG. 3C and table S5), indicating that Dcr1 alone was sufficient to reduce these transcripts to wild-type levels. This mode of posttranscriptional down-regulation may be unique to palindromic transcripts, which can fold into hairpin structures that are ideal Dcr1 substrates but refractory to intermolecular pairing with Ago1-associated siRNAs.

Taken together, our results indicate that more than a thousand genomic loci in S. castellii generate siRNAs, but that the primary regulatory target of RNAi silencing is the Y′-element mRNA. Consequences of siRNAs derived from the widespread antisense and overlapping transcription in S. castellii are unknown. The loss of the RNAi machinery did not substantially affect the levels of mRNAs corresponding to these siRNAs (FIGS. 3A and S6), but perhaps in other growth conditions the regulatory impact of non-Y′ siRNAs might be more pronounced. The specificity for Y′-element regulation could arise from a requirement for both an abundant pool of antisense siRNAs and the ability to base pair with a target transcript. Although palindromic loci generate many siRNA reads, the favored unimolecular hairpin structure of these transcripts might make them unsuited for pairing with siRNAs, and although coding mRNAs are relatively unstructured, most generate only low levels of siRNAs. These two requirements would explain the observed impact of RNAi on the S. castellii transcriptome.

### Example 5

### Engineering RNAi in S. castellii

To confirm that siRNAs can silence a gene in S. castellii and to create tools for monitoring RNAi in budding yeast, we generated two constructs (strong and weak) designed to silence a reporter gene expressing the green fluorescent protein (GFP, FIG. 4A). Both constructs were under the control of an inducible promoter, and each was integrated into the chromosomes of wild-type, Δago1, and Δdcr1 strains expressing GFP. As measured on RNA blots, the two constructs and two induction conditions produced a gradient of GFP siRNAs (FIG. 4B). In cells containing both AGO1 and DCR1, the amount of GFP silencing, as measured by fluorescence-activated cell sorting (FACS), corresponded to the level of GFP siRNAs, with the highest level of siRNA production repressing fluorescence to background autofluorescence (FIG. 4C). As expected, silencing depended on DCR1 for siRNA production and on AGO1 for siRNA function (FIGS. 4B and C).

These results confirmed that siRNAs could function to silence a gene, and demonstrated that the targeted transcript could originate from a locus distinct from the locus producing the siRNAs. In S. pombe, heterochromatic siRNAs are reported to function exclusively in cis (29). However, in an engineered system resembling ours, repression is posttranscriptional and acts in trans (30). The uniform behavior of cells expressing intermediate levels of siRNAs also hinted at a posttranscriptional silencing mechanism in our engineered system. In a transcriptional silencing mechanism, intermediate siRNA production might be expected to induce silencing that is more binary than graded because a threshold level of siRNAs might be necessary for heterochromatin formation. Instead, the histograms showed the uniform, even distribution expected for a posttranscriptional mechanism, for which the concentration of effective silencing complexes determines the degree of silencing, with each cell silenced to a similar extent (FIG. 4C).

### Example 6

### Reconstitution of RNAi in S. cerevisiae

Our observation that some budding yeasts closely related to S. cerevisiae contain a functional RNAi pathway suggested that the S. cerevisiae lineage lost RNAi recently and that perhaps introducing the two RNAi proteins found in S. castellii—Ago1 and Dcr1—could restore the pathway. To test this possibility, we used a GFP-reporter system based on our S. castelliisystem. GFP-positive strains of S. cerevisiae were generated that expressed either the strong, the weak, or no silencing construct. Introducing Dcr1 was sufficient to generate abundant GFP siRNAs from the strong silencing construct and a few GFP siRNAs from the weak construct (FIG. 4D). When Ago1 and Dcr1 were both present, we observed intermediate silencing with the weak construct and robust silencing with the strong construct (FIG. 4E), with a >100-fold mRNA knockdown accompanying the fluorescence knockdown with the strong construct (FIGS. 4F and S10). For these strains with reconstituted silencing, deleting AGO1 restored GFP expression (data not shown), thereby confirming small-RNA-based Argonaute-dependent silencing. As with the S. castellii reporter system, the fluorescence of the cell populations shifted collectively under silencing conditions, consistent with a posttranscriptional silencing mechanism. In the presence of both Ago1 and Dcr1, a hairpin construct targeting URA3 reduced growth in the absence of uracil and enabled growth on 5-fluoroorotic acid (5-FOA), demonstrating that the RNAi pathway reconstituted in S. cerevisiae can silence an endogenous gene with phenotypic consequences (FIG. 4G).

The ability to reconstitute RNAi in S. cerevisiae using only Ago1 and Dcr1 raised the possibility that the S. castellii RNAi pathway requires only these two proteins. This simplicity would make budding-yeast RNAi distinct from all known RNAi pathways, which use additional proteins involved in, for example, Argonaute loading (e.g. R2D2 in Drosophila melanogaster (1, 22)) or maturation of the silencing complex (e.g. QIP in N. crassa (31)). The four dsRBDs that would be present in a Dcr1 homodimer might explain the absence of a separate loading factor. Alternatively, overexpression of Ago1, Dcr1, and a hairpin precursor might be sufficient to enact RNAi in S. cerevisiae, but they might require additional factors for efficient silencing when expressed at physiological levels in S. castellii. Another possibility is that the reconstituted pathway uses components that have been maintained in S. cerevisiae since its recent loss of RNAi.

### Example 7

### RNAi and Transposon Silencing

The Δago1 and Δdcr1 mutants of S. castellii were viable, with no growth disadvantage observed when cultured in minimal or rich media at a range of temperatures, no observed decrease in mating, sporulation, or chromosome stability, and no altered sensitivity to a replication inhibitor (hydroxyurea) or to microtubule destabilizing agents (thiobendazole and benomyl). However, both Δago1 and Δdcr1 mutants had difficulty retaining introduced plasmids (fig. S11), demonstrating that the loss of RNAi has detectable phenotypic consequences.

We suspected that budding-yeast RNAi might also silence transposable elements. RNAi and related processes silence and eliminate transposons in other eukaryotes (2), and a large fraction of our budding-yeast siRNAs corresponded to transposable elements. For example, over half of the S. castellii siRNAs mapped to fragments of Ty retrotransposons (FIG. 1C). Despite the abundance of these and other Ty fragments, indicative of former activity in the S. castellii lineage (fig. S12), we have not yet found an active retrotransposon in the current, albeit incomplete, S. castellii genome sequence. Therefore, to test the effect of RNAi on transposition, we turned to the S. cerevisiae strains, monitoring transposition of a galactose-inducible Ty1 element engineered to complement histidine auxotrophy after its defective his3 gene is repaired during the process of transposing through a spliced RNA intermediate (32).

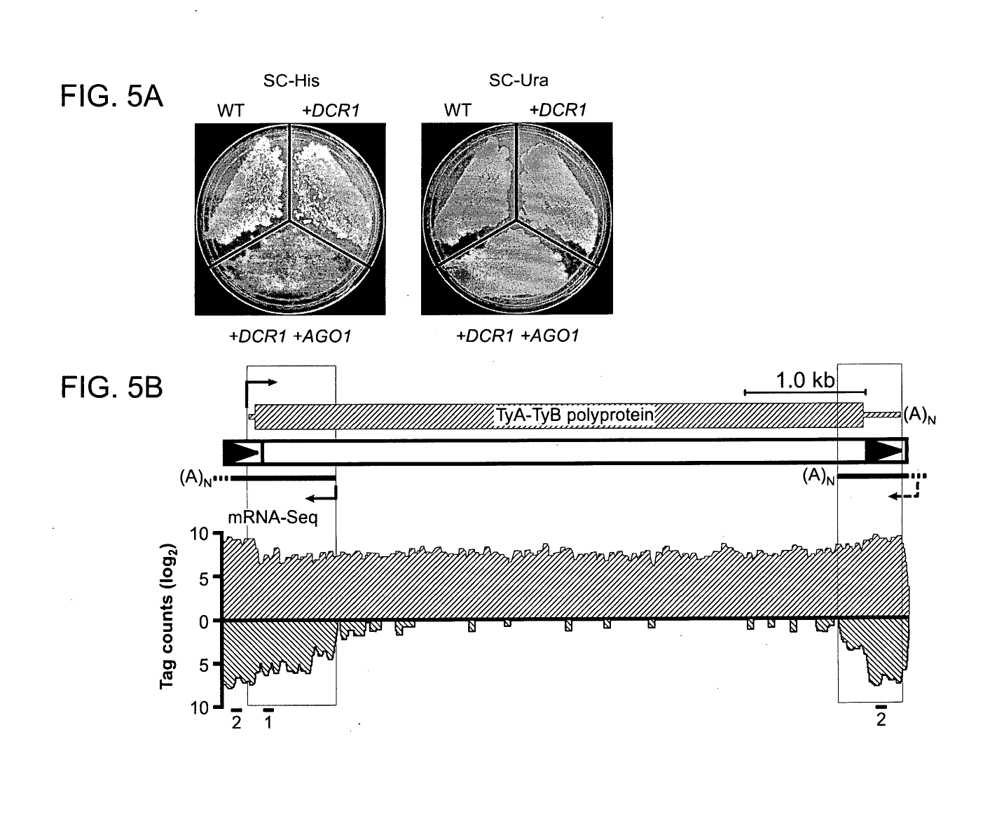

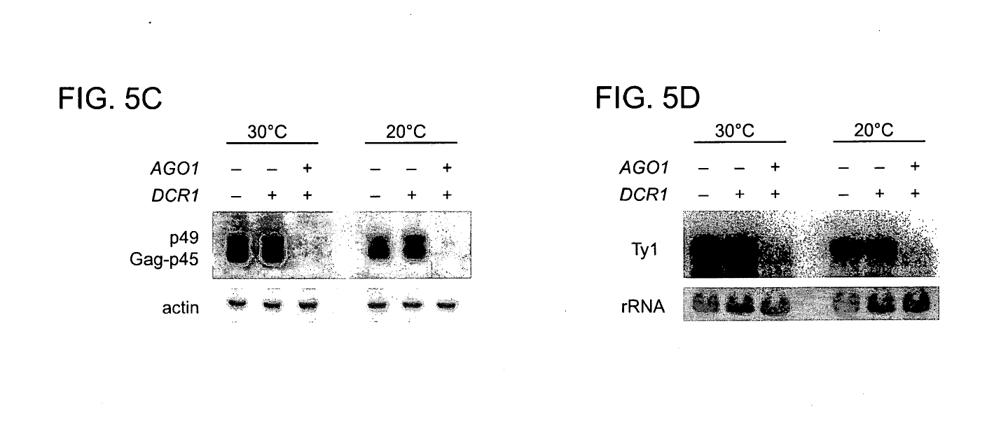

Compared to the strain that had no RNAi genes or the one that had only DCR1, transposition in the RNAi-competent strain was greatly diminished (FIG. 5A). Although the engineered Ty1 might be particularly sensitive to RNAi if the antisense transcript expressing the defective his3 is produced prior to transposition, dsRNA triggering Ty silencing could also come from endogenous sources. Neighboring elements or their remnants can be oriented such that they comprise palindromes that generate hairpin transcripts. Elements can also generate dsRNA in the form of bimolecular duplexes: the S. cerevisiae Ty 1 elements express their own antisense transcripts (33), and any transposon can land within a transcription unit in a convergent orientation suitable for producing overlapping transcripts. Our analysis of published mRNA-Seq data (34) demonstrated that endogenous antisense and convergent transcripts are abundantly expressed in S. cerevisiae (FIG. 5B). To test if these endogenous dsRNA sources were sufficient to trigger silencing of native elements, we monitored the levels of Ty1 Gag protein and Ty1 mRNA. Accumulation of both was robust in strains lacking RNAi but greatly diminished in the RNAi-competent strain (FIGS. 5C and D).

Our results show that adding the minimal components needed for reconstituting RNAi to a species normally lacking the pathway confers transposon silencing. That the recipient strain remained viable, without severe abnormalities, while the transposon protein and mRNA were so thoroughly reduced illustrates the specificity of the pathway for transposable elements. That no other added components were required for S. castellii proteins to guide silencing of an element from a different species illustrates the generality of the pathway, potentially able to target any repetitive element requiring an RNA transcript—an elegant defense poised to exploit the intrinsic propensity of repetitive elements to generate hairpin and convergent transcripts as their genomic load increases (as well as the propensity of Ty1 and perhaps other elements to produce their own antisense transcripts). These results, combined with our observation that many siRNAs in Argonaute-containing species correspond to transposons (FIG. 1C), indicate that a major role of budding-yeast RNAi is to silence transposons.

In summary, we have uncovered an RNAi pathway present in several different budding-yeast species that appears distinct from the well-characterized pathway of fission yeast. The two known components of the pathway have a patchy phylogenetic distribution among budding yeasts (FIG. 1A), which indicates that natural selection for maintaining the pathway can be lost easily. Indeed, if transposon silencing is the critical function of the RNAi pathway, then the system is in danger of putting itself out of business if it is too efficient. A species in which transposons have been completely silenced for a long evolutionary period is likely to lose all intact elements (because intactness is selected for only when an element transposes) and thereby lose selection to retain the RNAi pathway, opening the door to re-invasion. Perhaps also contributing to RNAi loss is its potential inhibition of dsRNA viruses and their associated satellite dsRNAs. In S. cerevisiae, the M satellite element of the reovirus-like L-A virus encodes a secreted toxin that kills neighboring cells lacking element-encoded immunity (35). If cells that have lost RNAi are better able to retain this system they might have a selective advantage despite having lost an efficient transposon-defense pathway.

### Example 8

### Production of siRNA from dsRNA In Vitro by Budding Yeast Dicer

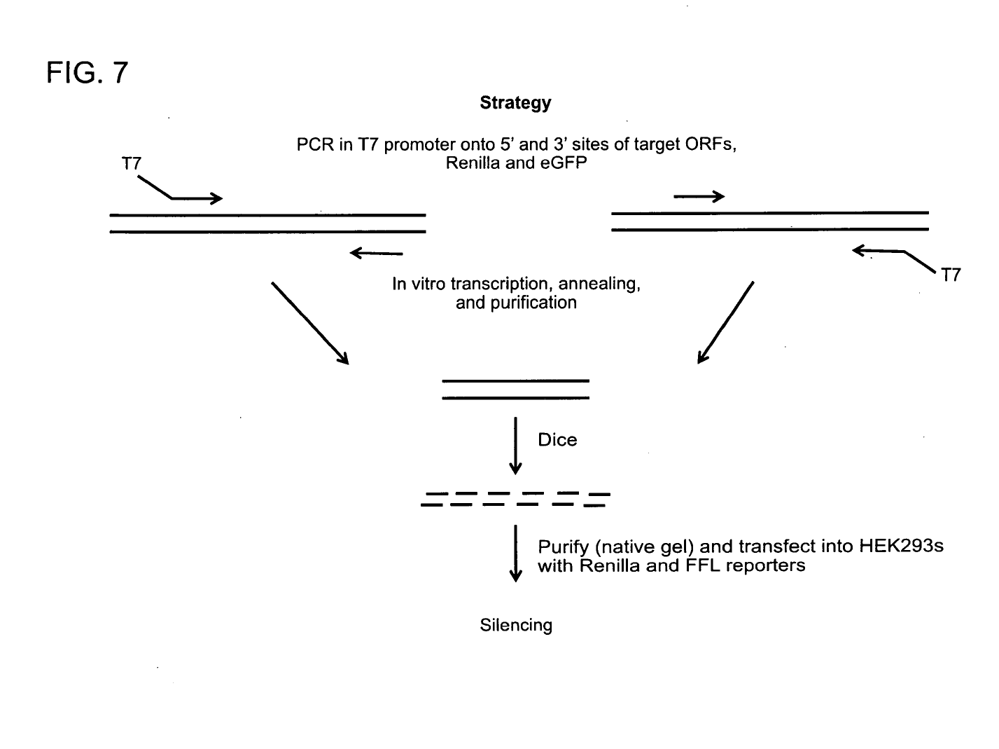

We confirmed the ability of budding yeast Dicer to generate siRNA in vitro by cleavage of dsRNA. As described in Example 9, we then assessed the ability of the resulting siRNA pool to silence a target gene (Renilla luciferase gene) when transfected into mammalian cells. An overview of the experimental strategy of Examples 8 and 9 is presented in FIG. 7, and further details are provided below in Materials and Methods.

Transcription templates containing 488-nt from the Renilla luciferase gene or from the GFP gene were generated by PCR. dsRNA substrates were prepared by annealing of ssRNA generated by in vitro transcription from each template. Annealed dsRNA was fractionated on a urea gel and isolated.

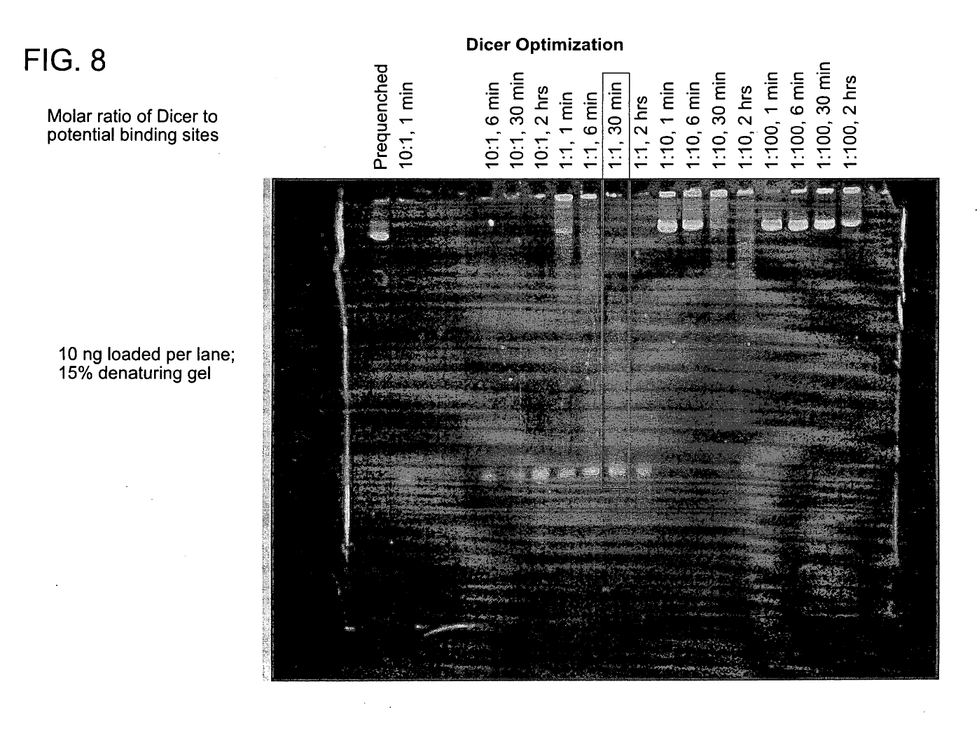

The dsRNA substrate was then incubated with a polypeptide containing amino acids 15-355 of K. polysporus Dicer that had been recombinantly produced in E. coli and purified. The polypeptide was produced with N-terminal 6× His and SUMO tags, followed by a Upl1 protease cleavage site to allow removal of the tags following purification (see Materials and Methods). Multiple reactions were performed in which (i) the molar ratio of Dicer polypeptide to potential Dicer binding sites in the dsRNA; and (ii) the duration of incubation were varied. (The number of potential binding sites was defined as the length of the dsRNA divided by 23, i.e., about 21.) The resulting products were fractionated on a denaturing gel, and RNA was detected using a fluorescent dye. As shown in FIG. 8, short RNA cleavage products were readily detected. A molar ratio of 1:1 and an incubation time of 30 minutes produced optimum results among the ratios and times tested in these experiments, though other ratios and incubation times also gave satisfactory results.

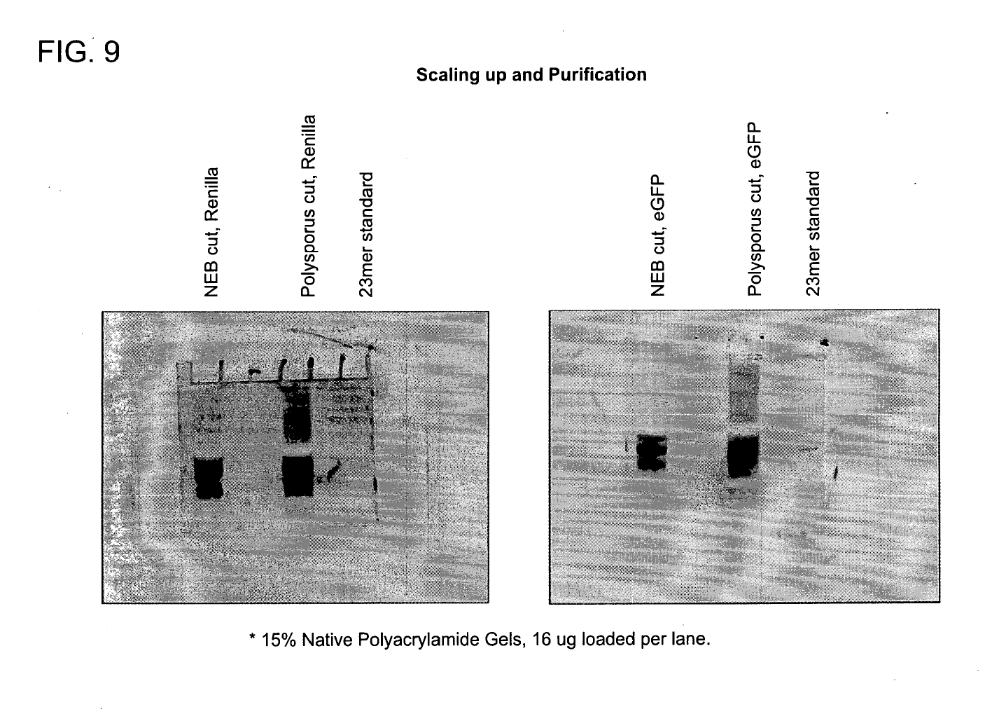

The ability of K. polysporus Dicer (amino acids 15-355) to cleave dsRNA was compared with that of a commercially available preparation of E. coli RNAse III (New England Biolabs, Ipswich, Mass.). Briefly, either K. polysporus Dicer fragment or E. coli RNAse III was incubated with either Renilla or GFP dsRNA, and the resulting products were fractionated on a native polyacrylamide gel with a 23 nucleotide standard. As shown in FIG. 9, K. polysporus Dicer was highly effective, generating siRNA with an efficiency at least as great as that of E. coli RNAse III. These results confirm that cleavage of dsRNA in vitro by budding yeast Dicer provides an effective means to generate of mixtures (pools) of siRNA.

### Example 9

### Silencing of a Target Gene by siRNA Produced In Vitro with Budding Yeast Dicer

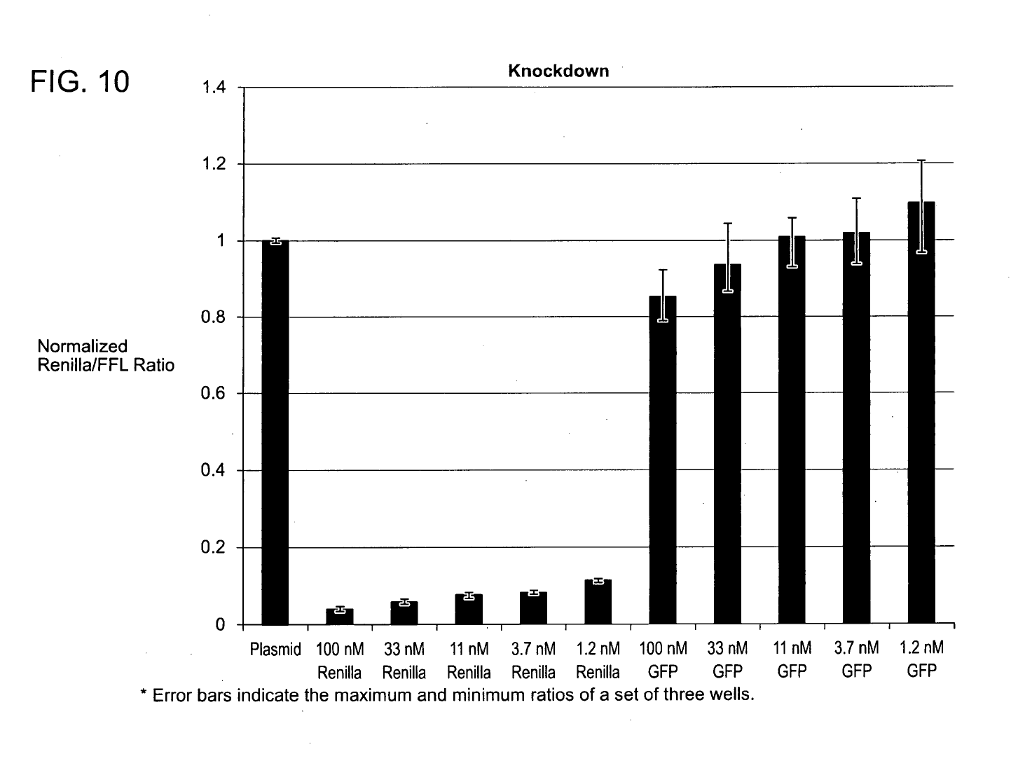

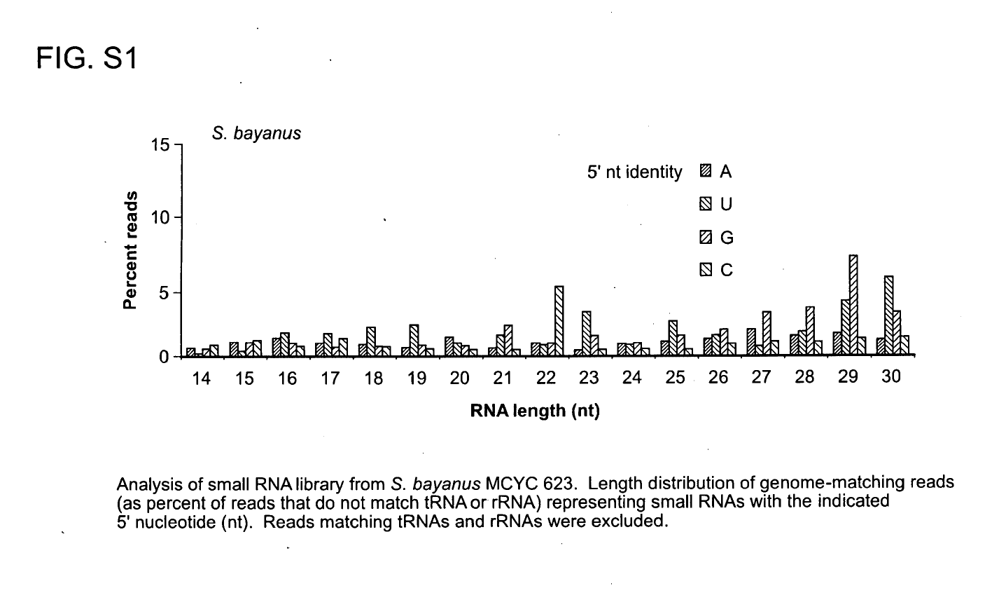

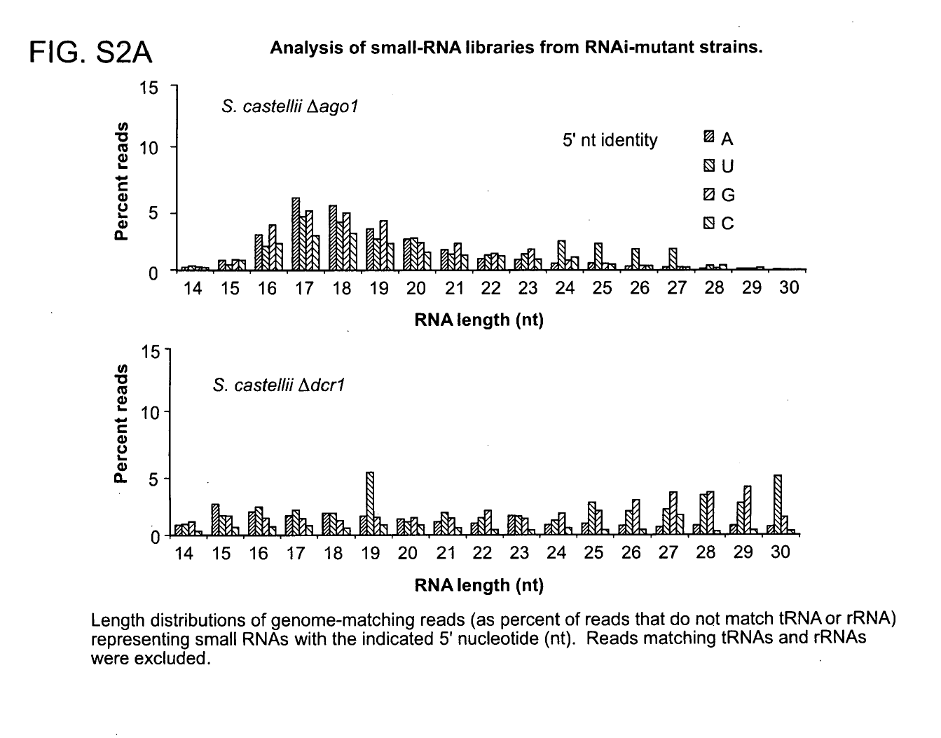

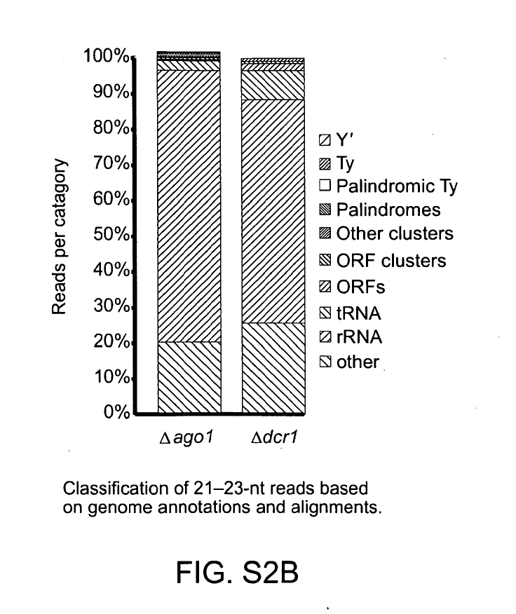

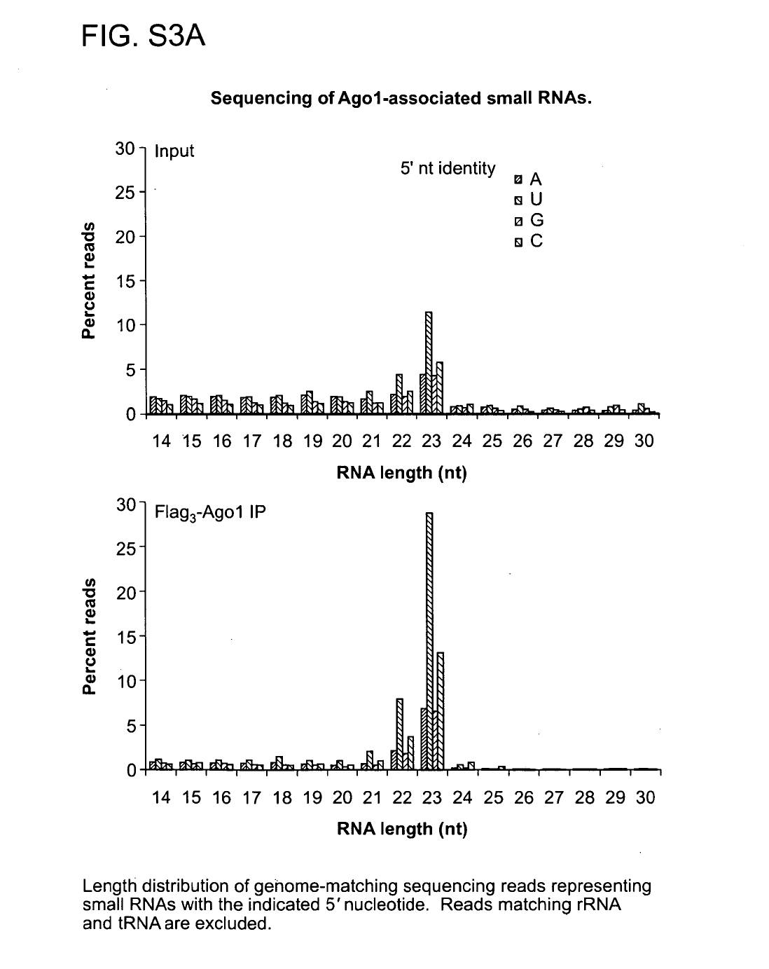

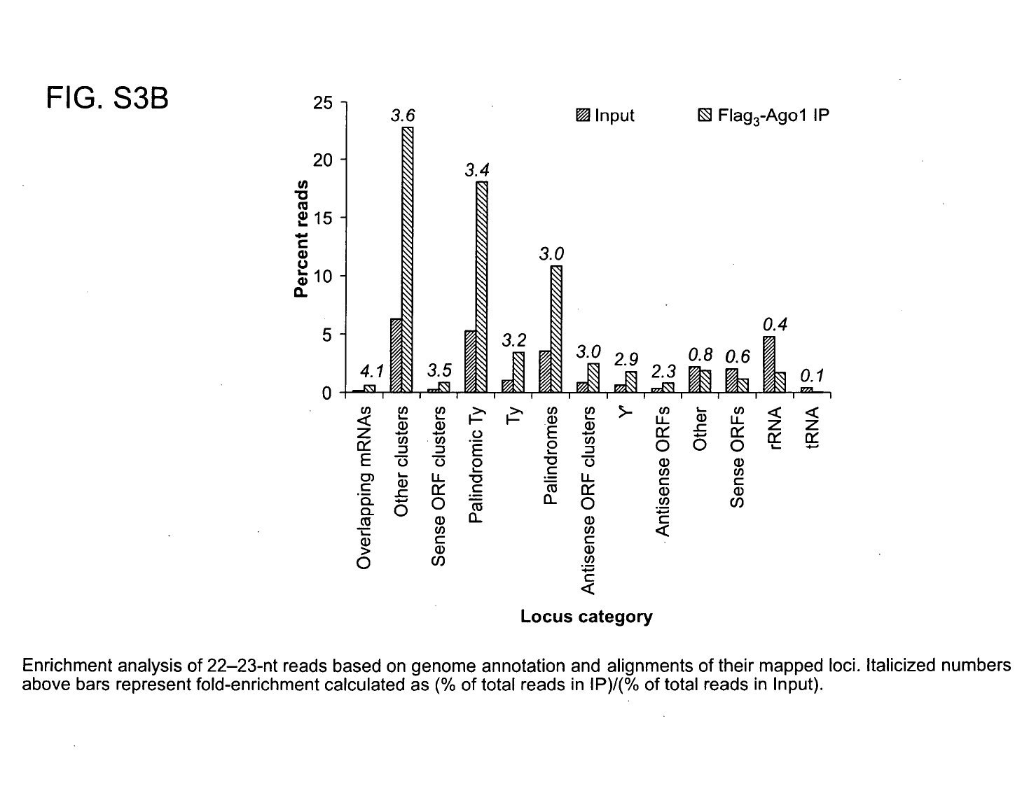

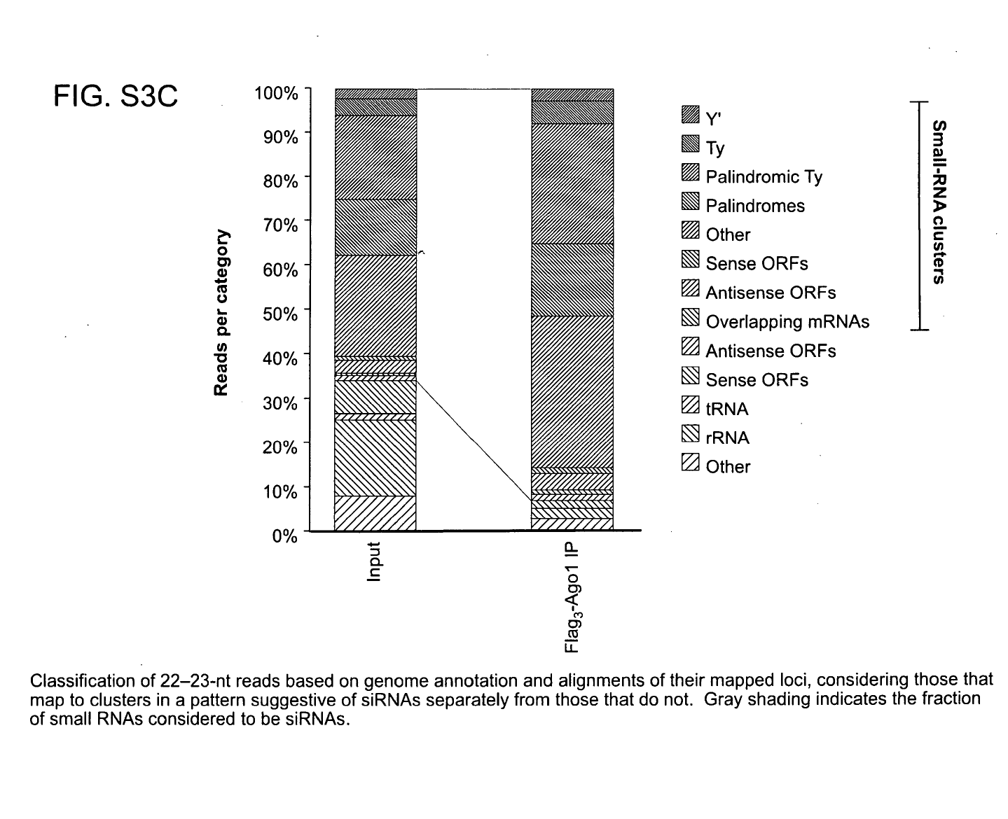

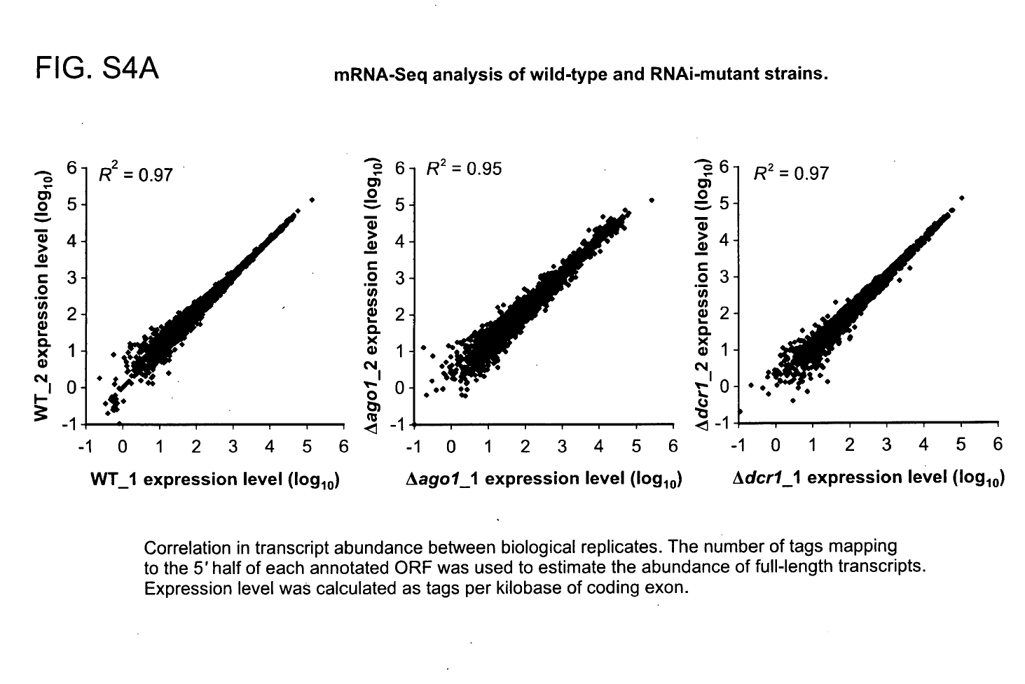

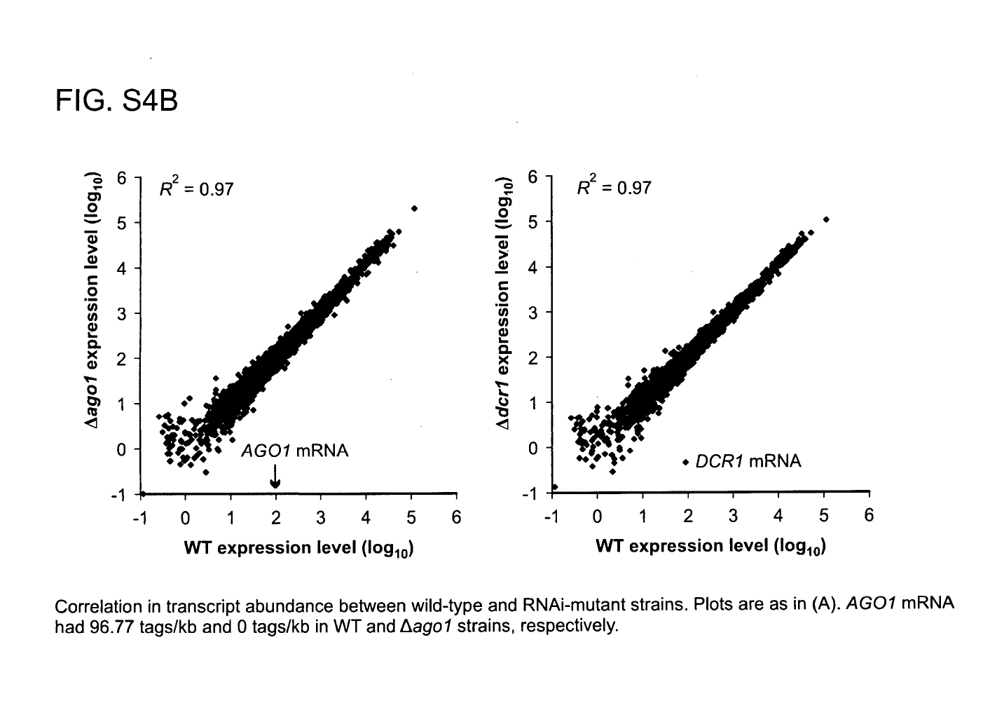

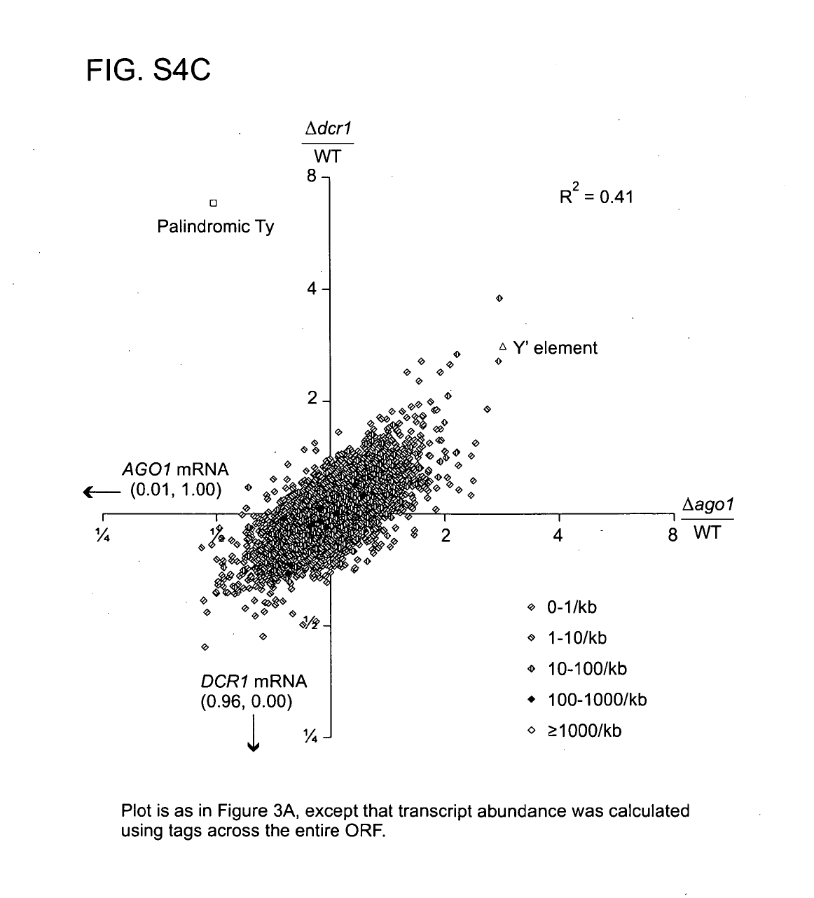

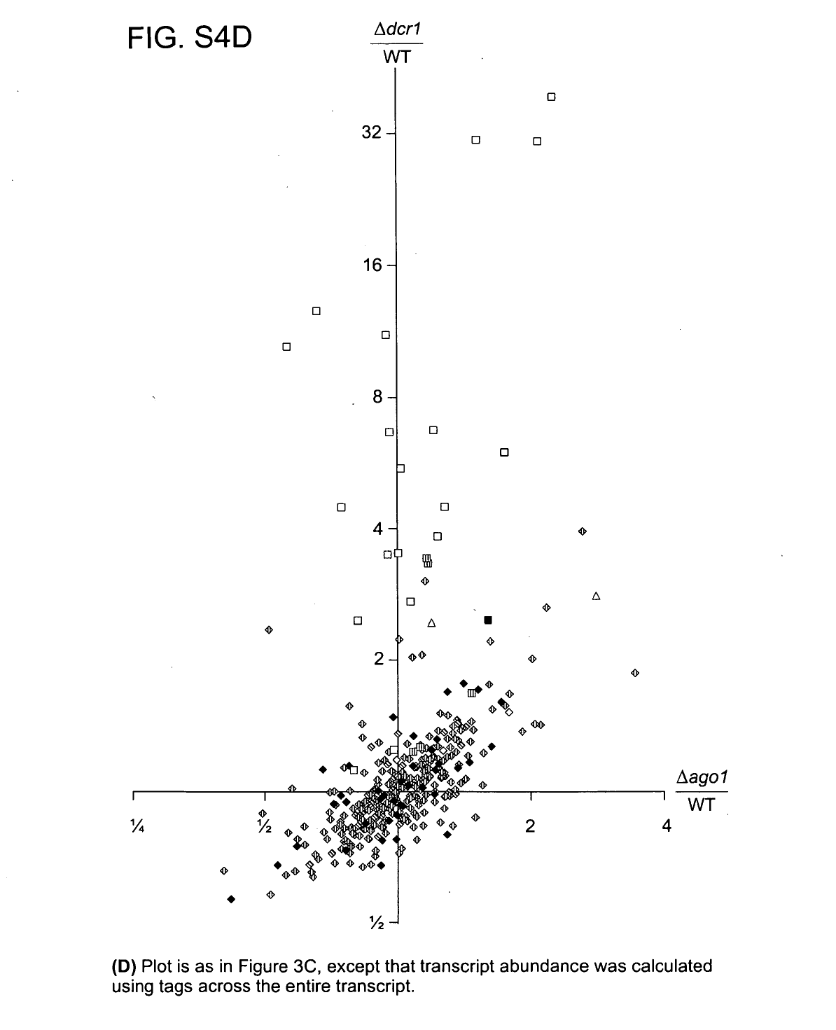

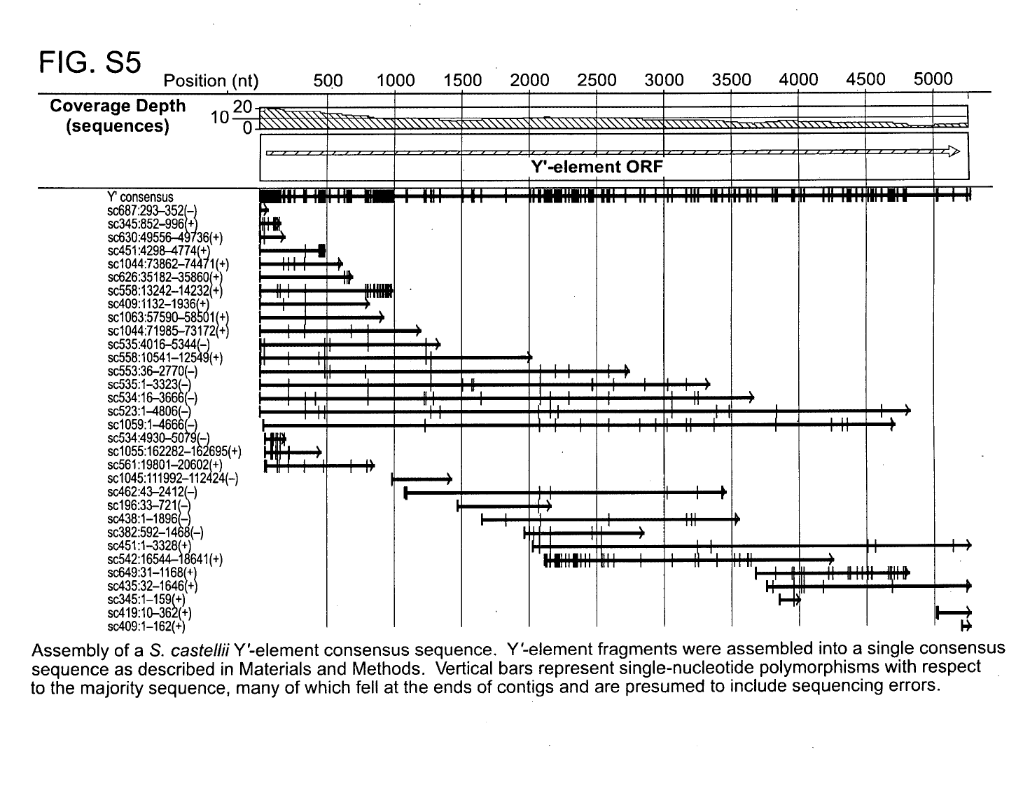

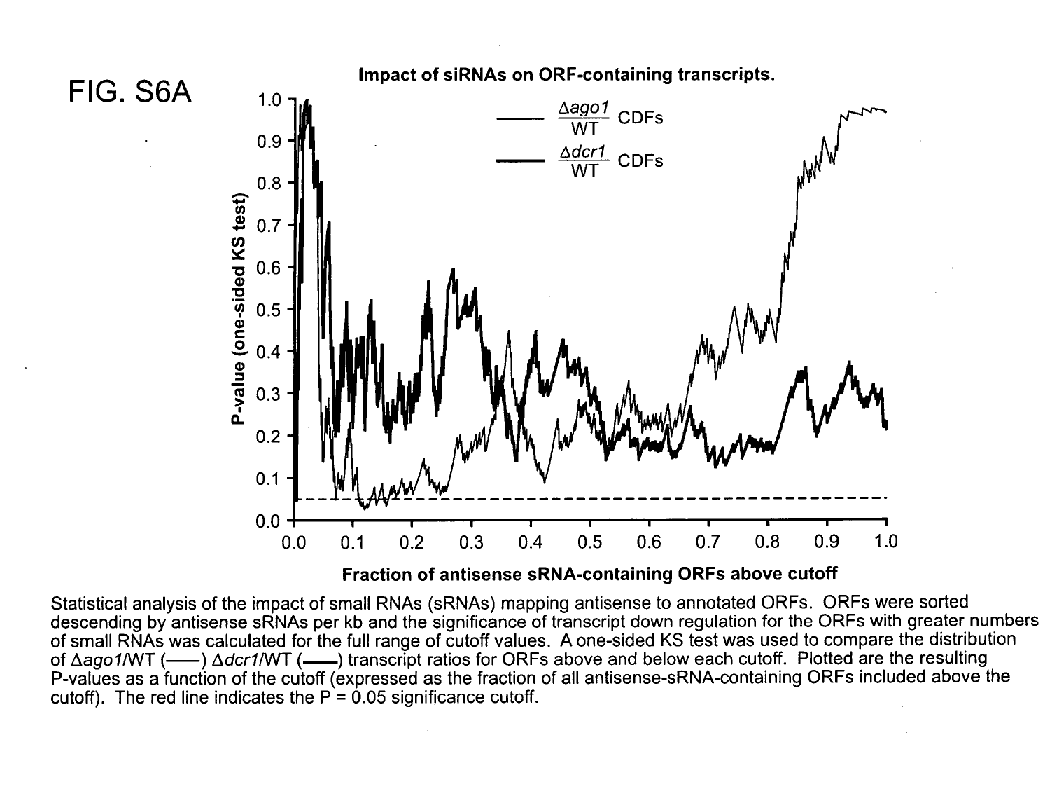

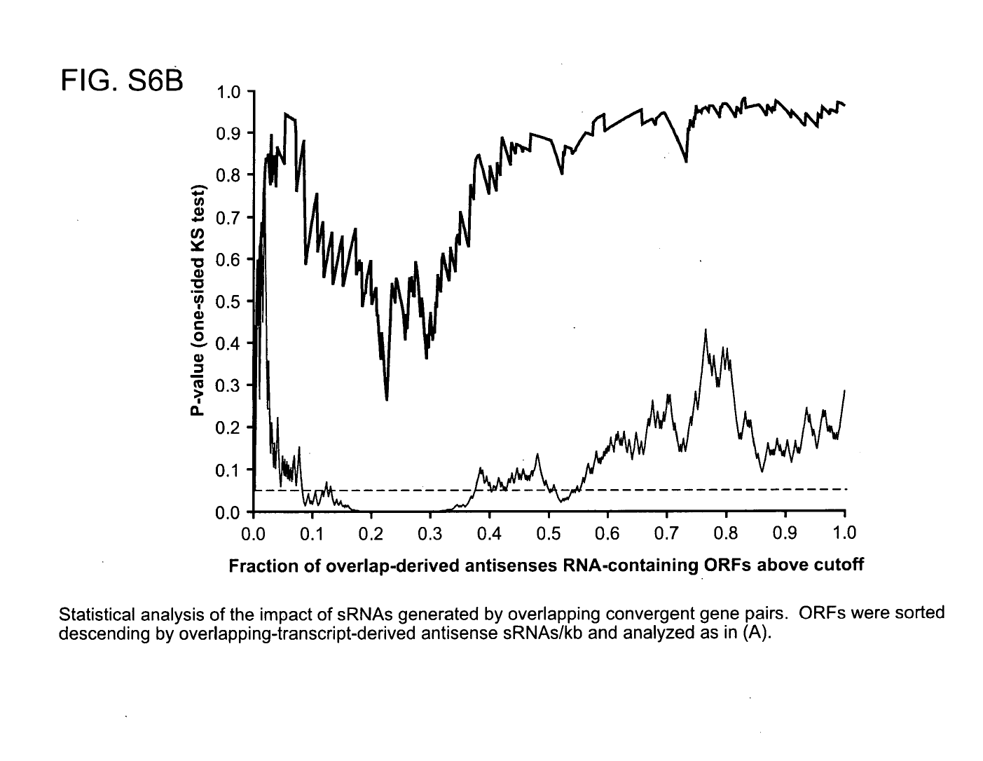

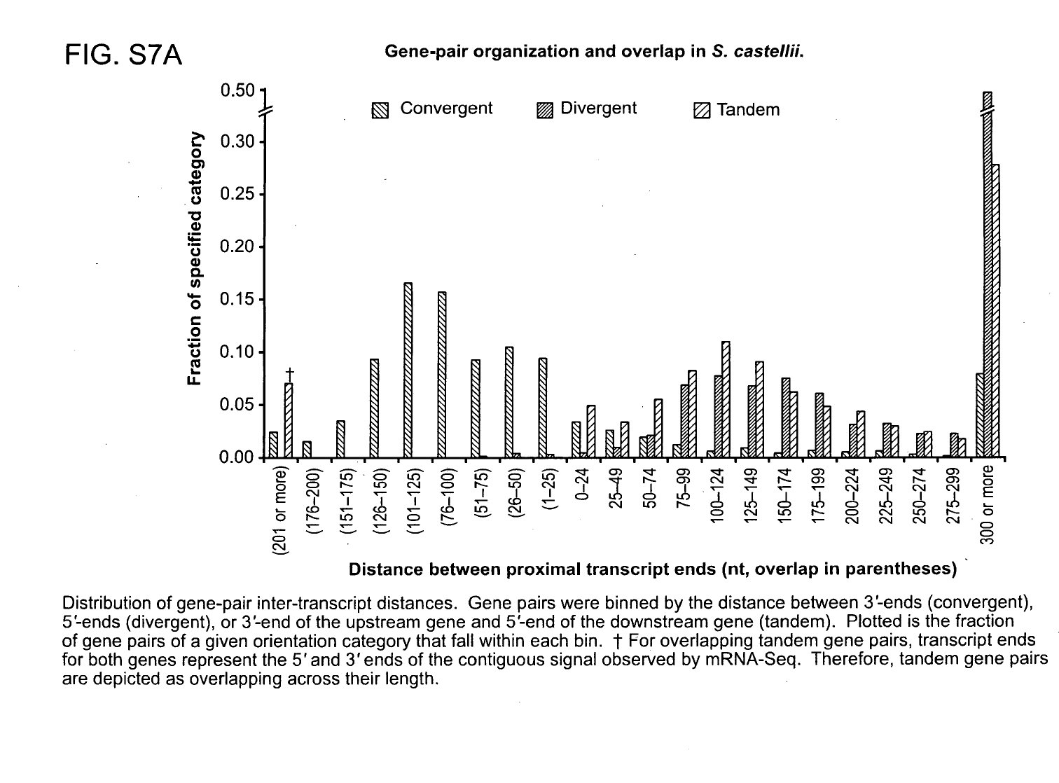

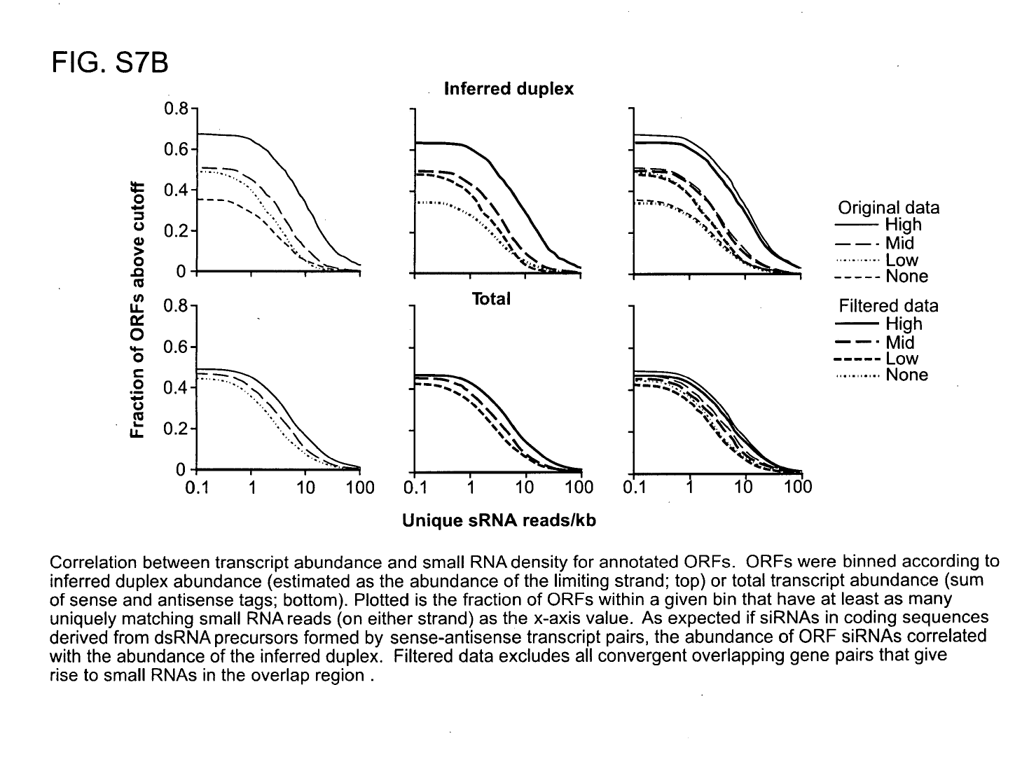

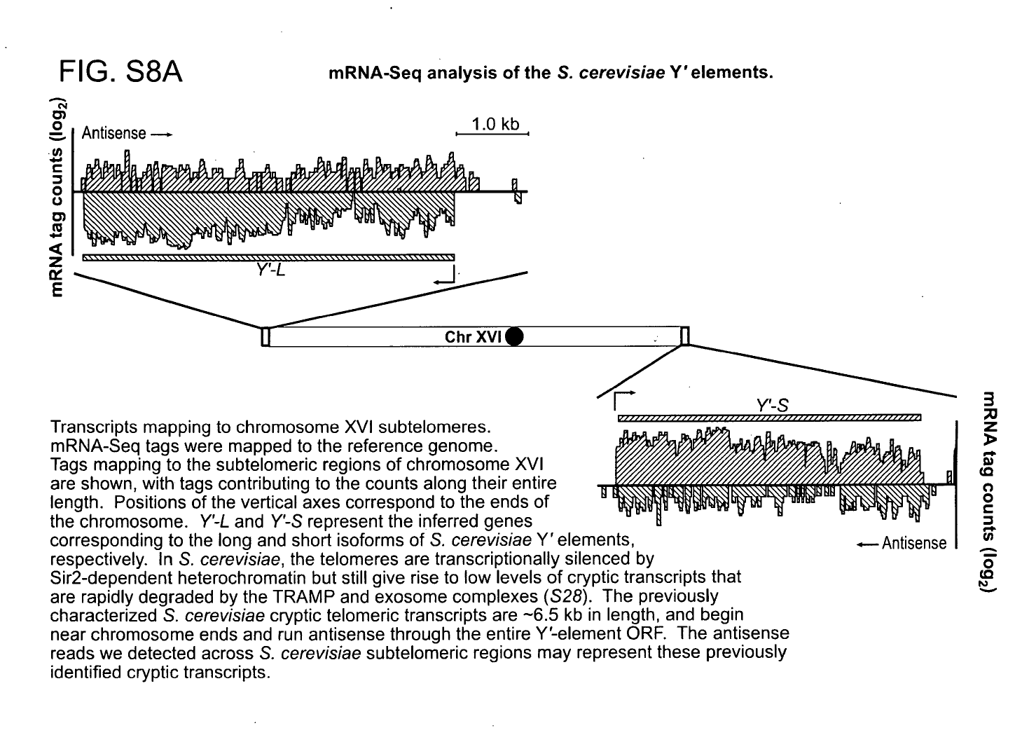

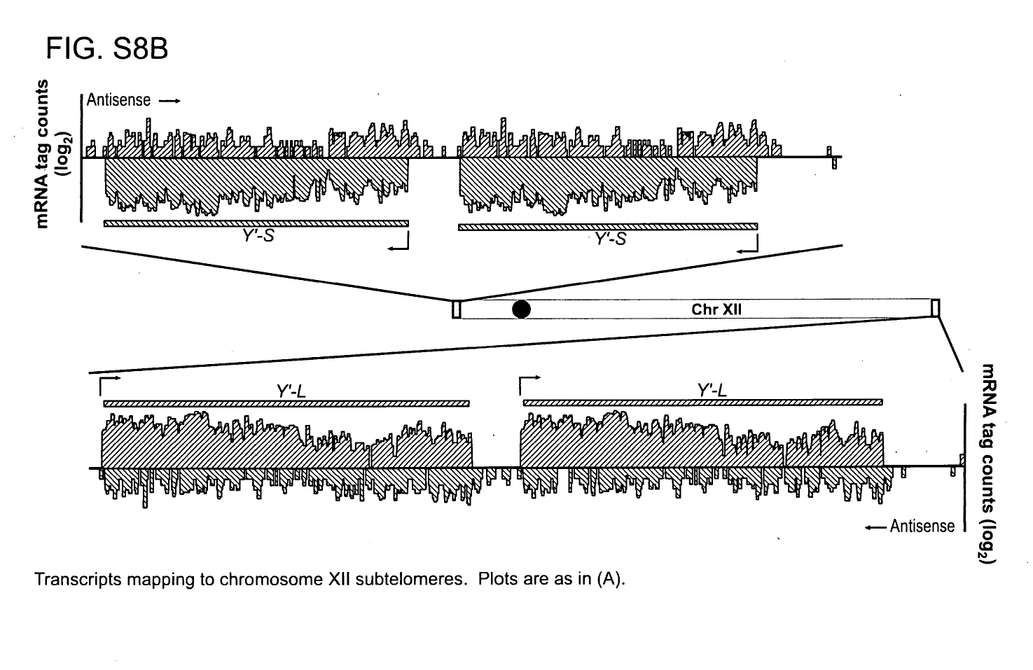

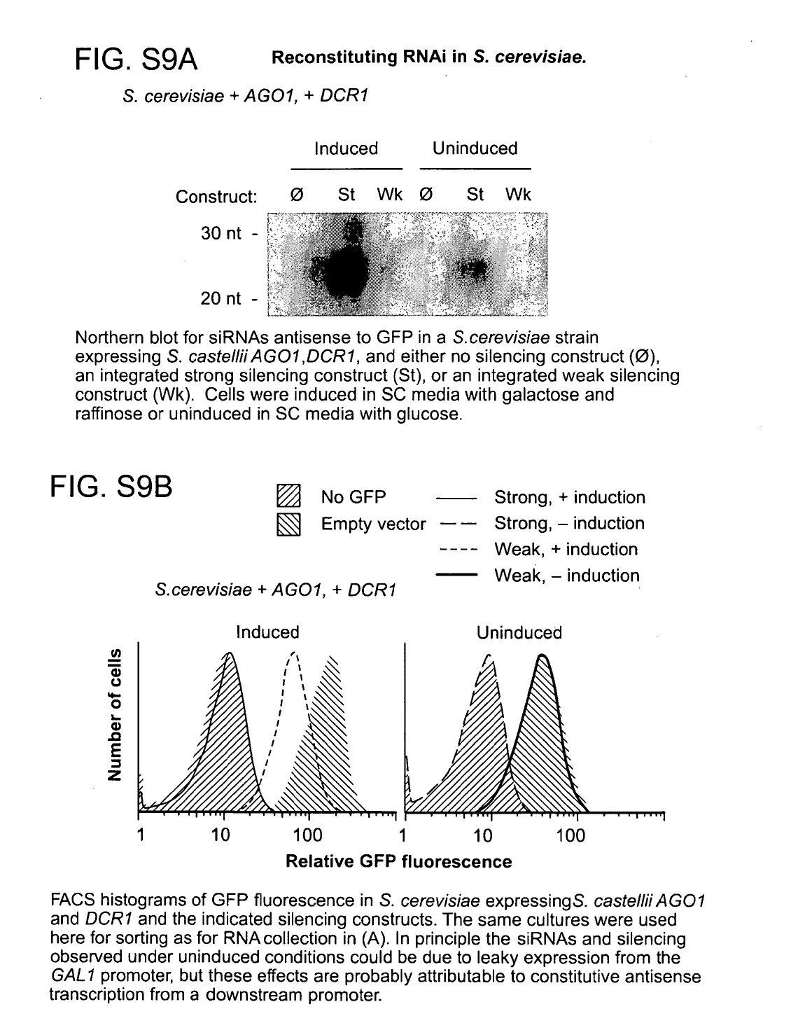

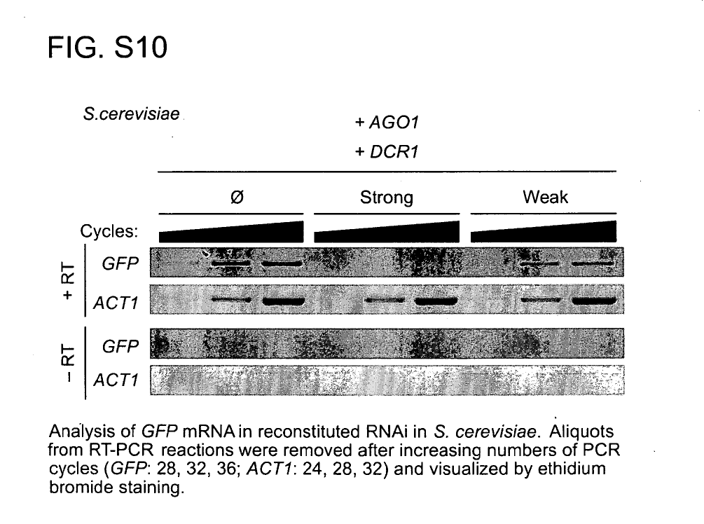

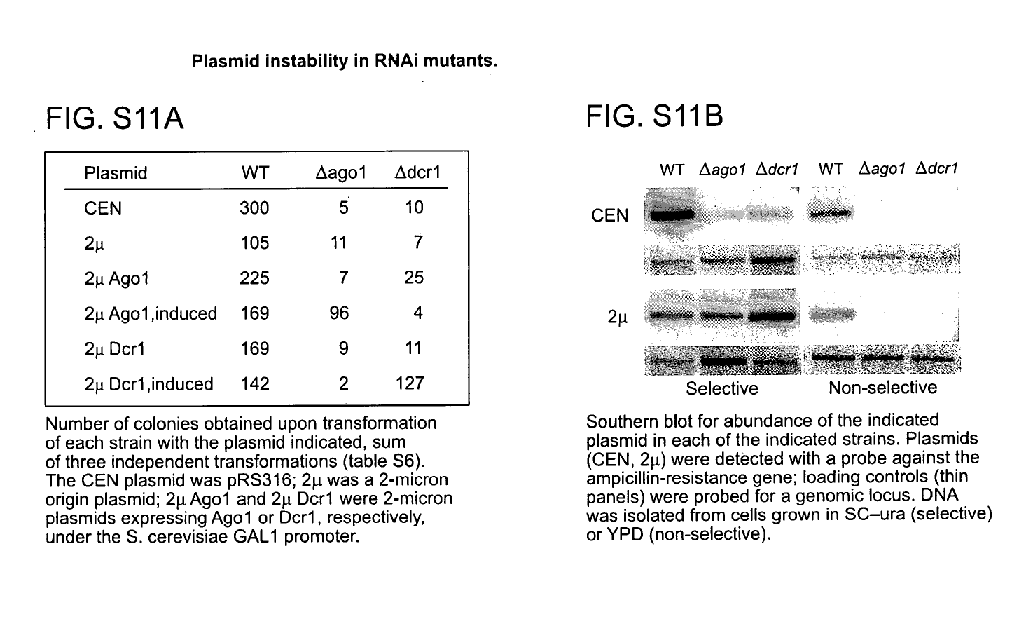

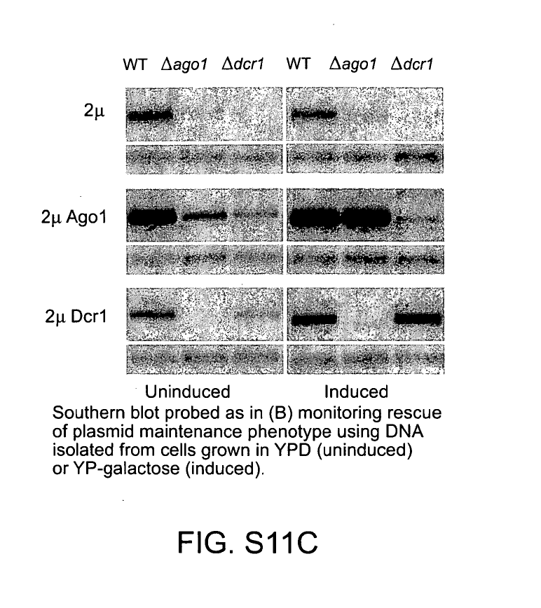

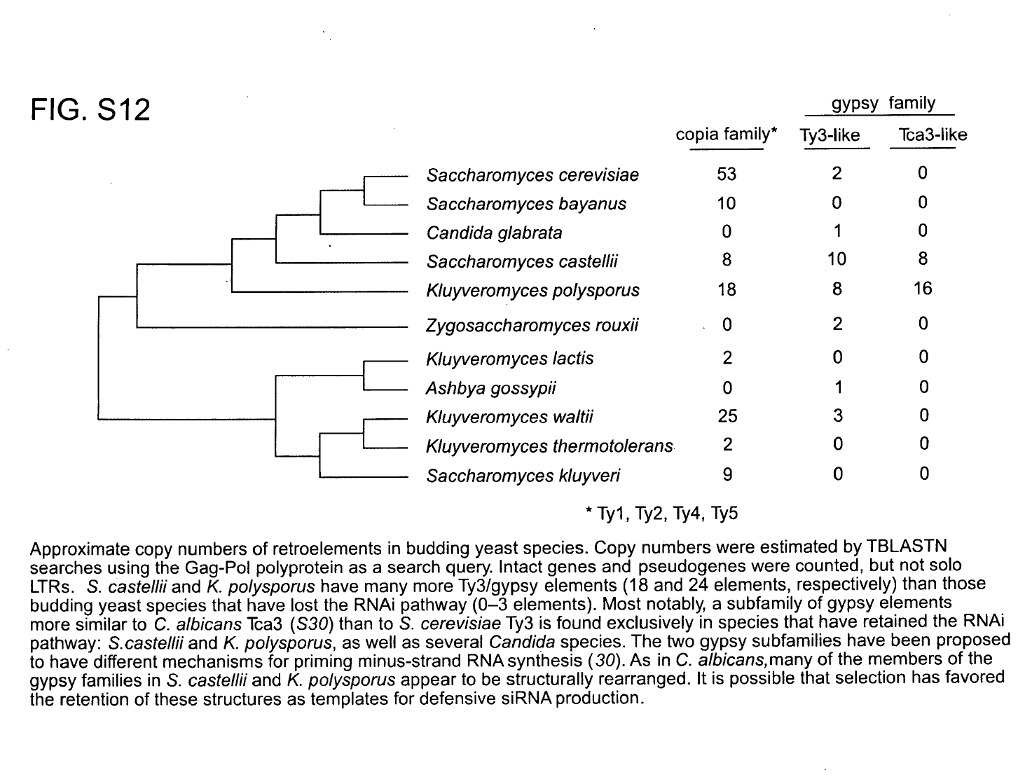

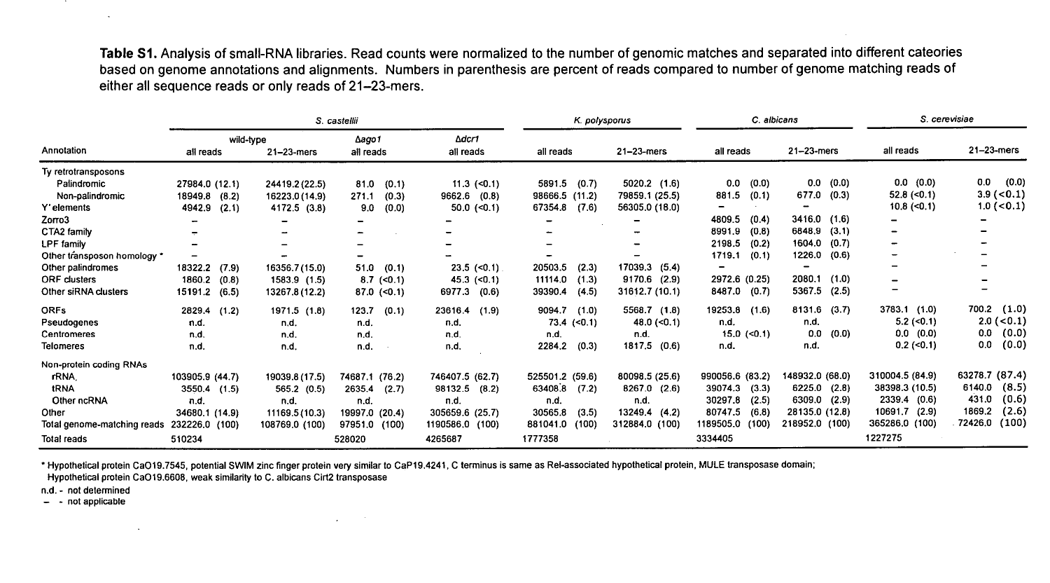

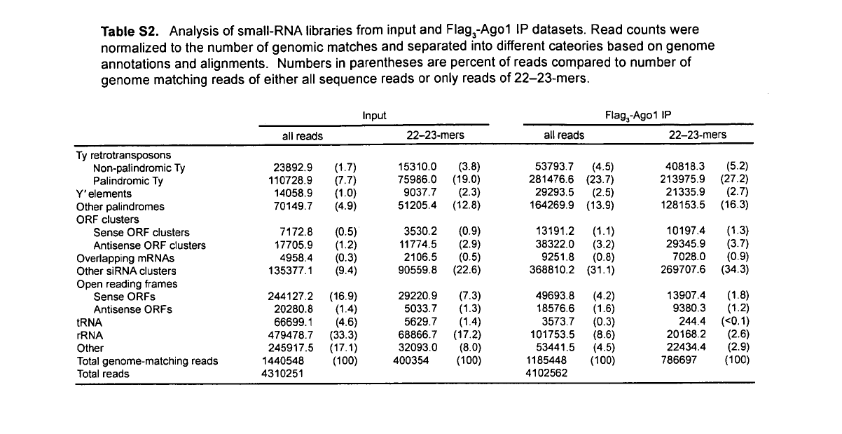

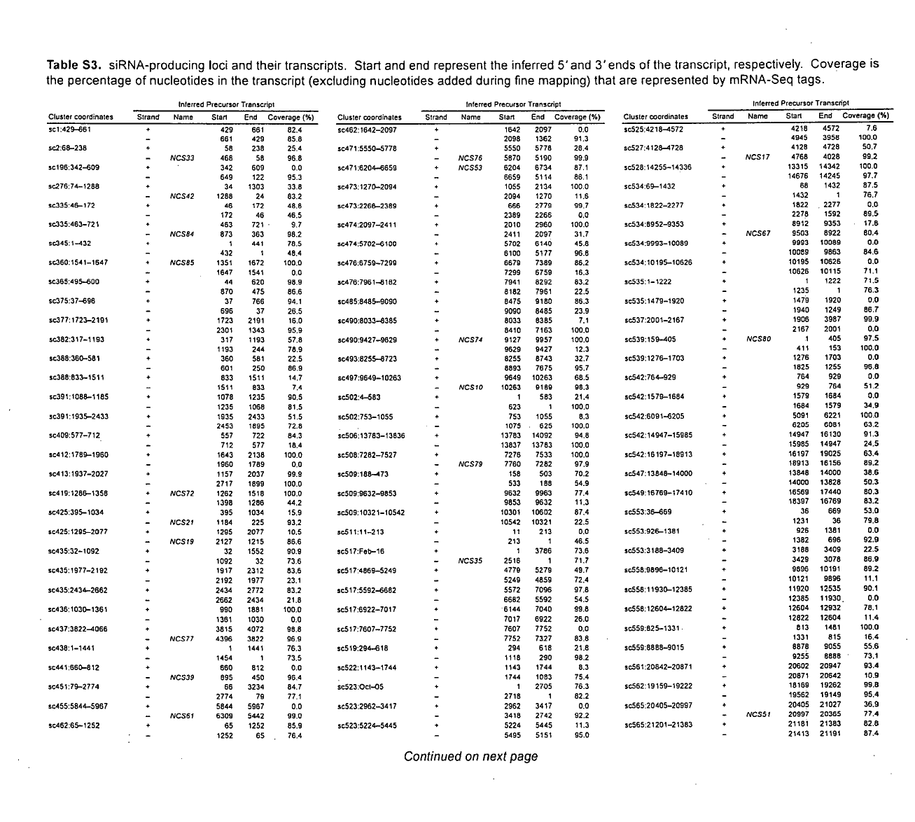

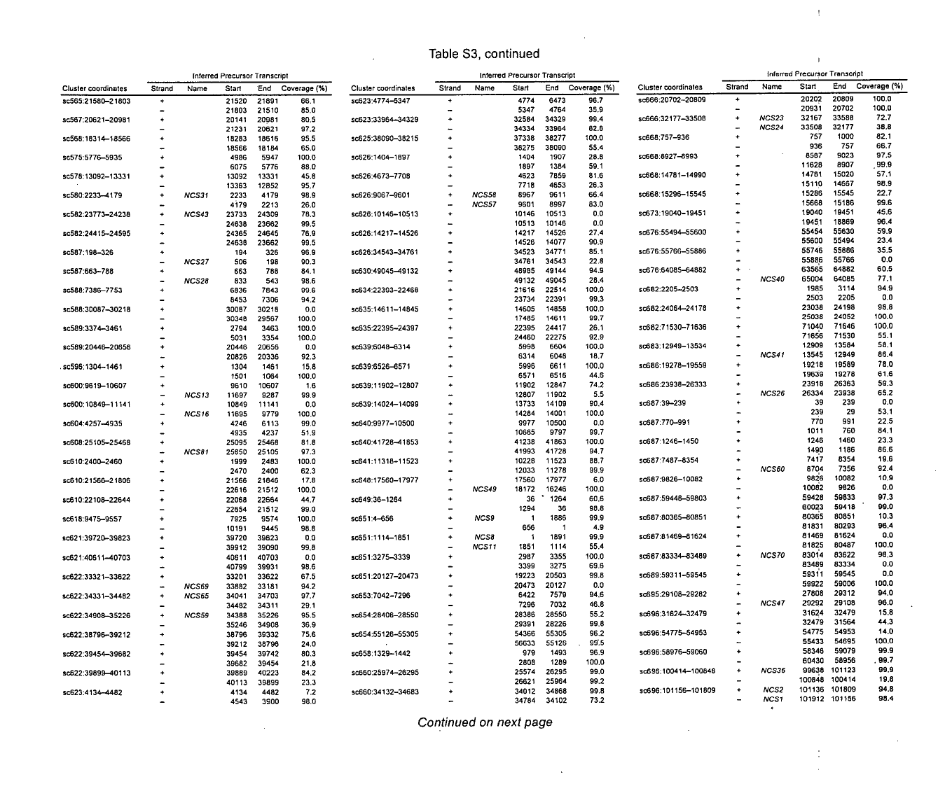

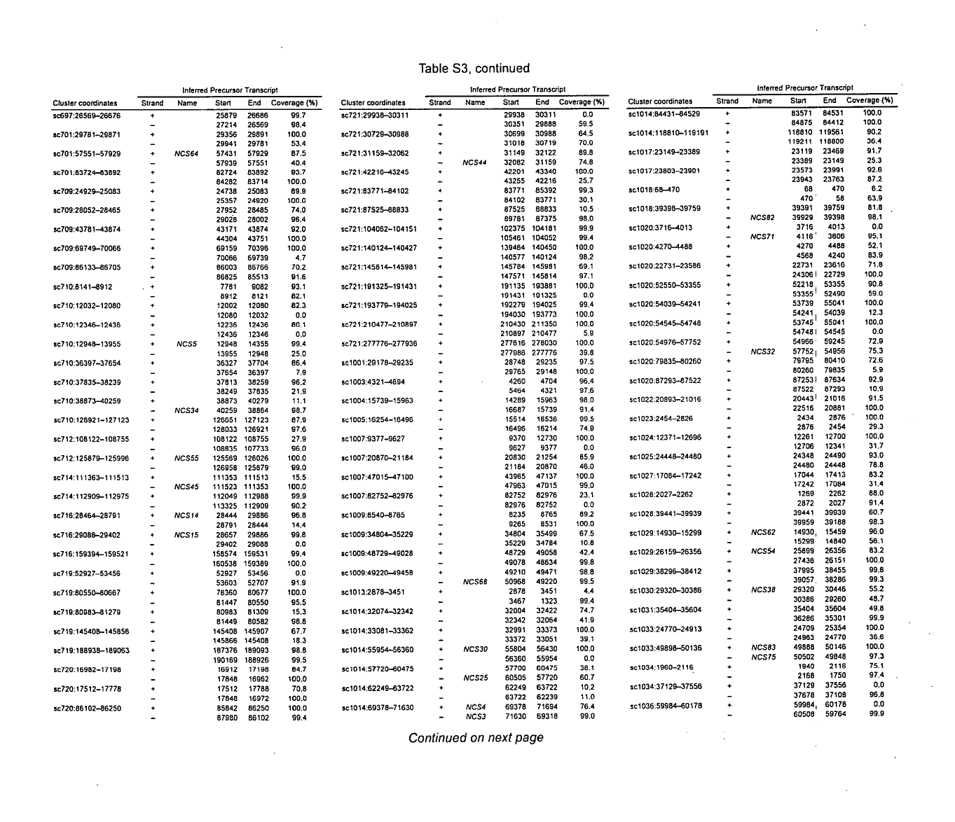

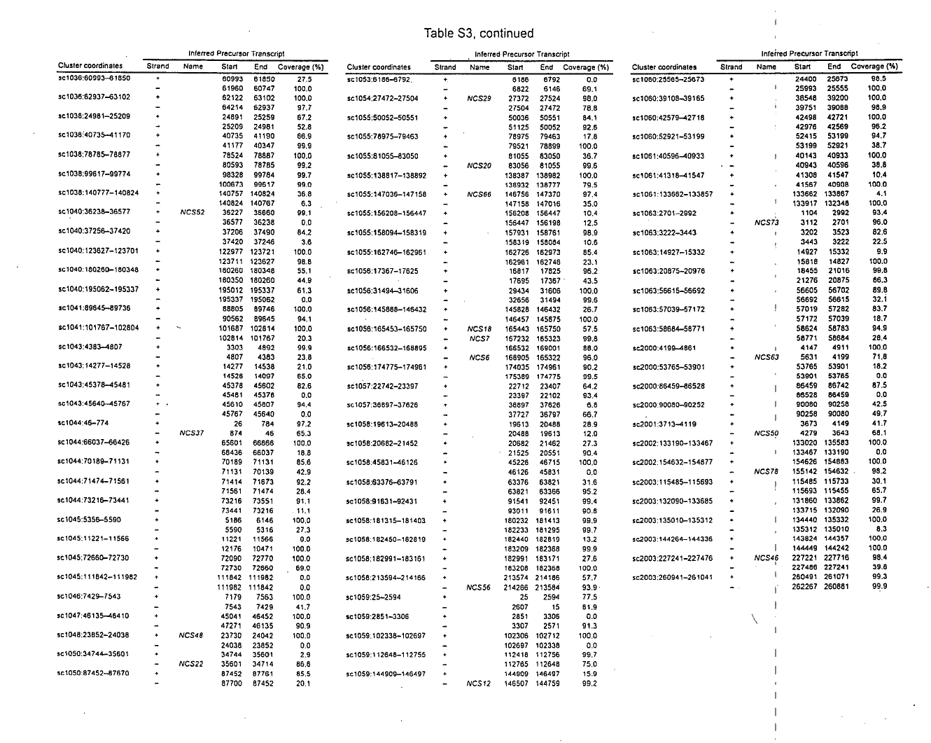

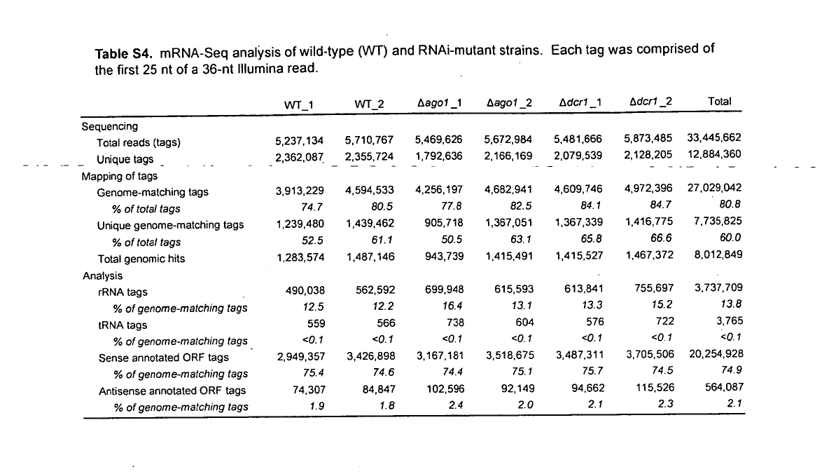

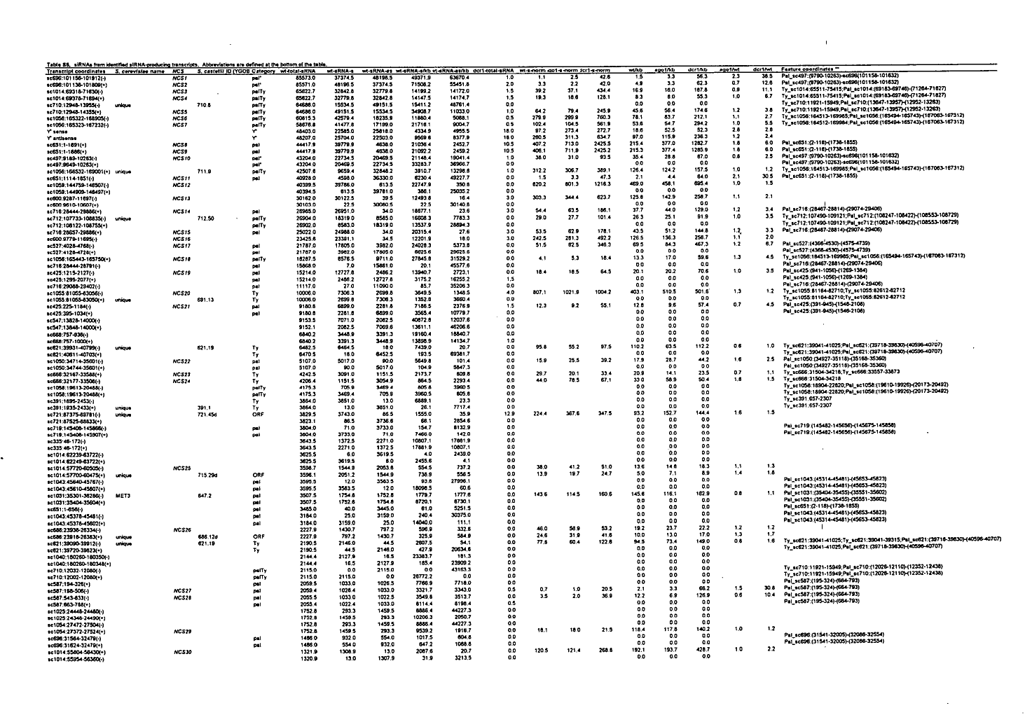

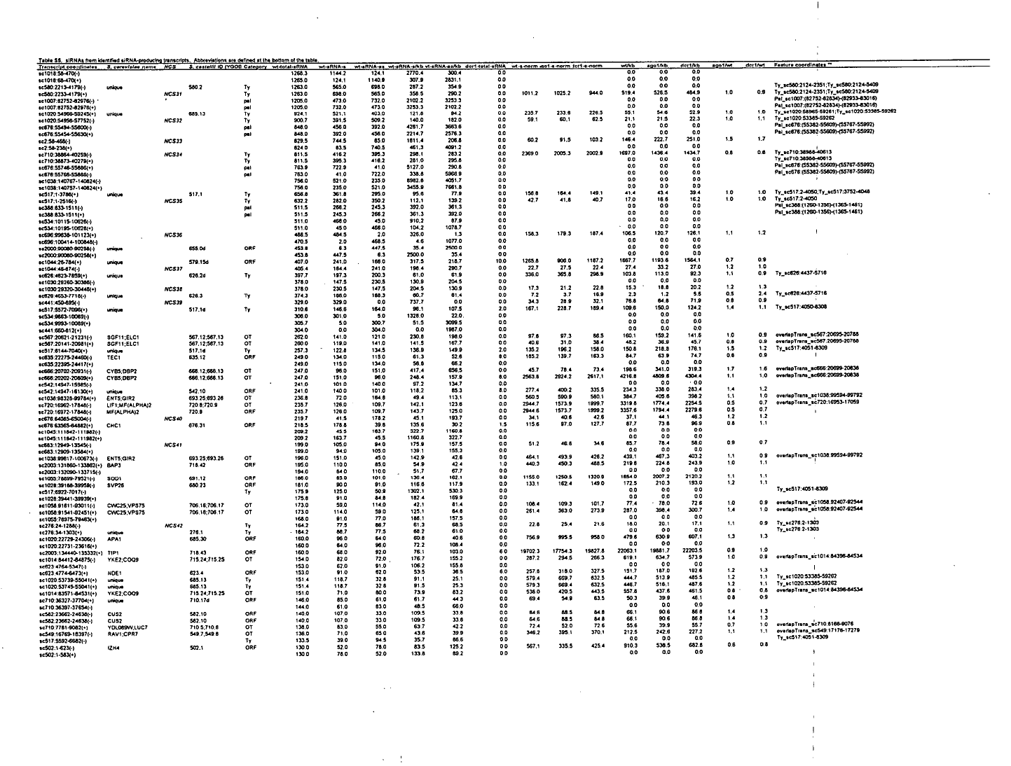

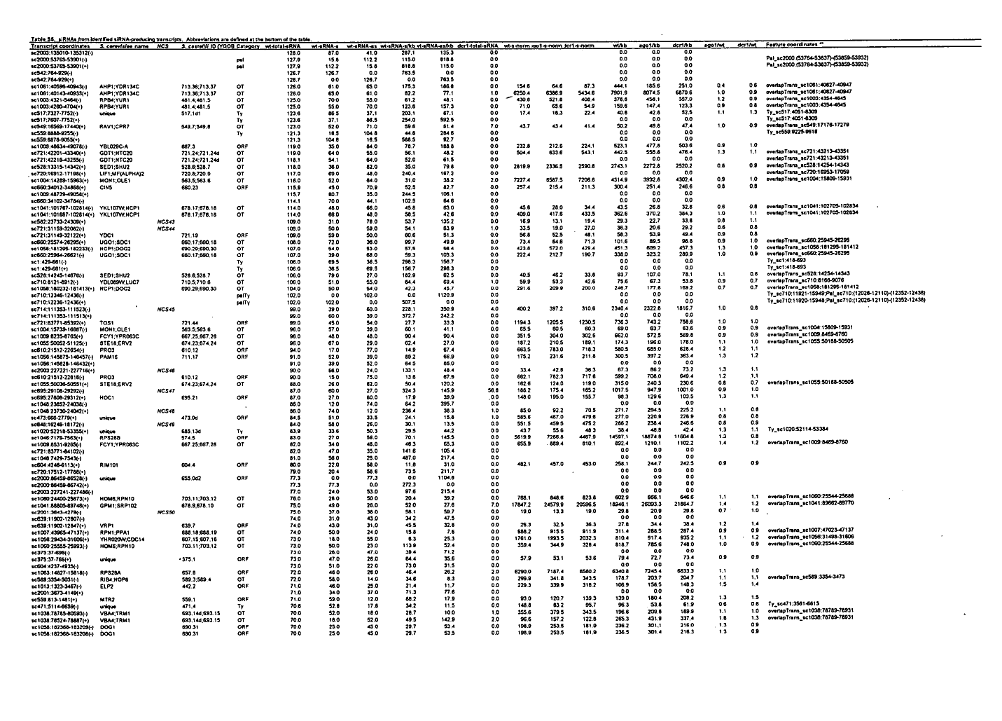

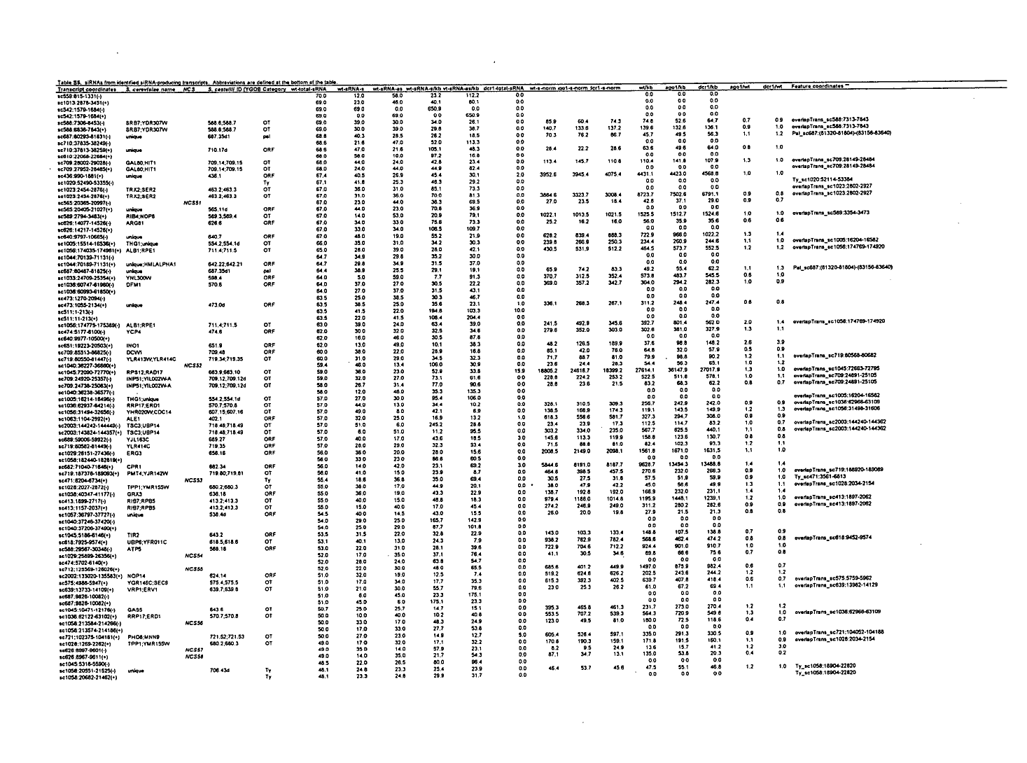

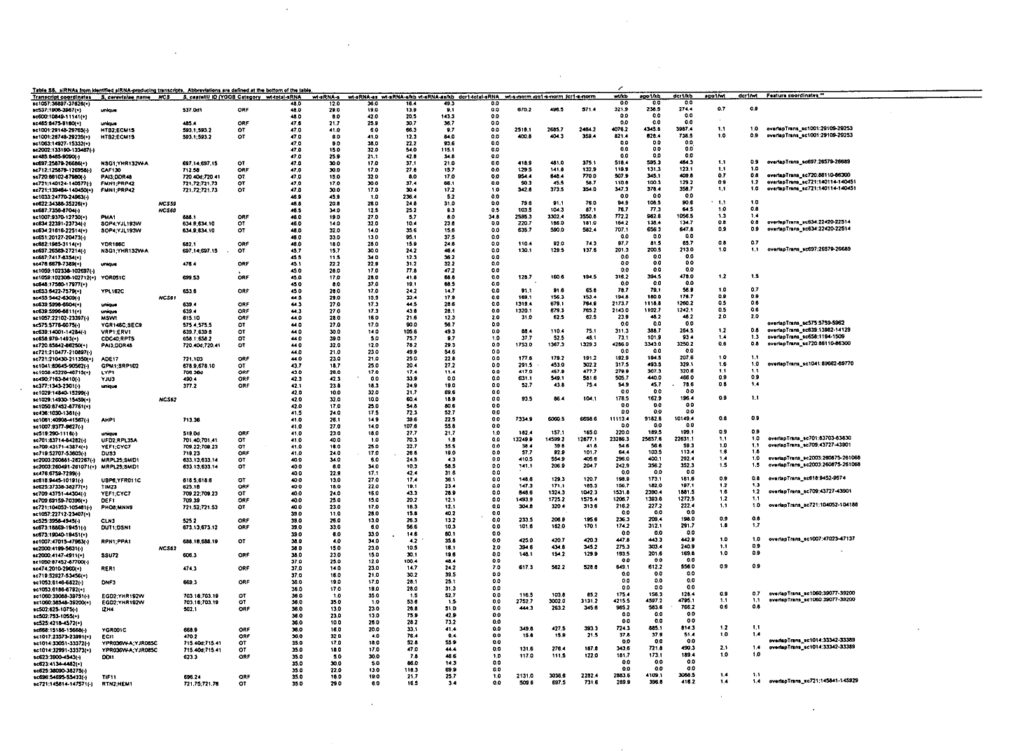

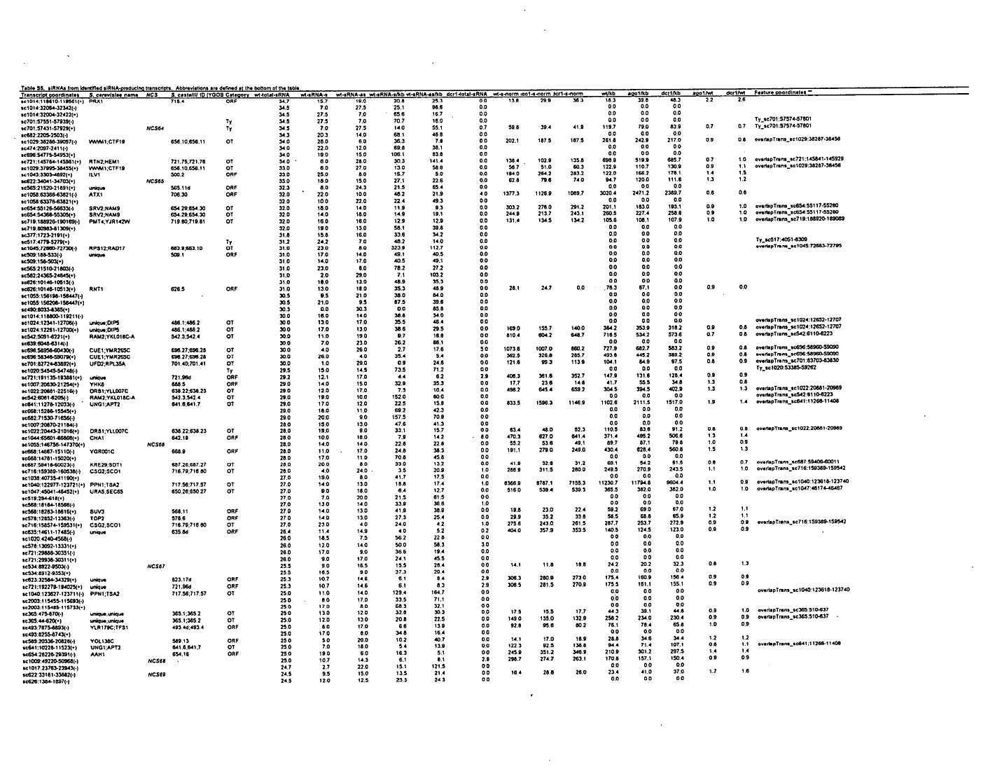

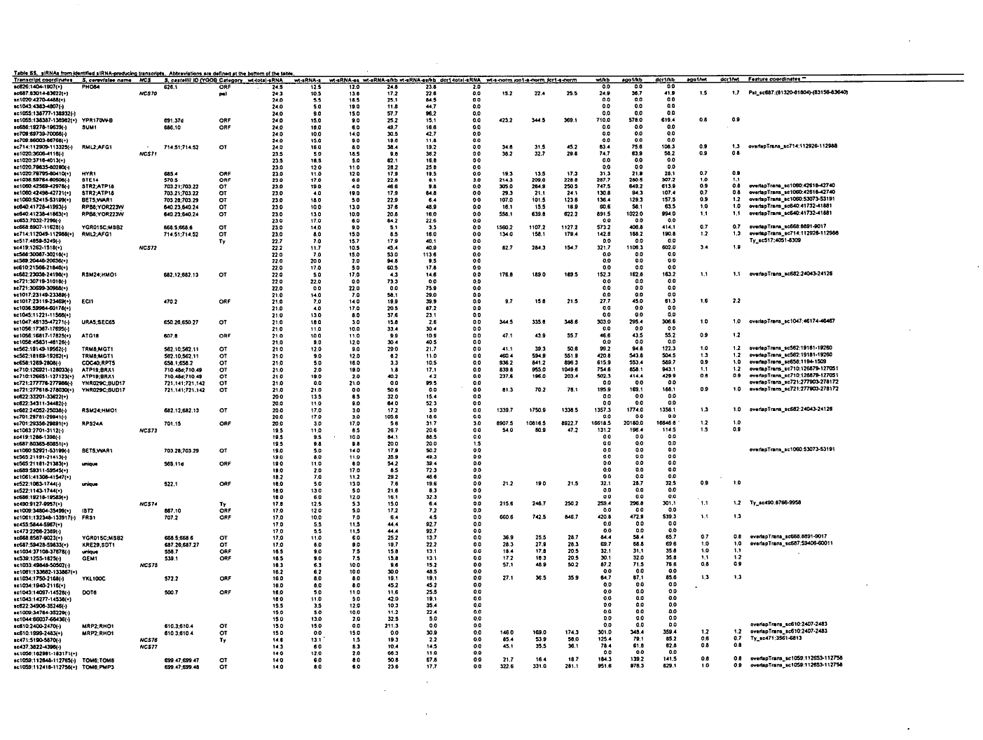

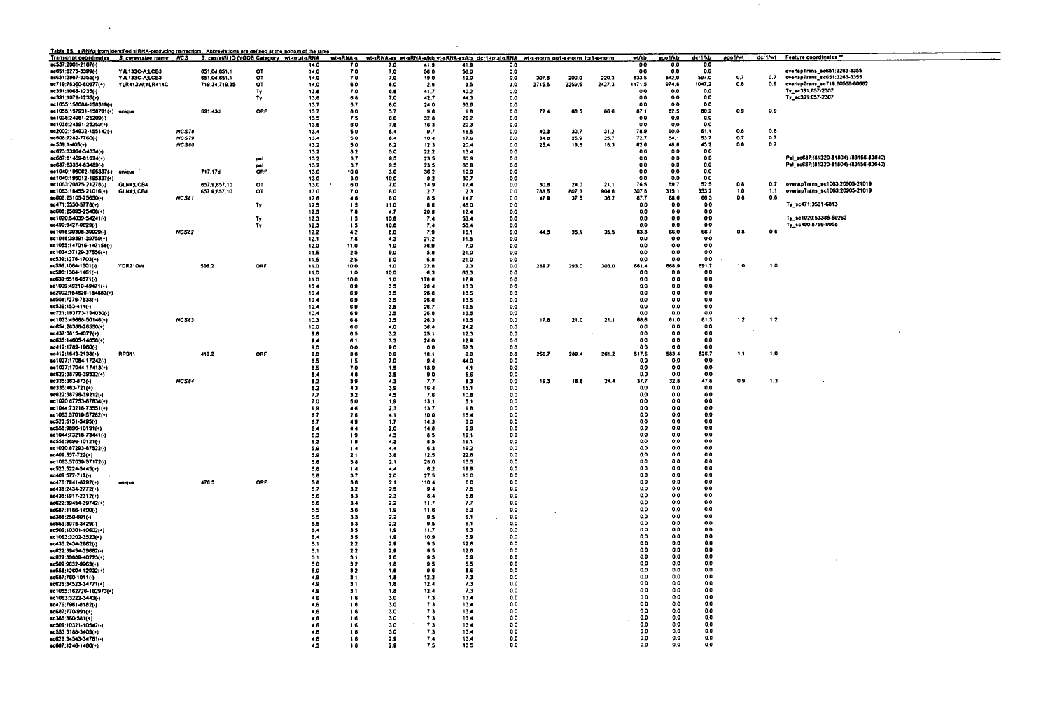

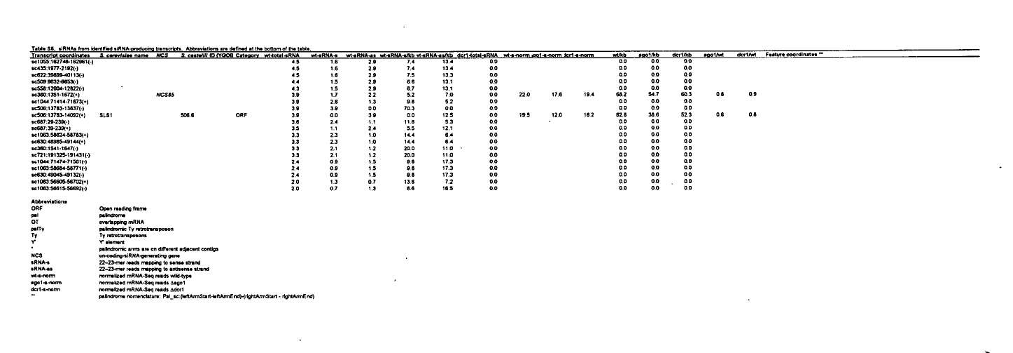

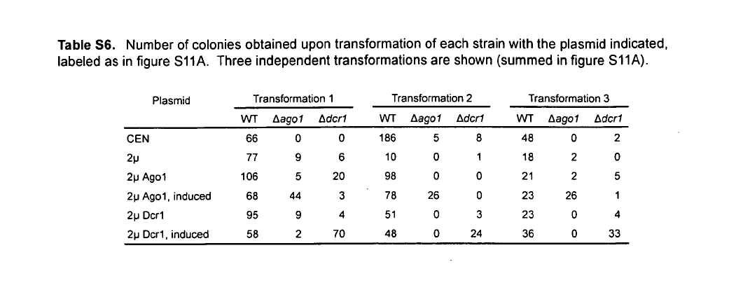

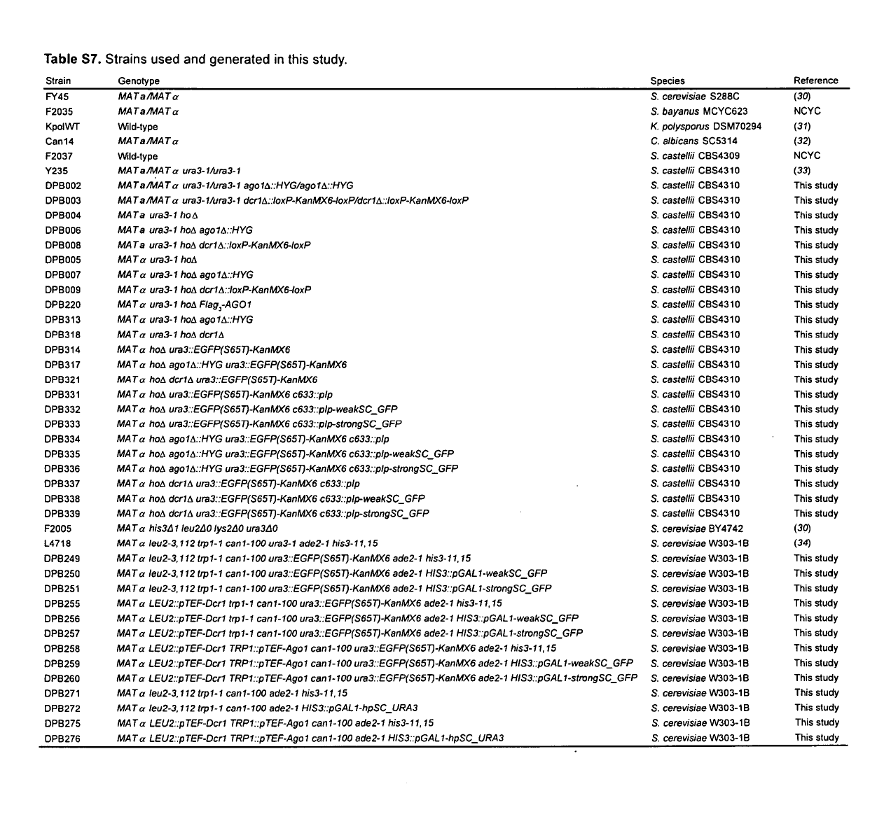

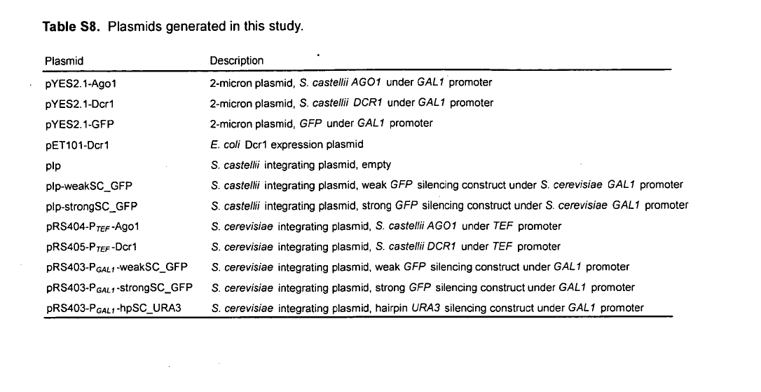

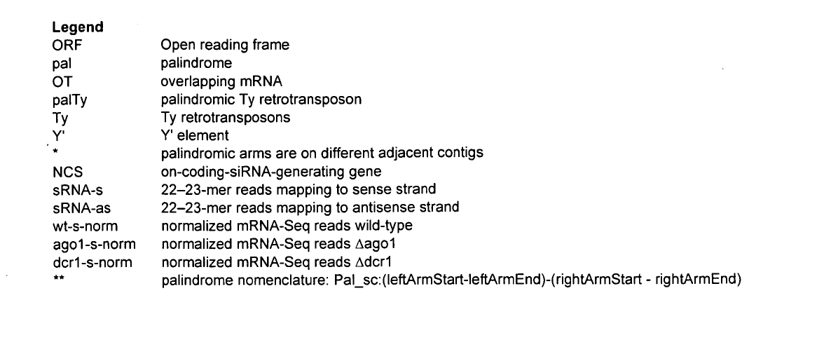

The ability of a pool of siRNAs generated by K. polysporus Dicer-mediated digestion of Renilla luciferase dsRNA to silence a target gene in mammalian cells was tested. In this case the dsRNA was produced using a fragment consisting of amino acids 1-384 of K. polysporus Dicer. (It should be noted that fragments 1-398, 1-376, 11-355, and 15-355 have also been tested and all retain dicing activity.) HEK293 cells were transfected with firefly luciferase control reporter plasmid, Renilla luciferase reporter plasmid, and various concentrations of siRNA ranging between 1.2 nM and 100 nM. Firefly and Renilla luciferase activities were measured approximately 24 hours after transfection. Renilla luciferase activity was normalized to firefly luciferase activity to control for transfection efficiency. As shown in FIG. 10, siRNA concentrations as low as 1.2 nM produced robust silencing (>85% knockdown). In contrast, cotransfection of siRNA generated by Dicer-mediated cleavage of GFP dsRNA produced little or no silencing even at concentrations as high as 100 nM. These results confirm the ability of siRNA pools generated by budding yeast Dicer in vitro to effectively and specifically silence target genes in mammalian cells.

### Example 10

### Silencing of an Endogenous Gene by siRNA Produced in Vitro with Budding Yeast Dicer

Examples 8 and 9 are repeated using an endogenous mammalian gene as the target.

### REFERENCES

1. Y. Tomari, P. D. Zamore, Genes Dev 19, 517 (2005).
2. C. D. Malone, G. J. Hannon, Cell 136, 656 (2009).
3. T. A. Farazi, S. A. Juranek, T. Tuschl, Development 135, 1201 (2008).
4. V. Fulci, G. Macino, Curr Opin Microbiol 10, 199 (2007).
5. S. I. Grewal, S. Jia, Nat Rev Genet. 8, 35 (2007).
6. H. Nakayashiki, N. Kadotani, S. Mayama, J Mol Evol 63, 127 (2006).
7. J. D. Laurie, R. Linning, G. Bakkeren, Curr Genet. 53, 49 (2008).
8. J. Berman, P. E. Sudbery, Nat Rev Genet. 3, 918 (2002).
9. D. R. Scannell et al., Proc Natl Acad Sci USA 104, 8397 (2007).
10. M. Axelson-Fisk, P. Sunnerhagen, Comparative Genomics: Using Fungi as Models 15, 1 (2006).
11. T. M. Hall, Structure 13, 1403 (2005).
12. A. Grimson et al., Nature 455, 1193 (2008).
13. N. C. Lau, L. P. Lim, E. G. Weinstein, D. P. Bartel, Science 294, 858 (2001).
14. T. A. Montgomery et al., Cell 133, 128 (2008).
15. M. Buhler, N. Spies, D. P. Bartel, D. Moazed, Nat Struct Mol Biol 15, 1015 (2008).
16. Materials and methods are available as supporting material on Science Online.
17. B. Lamontagne, S. Larose, J. Boulanger, S. A. Elela, Curr Issues Mol Biol 3, 71 (2001).
18. E. Astromskas, M. Cohn, Yeast 24, 499 (2007).
19. E. Bernstein, A. A. Caudy, S. M. Hammond, G. J. Hannon, Nature 409, 363 (2001).
20. I. J. MacRae, J. A. Doudna, Curr Opin Struct Biol 17, 138 (2007).
21. H. Zhang, F. A. Kolb, L. Jaskiewicz, E. Westhof, W. Filipowicz, Cell 118, 57 (2004).
22. Q. Liu et al., Science 301, 1921 (2003).
23. F. Vazquez, V. Gasciolli, P. Crete, H. Vaucheret, Curr Biol 14, 346 (2004).
24. T. P. Chendrimada et al., Nature 436, 740 (2005).
25. R. Lister et al., Cell 133, 523 (2008).
26. E. J. Louis, J. E. Haber, Genetics 131, 559 (1992).
27. M. Yamada, N. Hayatsu, A. Matsuura, F. Ishikawa, J Biol Chem 273, 33360 (1998).
28. U. Nagalakshmi et al., Science 320, 1344 (2008).
29. M. Buhler, A. Verdel, D. Moazed, Cell 125, 873 (2006).
30. A. Sigova, N. Rhind, P. D. Zamore, Genes Dev 18, 2359 (2004).
31. M. Maiti, H. C. Lee, Y. Liu, Genes Dev 21, 590 (2007).
32. M. J. Curcio, D. J. Garfinkel, Proc Natl Acad Sci U S A 88, 936 (1991).
33. J. Berretta, M. Pinskaya, A. Morillon, Genes Dev 22, 615 (2008).
34. N. T. Ingolia, S. Ghaemmaghami, J. R. Newman, J. S. Weissman, Science 324, 218 (2009).
35. R. B. Widmer, Microbiol Rev 60, 250 (1996).
36. S. M. Hedtke, T. M. Townsend, D. M. Hillis, Syst Biol 55, 522 (2006).
37. D. A. Fitzpatrick, M. E. Logue, J. E. Stajich, G. Butler, BMC Evol Biol 6, 99 (2006).
38. M. Zuker, P. Stiegler, Nucleic Acids Res 9, 133 (1981).
39. K. Kawakami et al., Genetics 135, 309 (1993).

Materials and Methods Used in Examples 1-7

Growth Conditions and Genetic Manipulations

S. castellii was grown at 25° C. on standard S. cerevisiae plate and liquid media (e.g., YPD and SC). Transformations were performed as described (1) with some modifications. Either 0.5-2 μg plasmid DNA or 1-7 μg linear DNA was added to 5 μl single-stranded DNA (10 mg/ml salmon sperm DNA, Sigma D7656), mixed with 50 μl yeast (˜3×10⁸ cells in 100 mM lithium acetate), and added to transformation buffer (a mixture of 240 μl 40% PEG 3350 and 36 μl 1 M lithium acetate). After incubation at 25° C. for 30-90 min, 35 μl of DMSO was added, and the entire mixture was incubated at 42° C. for 10 min, resuspended, and then plated on selective media.

Other Species.

Growth temperatures were as follows, unless otherwise noted: K polysporus, 25° C.; S. cerevisiae, S. bayanus, and C. albicans, 30° C.; E. coli, 37° C.

Strain Construction

A list of strains used and generated in this study is provided in table S7.

Heterothallic Strains.

Most of our strains started with the homothallic S. castellii strain Y235 (ura3-1/ura3-1, HO/HO), generously provided by M. Cohn (ura3-1 is a point mutation G541A that creates the amino acid substitution G181R). To delete the Ho endonuclease, the loxP-KanMX6-loxP module of plasmid pUG6 (2) was used as a template to amplify the disruption cassette by fusion PCR (3), with ˜400-bp targeting arms on both sides of the cassette (primers 5′-TGATCGAAGAAGGCACTAGAA and 5′-CAGATCCACTAGTGGCCTATGCGGCCGCTGTCATTGAAAATCGCCAAA, 5′-GCGTACGAAGCTTCAGCTGGCGGCCGCGGCCAAATTCTTCCTGCAACT and 5′-TTTTCGGACTTCACGAGCTT). The resulting heterozygous strain (ura3-1/ura3-1, HO/ho::loxP-KanMX-loxP) was transformed with pSH47 (2), which encodes the Cre recombinase under the control of the S. cerevisiae GAL1 promoter. The expression of Cre was induced for 2 h in liquid culture, and strains sensitive to G418 were isolated. This strain was transferred to sporulation medium (1% potassium acetate, 0.1% yeast extract, 0.05% glucose) for 4 days, and tetrads were dissected. Although sporulation efficiency and spore viability were generally low in Y235, stable heterothallic strains of mating type a and a (DPB004 and DPB005, respectively) could be derived from a tetrad with four viable spores, showing that S. castellii HO deletion strains could not switch mating type.

Deletion of AGO1 and DCR1. AGO1 and DCR1 were deleted using the hygromycin cassette of pAG32 (4) and the loxP-KanMX6-loxP cassette of pUG6 as dominant selection markers, respectively. For diploids, homozygous deletions (DPB002 and DPB003) were generated first by deleting one copy in Y235, sporulating the resulting heterozygotes, and allowing isolated spores to grow, switch mating types, and mate. AGO1 and DCR1 were deleted in DPB004 and DPB005 to generate DPB006, DPB007, DPB008, DPB009, and DPB313. The AGO1 disruption construct was created as follows: AGO1 was amplified from genomic DNA (5′-TGAACGTGTGGAAGACCAAA and 5′-AGTGGCTAACGGCAACATATCAGACA) and cloned into pCR4Blunt-TOPO (Invitrogen); the hygromycin cassette was then inserted between the HindIII and AgeI restriction sites within the AGO1 genomic fragment; the AGO1 disruption construct was then amplified with the same primers used for AGO1 cloning. Deletion of DCR1 was analogous to deletion of HO (fusion PCR primers 5′-TTCAACACCTCCAGCAACAG and 5′-CAGATCCACTAGTGGCCTATGCGGCCGCAGGCATTGCAACAATCTGTG, 5′-GCGTACGAAGCTTCAGCTGGCGGCCGCGCTGTTGCTGGAGGTGTTGAA and 5′-TTTACCACCATACCATGAGTTTTT).

Tagged Ago1 strain for immunoprecipitation. A haploid strain expressing Flag₃-tagged Ago1 from its native promoter (DPB220) was constructed by two-step homologous recombination in DPB005, as follows: a S. cerevisiae URA3 expression cassette (amplified from pYES2.1, Invitrogen) was used to replace the start codon of AGO1 by transformation and selection of transformants on SC-ura plates; the URA3 cassette was subsequently replaced by a Flag₃ tag (amplified with a start codon from pQCXIP, gift of D. Sabatini) by transformation and selection on 5-FOA.

S. castellii GFP reporter strains. The loxP-KanMX6-loxP cassette in DPB009 was removed by Cre expression as described above to generate DPB318. The GFP(S65T)-KanMX6 module from pFA6a (5) was then integrated at the endogenous ura3 locus in DPB005, DPB313, and DPB318 (such that GFP was fused in-frame directly after the ATG start codon of ura3) to generate GFP-expressing strains DPB314, DPB317, and DPB321. The silencing constructs (pIp, pIp-weakSC_GFP, and pIp-strongSC_GFP) were integrated upstream of the ORF annotated as Scas_{—}633.2 in DPB314, DPB317, and DPB321 to create strains DPB331-DPB339. For these integrations, each silencing construct was linearized by digestion with Sad, and 1.5 μg was transformed. Transformants were selected on SC-ura plates.

S. cerevisiae RNAi reporter strains. The GFP(S65T)-KanMX6 module from pFA6a was integrated at the endogenous ura3 locus in L4718 to create DPB249. Integration of Ago1 and Dcr1 expression vectors (pRS404-P_{TEF}-Ago1 and pRS405-P_{TEF}-Dcr1) and GFP silencing construct vectors (pRS403-P_{GAL1}-weakSC_GFP and pRS403-P_{GAL}/-strongSC_GFP) into the genome was done by linearization and transformation using standard protocols (6) to create DPB250, DPB251, and DPB255-DPB260. To generate strains useful for URA3 silencing, DPB249 and DPB258 were transformed with functional URA3 coding sequence amplified from pRS406 to create the URA3 prototrophs DPB271 and DPB275, respectively. Integration of the silencing construct pRS403-P_{GAL1}-strongSC_URA3 into DPB271 and DPB275 generated DPB272 and DPB276, respectively.

Plasmid Construction

A list of plasmids generated in this study is provided in table S8.

Yeast Ago1 and Dcr1 expression plasmids. S. castellii AGO1 or DCR1 was cloned into pYES2.1 (Invitrogen) to produce the galactose-inducible Ago1 and Dcr1 expression plasmids pYES2.1-Ago1 and pYES2.1-Dcr1, respectively. GFP was also cloned into pYES2.1 (creating pYES2.1-GFP) as a negative control.

E. coli recombinant expression plasmids. For recombinant expression of Dcr1 in E. coli, DCR1 was cloned into pET101/D-TOPO, creating pET101-Dcr1. pET101-lacZ was supplied by the manufacturer (Invitrogen).

S. castellii GFP silencing constructs. A multiple cloning site containing XhoI and EcoRI restriction sites was cloned between the PvuII and XbaI restriction sites of pYES2.1. For the strong silencing construct, 275 by of GFP sequence from pFA6a was then cloned in the sense orientation between PvuII and XhoI sites, and in the antisense orientation between EcoRI and XbaI sites, in E. coli SURE (Stratagene). The weak silencing construct was made identically, except without GFP sequence in the antisense orientation. A 73-bp sequence spanning intron 1 from S. pombe rad9 was then added between XhoI and EcoRI sites (modeled after (7)). To convert these episomal plasmids into integrating plasmids, the 2-micron and f1 origins were then replaced (using NheI and SpeI sites) by sequence from S. castellii sc633:288301-289016 (amplified from genomic DNA with 5′-AAAAGCTAGCGATCCCTTATCAAATATGGTAC and 5′-AAAAACTAGTGTAGAATCCAGAGAATAGAATC).

These resulting S. castellii integrating plasmids expressing weak and strong GFP silencing constructs are pIp-weakSC_GFP and pIp-strongSC_GFP, respectively. The pIp empty vector was created by replacing the hairpin of pIp-strongSC_GFP with XhoI and EagI sites.

S. cerevisiae reconstitution and silencing constructs. Vectors pRS404-P_{TEF}-Ago1and pRS405-P_{TEF}-Dcr1 were constructed by insertion of the coding sequence of the respective S. castellii genes between the TEF promoter and CYC1 terminator (cloned from pRS416-P_{TEF} (8)) of the appropriate vector (9) using SpeI and XhoI sites (Ago 1) or XbaI and XhoI sites (Dcr1). To generate vectors pRS403-P_{GAL1}-strongSC_GFP and pRS403-P_{GAL1}-weakSC_GFP, an expression cassette containing the GAL1 promoter, CYC1 terminator, and GFP silencing construct sequence was cloned out of the appropriate episomal pYES2.1 silencing construct into the NotI and SalI sites of pRS403. To generate the URA3 silencing vectors, 339 by of URA3 sequence from pRS406 was initially cloned into the episomal pYES2.1 GFP weak silencing construct in the sense orientation between PvuII and XhoI sites (thereby replacing the GFP sequence), and in the antisense orientation between EcoRI and XbaI sites. pRS403-P_{GAL1}-strongSC_URA3 was then created by cloning an expression cassette containing the GAL1 promoter, CYC1 terminator, and URA3 silencing construct sequence out of this pYES2.1 plasmid into the NotI and SalI sites of pRS403.

In Vitro dsRNase Assays

Substrates.

Blunt-ended dsRNA substrate was prepared by simultaneous in vitro transcription from two PCR templates carrying T7 promoter sequences at opposite ends. Reactions were assembled using the MegaScript Kit (Ambion) with a 32:1 molar ratio of UTP:[α-³²P]UTP (800 Ci/mmol) according to the manufacturer's directions. Control ssRNA was prepared similarly, except that a single PCR template was included in the transcription reaction. DNase-treated RNA was fractionated on a 4% urea gel, eluted from gel slices in 0.3 M NaCl overnight at 4° C., and ethanol precipitated.

Strains.

Wild-type strains used in FIG. 2A were S. castellii Y235, K. polysporus KpolWT, C. albicans Can14, and S. cerevisiae FY45. Strains used in FIG. 2E were as follows: S. castellii, DPB005, DPB318, and DPB318 transformed with pYES2.1-Dcr1; S. cerevisiae, F2005 and F2005 transformed with pYES2.1-Dcr1 or pYES2.1-GFP; E. coli BL21 Star(DE3) (Invitrogen) transformed with pET101-lacZ or pET101-Dcr1.

Extracts.

Strains in FIG. 2A were grown in YPD to OD₆₀₀ 1.2-1.6; yeast strains in FIG. 2E were grown similarly, except P_{GAL1} strains were grown in SC-ura with galactose/raffinose and all strains were grown at 25° C.; E. coli were grown in LB with 100 μg/ml ampicillin to OD₆₀₀ 0.6 and induced (1 mM IPTG) for 4 h. Cells were harvested by centrifugation and flash frozen in 100-200 mg aliquots. Aliquots were thawed on ice, resuspended in 1 μl/mg lysis buffer [50 mM HEPES pH 7.6, 5 mM MgCl₂, 0.1 mM EDTA, 0.1 mM EGTA, 300 mM sodium acetate, 5% glycerol, 0.25% NP-40, protease inhibitor cocktail (Roche), 1 mM PMSF], and vortexed four times for 45 s at 4° C. with an equal volume of glass beads. Lysates were clarified by centrifugation at 10,000×g for 5 min. Extract concentrations were normalized according to absorbance at 260 nm and stored at −80° C.

Reactions.

The 20 μl reactions contained 10 μl extract (or 10 μl lysis buffer for “Buffer only” control), 4 μl 5× reaction buffer (125 mM HEPES pH 7.2, 10 mM magnesium acetate, 10 mM DTT, 5 mM ATP), and 10,000 cpm radiolabeled substrate. In FIG. 2A, reactions were incubated at 25° C. (S. castellii and K. polysporus) or 30° C. (all others) for 2 h; in FIG. 2E all reactions were incubated at 25° C. Reactions were quenched with AE Buffer (50 mM sodium acetate pH 5.5, 10 mM EDTA) and phenol extracted.

RNA Blots

Total RNA was isolated using the hot phenol method. Small RNA blots were performed using 10-15 μg total RNA per lane and carbodiimide-mediated cross-linking to the membrane (10), with the following DNA probes radiolabeled at their 5′ termini: S. castellii siRNA sc1056, 5′-CTATCTTCATCGATTACCATCTA; S. castellii U6 small nuclear RNA, 5′-TATGCAGGGGAACTGCTGAT; GFP siRNA, 5′-ACCATTATCAACAAAATACTCCAATTGGCGATGGCCCTGTCCTTTTACCA. mRNA blots were performed using 4-5 μg DNase-treated total RNA per lane and UV crosslinking. GFP and Ty1 (11) body-labeled antisense riboprobes were prepared by using PCR products as templates for in vitro transcription (MaxiScript kit, Ambion). A radiolabeled PYK1 (CDC19) DNA probe was prepared by random priming (Prime-It II, Stratagene).

Strains used in FIG. 2B were Y235, DPB002, DPB002 transformed with pYES2.1-Ago1, DPB003, and DPB003 transformed with pYES2.1-Dcr1. Strains used in FIG. 4B were DPB331-DPB339. Strains used in FIGS. 4D and 4F were DPB249-DPB251, and DPB255-DPB260.

RT-PCR

Reverse transcription reactions were performed with 100 ng total RNA using Superscript III according to the manufacturer's instructions (Invitrogen) with the following gene-specific primers in the same reaction: GFP, 5′-TGTGGTCTCTCTTTTCGTTGG; ACT1, 5′-TCAAAGAAGCCAAGATAGAACCA. PCR reactions were assembled in 100 μl with 2 μl RT reaction using the following primers: GFP, 5′-TTTCACTGGAGTTGTCCCAAT and 5′-GAAAGGGCAGATTGTGTGG; ACT1,5′-ACGTTGGTGATGAAGCTCAA and 5′-ATACCTGGGAACATGGTGGT. After the indicated number of cycles, a 15 μl aliquot was removed and combined with 3 μl 6×DNA loading dye. 6 μl was loaded onto a 1.5% agarose gel, and DNA was visualized by EtBr staining.

Plasmid Loss

DPB005, DPB313, and DPB008 were transformed with 1.5 μg pRS316 (8), pYES2.1-weakSC, pYES2.1-Ago1, or pYES2.1-Dcr1. Transformants were plated directly on SC-ura plates containing 2% glucose (uninduced) or 2% galactose (induced). To analyze plasmid loss, cells from colonies were inoculated in 5 ml of the medium indicated in Figure S11 and passaged once a day for 4 days.

Southern Blots

Each lane contained 2 μg of RNA-free DNA isolated as described in (12) and digested with XbaI. Plasmids were detected using a probe with the ampicillin-resistance gene sequence (amplified using primers 5′-CCATGAGTGATAACACTGCG and 5′-GGCACCTATCTCAGCGATC). The genomic locus was detected using a probe with sequence from S. castellii sc718:138001-138427 (amplified using primers 5′-GCATAAGCTGTGCTTTAGACT and 5′-CTTGTAACGGTTCAATTCTAGC).

FACS Analysis

Two biological replicates of each strain were inoculated in SC, either noninducing (2% glucose) or inducing (S. castellii, 2% galactose; S. cerevisiae, 1% galactose and 1% raffinose), and grown overnight. Fresh cultures were then seeded from the overnight cultures and cells were grown to log-phase. Cells were analyzed using FACSCalibur (BD Biosciences); data were processed with CellQuest Pro (BD Biosciences) and FlowJo (Tree Star). The same samples were used for RNA and GFP analyses.

URA3 Silencing

Strains (DPB249, DPB271-DPB272, DPB258, DPB275-DPB276) were inoculated in SC under inducing conditions (1% galactose and 1% raffinose) and grown for 1 day. Cells were diluted to OD₆₀₀ of 1.0, and 1:10 serial dilutions were spotted onto the appropriate plates (SC, SC-ura, or 5-FOA; all containing 1% galactose and 1% raffinose) and grown at 30° C. for 3 days.

S. cerevisiae Ty1 Analysis

Transposition assay. S. cerevisiae strains were transformed with 1 μg of pGTy1his3AI (galactose-inducible Ty1) (13) and selected on SC-ura plates. Transformants were streaked out on SC-ura with 2% galactose plates and grown at 20° C. for 2 days to induce transposition. Cells were then replica-plated onto SC-his plates (to select for transposition) or SC-ura plates (for control growth) and grown at 30° C. for 2-3 days.

RNA and Protein Analysis.

Strains were inoculated in SC containing 2% glucose and grown overnight. For non-transposition-inducing conditions, cells were diluted to OD 0.125 and grown at 30° C. to OD₆₀₀ 0.9-1.0. For transposition-inducing conditions, cells were diluted to 100 cells/ml and grown at 20° C. to OD₆₀₀ 0.9-1.0. Cells were harvested by centrifugation and flash frozen.

Immunoblotting.

Three OD₆₀₀ units of cells were resuspended in 100 ml H₂O. After adding 160 μl of extraction buffer (1.85 M NaOH, 7.4% β-mercaptoethanol), cells were incubated on ice for 10 min. 160 μl of 50% trichloroacetic acid was added and cells were incubated on ice for an additional 10 min. Precipitated material was collected by centrifugation and the supernatant was discarded. The tube was washed with 500 μl of 1 M Tris pH 8.0, centrifuged briefly, and the supernatant was discarded. The pellet was vigorously resuspended in 150 μl of 1× Laemlli sample buffer and boiled for 4′ min. Samples (12 μl each) were resolved by SDS-PAGE, transferred to poly(vinylidene difluoride) in CAPS-ethanol pH 10, and probed sequentially with Ty1-VLP antiserum (14, 15) and anti-actin (Abeam, ab8224). Immunoblots were developed with HRP-conjugated anti-rabbit or anti-mouse antibodies and enhanced chemiluminescence (Amersham).

Small-RNA Sequencing and Analysis

Library Preparation.

Total RNA was isolated using hot phenol from log-phase YPD cultures of S. castellii F2037, K. polysporus KpolWT, S. cerevisiae FY45, S. bayanus F2035, and C. albicans Can14. Small-RNA cDNA libraries were prepared as described (16) and sequenced using the Illumina SBS platform. Libraries were also prepared and sequenced from RNAi deletion strains (DPB002 and DPB003).

Ago1 Immunoprecipitation.

A saturated overnight culture of DPB249 was diluted to OD₆₀₀ 0.3 in 150 ml YPD and grown to OD₆₀₀ 1.5. Extracts were prepared as for in vitro dsRNase assays. For the input fraction, one-fifth of the extract was removed and added to AE buffer. Anti-Flag M2 agarose (Sigma) was incubated with the remaining extract at 4° C. for 1.5 h. Beads were washed with lysis buffer four times, after which the remaining buffer was removed and AE buffer was added. Small RNA libraries were prepared as described above.

Read Processing.

After removing the adaptor sequences, reads representing the small RNAs were collapsed to a non-redundant set, and 14-30-nt sequences were mapped to the appropriate genome, allowing no mismatches and recovering all hits (table 51). When counting the reads matching a locus, the count was hit-normalized, i.e., normalized to the number of times that a small-RNA sequence matched the genome. For example, a small RNA sequenced twice that mapped to the genome five times contributed 0.4 read counts to each genomic locus. Sequence and feature files for S. cerevisiae S288C and C. albicans SC5314 were obtained from the Saccharomyces Genome Database (SGD) on Sep. 10, 2007 and the Candida Genome Database Assembly 21. Sequence files for S. bayanus MCYC623 that were current as of Jan. 18, 2009 were downloaded from NCBI. Sequence and feature files for S. castellii CBS 4309 and K. polysporus DSM 70294 were obtained from the Yeast Gene Order Browser (YGOB) (17). Using the set of S. cerevisiae tRNA and rRNA sequences as queries for blastn alignments (e-value cutoff, e-10), genomic loci mapping to tRNA and rRNA in S. castellii, K. polysporus, and S. bayanus were identified. In K. polysporus, tRNA and rRNA annotations were available in the GenBank flatfile obtained from YGOB and used to supplement the alignments.

Initial Identification of siRNA Clusters.

For the small RNAs sequenced from total RNA, genomic regions giving rise to siRNAs were identified by parsing the genome files from S. castellii, K. polysporus, and C. albicans into non-overlapping windows of 500 bp. Windows with high levels of siRNA expression (22-23-nt sequences for S. castellii and K. polysporus, 21-22-nt sequences for C. albicans; excluding tRNA and rRNA reads) were selected by applying read and sequence density cutoffs manually adjusted based on the data set (S. castellii, ≧10 reads/kb or ≧10 genome matches/kb; K. polysporus, ≧50 reads/kb or ≧50 genome matches/kb; C. albicans, ≧40 reads/kb or ≧40 genome matches/kb). Adjacent windows passing the density cutoffs were concatenated. The small-RNA profile of each of these clusters was manually inspected for adherence to properties, including length (23 nt for S. castellii and K polysporus; 22 nt for C. albicans) and 5′-nt biases (U for S. castellii and K. polysporus; A or U for C. albicans).

Refined identification of siRNA clusters in S. castellii. Using sequencing reads of small RNAs co-purifying with Ago1, a hidden Markov model (HMM) was constructed with two states, “C” (giving rise to siRNAs) and “N” (not giving rise to siRNAs). The ratio of 23-mer reads relative to all reads (excluding 22-mer reads) was calculated in 10-bp windows (apportioning hit-normalized counts to the windows based on the fraction of its nucleotides covered by the small RNA) to define two types of emissions: 0) ratio ≧0.45 and 1) ratio<0.45. Emission probabilities were generated by training on the initially identified siRNA clusters to represent the “C” state, and training on five supercontigs (sc1014, sc621, sc542, sc534 and sc587) to represent the “N” state. Transition probabilities for the given window size were estimated using the median length of these siRNA clusters (250 bp) that map to Y′ elements and palindromic arms, or the average length of the intervening genomic sequence between two clusters, i.e. the difference derived from the total length of all contigs (11,354,548 bp) divided by the number of clusters identified in the initial analysis (100). Initial state probabilities were calculated based on the proportion of contigs in “C” state, i.e. total length of siRNA clusters (25,000 bp) divided by the total length of all contigs. Using the Viterbi algorithm, the contigs were parsed over non-overlapping 10-bp windows. The parse yielded 379 clusters (table S3) with the three regions that map to rRNA excluded. The cluster boundaries were adjusted to include the full sequence of all small RNAs with at least one nucleotide mapping to the cluster and to exclude terminal nucleotides not covered by a small RNA.

Cluster Annotation.

Clusters were further characterized based on previous genome annotations and alignments. Reads for FIG. 1C (21-23 nt) and for figure S3 (22-23 nt) were classified into categories. Reads of siRNA clusters that mapped to annotated ORFs in either sense or antisense orientation were categorized as “cluster ORF.” Using the Flag₃-Ago1 IP dataset, siRNA reads in clusters overlapping ORFs were further separated into “clusters sense to ORF” and “clusters antisense to ORF.” siRNA reads that mapped to convergent overlapping ORF transcripts (annotated using the mRNA-Seq dataset) were categorized as “overlap.”

The DNA sequences of the siRNA clusters from the S. castellii and K. polysporus datasets were aligned against the S. cerevisiae protein dataset (NCBI) using blastx (e-value cutoff 0.001). Significant alignments to Ty elements were extended 300 nt on both sides, and reads overlapping these extended alignments were classified as Ty-proximal siRNA reads. Additional Ty elements could be identified using annotated Ty elements from (18) as blastx queries. More careful Ty annotations for S. castellii could then be made by identifying S. castellii Ty LTR, gag, and pol sequences based on the initial blastx matches and other Ty sequence signatures (18-20) and references therein). Similarly, siRNA clusters derived from Y′ elements were detected. For cases in which siRNA expression exceeded the boundaries of the annotated Y′ element ORF in a processive, un-gapped fashion, those siRNAs were still classified as Y′-element-proximal siRNAs. siRNA clusters in C. albicans were annotated based on the C. albicans genome annotation and blastx alignments against the set of protein sequences downloaded from NCBI (e-value cutoff 0.001).

Palindromes were predicted using the IRF program (21) with the following parameters: Alignment Parameters, 2, 3, and 5 (match, mismatch, and indels, respectively); minimum Alignment Score To Report Repeat, 100; T4 small palindromes (20-80+ nt) loop length, 100 nt; 15 medium palindromes (80-300+ nt) loop length, 1000 nt; T7 large palindromes (300-2400+ nt) loop length, 5000 nt; maximal loop length, 5000 nt; maximal stem length, 10,000 nt; allow GT matches. The following numbers of palindromes were identified: 66 in S. castellii, 222 in K. polysporus, 61 in C. albicans, and 390 in S. cerevisiae. These palindromes were compared to our lists of siRNA-generating loci. In most cases when overlap was observed, the 22-23-nt RNAs were enriched in the inverted-repeat regions rather than the intervening region or surrounding regions. In some cases the palindromes overlapped with each other and the one with 22-23-nt RNAs mapping to the repeats was the one chosen. In a few cases (10 of 43 for S. castellii, and 42 of 90 for K. polysporus), the overlap of 22-23-nt RNAs was not preferentially at the repeats; these were not classified as palindromic clusters. Using the initial datasets, these analyses revealed 19 palindromic siRNA clusters in S. castellii and 29 in K. polysporus, all of which either overlapped or were contained within the set of siRNA clusters identified by the sliding window approach. The refined cluster identification based on the Flag₃-Ago1 IP dataset from S. castellii revealed 23 palindromic siRNA clusters (table S5).

Phasing Analysis.

The frequency of distances separating pairs of 23-mer 5′ ends mapping to the same DNA strand was calculated using the following equation:

Frequency_{D}=Σ(Reads₂₃₋ₘₑᵣ₁×Reads₂₃₋ₘₑᵣ₂)_{D}

where D=distance between sRNA 5′ ends

refined cluster identification based on the Flag₃-Ago1 IP dataset from S. castellii revealed 23 palindromic siRNA clusters (table S5).

The frequency of distances separating pairs of 23-mer 5′ ends mapping to opposite strands of DNA was calculated separately using the same equation.

Phylogenetic Analysis

Psi symbols for S. pastorianus (FIG. 1A) indicate a highly degraded pseudogene of AGO1 and a DCR1 pseudogene that is intact except for a single internal stop codon. The intact S. bayanus DCR1 gene shows conservation of amino acid sequence relative to the S. pastorianus pseudogene (dN/dS ratio 0.3) despite the absence of intact AGO1 in both species. The AGO1 and DCR1 loci are syntenic among S. castellii, K. polysporus, S. pastorianus, and S. bayanus.

A maximum-likelihood (ML) tree of RNaseIII domains was constructed using the PHYLIP software package (http://evolution.genetics.washington.edu/phylip.html). RNaseIII domains were predicted using SMART (22, 23). The amino acid sequences of the RNaseIII domains were used to compute a multiple sequence alignment using TCOFFEE (24). A consensus ML tree was built by running DNAML (PHYLIP) on the amino acid alignment after bootstrap re-sampling (500 replicates) of the data set using SEQBOOT (PHYLIP). The phylogenetic tree was displayed using TreeView (http://taxonomy.zoology.gla.ac.uk/rod/treeview.html).

Protein name/accession numbers used in FIG. 2D are as follows: At1, A. thalania DCL1; At2, A. thalania DCL2; Ca2, C. albicans XP_{—}717277; Ca1, C. albicans EAK98282; Ct1, C. tropicalis AAFN01000070; Ct2, C. tropicalis AAFN01000057; Cn1, C. neoformans XP_{—}569593.1; Cn2, C. neoformans XP_{—}569797.1; Dh1, D. hansenii XP_{—}457483.1; Dh2, D. hansenii XP_{—}457193.1; Hs, H. sapiens DICER1; K1, K. lactis F2416; Kp2, K. polysporus 455 μl; Kp1, K. polysporus 1045 μl; Mg1, M. grisiae XP_{—}363615; Mgt, M. grisiae XP_{—}367242; Mg3, M. grisiae XP_{—}367242; Nc1, N. crassa Sms3; Nc2, N. crassa Dc12; Nc3, N. crassa NCU01762; Sb1, S. bayanus 671p65; Sb2, S. bayanus 643p2; ScaI, S. castellii 696p6; Sca Dcr1, S. castellii 626p5; Sc, S. cerevisiae Rnt1; Spl, S. pombe Dcr1; Sp2, S. pombe Pac1.

To identify the PAZ domain in S. pombe Dcr1, the full-length Dcr1 protein sequence was submitted as a query to the HHpred server using default parameters (25). A region of conserved secondary structure (E-value=1.5e-13, identity=17%) was detected that corresponded to a crystallized Dicer derived from Giardia intestinalis (26). This region (amino acids 686-1233 of Dcr1) was inclusive of a PAZ domain, two RNAse III domains, and surrounding linker sequences. An equivalent approach for S. castellii Dcr1p, searching against all available HMM databases, revealed no detection of a PAZ domain.

mRNA Sequencing and Analysis

Strand-Specific mRNA-Seq.

Two biological replicates of DPB005 (WT), DPB007 (Δago1), and DPB009 (Δdcr1) were grown in YPD to OD₆₀₀ 0.6-0.8. Total RNA isolated using hot phenol was treated with DNaseI (RiboPure-Yeast Kit, Ambion). Poly-(A)⁺ mRNA was purified from 75 μg total RNA using magnetic oligo-dT DynaBeads (Invitrogen) according to the manufacturer's instructions, and then fragmented by alkaline hydrolysis (27). Trace amounts of synthetic 3′-pCp[5′-³²P]-labeled 26-nt and 32-nt RNA size markers were added to monitor the subsequent steps. RNA fragments (25-45 nt) were gel-purified and 3′-dephosphorylated in a 25 μl reaction containing 12.5 units T4 PNK (New England Biolabs) and MES-NaOH buffer (100 mM MES-NaOH pH 5.5, 10 mM MgCl₂, 10 mM β-mercaptoethanol, 300 mM NaCl) for 6 h at 37° C. After phenol extraction and precipitation, RNA was ligated to pre-adenylated adaptor DNA as described (16). Gel-purified ligation products were 5′-phosphorylated in a 14 μl reaction containing 15 units T4 PNK and PNK buffer for 30 mM at 37° C. After phenol extraction and precipitation, RNA was ligated to a 5′-adaptor RNA, gel-purified, converted to cDNA, amplified, and sequenced as described (16).

Read Processing.

The first 25 nt of each 32-nt read were isolated and collapsed into a non-redundant set of 25-nt sequences with occurrence counts (table S4). Sequences were mapped to the reference genome, allowing no mismatches and recovering all hits. Transcript-specific analysis of small-RNA data (e.g., FIG. 3A) was based on 22-23-nt reads from the Flag₃-Ago1 IP dataset, unless indicated otherwise.

Exon annotations were downloaded from YGOB (introns less than 10 nt were considered sequencing errors and assigned as exons). Sense mRNA, antisense mRNA, and antisense small-RNA read counts were calculated individually for each gene by summing the hit-normalized reads mapping either to the 5′-half of the ORF (mRNA tags, half-ORF analysis) or across all of the ORF (small-RNA reads); a sequence contributed N*nt/25 reads to a gene (N=hit-normalized read number; nt=number of nt in the 25-nt sequence overlapping the ORF). In parallel, mRNA tag counts were also calculated across the entire ORF (full-ORF analysis, fig. S4).

For each gene, mRNA-Seq tag counts from biological replicates were averaged. Genes for which none of the three strains had an average tag count 20 (half-ORF analysis) or above 30 (full-ORF analysis), and ORFS corresponding to Y′ element fragments, were excluded from all analyses except in figures S4A and S4B. mRNA abundance was calculated by dividing tag counts by kb of mapped exon. mRNA-Seq tag counts from Δago1 were normalized to those of WT by first ranking genes based on the ratio of tags in Δago1 versus WT, and then multiplying the WT tag counts by a factor such that the median ranked gene had a transcript abundance ratio of 1. An analogous normalization procedure was also applied to Δdcr1. The final normalization factors were 0.8847 for WT, 1.0000 for Δago1, and 0.8440 for Δdcr1. The same normalization factors were applied to the single-nucleotide-resolution mRNA-Seq plots for the Y′ element consensus (FIG. 3B).

Consensus Y′ element of S. castellii. An initial set of Y′-element fragments was obtained by extending and combining annotated Y′-element ORFS and Y′-element fragments manually identified in the course of annotating siRNA clusters. These fragments were assembled into a single contig using SeqMan Pro (DNASTAR Lasergene). The resulting majority sequence was used as a query for blastn against the genome (e-value cutoff 10⁻¹⁰, MegaBlast option). All additional Y′ element fragments obtained from this search were added to the consensus, bringing to 32 the total number of unique contributing genomic fragments (fig. S5). mRNA tags and small-RNA reads were mapped to the consensus Y′ element independently of the genome. Each library was initially mapped to the set of Y′ element fragments, allowing no mismatches and recovering all hits. Mapped nucleotide positions with respect to fragments were converted into positions with respect to the consensus, with a requirement that each unique sequence mapped only once to the consensus. Mapping data was normalized using the above factors and used to generate single-nucleotide-resolution plots of the consensus Y′ element (FIG. 3B). Y′ element transcript and siRNA abundances were the sum of read and tag nucleotides across the region of interest divided by the appropriate length (25 nt for mRNA; 22 or 23 nt for siRNA).

Comparing ORF-Derived siRNA Levels with Transcript Levels.

For each annotated protein-coding gene, mRNA tags and small-RNA reads mapping across its ORF were determined as above, except only uniquely mapping sequences were included. For each ORF, sense and antisense transcript abundances were estimated separately as the sum of tags from all six mRNA-Seq libraries (without normalization), and siRNA abundance was estimated as the sum of sense and antisense small RNA reads. Genes with no unique tags mapping to the coding strand were excluded. Genes were ranked by total transcript abundance (sum of sense and antisense) and by inferred duplex abundance (minimum of sense and antisense). Genes with non-zero abundance were divided into three equally sized bins (high, mid, low), and genes with zero abundance formed a fourth bin for inferred duplex analysis.

Transcripts Corresponding to siRNA-Generating Loci.

For each siRNA cluster identified using the HMM, two transcripts—one on each strand—were initiated and assigned the coordinates of the cluster. Tags from Δscr1 mRNA-Seq libraries were used to extend cluster transcripts as follows. The transcript was extended 10 nt in the 5′ direction if that 10-nt window had a tag density within 10-fold (above or below) of that of the initially assigned transcript. This process was iterated using the average tag density of the extended transcript. Once a window failing this criterion was reached, the transcript was terminated before the window. Then, the 3′ end was also thus extended, beginning with the average tag density of the transcript that included the extended 5′ end. Transcript extension was also tried first in the 3′ then in the 5′ direction; when the transcript ends disagreed between these two orders, the combination of 5′ and 3′ ends forming the largest transcript was used. The ends were then more finely mapped by identifying the first nucleotide upstream and last nucleotide downstream that corresponded to any tags (in Δdcr1 mRNA-Seq libraries), with a maximum extension of 10 additional nucleotides. Coordinates of inferred transcripts are presented in table S3. Transcripts that had mRNA-Seq tags mapping to them but that did not overlap any previous annotations were annotated as non-coding-siRNA-generating genes (NCS, table S3).

Transcript abundance in each mRNA-Seq library and siRNA abundance were determined as with coding transcripts, with the following exceptions: intron annotations were ignored, and an average read cutoff of 15 tags (half-transcript analysis) or 20 tags (full-transcript analysis) in any strain was applied. Y′-element fragments were removed and replaced with the consensus, except in table S3.

Protein-Coding Transcript Extension and Overlap.

Of 5693 annotated ORFs, 5297 (93%) had mRNA-Seq tags mapping to at least 70% of the ORF nucleotides (combining tags from all three strains) and were carried forward for further analysis. For each ORF, the 5′ and 3′ boundaries of the transcript were extended using the mRNA-Seq tags, requiring contiguous tag coverage outward from the ORF boundaries and assigning the revised 5′ and 3′ boundaries to the most distal nucleotides represented by these mRNA-Seq tags.

A gene pair was defined as a gene and its right neighbor (according to YGOB annotations). The 5297 ORFs were parsed into 4776 gene pairs, with the loss of pairs attributable mainly to genes located at the ends of contigs. The number of convergent overlapping transcripts giving rise to DCR1-dependent siRNAs was calculated comparing 22-23-nt reads from the Flag₃-Ago1 input and Δdcr1 datasets. 467 convergent overlapping loci had uniquely mapping small RNA reads in the Flag₃-Ago1 input dataset. The Δdcr1 dataset was then used to adjust this number to account for the loci for which small RNAs represented DCR1-independent mRNA degradation intermediates. Because RNA degradation intermediates would be overrepresented in the Δdcr1 small RNA dataset due to the absence of siRNAs, the Δdcr1 dataset was normalized to the Flag₃-Ago1 input dataset based on the number of rRNA and tRNA reads. Three normalized Δdcr1 datasets were constructed from the complete dataset by random sampling without replacement. In these three datasets, a median of 30 convergent overlapping loci had uniquely mapping Δdcr1 small RNA reads, which indicated that at least 437 convergent overlapping loci (43%) gave rise to DCR1-dependent uniquely mapping siRNAs.

To compare overlapping transcripts between S. castellii and S. cerevisiae, a list of gene pairs with opposite and overlapping transcripts in S. cerevisiae was downloaded from http://www.yale.edu/snyder/(28). The genes comprising these 828 unique gene pairs were mapped to their corresponding S. castellii genes based on YGOB annotations. 398 pairs corresponded to annotated convergent gene pairs in S. castellii. These pairs were cross-referenced with the list of S. castellii overlapping convergent gene pairs to determine the number producing overlapping transcripts in both species. Of the convergent gene pairs syntenic between these two genomes and reported to generate overlapping mRNAs in S. cerevisiae, 84% generated overlapping mRNAs in S. castellii.

S. cerevisiae mRNA-Seq analysis. Strand-specific mRNA-Seq data from S. cerevisiae (27) was downloaded from the Gene Expression Omnibus (samples GSM346117 and GSM346118) and processed as for S. castellii. Telomere (TEL16L, TEL16R, TEL12L, and TEL12R) and Ty element (YDRWTy1-5) annotations were downloaded from SGD, and hit-normalized tag counts were used to plot the abundance of mRNA-Seq tags at single-nucleotide resolution (i.e. tags contributed to counts along their entire length).

Materials and Methods Used in Examples 8-9

Preparation of dsRNA.

dsRNA substrates used in Examples 8 and 9 were prepared by annealing of single-stranded RNA (ssRNA) generated by in vitro transcription with T7 RNA Polymerase. Transcription templates containing 488-nt from the Renilla luciferase gene (amplified from pIS1) or from the gfp gene (amplified from pIRESneo-FLAG/HA EGFP) were generated by PCR. It is noted that there is another 6 nucleotides at each end of the RNA corresponding to the preferred T7 initiation sequence (GGGAGA) or its complement (TCTCCC) resulting in a total of 500-nt. DNase-treated ssRNA was fractionated by denaturing PAGE, eluted from gel slices in 0.3 M NaCl overnight at 4° C., and ethanol precipitated. Complementary RNAs were combined in dsRNA Annealing Buffer (30 mM Tris-HCl pH 7.5, 100 mM NaCl, 1 mM EDTA), heated to 90° C. for 1 min, and slowly cooled to room temperature over 4-5 hr. Annealed 500-bp dsRNA was fractionated on a 4% urea gel, dsRNA was eluted from gel slices in 0.3 M NaCl overnight at 4° C., ethanol precipitated, and stored in dsRNA Storage Buffer (10 mM Tris-HCl pH 7.5, 10 mM NaCl, 0.1 mM EDTA).

Dicer Constructs.

Dcr1ΔC=Dcr1 (15-355) was used for all experiments in Examples 8 and 9 except the siRNAs used in the luciferase knock-down experiment (Example 9) were prepared by dicing with Dcr1(1-384). Constructs containing amino acids 1-398, 1-376, 11-355 of K. polysporus Dicer were also tested and shown to retain dicing activity.

Transfection and Luciferase Assays.

siRNA transfections and luciferase assays were performed essentially as described in [Farh et al. (2005) The Widespread Impact of Mammalian MicroRNAs on mRNA Repression and Evolution, Science] with the following modifications. HEK293 cells were transfected using Lipofectamine 2000 (Invitrogen) in 24-well plates (15×10⁵ cells/well) with 20 ng firefly luciferase control reporter (pISO), 50 ng Renilla luciferase reporter (pIS1), and the indicated concentration of siRNA. Firefly and Renilla luciferase activities were measured 24 hours after transfection with the Dual-luciferase assay (Promega). Renilla activity was normalized to firefly activity to control for transfection efficiency.

Expression and Purification of Dcr1.

The gene encoding K. polysporus Dcr1AC was cloned into the modified pRSFDuet vector (Novagen) and overexpressed in E. coli BL21 (DE3) Rosetta2 (Novagen). The layout of the modified pRSFDuet vector was as follows: N-[His-tag]-[sumo-tag]-[Upl1 protease cleavage site]-[Dcr1]-C.

E. coli were grown at 37° C. to OD₆₀₀ 0.5, induced by addition of IPTG to 0.5 mM, and grown at 20° C. overnight (˜12 hr). Cells were lysed by sonication in 10 mM Na/K-phosphate buffer (pH 7.3), 640 mM NaCl, 10 mM beta-mercaptoethanol and 1 mM phenylmethylsulphonyl fluoride, then centrifuged. The Dcr1ΔC protein was purified by Ni-affinity, ion-exchange, hydrophobic-interaction, Heparin-affinity and gel-filtration columns. The His-tag was cleaved during dialysis just after Ni-affinity column chromatography using ubiquitin-like protein 1 (Ulp1) SUMO protease. After purification, recombinant K. polysporus Dcr1ΔC was dialyzed against 1× Protein Storage Buffer (10 mM Tris-HCl pH 7.5, 200 mM NaCl, 5 mM DTT) and stored at −80° C. After purification, recombinant K. polysporus Dcr1ΔC proteins were stored in 1× Protein Storage Buffer (10 mM Tris-HCl pH 7.5, 200 mM NaCl, 5 mM DTT) at −80° C. For biochemical assays, proteins were diluted and stored at −20° C. in Protein Dilution Buffer (5 mM Tris-HCl pH 7.5, 100 mM NaCl, 2.5 mM DTT, 50% glycerol, 1 mg/ml Ultrapure BSA [Ambion]). All Dcr1 protein concentrations are expressed in terms of dimer concentration. 10 μl reactions contained 2 μl 5× reaction buffer (150 mM Tris-HCl pH 7.5, 150 mM NaCl, 25 mM MgCl₂, 5 mM DTT, 0.5 mM EDTA), 1 μl protein, and 1 μl RNA substrate. The reaction was just scaled up proportionally for larger reactions. For optimization of reaction conditions, reactions were incubated at room temperature (22-24° C.) for the indicated time and quenched by addition to 10 μl 2× Formamide Loading Buffer (90% formamide, 18 mM EDTA, 0.025% sodium dodecyl sulfate, 0.1% xylene cyanol, 0.1% bromophenol blue). Samples were heated at 90° C. for 2 min, and products were resolved by 15% denaturing PAGE. Gels were stained with SYBR Gold (Invitrogen) according to the manufacturer's instructions.

For preparative dicing, 1.6 ml reactions were incubated at room temperature (22-24° C.) for 30 min and quenched by addition of 0.4 ml 5× Quench Buffer (40 mM EDTA, 1.5M NaCl). Quenched reactions were extracted with an equal volume of phenol-chloroform and precipitated. To recover duplex siRNA products, reaction products were resolved by 15% native PAGE run at 4° C. and stained with SYBR Gold. siRNA products were eluted from gel slices in 0.3 M NaCl overnight at 4° C., ethanol precipitated, and stored in dsRNA Storage Buffer. siRNA concentrations were determined by absorbance at 260 nm.

### REFERENCES FOR MATERIALS AND METHODS

1. E. Astromskas, M. Cohn, Yeast 24, 499 (2007).
2. U. Guldener, S. Heck, T. Fielder, J. Beinhauer, J. H. Hegemann, Nucleic Acids Res 24, 2519 (1996).
3. M. D. Krawchuk, W. P. Wahls, Yeast 15, 1419 (1999).
4. A. L. Goldstein, J. H. McCusker, Yeast 15, 1541 (1999).
5. M. S. Longtine et al., Yeast 14, 953 (1998).
6. R. D. Gietz, R. H. Schiestl, Nat Protoc 2, 31 (2007).
7. A. Sigova, N. Rhind, P. D. Zamore, Genes Dev 18, 2359 (2004).
8. D. Mumberg, R. Muller, M. Funk, Gene 156, 119 (1995).
9. R. S. Sikorski, P. Hieter, Genetics 122, 19 (1989).
10. G. S. Pall, C. Codony-Servat, J. Byrne, L. Ritchie, A. Hamilton, Nucleic Acids Res 35, e60 (2007).
11. M. J. Curcio, A. M. Hedge, J. D. Boeke, D. J. Garfinkel, Mol Gen Genet. 220, 213 (1990).
12. C. S. Hoffman, F. Winston, Gene 57, 267 (1987).
13. M. Bryk, M. Banerjee, D. Conte, Jr., M. J. Curcio, Mol Cell Biol 21, 5374 (2001).
14. S. E. Adams et al., Cell 49, 111 (1987).
15. S. D. Youngren, J. D. Boeke, N. J. Sanders, D. J. Garfinkel, Mol Cell Biol 8, 1421 (1988).
16. A. Grimson et al., Nature 455, 1193 (2008).
17. J. L. Gordon, K. P. Byrne, K. H. Wolfe, PLoS Genet. 5, e1000485 (2009).
18. C. Neuveglise, H. Feldmann, E. Bon, C. Gaillardin, S. Casaregola, Genome Res 12, 930 (2002).
19. C. Llorens, R. Futami, D. Bezemer, A. Moya, Nucleic Acids Res 36, D38 (2008).
20. E. R. Havecker, X. Gao, D. F. Voytas, Genome Biol 5, 225 (2004).
21. Y. Gelfand, A. Rodriguez, G. Benson, Nucleic Acids Res 35, D80 (2007).
22. J. Schultz, F. Milpetz, P. Bork, C. P. Ponting, Proc Natl Acad Sci U S A 95, 5857 (1998).
23.1. Letunic, T. Doerks, P. Bork, Nucleic Acids Res 37, D229 (2009).
24. C. Notredame, D. G. Higgins, J. Heringa, J Mol Biol 302, 205 (2000).
25. J. Soding, A. Biegert, A. N. Lupas, Nucleic Acids Res 33, W244 (2005).
26.1. J. MacRae et al., Science 311, 195 (2006).
27. N. T. Ingolia, S. Ghaemmaghami, J. R. Newman, J. S. Weissman, Science 324, 218 (2009).
28. U. Nagalakshmi et al., Science 320, 1344 (2008).
29. J. Houseley, K. Kotovic, A. El Hage, D. Tollervey, Embo J 26, 4996 (2007).
30. T. J. Goodwin, D. E. Dalle Nogare, M. I. Butler, R. T. Poulter, Yeast 20, 493 (2003).
31. C. B. Brachmann et al., Yeast 14, 115 (1998).
32. D. R. Scannell et al., Proc Natl Acad Sci USA 104, 8397 (2007).
33. W. A. Fonzi, M. Y. Irwin, Genetics 134, 717 (1993).
34. R. F. Petersen et al., J Mol Biol 318, 627 (2002).
35. B. J. Thomas, R. Rothstein, Cell 56, 619 (1989).
36. Farh et al., Science 310(5755):1817-21 (2005).

Those skilled in the art will recognize, or be able to ascertain using no more than routine experimentation, many equivalents to the specific embodiments of the invention described herein. The scope of the present invention is not intended to be limited to the embodiments described above, but rather is as set forth in the claims. The invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Articles such as “a” and “an”, and the like, may mean one or more than one unless indicated to the contrary or otherwise evident from the context.

The phrase “and/or” as used herein in the specification and in the claims, should be understood to mean “either or both” of the elements so conjoined. Multiple elements listed with “and/or” should be construed in the same fashion, i.e., “one or more” of the elements so conjoined. Other elements may optionally be present other than the elements specifically identified by the “and/or” clause. As used herein in the specification and in the claims, “or” should be understood to have the same meaning as “and/or” as defined above. For example, when used in a list of elements, “or” or “and/or” shall be interpreted as being inclusive, i.e., the inclusion of at least one, but optionally more than one, of list of elements, and, optionally, additional unlisted elements. Only terms clearly indicative to the contrary, such as “only one of” or “exactly one of” will refer to the inclusion of exactly one element of a number or list of elements. Thus claims that include “or” between one or more members of a group are considered satisfied if one, more than one, or all of the group members are present, employed in, or otherwise relevant to a given product or process unless indicated to the contrary. The invention provides embodiments in which exactly one member of the group is present, employed in, or otherwise relevant to a given product or process. The invention also provides embodiments in which more than one, or all of the group members are present, employed in, or otherwise relevant to a given product or process. It is to be understood that the invention encompasses embodiments in which one or more limitations, elements, clauses, descriptive terms, etc., of a claim is introduced into another claim. For example, a claim that is dependent on another claim can be modified to include one or more elements or limitations found in any other claim that is dependent on the same base claim.

Where the claims recite a composition, it is understood that methods of using the composition as disclosed herein are provided, and methods of making the composition according to any of the methods of making disclosed herein are provided. Where the claims recite a method, it is understood that a composition for performing the method is provided. Where elements are presented as lists or groups, each subgroup is also disclosed. It should also be understood that, in general, where the invention, or aspects of the invention, is/are referred to as comprising particular elements, features, etc., certain embodiments of the invention or aspects of the invention consist of, or consist essentially of, such elements, features, etc.

It should also be understood that, unless clearly indicated to the contrary, in any methods claimed herein that include more than one step or act, the order of the steps or acts of the method is not necessarily limited to the order in which the steps or acts of the method are recited.

Where ranges are given herein, the invention provides embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. In any embodiment of the invention in which a numerical value is prefaced by “about”, the invention provides an embodiment in which the exact value is recited. In any embodiment of the invention in which a numerical value is not prefaced by “about”, the invention provides an embodiment in which the value is prefaced by “about”. Where the phrase “at least” precedes a series of numbers, it is to be understood that the phrase applies to each number in the list (it being understood that, depending on the context, such as in describing percent identity, 100% of a value may be an upper limit.) It is also understood that any particular embodiment, feature, or aspect of the present invention may be explicitly excluded from any one or more of the claims.

1. A yeast cell that comprises a nucleic acid segment that encodes a non-endogenous RNAi pathway polypeptide that is functional in the yeast cell.

2. The yeast cell of claim 1, wherein the yeast cell lacks an endogenous RNAi pathway.

3.-8. (canceled)

9. The yeast cell of claim 1, wherein the yeast cell is a budding yeast cell.

10.-14. (canceled)

15. The yeast cell of claim 1, wherein the non-endogenous RNAi pathway protein is derived from a budding yeast species that has a functional RNAi pathway.

16.-18. (canceled)

19. The yeast cell of claim 1, wherein the non-endogenous RNAi pathway protein is a Dicer polypeptide.

20.-22. (canceled)

23. The yeast cell of claim 1, wherein the yeast cell comprises a non-endogenous nucleic acid segment that can be transcribed to yield dsRNA that has sequence correspondence to mRNA of a gene.

24.-37. (canceled)

38. A budding yeast cell that lacks an endogenous RNAi pathway, wherein the budding yeast cell is genetically engineered so that it has a functional RNAi pathway.

39. The budding yeast cell of claim 38, wherein the budding yeast cell lacks a functional endogenous Dicer polypeptide and is genetically engineered to contain a nucleic acid that encodes a functional Dicer polypeptide.

40. The budding yeast cell of claim 36, wherein the budding yeast cell has a functional endogenous Dicer polypeptide but lacks a functional endogenous Argonaute polypeptide and is genetically engineered to contain a nucleic acid that encodes a functional Argonaute polypeptide.

41.-42. (canceled)

43. A budding yeast cell that has a functional RNAi pathway, wherein the budding yeast cell is genetically engineered to contain a nucleic acid segment that can be transcribed to yield a dsRNA that has sequence correspondence to mRNA of a gene.

44. The budding yeast cell of claim 43, wherein the gene is an endogenous gene.

45.-47. (canceled)

48. The budding yeast cell of claim 43, wherein the budding yeast cell lacks a functional endogenous RNAi pathway.

49.-65. (canceled)

66. A method of silencing a gene in a budding yeast cell comprising: (a) providing a budding yeast cell that has a functional RNAi pathway; and (b) delivering siRNA to the budding yeast cell, thereby resulting in silencing of the gene.

67. The method of claim 66, wherein the budding yeast cell lacks a functional endogenous RNAi pathway and is genetically engineered to have a functional RNAi pathway.

68. (canceled)

69. The method of claim 66, wherein the budding yeast cell comprises a nucleic acid that can be transcribed to yield a dsRNA that has sequence correspondence to mRNA of the gene; and step (b) comprises maintaining the cell under conditions in which the dsRNA is expressed and is cleaved to siRNA, thereby resulting in silencing of the gene.

70.-125. (canceled)

126. An isolated nucleic acid comprising a polynucleotide that has a sequence at least 80% identical to the sequence of a naturally occurring polynucleotide that encodes an RNase III domain of functional budding yeast Dicer polypeptide.

127.-144. (canceled)

145. An isolated polypeptide comprising a polypeptide that has a sequence at least 80% identical to the sequence of an RNase III domain of a functional budding yeast Dicer polypeptide.

146.-148. (canceled)

149. The isolated polypeptide of claim 145, further comprising a dsRNA binding domain.

150. (canceled)

151. The isolated polypeptide of claim 145, comprising a polypeptide that has a sequence at least 80% identical to the sequence of a functional budding yeast Dicer polypeptide.

152.-203. (canceled)

204. An isolated nucleic acid comprising a polynucleotide that encodes a polypeptide that has a sequence at least 80% identical to the sequence of a functional budding yeast Argonaute polypeptide, wherein the polynucleotide is operably linked to an expression control element capable of directing transcription in a cell that lacks a functional endogenous Argonaute polypeptide.

205.-232. (canceled)
